(19) United States
(12) Patent Application Publication
(10) Pub. No.: US 2025/0320494 A1
(43) Pub. Date: Oct. 16, 2025

(54) INHIBITORS OF EXPRESSION AND/OR FUNCTION

(71) Applicant: E-Therapeutics PLC, London (GB)

(72) Inventors: Alan Victor WHITMORE, London (GB); Julie BORGEL, London (GB); Amy MCCARTHY, London (GB); Graham CRAGGS, London (GB); James LONGDEN, London (GB); Ines DE SANTIAGO, London (GB); Duncan BROWN, London (GB); Ahmad Ali MORTAZAVI, London (GB); Viviana MANNELLA, London (GB); Muthusamy JAYARAMAN, London (GB); Alexandre DEBACKER, London (GB); Adrian MOGG, London (GB)

(21) Appl. No.: 18/869,034

(22) PCT Filed: Jun. 1, 2023
(86) PCT No.: PCT/EP2023/064754

§ 371 Date: Nov. 25, 2024

Related U.S. Application Data
(60) Provisional application No. 63/369,634, filed on Jul. 27, 2022.

(30) Foreign Application Priority Data
Jun. 1, 2022 (GB) 2208122.8
Feb. 6, 2023 (EP) 23155114.4

(51) Int. Cl. C12N 15/113; A61K 31/713; A61P 7/04

(52) U.S. Cl. CPC C12N 15/113; A61K 31/713; A61P 7/04; C12N 2310/14; C12N 2310/315; C12N 2310/322; C12N 2310/351; C12N 2310/3521; C12N 2310/3533

(57) ABSTRACT

The present invention relates to inhibitors, and compositions containing inhibitors, and uses of the same in the treatment or prevention of a disease related to a disorder of haemostasis, such as haemophilia.

### FIELD

The present invention provides inhibitors, such as nucleic acid compounds, such as siRNA, suitable for therapeutic use. Additionally, the present invention provides methods of making these compounds, as well as methods of using such compounds for the treatment of various diseases and condition

### BACKGROUND OF THE INVENTION

Inhibitors, such as oligonucleoside/oligonucleotide compounds which are inhibitors of gene expression and/or expression or function of other targets such as LNCRNAs, can have important therapeutic applications in medicine. Oligonucleotides/oligonucleosides can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. siRNA, antisense RNA, and micro-RNA are oligonucleoside/oligonucleotides that prevent the formation of proteins by gene-silencing.

A number of modified siRNA compounds in particular have been developed in the last two decades for diagnostic and therapeutic purposes, including siRNA/RNAi therapeutic agents for the treatment of various diseases including central-nervous-system diseases, inflammatory diseases, metabolic disorders, oncology, infectious diseases, and ocular diseases.

The present invention relates to inhibitors, such oligomers e.g. nucleic acids, e.g. oligonucleoside/oligonucleotide compounds, and their use in the treatment and/or prevention of disease.

### STATEMENTS OF INVENTION

The invention is defined as in the claims and relates to, inter alia: In one aspect, the invention relates to an inhibitor of expression and/or function of ZPI, wherein said inhibitor is conjugated to one or more ligand moieties.

In a further aspect, the invention relates to an inhibitor according to the invention, wherein said inhibitor is an siRNA oligomer.

In another aspect, the invention relates to an inhibitor of expression and/or function of ZPI, wherein said inhibitor is an siRNA oligomer.

In a further aspect, the invention relates to an inhibitor according to the invention, wherein said inhibitor comprises an siRNA oligomer conjugated to one or more ligand moieties.

In a further aspect, the invention relates to an inhibitor according to the invention, for use in prevention or treatment of a disease related to a disorder of haemostasis, such as haemophilia.

In a further aspect, the invention relates to an inhibitor according to the invention, wherein said one or more ligand moieties comprise one or more GalNAc ligands or comprise one or more GalNAc ligand derivatives.

In a further aspect, the invention relates to an inhibitor for use according to the invention, wherein said one or more ligand moieties comprise one or more GalNAc ligand derivatives.

In a further aspect, the invention relates to an inhibitor for use according to the invention, wherein the target of the inhibitor is ZPI.

In a further aspect, the invention relates to an inhibitor or an inhibitor for use according to the invention, which is an siRNA oligomer having a first and a second strand wherein:
i) the first strand of the siRNA has a length in the range of 15 to 30 nucleosides, preferably 19 to 25 nucleosides, more preferably 23 or 25; even more preferably 23; and/or
ii) the second strand of the siRNA has a length in the range of 15 to 30 nucleosides, preferably 19 to 25 nucleosides, more preferably 21 nucleosides.

In a further aspect, the invention relates to an inhibitor or an inhibitor for use according to the invention, wherein the second sense strand further comprises one or more abasic nucleosides in a terminal region of the second strand, and wherein said abasic nucleoside(s) is/are connected to an adjacent nucleoside through a reversed internucleoside linkage.

In a further aspect, the invention relates to an inhibitor or an inhibitor for use according to the invention, wherein the second strand comprises:
i) 2, or more than 2, abasic nucleosides in a terminal region of the second strand; and/or
ii) 2, or more than 2, abasic nucleosides in either the 5′ or 3′ terminal region of the second strand; and/or
iii) 2, or more than 2, abasic nucleosides in either the 5′ or 3′ terminal region of the second strand, wherein the abasic nucleosides are present in an overhang as herein described; and/or
iv) 2, or more than 2, consecutive abasic nucleosides in a terminal region of the second strand, wherein preferably one such abasic nucleoside is a terminal nucleoside; and/or
v) 2, or more than 2, consecutive abasic nucleosides in either the 5′ or 3′ terminal region of the second strand, wherein preferably one such abasic nucleoside is a terminal nucleoside in either the 5′ or 3′ terminal region of the second strand; and/or
vi) a reversed internucleoside linkage connects at least one abasic nucleoside to an adjacent basic nucleoside in a terminal region of the second strand; and/or
vii) a reversed internucleoside linkage connects at least one abasic nucleoside to an adjacent basic nucleoside in either the 5′ or 3′ terminal region of the second strand; and/or
viii) an abasic nucleoside as the penultimate nucleoside which is connected via the reversed linkage to the nucleoside which is not the terminal nucleoside (called the antepenultimate nucleoside herein); and/or
ix) abasic nucleosides as the 2 terminal nucleosides connected via a 5′-3′ linkage when reading the strand in the direction towards that terminus;
x) abasic nucleosides as the 2 terminal nucleosides connected via a 3′-5′ linkage when reading the strand in the direction towards the terminus comprising the terminal nucleosides;
xi) abasic nucleosides as the terminal 2 positions, wherein the penultimate nucleoside is connected via the reversed linkage to the antepenultimate nucleoside, and wherein the reversed linkage is a 5-5′ reversed linkage or a 3′-3′ reversed linkage;
xii) abasic nucleosides as the terminal 2 positions, wherein the penultimate nucleoside is connected via the reversed linkage to the antepenultimate nucleoside, and wherein either
   (1) the reversed linkage is a 5-5′ reversed linkage and the linkage between the terminal and penultimate abasic nucleosides is 3′5′ when reading towards the terminus comprising the terminal and penultimate abasic nucleosides; or
   (2) the reversed linkage is a 3-3′ reversed linkage and the linkage between the terminal and penultimate abasic nucleosides is 5′3′ when reading towards the terminus comprising the terminal and penultimate abasic nucleosides.

In a further aspect, the invention relates to an inhibitor or an inhibitor for use according to the invention, wherein the reversed internucleoside linkage is at a terminal region which is distal to the 5′ terminal region of the second strand, or at a terminal region which is distal to the 3′ terminal region of the second strand.

In a further aspect, the invention relates to an inhibitor or an inhibitor for use according to the invention, wherein the reversed internucleoside linkage is a 3′3 reversed linkage.

In a further aspect, the invention relates to an inhibitor or an inhibitor for use according to the invention, wherein the reversed internucleoside linkage is a 5′5 reversed linkage.

In a further aspect, the invention relates to an inhibitor or an inhibitor for use according to the invention, wherein one or more nucleosides on the first strand and/or the second strand is/are modified, to form modified nucleosides.

In a further aspect, the invention relates to an inhibitor or an inhibitor for use according to the invention, wherein the modification is a modification at the 2′-OH group of the ribose sugar, optionally selected from 2′-Me or 2′-F modifications.

In a further aspect, the invention relates to an inhibitor or an inhibitor for use according to the invention, wherein the first strand comprises a 2′-F at any of position 14, position 2, position 6, or any combination thereof, counting from position 1 of said first strand.

In a further aspect, the invention relates to an inhibitor or an inhibitor for use according to the invention, wherein the second strand comprises a 2′-F modification at position 7 and/or 9, and/or 11 and/or 13, counting from position 1 of said second strand.

In a further aspect, the invention relates to an inhibitor or an inhibitor for use according to the invention, wherein the first and second strand each comprise 2′-Me and 2′-F modifications.

In a further aspect, the invention relates to an inhibitor or an inhibitor for use according to the invention, which is an siRNA, wherein the siRNA comprises at least one thermally destabilizing modification, suitably at one or more of positions 1 to 9 of the first strand counting from position 1 of the first strand, and/or at one or more of positions on the second strand aligned with positions 1 to 9 of the first strand, wherein the destabilizing modification is selected from a modified unlocked nucleic acid (UNA) and a glycol nucleic acid (GNA), preferably a glycol nucleic acid.

In a further aspect, the invention relates to an inhibitor or an inhibitor for use according to the invention, wherein the siRNA comprises at least one thermally destabilizing modification at position 7 of the first strand, counting from position 1 of the first strand.

In a further aspect, the invention relates to an inhibitor or an inhibitor for use according to the invention, which is an siRNA, wherein the siRNA comprises 3 or more 2′-F modifications at positions 7 to 13 of the second strand, such as 4, 5, 6 or 7 2′-F modifications at positions 7 to 13 of the second strand, counting from position 1 of said second strand

In a further aspect, the invention relates to an inhibitor or an inhibitor for use according to the invention, which is an siRNA, wherein said second strand comprises at least 3, such as 4, 5 or 6, 2′-Me modifications at positions 1 to 6 of the second strand, counting from position 1 of said second strand.

In a further aspect, the invention relates to an inhibitor or an inhibitor for use according to the invention, which is an siRNA, wherein said first strand comprises at least 5 2′-Me consecutive modifications at the 3′ terminal region, preferably including the terminal nucleoside at the 3′ terminal region, or at least within 1 or 2 nucleosides from the terminal nucleoside at the 3′ terminal region.

In a further aspect, the invention relates to an inhibitor or an inhibitor for use according to the invention, which is an siRNA wherein said first strand comprises 7 2′-Me consecutive modifications at the 3′ terminal region, preferably including the terminal nucleoside at the 3′ terminal region.

In a further aspect, the invention relates to an inhibitor or an inhibitor for use according to the invention, wherein the siRNA oligomer further comprises one or more phosphorothioate internucleoside linkages.

In a further aspect, the invention relates to an inhibitor or an inhibitor for use according to the invention, wherein said one or more phosphorothioate internucleoside linkages are respectively between at least three consecutive positions in a 5′ or 3′ near terminal region of the second strand, whereby said near terminal region is preferably adjacent said terminal region wherein said one or more abasic nucleosides of said second strand is/are located as defined herein.

In a further aspect, the invention relates to an inhibitor or an inhibitor for use according to the invention, wherein said one or more phosphorothioate internucleoside linkages are respectively between at least three consecutive positions in a 5′ and/or 3′ terminal region of the first strand, whereby preferably a terminal position at the 5′ and/or 3′ terminal region of said first strand is attached to its adjacent position by a phosphorothioate internucleoside linkage.

In a further aspect, the invention relates to an inhibitor or an inhibitor for use according to the invention, wherein the oligomer is an siRNA and the second strand of the siRNA is conjugated directly or indirectly to one or more ligand moiety(s), wherein said ligand moiety is typically present at a terminal region of the second strand, preferably at the 3′ terminal region thereof.

In a further aspect, the invention relates to an inhibitor or an inhibitor for use according to the invention, wherein the ligand moiety comprises
i) one or more GalNAc ligands; and/or
ii) one or more GalNAc ligand derivatives; and/or
iii) one or more GalNAc ligands and/or GalNAc ligand derivatives conjugated to said SiRNA through a linker.

In a further aspect, the invention relates to an inhibitor or an inhibitor for use according to the invention, wherein said one or more GalNAc ligands and/or GalNAc ligand derivatives are conjugated directly or indirectly to the 5′ or 3′ terminal region of the second strand of the siRNA oligomer, preferably at the 3′ terminal region thereof.

In a further aspect, the invention relates to an inhibitor or an inhibitor for use according to the invention, wherein the ligand moiety comprises

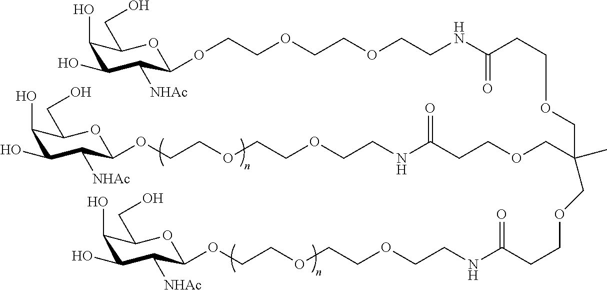

In a further aspect, the invention relates to an inhibitor or an inhibitor for use according to the invention, having the structure:

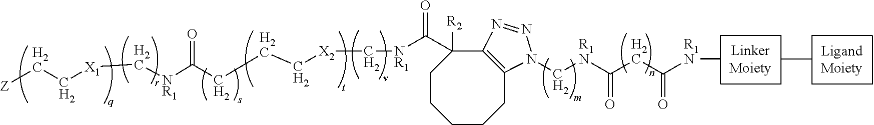

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, y are independently integers from 0 to 4, with the proviso that:
(i) q and r cannot both be 0 at the same time; and
(ii) s, t and v cannot all be 0 at the same time;
Z is an oligomer

In a further aspect, the invention relates to an inhibitor or an inhibitor for use according to the invention, having the structure

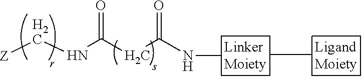

wherein:
r and s are independently an integer selected from 1 to 16; and
Z is an oligomer.

In a further aspect, the invention relates to an inhibitor or an inhibitor for use according to the invention, formulated as a pharmaceutical composition with an excipient and/or carrier.

In another aspect, the invention relates to a pharmaceutical composition comprising an inhibitor according to one or more preceding aspects, in combination with a pharmaceutically acceptable excipient or carrier.

In a further aspect, the invention relates to a pharmaceutical composition comprising an inhibitor according to the invention, in combination with a pharmaceutically acceptable excipient or carrier, for use in the treatment of a disease related to a disorder of haemostasis, such as haemophilia.

In another aspect, the invention relates to the use of ZPI as a target for identifying one or more therapeutic agents for the treatment of a disease related to a disorder of haemostasis, such as haemophilia.

In another aspect, the invention relates to a method of treating or preventing a disease related to a disorder of haemostasis, such as haemophilia, which comprises administering to a patient an inhibitor of ZPI, such as an inhibitor as defined according to one or more preceding aspects.

In another aspect, the invention relates to ZPI for use as a biomarker of a disease related to a disorder of haemostasis, such as haemophilia.

In another aspect, the invention relates to ZPI for use in an in vivo method of predicting susceptibility to a disease related to a disorder of haemostasis, such as haemophilia, typically by monitoring the sequence and/or level of expression and/or function of ZPI in a sample obtained from a patient.

In another aspect, the invention relates to a method of predicting susceptibility to a disease related to a disorder of haemostasis, such as haemophilia, and optionally treating a disease related to a disorder of haemostasis, such as haemophilia, in a patient, said method comprising:
(a) obtaining a sample from the patient,
(b) detecting the sequence and/or expression and/or function of ZPI in said sample obtained from the patient,
(c) predicting susceptibility to a disease related to a disorder of haemostasis, such as haemophilia, based on the sequence and/or expression and/or function of ZPI in said sample obtained from the patient,
(d) preferably administering to the diagnosed patient an effective amount of an inhibitor of ZPI.

In another aspect, the invention relates to an inhibitor or composition according to the invention, in the preparation of a medicament for use in the treatment of a disease related to a disorder of haemostasis, such as haemophilia.

### FIGURES

FIG. 1a shows an exemplary linear configuration for a conjugate.

FIG. 1b shows an exemplary branched configuration for a conjugate.

FIGS. 2-5 show preferred oligomer-linker-ligand constructs of the invention.

FIG. 6 shows the detail of the formulae described in Sentences 1-101 disclosed herein.

FIG. 7 shows the detail of formulae described in Clauses 1-56 disclosed herein.

FIG. 8 shows, on the left (A), a typical pathway view of the coagulation cascade which is too simple to represent the biology realistically. On the right (B) is an example of a network model which captures the full complexity of haemostasis.

FIG. 9 shows an overview of the network biology workflow (top). (Middle) Haemostasis directed network. Directed network constructed using ETX proprietary interactome which includes directed interactions from NLP and curated pathway data. (Bottom) Target identification using KPA on the haemostasis network. Right panel is a bar chart showing the top ranked proteins using the KPA approach. While clinically validated haemophilia target AT3 ranks in the top 10 proteins, ZPI ranks higher by this chosen network metric. Heatmap panel on the left shows whether the proteins are expressed in hepatocytes and/or secreted.

FIG. 10 shows the visual bleeding score of mice in three different treatment groups (wild type control group, Haem A mice that received a vehicle (0.9% saline), and Haem A mice that received the GalNAc-siRNA construct ETXM1184) 3 days (FIG. 10A) and 10 days (FIG. 10B) post injury. Definition of the bleeding scores is provided below.

FIG. 11 shows (A) a comparison of knee diameters at day 3 and 10 post injury of mice in three different treatment groups (wild-type control group, Haem A mice receiving vehicle (0.9% saline), and Haem A mice receiving the GalNAc-siRNA construct ETXM1184) and (B) a comparison of skinned knee diameter at day 10 post injury of mice in the same three treatment groups.

FIG. 12 shows a comparison of the severity of (A) bone marrow hyperplasia, (B) osteoarthritis, (C) chondrocyte degeneration/necrosis, (D) haemorrhage, (E) haemosiderin deposition, (F) haematoma, (G) osteoclastogenic bone resorption, (H) osteolysis, (I) periostitis, (J) sub-chondral bone sclerosis, (K) tendon degeneration, (L) tendonitis and (M) tenosynovitis (FIG. 12M) in mice in three different treatment groups (wild-type control group, Haem A mice receiving vehicle (0.9% saline), and Haem A mice receiving the GalNAc-siRNA construct ETXM1184).

### DETAILED DESCRIPTION

The present invention, inter alia, provides inhibitors, for example oligomers such as nucleic acids, such as inhibitory RNA molecules (which may be referred to as iRNA or siRNA), and compositions containing the same which can affect expression of a target, for example by binding to mRNA transcribed from a gene. The target may be within a cell, e.g. a cell within a subject, such as a human. The inhibitors can be used to prevent and/or treat medical conditions associated with the e.g. the expression of a target gene.

In particular the present invention identifies inhibitors of ZPI as useful in the prevention and/or treatment of a disease related to a disorder of haemostasis, such as haemophilia.

Protein Z-dependent protease inhibitor (ZPI) is a protein circulating in the blood which inhibits factors Xa and XIa of the coagulation cascade. It is a member of the class of the serine protease inhibitors (serpins). In humans, ZPI is encoded by the ZPI gene (SEQ ID NO:1).

(ZPI)
SEQ ID NO: 1
AATGTGGGTTGGAGCCCCCATACAGAATCTCTATGGGGGCACTGCCTAGTGGAGCTGTGAGAAGACG
GCCACCGTCCTCCAGACCCCTGAATGGTAGATCCACCGACAGCTTGCGCCATTTATCCGGAAAAGCCA
CAGACACTCAACGCCAGCCCGTGAAAGCAGCCAGGAGGGAGGCTGTACCCTGCAAAGCCACAGGGGC
AGAGCTGCCCAAGACCAAGGGAAGCTACCTTTTGCATCAACGTGACCTGGACTCAAAGGAGATCATTT
TGGAGCTTTAAAATTTGACTGACCTGCTGGATTTCAGACTTGCATGGGCCCTGTAACCACTTCGTTTAG
GCCAATTTCTCCCATTTGGAACAGCCGTATTTACCCAATACCTGTAACCCCATTGTATCTAGGCAGTAA
CTAGCTTGCTTTTGATTTTACAGGCTCATAGGCAGAAGGGACTTGCCTTATCTCAGGTGAGACTTTGGA
TTGTGGACTTTTGGGTTAATGATGAAATGAGTTAAGACTTTGGGGGACTGTTGAGAAGGCATGATTGG
TTTTGAAATGTGAGGACATGAGATTTGGCAGGGCCAGAGGCGGAATGATATGGTTTGGCTCTGTATCC
CCACCCAAATCTCATCTTGAATTGTACTCCCATAATTCCCACATGTTGTGGGAAGGGACCCAGTGGGA
GATAATTTGAATCATGGGGGTGGTTCCGCCATACTGTTCTTGTGATAGTGAATAAGTCTCACAAGATCT
GATGCCTTTATTGGGGGTTTCTGCTTTTGCGTCTTCCTCATTTTCTCTTGCCGCCACCAGGTAAGCAGTG
CCTTTTGCCTCCCACCATGATTCTGAGGCCTCCCCAGCCACGTGGAGCTGTAAGTGCATTTAAACCTCT
TTCTCTTCCCAGTCTCGGGTATGTCTTTATCAGCGGCGTGAAAATGGACTAATACACTGTGGTTATGTA
TTATAGTCATATGATATTTTCATATTTTTGGAAGCTGGGTGAAGGGTAGATGTGGAGACCATGATTTTT
GCAAATTTTTTTAAGTTTAAAGTTATTTCTAAATTAGAAGTTTAAAAAGAAGAAATCACATAAGCCAT
AACACAATAGAAAGATGTCTTTAAAGTTCAAGGCAGGAGGGATGTCTGGAAATCAGCGAGAAATTTG
CACCTGTGTGTGCATGTGCATATGTGTGTGTGTATGTTGCAAGGACTTGGAAAGCCCTTTTTTTCCTAC
CTCTGTACTACTGTGGGGGGAGGCTAAACTTGACTTCTTCCCATCTTAGTTCTTTTTTGGGATAGACTC
CTGTAACAAAAGACAGACAAGAGAAAAATCAGCTTACAACATGGGCCATGCACTTCACACAGGAGAA
ACCTGCATGAAAAGTAACTCAAAATGGTGCCTTAGAACTCCACTTACCTTTAGTAAAGAGCAATAAAT
TAGCAGGAAAATCATGGATCGGGACAAGGGAAGTGGTTTTATGCTTCCAAGGGCAGGAAATCATGGA
AGGTAAATATATGGGAGGAAACTAAAGGAATAAGGCTTGTTTGCATATTCCTCTGATGCCATCTCTGG
GTTGATAAGAGTCTAGAGTCATTTCCAGTAAAGATGAATTTTTATCTGTCTTTAGGAAGAAAGGGGGA
AAGATAGAGAAAACTATTTCTCCATTTGCTGTTTCTTAATTACCTTCAGTTCAAAAATAATTTTTATATC
AGAAAGGCATATTTAGAGGTATGTTAGTTTATTTTCACACTGCTAATAAAGACATACCCAAGACTGGG
TAATTTATAAAGAAAAAGAGGTTTAATGGACTCACCGTTCCACATGGTTGGAGAGGCCTCACAATCAA
GGCAGGTCTTACATGGCAGCAGGCAAGAGGGAGAATGAGAGCCAAGCGAAAGGAATTTCCCCTTAAA
AATCCCCTTATAAAACCATCAGATCTCGTGAGACTTACTCACTACCACAAGAACAGTATGGGGGAAAC
CACCTCTATGATTCAATGATCTCCCACTGGGTACCCCCCAACAACACGTGGGAATTATGGGAGCTACA
ATTCAAGATAAGATTTGGGTGGGGACACAGACAGACCATATCAAGGGGTAACATAGTCTGGTTTCCTT
TACTACCCACCTACCCAAACACCCCCTTCATCTGATCCACACAAAGTAAACTCTTGCAGTTCTCTCACT
GTTTCCTGGAGTCTGCTTTTGGTCTCATAGGACTGCCCTAACGCTTGTTTTTCAGACGTTTAACCCTGTA
GGTCTCTGGACAAATTTGCTTTAGAAGCCCCTCGATGTCGCCCTGAAGAGTGGCTTTCAGAAGTTGTGC
CTCCTGCCTGAGGGGAGTTCCAGGAAGGGTTCTGCATCGCCTATGAGTTTATCTGGATCACCAGAGGC
CTTCCCGTCAGAGCTTTCCCAATCGTTTTTGGCCAAGGAGTGTGAGAAGCTAAAGTTCATAACAACTG
GAAGTCAGACAGCCTGGTCTATTCTGCTTTAACTCTAGCAGGAAAGGCCTTCATGGTGGGGCCTGAAT
ATCTTCCTTTATAAAATCAAAGCCTGGGGACAGGGTTACTTACTTCTGAGGTTCAATCTGGCTCTAAAA
TTATGCAACAAATGCCATTCCTTTAGCACTTCCTTCCTACCGGGCGAGATACTCAACTCCACAGGCACC
ACCTCAGTTCATCCTCTCAGAAGTCCTAACAGCTCAGCCTGGGGCACCCCATTTTACAGATTAGTAAAC
TGAGGCTAAGAGAGGTTAGGTAGCTTGTTCAGGGTCATGCTGCTGGTAAAAGAGCTCAGGCTACAGTG
CTATGCATTGAGTTTTCTCACTTTCCCATCTAACTGGAGGGCTAAAGGTCAAAGAGTGGGCAGCTCCCT
TGTTGGGAGCTGTACAGGAATAATGTCCTCCCTGAAGGAGGGGGACTTCTGAGCCACACCCTGGGGTC
CAGGGCTCACAGCCTTAGGAGCAAAATCGTCCACCCCCTTCCTGGTTCCTCGGTGCTGCAGAGATATT
CATAGGACAGAGTCTGAGTTCTGGCCACTTAACAGAGGAAGAAAGGCTGGCTCGGTGAGGTTAACTT
ACATCCCAGCAGCTAGGAACCGGGAGCAGAGGACCTCAGATTCACACCAGGGCAGGAGGCAATGGCC
TGGCTGAAGCCTTCACAATCTTCCCAATATACTCCGCTGCCTTCCTTTATAAGGATCCATTTCTGAAAC
CCTGTGCCCTGGCCAGGCACGGTGGCTCACACCTGTAATTCCAGTACTTTGGGAGGCCAAGGCAGGAG
GACCACGAGGTCAGGAGTTTGAGACCAGCCTGGCCAATATGGTGAAACCCCGTCTCTACTAAAAATAG
AAAAATTAGCGTGGTGGCAGGCGCCTGTAATCCCAGCTACTCGGGAGGCTGAGGCAGGAGAACTGTT
TGAACCTGGGAGGTGGAGGTCTCAGTGAGCTGAGACAGACAGTGCCTGGGTGACAGACAGAGACTCC
GTCTCAAAAAAAAAAAAAAAAAGAAAGAAACCCTGTGCCCTAAGACCTGCACACTCGCTGGCTCCGC
TCAGACATTTAGCAAAGCAGACACCTTCCCAGGCCTGGAGGAAACAGCCCCTGCTTTTTGGGAATCCA
CAAGCCCGCAGCTGCAGAGCTCGACCTGGATGGGCAGGCAAAGGCTGACTCCTGTGCGTGGTGTGAG
TCCAGCCTGGCCCCTCTACACCCTCACTTTCACCTCTTAAAGAACTGCCTATTAACAGAGCAGGTACTG
CCCAAAAGGAACACTCTGGAAACTTGTTGGGACACTTCTGCCTTTCACAAACGTTTGGGGGGAGTACT
ACTAGCATTTAAGGATTGAGGGTTAGCAATGCCAGACATACCAGAACACGCAGGGCAGTCTCCCATG
ATGAAGAGGCCGCCGGGTTCCCCAGGACTCACATGTCCACCTCAAGTTCACGTGGGATTATCTGAGCC
TAGACTGTCAGTCCTGGGGCTGCTTTATTTCATATAAAAATATAATATTTATCCAAGGTTTTACTACAC
ACTGCATTTTCTGTGAAGACAATGACCGTGTAAATCAGGGAAAGATCTATATTTTATTTTGTTTGAAAC
TTTACCAAGCATTATTTACCATTTCAAAAGCTCTATCCCTGGTAGTACCATTGGTTTTCTTGTTCACCGG
CCAGCAGTGAGCAGCACACAAGCGACCTCCCGTGGGCTCCACATTGGACAGCCTCACTGCACCTGCCC
AGGCCCTTAGGCCACAGCACTGCCATATTCAGGGACACATTATTCTCTTTTATTATGCCTCCATATTAT
CATTACAGCATTATCTTTTTTTTAATTTTGTGGGTAGATTATATTAGCTATACGTTTCACTTCAATGGTA
GTAGTAAGGGGCACATAACAAAATATTTACTTATATATATTAAAAAAGAGAGTCTGAGAAGTCTGAA
AAGTTTTGCCATAAACGGTCTCCACCAGCCTCAACTCTGAGTGCCCGAGGATTCAGTCTCAAGTCCAG
CAACATTGTGAAGCAGGAAATTTACCTTGAAAGGAGCTATGTACTCTAAGTAGTGATTTACCTGTCTG
CCTCCCCCACTGGATTGACCAGTTCCTTGGGGGCTGAGAGAACAGGTCCTGAACATTTCTGCTGTGCCC
CCCAACCCACATCCTCATAGTGTCCAGTACCAGGCTGGGGACTCAGGAAGCATCCATGGGATCCCCCA
GTGCCTTCTTTCTCGAGGTGTTCAGCACCTAGAACAGCTCAAGACAAATTCCCCACACCCCACCCAGA
CAGAGCTGAATCTTACTGGGGCGAAGCCTTGAGTTGCAAGGCAGAAGCTCTCGTGATGGGATTTGGGT
CATATTCCGGGTTATAGGAGGAGCTGGGGAGTATGGGAAGCCTCCCACTTGGTCTTTGGTTTTCCAGA
AACTCCACCATCACAAGCAGGATGTTAATCAGTAACCGTCCCACAGGGGATCATACTTTGGAATAGCA
AATATTTGCTGAAGGTTCTGGGCTGCAAAGCTGAAGCTTTGGTTTCTGCTCTAAATGAAGGACTTTTCC
AGGACCCAAGGCCACACACTGGTAAGAGGCAGTGGGTTACAGGAGACCTTCAATGAGTCTAATCAGG
GAGGGACCGGGAAGGATGGTATCATCCCTGGGCGGGCTCCAACGTGAGGGCTGTGTGGCTGAGCAGT
GCAAAGACCTCCATCCTACACTCCACAGGGACTGTACATACAGATTGGGAGCTGGAGTGGGGTAAGA
GGCGAATTATAGACACAAGGGGCTCCTCTGCAGGAAGGAGGCCAAGGGAAAGAGGCTTGAAAGGCTT
GATATTTCACCCACCACCACTCACTGCCGGAGTAAGCAGGTCTCCCCTTCCCAGGGCTGAGGGGAGGC
AGGGATGTGTGCTGTCCCAGGGCTGAGAAGTGGCAGGTGAGCTGGTGATTCCTTACTGCCCAGGTTCT
GTCTAGGAAGGTGCGTCCTCACCATGCTGGATGGTGTCCTAGTCCAGGAGCACCCCCTGAGCTCCTGG
CCTAGACTCCAAAGGGTTGGGTAGATGAGCAAAGACTTTACAAAGACCTTAGGCGATATATGTCCAGG
AGCACCCAGGAATTACTGGGCTACCACTGCAGACTGCAGGACAAGCTCCAAGAACAGGAAGGTAAGA
CTCAGCATTTGGAGGTGGTGACATCTAGTTGGCGTGCTGGGCTAATTTCCTGACCATTGTACAGGGAG
AAGTAACCTTGAATTCAGGAGTATTCTGTGTGGTCTTAATGTAGAAAGTAGCACTAAATGATGCCACG
TAATCGTTTTAGCTCAGGCTCCTCTAACAAAACACCACAGGCTGGGTGGCTCCAACAGCCATTGATTTT
TCACAGTTTTGGAGGCTGAAAGTCCGAGTCAGGGTGCCAGCGTGGCCGGATTCTGGTAGGGCTGTCTT
CTTGGCTTGCAGATGGCCACCTTCGCACCGTGTCCTCCCATGGAGAGGAGGTGCGGAGGGGGACTCTG
CTCTCTTCTTATGACAGCACTAGTGCTATCACAGGGGCCTTGCCCTCACGACCTCATCTAAACCTAATC
ACCTCCCAAGCGCCCCAACTCTATTGCCATCACAATGGTGGTTCGGGCTTCAACTTATTAATTCTCAGG
GGACACATTCAGTCCATAACAATAAAAGCGTGAAACTGGGCTGCGTTTACACTGAAAGAGCTATTTAC
CCAACGTTTACAATACTTGGGTGACCTGTTGAATGCAGGCTTGCCATTTAGAGTCAAAAAGAGCTTCC
TCAACAGTGTCCTTTGGGAAACACAGTGGAAGTATTTCACTGCTTCTACAGGGGAGAGGGTAGTGCCG
TTCAGACTGCAGAGTGAGGCCCTGAATTCCGGGGTGCCATTCAGCCCGAGCAAGGGGCAACATGCTG
GGCCCTGGCGCTGGAGGCGGTTTTGTCCCAGGCATAGATAAGGACTCAGCCCCTGCATCAGGAAGAG
GCCTGGCAGCACCGCCTGTCAATACATTTTGCCGCAGGTGACCTTGGTCAAGAATAAGGGTCTCTGCT
GATGGGAACTACTGTGAGGCCGGCAGCATCCACCCTGCGCTCACTGGGCTGGGTGGCCTACCCCACCC
AGACCCTCCCAGGGCAGTGGGCCCAGAGAGAGGATGAGGGAGGGCAGGTGTCCCAGGGGTTCTGCCC
AGCCAGCCTCTGGGATCAGGCCTGCAGTGTGGCTGAACACCAGAACTGAGTTTGGACACAGCCAGGT
GGCCCAGGCCAGTCCCAAGCCATGTATTTGGATGGAAAACATGGAAGTATTCAGGAGCCAGGCTCTGT
GTCCAAGGATGTGGAGGGAGCCTAAAAGGCGACAGAGAAGGGGACAGCTAACGGTGAAGAAGTGTA
GCTCCCACACTGCAGCCTAGGACAGTGAGAACCGGCATGCAGCCCAGGTGGCTGAGGGCTCTATGAA
GCCACAGTGGAGGGAGCCCAGAAGTGGGTTGTATGAATTGCGGGGCCTCCTGCTACCCGGGAGCTGC
AGCTATAGGAAGGAAGGAAGGAAGGAAGACCTCCAAGGAACTGTGTAGCAGAGGTGCAGTGCAAAG
AGAATTTTGATAAAAAATCCAGGAAAGCTCCAATACTTTCCCCCTTCCTTGCCTAACGGGCATGCAGG
CACTCCAATCCCCAGCCAAACAGGGCACTGGGCAAGGCCGGCCACCCATCTGGATGGGCAGCCTGAC
GACCAGATGGTCAGGGCAGTGAATGAAGCAGATCAAGGAAAGGTGTGTGAGGACCCCTGATTCCACC
TGCTTGGACCCCCACCTTCTGTGCTGCCTCCTGCTCCCAGAGTGGACTCTCTTGCCCTGGCCCTCAGGG
AGGAGACGGGATGAATGAAAACGGGGTCAGGACTGAGAGCTGCCTGCCGGCCTGGCAGGGAATGGG
AACTGGAGGAGGTTTTGCTCTGTGAAATAATGTCCCCTCTTTGGGTGAGCAAATGTCACCCACACTTGC
TCTAGGTCTCCCTGGGGCAGGGCTAACCTACTTGAGCCACAGGAAGGAGGCAGGGTCCCTGAAGAAG
CTTTTACTATCCACAAAGACATTTTAGGAGGCATTAAAACCATCTCTATCCTCTCCTCTCCACAGGAAG
TCTTGCAGCTGAAGGGAGGCACTCCTTGGCCTCCGCAGCCGATCACATGAAGGTGGTGCCAAGTCTCC
TGCTCTCCGTCCTCCTGGCACAGGTGTGGCTGGTACCCGGCTTGGCCCCCAGTCCTCAGTCGCCAGAGA
CCCCAGCCCCTCAGAACCAGACCAGCAGGGTAGTGCAGGCTCCCAAGGAGGAAGAGGAAGATGAGCA
GGAGGCCAGCGAGGAGAAGGCCAGTGAGGAAGAGAAAGCCTGGCTGATGGCCAGCAGGCAGCAGCT
TGCCAAGGAGACTTCAAACTTCGGATTCAGCCTGCTGCGAAAGATCTCCATGAGGCACGATGGCAACA
TGGTCTTCTCTCCATTTGGCATGTCCTTGGCCATGACAGGCTTGATGCTGGGGGCCACAGGGCCGACTG
AAACCCAGATCAAGAGAGGGCTCCACTTGCAGGCCCTGAAGCCCACCAAGCCCGGGCTCCTGCCTTCC
CTCTTTAAGGGACTCAGAGAGACCCTCTCCCGCAACCTGGAACTGGGCCTCACACAGGGGAGTTTTGC
CTTCATCCACAAGGATTTTGATGTCAAAGAGACTTTCTTCAATTTATCCAAGAGGTATTTTGATACAGA
GTGCGTGCCTATGAATTTTCGCAATGCCTCACAGGCCAAAAGGCTCATGAATCATTACATTAACAAAG
AGACTCGGGGGAAAATTCCCAAACTGTTTGATGAGATTAATCCTGAAACCAAATTAATTCTTGTGGAT
TACATCTTGTTCAAAGGTACTTTGATAATGTTCTGCTCTCCCAAGGCCACAGGGCCCTACGATTGTCTC
TCCCTTTCCTTTCGTTAGGCCAGCATATGATTAACGCTACGTGATTTTCTATGAATGTGTTTTCACGTTT
CAAAAACAGATTGATACACATATTGAACAGTGCCAGACGCTGTCATTTGAGGCCCTTCCCTGGTATCC
TATGTGCTTGTAGTCCTTATTATTTTCAGAGCACTCTACATAGCTCCCCTCTGACACTTAGAAGCATAG
GGTCTTTCCAAAAAACAGGGGGCTGGGGGATTATCTGGGGGATTTAGGATTGCATCATTGCTCCTTCA
TTTTTACTTTTTGACCAACTCTCTGCCCTTAGATTCCTATTATAGAAAATAGGGACACTCCACCTACTAC
AGTGTTAGAGGCTAAATGAGACAATGAATGTAAAGTGCCCAGATGGGCTTGGCACATAGCAGACACT
GAGTATCTATTGTTTACTTGTTCTTCCAAACTGCCAATCAGCAGGTAGAGCAGGAGTTGTCTCCTTTCT
AAAGATGAAACCAGCTCAGAGACGTTAGCTTGATCAAGGTCACACAGTAAGTGGCAGAGGCAAAACC
CAAACAAGGGCCTCCTGACCCCCTGATCCTAGGTTCTGTCCAGCCCTGCCTCCCTAATGGGGCACTGG
ACGTGGGTTGGATGCCACTTTCGCAGAGCTGGCACCAGACTTACAAAGCCCCGGCAGGGGAAGCCAC
TTTACAACCAGCCAGGCCACACCCCCAGGGCAGACGTTTATGTAGAGAGTAATGTACCTGCCTGCTAG
TAGCCTCTGCATTGTGGGGCCTTCTCTCAGAACCACACTAAACAGTGGGTGGGTGAGAAGTGTCACTC
CTGCCACCTTGGACTCTGCATGTGCTTGTGCCTGGTGTGAATGAGACAAAGTGGCAGTCAGAGGTGCC
AGGCAAAGGCTTTTCTCTAAGCTGGAGCCAACTATGAGGGAACGACTGTGAATTCCGTTCAGGTCCAG
GACAATGAGAGGAGCCAGGGATTGTTAGGAAACATTTCCCTGCTTTCGTGTGCGATTCCCAATAGGGC
CTGCGAGTGGAGCTGCATTTTGCTAGCTGGGCTAGAGGACGGGGAAAATTTTGGGGAAATTTATTTTG
CCTGCCTGAGCTGTGGAAAAGCCAACCCAATTAGGGAACGCCTTTCCTAGTTGGAACGAGAAGACGA
GAAGTGAGAGAAGTGAGATAGAAGGCTCCCTCTCTATTATTTGAGCAAGAACAATGCTTTTCAAAGAG
GGAATTTCTGCAATGAGTTCTTCTCTTACTTGTTCAGGGAAATGGTTGACCCCATTTGACCCTGTCTTC
ACCGAAGTCGACACTTTCCACCTGGACAAGTACAAGACCATTAAGGTGCCCATGATGTACGGTGCAGG
CAAGTTTGCCTCCACCTTTGACAAGAATTTTCGTTGTCATGTCCTCAAACTGCCCTACCAAGGAAATGC
CACCATGCTGGTGGTCCTCATGGAGAAAATGGGTGACCACCTCGCCCTTGAAGACTACCTGACCACAG
ACTTGGTGGAGACATGGCTCAGAAACATGAAAACCAGGTACAACTCTTGCCCACACCCTATACAAACT
CTACCTTTCTGTACTGGCAAACGCTCAGCACAATTTCATTGAATGCACCGTGATTTAATGTCTCCTCCA
GTGAGCTATAAGTTTCCTGAAGGCAGGGCAGCATTTGTCTTTTTTTCCACTCTATCCCCAGCATCTGTC
ACAGGGTGCCTGGCTGATTCATTCATTGAGTCCATCAGTATTTTACGTTCTGCGACTGTGATAAATATA
TGATGCCAGGGATCCATCAGCAAACAAAACAGGCAAAATTAGTCTGCCCTCATGCAGCTTACATTCTA
TTGAAGGAAGACAAAGAGTAAATTAAAAATAGGTAATAATGCAGGGAAGGGGACAAGAAGCATCAT
CAGGATGCAGATGGAGGTTAGACAAGGCCTCTCCAAGAAGGTAACAGTAAGCAAACATCTGAAGATG
AAGGATAAACCATGTGGATATATTCGGGGAGAGAAGTGTTATGTTACAGGCAGAAGTGTACAAGTTCT
GGGATGGGAGTGTACCTGGTGGGTTTGAAGAACATCAAGGAGACAAGTGTGGCTTCAGCAGTTGGAG
ATAAAATCAGAGAGGAAACAGGGGCCCAGTCCCCAGAAAAGACTTGGGCTTTCCTGAGAGAGGCAGG
AAGCCACTGGATGGTTCTGAGTAGAGGAGCAACCTGATTTTGACTTCTGTTTTTAAAGGATCACATAA
GCTCCTGTGTTGAGAAAAGACACTAGGGGGTAAGGATGGAAGCAAGGGAGAGTGGTTAGAAAGTTAC
TAGCAATCCAGGTAGAGATGCTGCTACCTGGACTGCGGTGGTGGTAGTGGAAGTGGTGAGAAGTGGC
TGGATTCTGGATCTATTAGGAAGTGCAGGATCTGCTAATCGATTGGATGTGGGTGAGAGAGGTGTCAA
AGGTGATCACAAAGTTTTTGGCCTTAGCAACTGGAAAGACGGATTTGCCATTTACTGAAAGGGGGAGG
AACAGGTCTGGGGTAAGTGCAGAAGTTCAGTCTTAAACACTTGGATCAGAAATATCTATTAGACATCC
AAGTTGAGATGTCAAGACGACAGGTGGATCTGGAGTCTAGGGTGAGGTCCAGGCCGGAGATATAAAT
TCGGTCATCAACACAGAACTAGAATCTAGACACATGACAGGGTTGGGGTCTGTAAATATAGAGGAGA
GGAAAAGAAAGCACAGAGTGGGCACTGAAATGTCTGCCCAATAAATTAATCCACCTATTGGAGTACA
AGGAAAATGGCTGCAATACGAATTCCATGGCTATGGCTTCTGAATCCTGTGACTCAGATTTTGGCAGA
CAAGTGCAGCTAAAGGTCCCCAGGGTTAGTTTTATCTTCATTATTCTTCTTTCATTTTTCTTCATATCTT
TAGCACCTAACAATGAACCCCAAACATCATAAGCCCTCAAGTAATGTTTGCTGAATGAATAACTTTTT
AAATTAATCTTCAAGACACGTCATGTCCTCAATTATTTTTAAATAAATAAAAAAATTTTATTTTGAGCC
ACAGAACTCATCTTTTCAAGCAACATATTTTCAAAGGAGGACTCCAGTATACAAAATAGATGGTATCA
GAGCTTCTCTGGCTAAAGACGGGTAGGGGTTGAAAGTTTTCTTTGCTCCCCTCCCCATCCATCCCCAGA
CTCCTCGGGTCTGCAGAATCCAGGAGCTGAAAACAGCCATCATCCAGGAGGCTGCAGGACTGCTGAA
AGCAGCTGTTAACTCAGGTTTTTTTTAAAATATAGGGAAATGAACACATAAGTACTTTGCTAAAGAAA
ACGTGAGTCACTGGCTGAGGAATAAAACTCATTCACTGAAGCTGAAGTACTATTTGATAAGCTAGAAA
TATTTTCCCTGAGTAGACCACTGTAAAAGAATGGCATGAACTACATAGTCAACTGAAAGACTCATTAA
TGGAAATAATCTTAAAGAACAAAAATTGTGACCTTTTTGGTGTCCACAGACTAGGGCTTTGTCTACATT
TCACCATCATCTGTTCTTGTACCACAGAAACATGGAAGTTTTCTTTCCGAAGTTCAAGCTAGATCAGAA
GTATGAGATGCATGAGCTGCTTAGGCAGATGGGAATCAGAAGAATCTTCTCACCCTTTGCTGACCTTA
GTGAACTCTCAGCTACTGGAAGAAATCTCCAAGTATCCAGGGTAAGTCAGGATCTTTCATCAGAGCCC
AACCTCAGCATGAAATGTCACCAAAACAAATGCTTTTACAAACCATTTAACTTTGATAAAATACCTAA
TTGTAGTGGAAAATTAGATTTAAGTCCCAAATACTTGAAATAGCACCCAGGTTGGATGTTTTAAGAAT
TTCAAGCAACTTCATTAAAATAACTTTTCAACTAATTTATTTTAAGCAGACCTCTCCCCCTCTGCTTAA
AGTGCTCAGGGAGAAATTTGACCCTGAAATAGAACTGGTTTACAGAGGCATCATCATTTATGTTGAAT
ACAACTTGAATAGTTCATGAAATTACACCACCTTTACAATGAAACAAACCCCTAGACATCATCTAGCC
CAACTTCTCCCTCCTTGTGGAAATCCCCTCCATAGCCCTACGAAATAGCCCTCCAACTTCTCTTCCTCTT
CATGCTTCCAGTGACATCAAACTCACCATTTCTTTGAAGAGCTGCCCAATCCACAAATAGCTAAAATT
GTTATATGTATATATATATATGTGTGTATATATATGTATATATGTATGTGTGTATAAATGTATATGTGTG
TATATGTGTGTGTGTATATATATATACACACACATATATATATATATGGAGAGAGACATACATATATAT
ATGGAGAGAGAGAGAGAGAGAGTCCTGTAACTTCTGATTCATACTTTTTGGTCCTAGTTCTATCTCTAA
AACTTCTAAGAACAAGTTTAGTCACCATCCACATAGAATCCCTTCAGTTACTCAGTGTTTCTCAGTGGA
AGGGTTCTTGGTTTTGAGGGGAACTGCTTGTTGTCCAGAGCAGTTGTGCATGTTGCAGGGAACTGGTT
AGCATTGCTGGCCCATGTTCACTAATGCCAGTAGGAAACTCCAGTCATCACTATAAAAATGCTCCCAC
ACATTTCCAAATGGCAGCTACATCTCTCTACATTCTTCCTTAGCTGTGTGGTTTAATATTTTCTTATACA
ATTGCAATTTTCAATTCCAAGAGAGACTAAAAATGGCATCCACTTAAGTAGGACACAGTAGGGTAACT
GTGGCCTGGAATCAGGTCTTACAACCTCAAGAGAGGTAAGACAATTAAATAAAACAATCCGTCAGAC
CAGCACCTGAAAGTGTTTCTGCTATGAACACATGAAAAACTGAAATGCGCTGCTGCTTTATGAAGGGT
CATCATGAAATTTAAACTGTAAATGATTAAATATTCTCCCTCTGTTTGCTCTGGGGAATTAATTTTCCTC
TAGGAAATCAGGGAATTTCCTGGAGTGAAAATCAGTGTAATTACATGTTATGTTTTCATTATCTCTTAT
AACACAGTAATTATATAGGTACATCACTCATATCACATCTTGTTTCTGTAAAAAAGGGCCTCCCAAAC
ATAGCAAGCAGCCACAGTATAGGCAGCCAGAATTCAGGAAGGCTCCAGGGACCCCTGGGCTTGGCCC
AGAAAAATGCCTCAGAGTAGTACCAGGTGCTGGGAAGCTGCTACAGAAGACTAGCCATTCCCTGCCTC
CACCTTGCCTGCCAAAAGGAAAGTCAGAGGACTCAAGGGATCCAGGGATCAAGGGATCCAGGCAGCT
TGAAAACCTTTTAGGAGCACCAGCTCAGCTCAAGAATTAGTAGCATAAATTACATGCTCAATAAAGAT
TTGATGCATGAGTGCATCCTGAGTCCATGCCCGGAATGTGTTTCACATATTCCACAATACTTCACATTG
GGTTCCTGAGGTCTCCTGGTATTGTTTAAGACTCCTGTGGCAGTCCCTGGTGCAACCCCAGACCACTCC
TCTTAACGTAGATGGGCCTGCTCCACTAAATCCCAGGAGCATGACCCCATGGGTAGGACCACTGTGAA
GAATTTCAAGGGGCTCATTTAATTCCTCCTTTGCACTGCCACACAAATGGTTTTTCACATTATTTCCTTT
TTCCAGGTTTTACAAAGAACAGTGATTGAAGTTGATGAAAGGGGCACTGAGGCAGTGGCAGGAATCTT
GTCAGAAATTACTGCTTATTCCATGCCTCCTGTCATCAAAGTGGACCGGCCATTTCATTTCATGATCTA
TGAAGAAACCTCTGGAATGCTTCTGTTTCTGGGCAGGGTGGTGAATCCGACTCTCCTATAATTCAGGA
CACGCATAAGCACTTCGTGCTGTAGTAGATGCTGAATCTGAGGTATCAAACACACACAGGATACCAGC
AATGGATGGCAGGGGAGAGTGTTCCTTTTGTTCTTAACTAGTTTAGGGTGTTCTCAAATAAATACAGTA
GTCCCCACTTATCTGAGGGGGATACATTCAAAGACCCCCAGCAGATGCCTGAAACGGTGGACAGTGCT
GAACCTTATATATATTTTTTCCTACACATACATACCTATGATAAAGTTTAATTTATAAATTAGGCACAG
TAAGAGATTAACAATAATAACAACATTAAGTAAAATGAGTTACTTGAATGCAAGCACTGCAATACCAT
AACAGTCAAACTGATTATAGAGAAGGCTACTAAGTGACTCATGGGCGAGGAGCATAGACAGTGTGGA
GACATTGGGCAAGGGGAGAATTCACATCCTGGGTGGGACAGAGCAGGACAATGCAAGATTCCATCCC
ACTACTCAGAATGGCATGCTGCTTAAGACTTTTAGATTGTTTATTTCTGGAATTTTTCATTTAATGTTTT
TGGACCATGGTTGACCATGGTTAACTGAGACTGCAGAAAGCAAAACCATGGATAAGGGAGGACTACT
ACAAAAGCATTAAATTGATACATATTTTTTAAGATGTTTGTGCAATCTGTCTGGTATTTTAAGCTTGTTT
CTAAGAACCTTAGTTACTTGGCTAAAGACTAGCTGGGTAGAATATCTTTTCTCTGTTGCTCACATATTT
TCATTTTTAAAAAGTTGCAGATGAGAACACTATGTCAAGATAAAGCCTTTGGGAGGAACACATGTAAA
CATTCTCCTTGAGTCATGTGCTTCTCTCTCTTTCCTTCTCTCTGGTGCAAAATAAGTGTTTTATTTTAATC
TATTACGGAGTCATTTCTTGCTGACTGACATCAGAAGAAAATAGCTCTAACCAGTCCTGATCACAGCA
TCTGCTTCCATGGTGCATCAAATCGCTTGGCAGAGGCATTGGCTGAATCACAGATCATCTAGTTCAATA
CCTTCATTTTACAAAGGAAAGAAAGAGGGACCCAGAAACAGGTCCATATTCTTACTTTCATGGGCCCT
AGGCACGTTTAACCTTGTAGACTCCTCCTTCCTTCATGAAGATATATATGTTCTATGGCTGCATTGGTA
GAAAGATGAATATATTCGTCTTTCAAAGTTGCATATCTAGCTTCAAAGTTATATGTCTAGCATATGGCA
ATAAGCAAAACACCTTCATGGGCCCTTACAGTACTGTCAGCCTTGGGCACTGTGTCTTCTGCATCTAGT
GGATAAGTCATACCTTATATACCAGTGGGAACAAAATACTTGTCCAAGGTCTTCCAGTGTGGCAATGG
CAGAGTCAGAAGCCTACCTTTCCTGAGTCTAGTCTCCAAGCCCTTTTTACTCTTCCTTCCATCTAAAAC
ATCTGATGGGGACCAGGTAAACAGCATGCACTACAGCTACCCATGGGGGTTAAACAGAATATAAGCA
TGAACTTTGTCCCAGGGTGAAAAGGAAAATCGTAAATATCCCTGATCTTCCTTAGGCAGTTATTTTCTG
TCACAGAAACAGAAAAGACTATATTCAGAGAATCCTGAATAGAGCTGATTTACAGTGTGAACTATGTT
AACTAAATGCCTAATTGGATTTCTGTCTGTCTGCTATCTAATGTTTAAAAAAACCTAAAATTCATTTAT
TGATTAGTTGTTTAATATAATTCAGAGTAATGTGAATAGGTAATAATATTAATATGCAGTCTAAATACT
GACTTTTCATCATTCCATAACCTGGACTGATGAAAAGTCAGTATTTAGACTGCATATTAATAAAATAA
AATTCATTCCTGTATTCATTCCAAGAGTACTAATTGACACTTATGAAGGGACAGGCAATTCTAGGCCCT
AGAGGGCCAAAGACAGAGGACTAACTCTATCTGACATTCTTAAGTCACCTTGTTTGTGTTCAATTAGTC
AGATTTGTTTGTGGAAAAATAGTAGAAAGAGGAATAAAGTAGCATCCAGTCCAATTTCCCACTTTTAA
GAGATGAAATCTGGAAAAATAAGTCTGTGAGAGCACAATACTCACTGAAATCAATATGGCCAAACCC
AGTAATAAAAAGGTACATTATTATTGAAGGATTCATATAGCATGCAGATAAAAAACTCCTGCCTTCTT
CCCACCACATACACTGCAAAGCAACAACAGCATAATAATTGTATTTAATATACTACTCTTTAAGGTAG
AAAATGGACCTATTCTATATTTTAAATATACTTTTTAATGTTCCCTCACATTTGCTTTAAGAAGTTCCTA
AGACACTCAGTTTCAGATTTCCCAAGTACACAGGCATGACAGAAAAACGCAGACCAATAAAAAATGT
AACTTACCTTACACAAATACATACACACAAATTCAGGGTTTCCAACCGAGCGGGGGAAATCTTAACAT
TGTAGAAGTCTTCACTATATATGTGTCGAGTTTTTGTTTTTGTTTTTGTTTTTGTTTTGAGACAGAGTCTT
GCTCTGTCACCCAGGCTGGAGTGCAGTGGTGCGATCTCAGCTCACTGCAACCTCCACCTCCCGGGTTC
GTGCCATTCTCCTGCCTCAGCCTCCCGAGTAGCTGGGACTACAGGCACCTGCCACCACGACCGGCTAA
TTTTTTGTATTTTAAGTAGAGATGGGGTTTCACTGTGTTAGTCAGGATGGTCTTGATCTCCTGACCTTGT
GATCTGCCCGCCTCGGCCTCCCAAAGTGCTGGGATTACAGGCATGAGCCACCACGCCCGGCCAAGTGT
CGAGTCTTAAAAATTGTTCCTACACAGACACACTCAACCACACGTTCTCACATATATATGCTGTAACA
ACTGAGAACAGGTTACTGACTTAATCTAATTCATTCTATCTTCATTGTAAAACTTCCACTCCAGCTGAA
GAGCCTGTTTCATTTCAATTCAAAGATTTCTCATATATCCACTAATTGTATGGCAAAACTGACTCATCT
CCAGACTAAGATATTCAAGCTCAGGAAGTCAAATAATAGAAATGATTTTTTAAATGTGTAAGAGGTTA
TAAAGAAAAACTTTATGTGCTCCTTATTTAACCTCTATTAAGTAAAATCCTTTATAGACCTATCTCCAT
TTCTGCAGTAAAAGTGAGCTCTACAGTTAGCTTGTAAGGCTAACTAGTGAAATTCCTGGACTTGTTCTT
AAAAATGCAAGTTTTAGTAATTAACAAAATGATGATGAAGATGTCCCTTTTCCCTACAACTACAGATG
GAGGGAGATTTTTCTTTGCCATACAACTAGCTTAAAGGATTAATTTGATAAGTTGTTAAACTGAGAACT
TTCACAAAAGTATCCATCTTGTTTTTGATATAAATGGAGATACATGTAGTTATTCATAACTGTCAGTAA
TTTGCTGTTTATCCTGTTTCTATATATCTGTCCTTGAGAGTATAATTTTAATAAATATTTCAAAGATTTT
AGGAAATGTCATGTTCTGTTAAAAAACTTCCAAAAGTAATTTTGATGAACAGTTTTGATAACTTAGTAC
TAACTAGGACTAAGACTGCAATTGACTGCTCTACATTCCTGAACTTTATAAGCAGTAGTTGTTTCTCTC
TGTCAAATCAGTGTCCCCTTTTCCCATTTGCATCATGGGAAAGTGAAACCTTATAATTCTGCTAAATTT
ATTATAACAAATACATTGAAATTCTCCATTTTATTAAATTAATAGAATGTTATGAATCAAAGCACCAA
AAAAACTGATGCAATTTTGATGTCTCGTTCTGTACCACATTCTCCAGATCTTAATATATTCAGTTCCAC
ATTATTGGTGCTAGTAGGAGACATAATGAAAACAGTTAAATGAAATCCACAGCGAGTATACTGATTAA
CCAGTACTGTCAAATTTCTCATACCTATTGAATTTTAACTACTGACAAAATGAGCAGTAACAATTCCAT
TTACCTGATTGTCCTTTGGCAAAGGATATTATTAAGAATCACTAAAAATAGCCATAAAGAAGCCATAT
GGAAGGAAGAAGGAAAACAAATGGCATGAAAAGGTCTCTCACTGAGTAACTATGCTCTTATAGTTGA
CGCTGGTATATTTCTTTTATTCACTACCTAAAAATGAACTATCTTACTCTTTAATTATAGAATAAAAAC
TGCAGGAAAGTATTTAAGACTTTTTTTCACAAACACAGGTATCTCATTAACCTATGTTTTATTTTGAGT
AAATTCATTATTCATTATTTCACATTATAAAAAGTAACCACACATACATATGCATTCACAAATTAGATC
ATCTTTATCATACATCAATATATTTTAAAAAACAAATATCTTCTAATATCAATATAGTTATATGCTGAT
TGCATTTTGAAATAGAGAAGCTGACAATAGCTTCACACGGTATATCTCAAGAACTGACAGTTTAAAAT
TAAGAACTGTATATATTCCACAGGCAAATTTTGATGGAAATATTAGCATTAGTACAAATAAATGCTGT
TGACATAGCTTAAGCATGATAGCTTGGAATAACAGCTGATTCAGACTAGATTCATCATTTTAAATAAA
GACAAGTACAATCTAAAATGTAAACAAAGTATTTATAAAATAAATTCTCTAGGAAATAAAGAAAATC
ATCAATCTATTATTTTTAAGGTATTTATAGCTCAAAGTTACCAGAAATCTTTGTGGAATTTTCACTGCC
AAATTTAAATTTGGGAATGTCCGGGTACAACATATTGTCACCACAATCCGGAGGGCCGCCAAAATCGC
AGACGGCTATTTGCATCCTTTCAGTGTGACTTTTCAAGTGGGCTTGGAGACTCATGAGAAAATGCAGT
ATCTTTCTCACCTTCCAAGTCCCCCTCCAAGTGCTTATCAAGCTAGGACAATTCAGCTGATGTAGACTT
TCATACGATTTTTAAATGCTAAAACTCTAGAACAATTAAATGGCTGGTTTCCTGCACAAATAAATGCA
GACTTGTCTCTTTTGCAGCAGTGGTTAAAGCACATTCCTAGAGATGTTTTTCATTACACTTCACTATAA
CATTGGAATTCCGTAACCACATTATTACTCAAGAAATATATATTATACCTCCTAGGGAATCTAATTTGA
AATATGAAAAGTTTAACATCAGCTGTCATTATGTCTCTCTTTCTGCTCATTAACAACAACAAAAAAAA
AAACCCAAAATTTAAAAACAAAGCCCCAGCCACTGCTTTAGCTTTTGTGTACCAATCACATTATCTCCT
GCTGCCTTTGTTTTGCCTCCTTCATCAAGCAGTTGATTTAAGGATTGGATTTTCTGGATTTTCTTTGGGA
AGAAAGAAATGAAGGAAGAGAGGGAGGGTGGGGAAGGAGGGAGTGAGAAAGGGAGAAAAAGAAAA
AAATATGAAAAATGTTATTCATATAATGTGTACAAAGTAAATTAAAAATATATAGATACTCTACTTTG
AATAATTCTAATATATGAGAAGT

The inventors employed network analysis that allows them to allocate multiple genes or proteins to a smaller number of driver processes; and to mine these processes for impactful drug targets. The approach takes advantage of information that is usually ignored in standard gene set analyses—the known and predicted interactions between genes (and proteins) and the inclusion of other genes in the same or related pathways. In particular, the inventors analysed the blood coagulation process in humans using network models which highlighted ZPI as a preferred target for haemophilia, as well as highlighting an existing drug target-antithrombin 3 (AT3, the target of Fitusiran), thereby also validating the network approach. Experimental confirmation that inhibition of ZPI is indeed a viable strategy for the treatment of haemophilia is provided in Example 9.

The inhibition disclosed herein may be of the gene or protein resulting from expression of the gene and reference to ZPI hereby explicitly incorporates a reference to inhibition of the expression or function of the gene and, separately, of the protein product.

### Definitions

The “first strand”, also called the antisense strand or guide strand herein and which can be used interchangeably herein, refers to the nucleic acid strand, e.g. the strand of an siRNA, e.g. a dsiRNA, which includes a region that is substantially complementary to a target sequence, e.g. to an mRNA. As used herein, the term “region of complementarity” refers to the region on the antisense strand that is substantially complementary to a sequence, for example a target sequence. Where the region of complementarity is not fully complementary to the target sequence, the mismatches can be in the internal or terminal regions of the molecule. In some embodiments, a double stranded nucleic acid e.g. an siRNA agent of the invention includes a nucleotide mismatch in the antisense strand.

The “second strand” (also called the sense strand or passenger strand herein, and which can be used interchangeably herein), refers to the strand of a nucleic acid e.g. siRNA that includes a region that is substantially complementary to a region of the antisense strand as that term is defined herein.

In the context of molecule comprising a nucleic acid provided with a ligand moiety, optionally also with a linker moiety, the nucleic acid of the invention may be referred to as an oligonucleotide moiety or oligonucleoside moiety

Oligonucleotides are short nucleic acid polymers. Whilst oligonucleotides contain phosphodiester bonds between the nucleoside component thereof (base plus sugar), the present invention is not limited to oligonucleotides always joined by such a phosphodiester bond between adjacent nucleosides, and other oligomers of nucleosides joined by bonds which are bonds other than a phosphate bond are contemplated. For example, a bond between nucleotides may be a phosphorothioate bond. Therefore, the term “oligonucleoside” herein covers both oligonucleotides and other oligomers of nucleosides. An oligonucleoside which is a nucleic acid having at least a portion which is an oligonucleotide is preferred according to the present invention. An oligonucleoside having one or more, or a majority of, phosphodiester backbone bonds between nucleosides is also preferred according to the present invention. An oligonucleoside having one or more, or a majority of, phosphodiester backbone bonds between nucleosides, and also having one or more phosphorothioate backbone bonds between nucleosides (typically in a terminal region of the first and/or second strands) is also preferred according to the present invention.

In some embodiments, a double stranded nucleic acid e.g. siRNA agent of the invention includes a nucleoside mismatch in the sense strand. In some embodiments, the nucleoside mismatch is, for example, within 5, 4, 3, 2, or 1 nucleosides from the 3′-end of the nucleic acid e.g. siRNA.

In another embodiment, the nucleoside mismatch is, for example, in the 3′-terminal nucleoside of the nucleic acid e.g. siRNA.

A “target sequence” (which may be called a target RNA or a target mRNA) refers to a contiguous portion of the nucleoside sequence of an mRNA molecule formed during the transcription of a gene, including mRNA that is a product of RNA processing of a primary transcription product, or can be a contiguous portion of the nucleotide sequence of any RNA molecule such as a LNCRNA which it is desired to inhibit.

The target sequence may be from about 10-35 nucleosides in length, e.g., about 15-30 nucleosides in length. For example, the target sequence can be from about 15-30 nucleosides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleosides in length. Ranges and lengths intermediate to the above recited ranges and lengths are also contemplated to be part of the invention.

The term “ribonucleoside” or “nucleoside” can also refer to a modified nucleoside as further detailed below.

A nucleic acid can be a DNA or an RNA, and can comprise modified nucleosides. RNA is a preferred nucleic acid.

The terms “iRNA”, “siRNA”, “RNAi agent,” and “iRNA agent,” “RNA interference agent” as used interchangeably herein, refer to an agent that contains RNA, and which mediates the targeted cleavage of an RNA transcript via an RNA-induced silencing complex (RISC) pathway. siRNA directs the sequence-specific degradation of mRNA through RNA interference (RNAi).

A double stranded RNA is referred to herein as a “double stranded siRNA (dsiRNA) agent”, “double stranded siRNA (dsiRNA) molecule”, “double stranded RNA (dsRNA) agent”, “double stranded RNA (dsRNA) molecule”, “dsiRNA agent”, “dsiRNA molecule”, or “dsiRNA”, which refers to a complex of ribonucleic acid molecules, having a duplex structure comprising two anti-parallel and substantially complementary nucleic acid strands, referred to as having “sense” and “antisense” orientations with respect to a target RNA. The majority of nucleosides of each strand of the nucleic acid, e.g. a dsRNA molecule, are preferably ribonucleosides, but in that case each or both strands can also include one or more non-ribonucleosides, e.g., a deoxyribonucleoside or a modified ribonucleoside. In addition, as used in this specification, an “siRNA” may include ribonucleosides with chemical modifications.

The term “modified nucleoside” refers to a nucleoside having, independently, a modified sugar moiety, a modified internucleoside linkage, or modified nucleobase, or any combination thereof. Thus, the term modified nucleoside encompasses substitutions, additions or removal of, e.g., a functional group or atom, to internucleoside linkages, sugar moieties, or nucleobases. Any such modifications, as used in a siRNA type molecule, are encompassed by “iRNA” or “RNAi agent” or “siRNA” or “siRNA agent” for the purposes of this specification and claims.

The duplex region of a nucleic acid of the invention e.g. a dsRNA may range from about 9 to 40 base pairs in length such as 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length.

The two strands forming the duplex structure may be different portions of one larger molecule, or they may be separate molecules e.g. RNA molecules.

The term “nucleoside overhang” refers to at least one unpaired nucleoside that extends from the duplex structure of a double stranded nucleic acid. A ds nucleic acid can comprise an overhang of at least one nucleoside; alternatively the overhang can comprise at least two nucleosides, at least three nucleosides, at least four nucleosides, at least five nucleosides, or more. A nucleoside overhang can comprise or consist of a nucleoside analog, including a deoxynucleoside. The overhang(s) can be on the sense strand, the antisense strand, or any combination thereof. Furthermore, the/nucleoside(s) of an overhang can be present on the 5′-end, 3′-end, or both ends of either an antisense or sense strand.

In certain embodiments, the antisense strand has a 1-10 nucleoside, e.g., 0-3, 1-3, 2-4, 2-5, 4-10, 5-10, 1, 2, 3, 4, 5, 6, 7, 8, 9, or 10 nucleoside overhang at the 3′-end or the 5′-end.

“Blunt” or “blunt end” means that there are no unpaired nucleoside at that end of the double stranded nucleic acid, i.e., no nucleoside overhang. The nucleic acids of the invention include those with no nucleoside overhang at one end or with no nucleoside overhangs at either end.

Unless otherwise indicated, the term “complementary,” when used to describe a first nucleoside sequence in relation to a second nucleoside sequence, refers to the ability of an oligonucleoside comprising the first nucleoside sequence to hybridize and form a duplex structure under certain conditions with an oligonucleoside or polynucleoside comprising the second nucleoside sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press).

Complementary sequences within nucleic acid e.g. a dsiRNA, as described herein, include base-pairing of the oligonucleoside or polynucleoside comprising a first nucleoside sequence to an oligonucleoside or polynucleoside comprising a second nucleoside sequence over the entire length of one or both nucleoside sequences. Such sequences can be referred to as “fully complementary” with respect to each other herein. However, where a first sequence is referred to as “substantially complementary” or “partially complementary” with respect to a second sequence herein, the two sequences can be fully complementary, or they can form one or more mismatched base pairs, such as 2, 4, or 5 mismatched base pairs, but preferably not more than 5, while retaining the ability to hybridize under the conditions most relevant to their ultimate application, e.g., inhibition of gene expression via a RISC pathway. Overhangs shall not be regarded as mismatches with regard to the determination of complementarity. For example, a nucleic acid e.g. dsRNA comprising one oligonucleoside 17 nucleosides in length and another oligonucleoside 19 nucleosides in length, wherein the longer oligonucleoside comprises a sequence of 17 nucleosides that is fully complementary to the shorter oligonucleoside, can yet be referred to as “fully complementary”.

“Complementary” sequences, as used herein, can also include, or be formed entirely from, non-Watson-Crick base pairs or base pairs formed from non-natural and modified nucleosides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially/partially complementary” herein can be used with respect to the base matching between the sense strand and the antisense strand of a nucleic acid e.g. dsiRNA, or between the antisense strand of a double stranded nucleic acid e.g. siRNA agent and a target sequence.

Within the present invention, the second strand of the nucleic acid according to the invention, in particular a dsiRNA for inhibiting ZPI, is at least partially complementary to the first strand of said nucleic acid. In certain embodiments, a first and second strand of a nucleic acid according to the invention are partially complementary if they form a duplex region having a length of at least 17 base pairs and comprising not more than 1, 2, 3, 4, or 5 mismatched base pairs.

In certain embodiments, a first and second strand of the nucleic acid according to the invention are partially complementary if they form a duplex region having a length of 19 base pairs and comprising not more than 1, 2, 3, 4, or 5 mismatched base pairs. In certain embodiments, a first and second strand of the nucleic acid according to the invention are partially complementary if they form a duplex region having a length of 21 base pairs comprising not more than 1, 2, 3, 4, or 5 mismatched base pairs.

Alternatively, a first and second strand of the nucleic acid according to the invention are partially complementary if they form a duplex region having a length of at least 17 base pairs, wherein at least 14, 15, 16 or 17 of said base pairs are complementary base pairs, in particular Watson-Crick base pairs.

In certain embodiments, a first and second strand of the nucleic acid according to the invention are partially complementary if they form a duplex region having a length of 19 base pairs, wherein at least 14, 15, 16, 17, 18 or all 19 base pairs are complementary base pairs, in particular Watson-Crick base pairs. In certain embodiments, a first and second strand of the nucleic acid according to the invention are partially complementary if they form a duplex region having a length of 21 base pairs, wherein at least 16, 17, 18, 19, 20 or all 21 base pairs are complementary base pairs, in particular Watson-Crick base pairs. As used herein, a nucleic acid that is “substantially complementary” or “partially complementary” to at least part of a messenger RNA (mRNA) refers to a polynucleoside that is substantially or partially complementary to a contiguous portion of the mRNA of interest (e.g., an mRNA encoding a gene). In certain embodiments, the contiguous portion of the mRNA is a sequence as listed in Table 1, i.e., any one of SEQ ID NOs:2-121. For example, a polynucleoside is complementary to at least a part of an mRNA of a gene of interest if the sequence is substantially or partially complementary to a non-interrupted portion of an mRNA encoding that gene.

Accordingly, in some preferred embodiments, the antisense oligonucleosides as disclosed herein are fully complementary to the target gene sequence.

In other embodiments, the antisense oligonucleosides disclosed herein are substantially or partially complementary to a target RNA sequence and comprise a contiguous nucleoside sequence which is at least about 80% complementary over its entire length to the equivalent region of the target RNA sequence, such as at least about 85%, 86%, 87%, 88%, 89%, about 90%, 91%, 92%, 93%, 94%, 95%, 96%, 97%, 98%, or 99% complementary or 100% complementary.

In certain embodiments, the first (antisense) strand of a nucleic acid according to the invention is partially or fully complementary to a contiguous portion of RNA transcribed from the ZPI gene. In certain embodiments, the first strand of the nucleic acid according to the invention is partially or fully complementary to a contiguous portion of at least 17 nucleosides of the ZPI mRNA. In certain embodiments, the first strand of the nucleic acid according to the invention is partially or fully complementary to a contiguous portion of 17, 18, 19, 20, 21, 22 or 23 nucleosides of the ZPI mRNA. In certain embodiments, the first strand of the nucleic acid according to the invention is partially or fully complementary to a contiguous portion of 17, 18, 19, 20, 21, 22 or 23 nucleosides of any one of the sequences as listed in Table 1, i.e., any one of SEQ ID NOs: 2-121.

In certain embodiments, the first (antisense) strand of the nucleic acid according to the invention is partially complementary to a contiguous portion of the ZPI mRNA if it comprises a contiguous nucleoside sequence of at least 17 nucleosides, wherein at least 14, 15, 16 or 17 nucleosides of said contiguous nucleoside sequence are complementary to a contiguous portion of the ZPI mRNA. In certain embodiments, the first strand of the nucleic acid according to the invention comprises a contiguous nucleoside sequence of at least 17 nucleosides, wherein at least 14, 15, 16 or 17 nucleosides of said contiguous nucleoside sequence are complementary to a contiguous portion of any one of the sequences listed in Table 1, i.e., any one of SEQ ID NOs: 2-121. In certain embodiments, the first strand of the nucleic acid according to the invention comprises a contiguous nucleoside sequence of 19 nucleosides, wherein at least 14, 15, 16, 17, 18 or all 19 nucleosides of said contiguous nucleoside sequence are complementary to a contiguous portion of any one of the sequences listed in Table 1, i.e., any one of SEQ ID NOs: 2-121. In certain embodiments, the first strand of the nucleic acid according to the invention comprises a contiguous nucleoside sequence of 23 nucleosides, wherein at least 18, 19, 20, 21, 22 or all 23 nucleosides of said contiguous nucleoside sequence are complementary to a contiguous portion of any one of the sequences listed in Table 1, i.e., any one of SEQ ID NOs: 2-101.

In some embodiments, a nucleic acid e.g. an siRNA of the invention includes a sense strand that is substantially or partially complementary to an antisense polynucleoside which, in turn, is complementary to a target gene sequence and comprises a contiguous nucleoside sequence which is at least about 80% complementary over its entire length to the equivalent region of the nucleoside sequence of the antisense strand, such as about 85%, 86%, 87%, 88%, 89%, 90%, 91%, 92%, 93%, 94%, 95%, 96%, 97%, 98%, or 99% complementary, or 100% complementary.

In some embodiments, a nucleic acid e.g. an siRNA of the invention includes an antisense strand that is substantially or partially complementary to the target sequence and comprises a contiguous nucleoside sequence which is at least 80% complementary over its entire length to the target sequence such as about 85%, 86%, 87%, 88%, 89%, 90%, 91%, 92%, 93%, 94%, 95%, 96%, 97%, 98%, or 99% complementary, or 100% complementary.

As used herein, a “subject” is an animal, such as a mammal, including a primate (such as a human, a non-human primate, e.g., a monkey, and a chimpanzee), or a non-primate or a bird that expresses the target gene, either endogenously or heterologously, when the target gene sequence has sufficient complementarity to the nucleic acid e.g. iRNA agent to promote target knockdown. In certain preferred embodiments, the subject is a human.

The terms “treating” or “treatment” refer to a beneficial or desired result including, but not limited to, alleviation or amelioration of one or more symptoms associated with gene expression. “Treatment” can also mean prolonging survival as compared to expected survival in the absence of treatment. Treatment can include prevention of development of co-morbidities, e.g.reduced liver damage in a subject with a hepatic infection.

“Therapeutically effective amount,” as used herein, is intended to include the amount of a nucleic acid e.g. an iRNA that, when administered to a patient for treating a subject having disease, is sufficient to effect treatment of the disease (e.g., by diminishing, ameliorating or maintaining the existing disease or one or more symptoms of disease or its related comorbidities).

The phrase “pharmaceutically acceptable” is employed herein to refer to compounds, materials, compositions, or dosage forms which are suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated.

Where a value or range of values of a parameter are recited, it is intended that values and ranges intermediate to the recited values are also intended to be part of this invention.

The articles “a” and “an” are used herein to refer to one or to more than one (i.e. to at least one) of the grammatical object of the article.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means+10%. In certain embodiments, about means+5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleosides of a 21 nucleoside nucleic acid molecule” means that 18, 19, 20, or 21 nucleosides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleosides” has a 2, 1, or 0 nucleoside overhang. When “no more than” is present before a series of numbers or a range, it is understood that “no more than” can modify each of the numbers in the series or range.

The terminal region of a strand is the last 5 nucleotides from the 5′ or the 3′ end.

A nucleobase sequence is the sequence of the bases of the nucleic acid in an oligomer.

Various embodiments of the invention can be combined as determined appropriate by one of skill in the art.

### Target

A target for inhibition disclosed herein may be, without limitation, an mRNA, polypeptide, protein, or gene.

These targets are a target the inhibition of which helps in the prevention or treatment of a disease related to a disorder of haemostasis, such as haemophilia.

The target for inhibition is ZPI, and inhibition may be effected by inhibition of expression or function of the ZPI gene or protein or both.

In a preferred embodiment, the target in an mRNA expressed from the ZPI gene. Exemplary target sequences on the ZPI mRNA are listed below in Table 1.

TABLE 1

| | Oligonucleotide mRNA target | Starting position |
|---|---|---|
| SEQ ID NO | sequence 5′→3′ | on NM_016186.3 |
| SEQ ID NO: 2 | UUUGCCUUCAUCCACAAGGAUUU | 991 |
| SEQ ID NO: 3 | GCUGCGAAAGAUCUCCAUGAGGC | 756 |
| SEQ ID NO: 4 | AUGCUGGUGGUCCUCAUGGAGAA | 1390 |
| SEQ ID NO: 5 | CUGCGAAAGAUCUCCAUGAGGCA | 757 |
| SEQ ID NO: 6 | AAGUAUGAGAUGCAUGAGCUGCU | 1531 |
| SEQ ID NO: 7 | CUGUUUGAUGAGAUUAAUCCUGA | 1156 |
| SEQ ID NO: 8 | GAUGAGAUUAAUCCUGAAACCAA | 1162 |
| SEQ ID NO: 9 | UUUGAUGAGAUUAAUCCUGAAAC | 1159 |
| SEQ ID NO: 10 | UGAUGAGAUUAAUCCUGAAACCA | 1161 |
| SEQ ID NO: 11 | UUGAUGAGAUUAAUCCUGAAACC | 1160 |
| SEQ ID NO: 12 | AACUGUUUGAUGAGAUUAAUCCU | 1154 |
| SEQ ID NO: 13 | AGUUUUGCCUUCAUCCACAAGGA | 988 |
| SEQ ID NO: 14 | UGCGAAAGAUCUCCAUGAGGCAC | 758 |
| SEQ ID NO: 15 | CAUGCUGGUGGUCCUCAUGGAGA | 1389 |
| SEQ ID NO: 16 | UGCCUUCAUCCACAAGGAUUUUG | 993 |
| SEQ ID NO: 17 | GCGAAAGAUCUCCAUGAGGCACG | 759 |
| SEQ ID NO: 18 | CCUUCAUCCACAAGGAUUUUGAU | 995 |
| SEQ ID NO: 19 | UGUUUGAUGAGAUUAAUCCUGAA | 1157 |
| SEQ ID NO: 20 | UUUUGCCUUCAUCCACAAGGAUU | 990 |
| SEQ ID NO: 21 | AAGAUCUCCAUGAGGCACGAUGG | 763 |
| SEQ ID NO: 22 | ACCAUGCUGGUGGUCCUCAUGGA | 1387 |
| SEQ ID NO: 23 | GUUUUGCCUUCAUCCACAAGGAU | 989 |
| SEQ ID NO: 24 | UUGCCUUCAUCCACAAGGAUUUU | 992 |
| SEQ ID NO: 25 | CCUACCAAGGAAAUGCCACCAUG | 1370 |
| SEQ ID NO: 26 | GUUUGAUGAGAUUAAUCCUGAAA | 1158 |
| SEQ ID NO: 27 | GCCUUCAUCCACAAGGAUUUUGA | 994 |
| SEQ ID NO: 28 | CGAAAGAUCUCCAUGAGGCACGA | 760 |
| SEQ ID NO: 29 | ACUGUUUGAUGAGAUUAAUCCUG | 1155 |
| SEQ ID NO: 30 | CCAUGCUGGUGGUCCUCAUGGAG | 1388 |
| SEQ ID NO: 31 | GAGUUUUGCCUUCAUCCACAAGG | 987 |
| SEQ ID NO: 32 | UGCCACCAUGCUGGUGGUCCUCA | 1383 |
| SEQ ID NO: 33 | GCCACCAUGCUGGUGGUCCUCAU | 1384 |
| SEQ ID NO: 34 | GAAAGAUCUCCAUGAGGCACGAU | 761 |
| SEQ ID NO: 35 | GGAGUUUUGCCUUCAUCCACAAG | 986 |
| SEQ ID NO: 36 | CCACCAUGCUGGUGGUCCUCAUG | 1385 |
| SEQ ID NO: 37 | AAAGAUCUCCAUGAGGCACGAUG | 762 |
| SEQ ID NO: 38 | CACCAUGCUGGUGGUCCUCAUGG | 1386 |
| SEQ ID NO: 39 | UUUGCCUCCACCUUUGACAAGAA | 1321 |
| SEQ ID NO: 40 | UGCCUCCACCUUUGACAAGAAUU | 1323 |
| SEQ ID NO: 41 | ACCAUUAAGGUGCCCAUGAUGUA | 1285 |
| SEQ ID NO: 42 | AACUGCCCUACCAAGGAAAUGCC | 1364 |
| SEQ ID NO: 43 | CUCAAACUGCCCUACCAAGGAAA | 1360 |
| SEQ ID NO: 44 | CCUCAAACUGCCCUACCAAGGAA | 1359 |
| SEQ ID NO: 45 | GCCUCCACCUUUGACAAGAAUUU | 1324 |
| SEQ ID NO: 46 | GUCGACACUUUCCACCUGGACAA | 1255 |
| SEQ ID NO: 47 | ACACUUUCCACCUGGACAAGUAC | 1259 |
| SEQ ID NO: 48 | UCAAACUGCCCUACCAAGGAAAU | 1361 |
| SEQ ID NO: 49 | AAACUGCCCUACCAAGGAAAUGC | 1363 |
| SEQ ID NO: 50 | GACACUUUCCACCUGGACAAGUA | 1258 |
| SEQ ID NO: 51 | GACCAUUAAGGUGCCCAUGAUGU | 1284 |
| SEQ ID NO: 52 | GUUUGCCUCCACCUUUGACAAGA | 1320 |
| SEQ ID NO: 53 | ACUUUCCACCUGGACAAGUACAA | 1261 |
| SEQ ID NO: 54 | UGUCCUCAAACUGCCCUACCAAG | 1356 |
| SEQ ID NO: 55 | CAAACUGCCCUACCAAGGAAAUG | 1362 |
| SEQ ID NO: 56 | GUCCUCAAACUGCCCUACCAAGG | 1357 |
| SEQ ID NO: 57 | AUGUCCUCAAACUGCCCUACCAA | 1355 |
| SEQ ID NO: 58 | UCGACACUUUCCACCUGGACAAG | 1256 |
| SEQ ID NO: 59 | AGUUUGCCUCCACCUUUGACAAG | 1319 |
| SEQ ID NO: 60 | CGACACUUUCCACCUGGACAAGU | 1257 |
| SEQ ID NO: 61 | CUUUCCACCUGGACAAGUACAAG | 1262 |
| SEQ ID NO: 62 | CACUUUCCACCUGGACAAGUACA | 1260 |
| SEQ ID NO: 63 | UCCUCAAACUGCCCUACCAAGGA | 1358 |
| SEQ ID NO: 64 | GAUUACAUCUUGUUCAAAGGGAA | 1198 |
| SEQ ID NO: 65 | AAUGCCACCAUGCUGGUGGUCCU | 1381 |
| SEQ ID NO: 66 | UUUAUCCAAGAGGUAUUUUGAUA | 1038 |
| SEQ ID NO: 67 | GGAAAUGCCACCAUGCUGGUGGU | 1378 |
| SEQ ID NO: 68 | GGAUUACAUCUUGUUCAAAGGGA | 1197 |
| SEQ ID NO: 69 | AUCUCCAUGAGGCACGAUGGCAA | 766 |
| SEQ ID NO: 70 | AUUCCAUGCCUCCUGUCAUCAAA | 1721 |
| SEQ ID NO: 71 | UUAUUCCAUGCCUCCUGUCAUCA | 1719 |
| SEQ ID NO: 72 | ACCAAGGAAAUGCCACCAUGCUG | 1373 |
| SEQ ID NO: 73 | GCUGGUGGUCCUCAUGGAGAAAA | 1392 |
| SEQ ID NO: 74 | ACAUCUUGUUCAAAGGGAAAUGG | 1202 |
| SEQ ID NO: 75 | CCAAGGAAAUGCCACCAUGCUGG | 1374 |
| SEQ ID NO: 76 | UUGCCUCCACCUUUGACAAGAAU | 1322 |
| SEQ ID NO: 77 | GGGAGUUUUGCCUUCAUCCACAA | 985 |
| SEQ ID NO: 78 | CUGCUGCGAAAGAUCUCCAUGAG | 754 |
| SEQ ID NO: 79 | CAAGUUUGCCUCCACCUUUGACA | 1317 |
| SEQ ID NO: 80 | AAGUUUGCCUCCACCUUUGACAA | 1318 |
| SEQ ID NO: 81 | UACAUCUUGUUCAAAGGGAAAUG | 1201 |
| SEQ ID NO: 82 | GGGGAGUUUUGCCUUCAUCCACA | 984 |
| SEQ ID NO: 83 | UGCUGCGAAAGAUCUCCAUGAGG | 755 |
| SEQ ID NO: 84 | UUCCAUGCCUCCUGUCAUCAAAG | 1722 |
| SEQ ID NO: 85 | UCUGUUUCUGGGCAGGGUGGUGA | 1794 |
| SEQ ID NO: 86 | CAGCCUGCUGCGAAAGAUCUCCA | 750 |
| SEQ ID NO: 87 | CAAACUGUUUGAUGAGAUUAAUC | 1152 |
| SEQ ID NO: 88 | CCAUGCCUCCUGUCAUCAAAGUG | 1724 |
| SEQ ID NO: 89 | GAAGUAUGAGAUGCAUGAGCUGC | 1530 |
| SEQ ID NO: 90 | AGAAGUAUGAGAUGCAUGAGCUG | 1529 |
| SEQ ID NO: 91 | CAUGUCCUCAAACUGCCCUACCA | 1354 |
| SEQ ID NO: 92 | AAACUGUUUGAUGAGAUUAAUCC | 1153 |
| SEQ ID NO: 93 | AAGACCAUUAAGGUGCCCAUGAU | 1282 |
| SEQ ID NO: 94 | AGACCAUUAAGGUGCCCAUGAUG | 1283 |
| SEQ ID NO: 95 | CUGUUUCUGGGCAGGGUGGUGAA | 1795 |
| SEQ ID NO: 96 | GCUUCUGUUUCUGGGCAGGGUGG | 1791 |
| SEQ ID NO: 97 | AGUUUUCUUUCCGAAGUUCAAGC | 1500 |
| SEQ ID NO: 98 | GUUUUCUUUCCGAAGUUCAAGCU | 1501 |
| SEQ ID NO: 99 | CUUCUGUUUCUGGGCAGGGUGGU | 1792 |
| SEQ ID NO: 100 | UCAUGUCCUCAAACUGCCCUACC | 1353 |
| SEQ ID NO: 101 | AGUCGACACUUUCCACCUGGACA | 1254 |
| SEQ ID NO: 102 | CCUUCAUCCACAAGGAUUU | 995 |
| SEQ ID NO: 103 | CGAAAGAUCUCCAUGAGGC | 760 |
| SEQ ID NO: 104 | UGGUGGUCCUCAUGGAGAA | 1394 |
| SEQ ID NO: 105 | GAAAGAUCUCCAUGAGGCA | 761 |
| SEQ ID NO: 106 | AUGAGAUGCAUGAGCUGCU | 1535 |
| SEQ ID NO: 107 | UUGAUGAGAUUAAUCCUGA | 1160 |
| SEQ ID NO: 108 | AGAUUAAUCCUGAAACCAA | 1166 |
| SEQ ID NO: 109 | AUGAGAUUAAUCCUGAAAC | 1163 |
| SEQ ID NO: 110 | GAGAUUAAUCCUGAAACCA | 1165 |
| SEQ ID NO: 111 | UGAGAUUAAUCCUGAAACC | 1164 |
| SEQ ID NO: 112 | GUUUGAUGAGAUUAAUCCU | 1158 |
| SEQ ID NO: 113 | UUGCCUUCAUCCACAAGGA | 992 |
| SEQ ID NO: 114 | AAAGAUCUCCAUGAGGCAC | 762 |
| SEQ ID NO: 115 | CUGGUGGUCCUCAUGGAGA | 1393 |
| SEQ ID NO: 116 | UUCAUCCACAAGGAUUUUG | 997 |
| SEQ ID NO: 117 | AAGAUCUCCAUGAGGCACG | 763 |
| SEQ ID NO: 118 | CAUCCACAAGGAUUUUGAU | 999 |
| SEQ ID NO: 119 | UGAUGAGAUUAAUCCUGAA | 1161 |
| SEQ ID NO: 120 | GCCUUCAUCCACAAGGAUU | 994 |
| SEQ ID NO: 121 | UCUCCAUGAGGCACGAUGG | 767 |

It is to be understood that SEQ ID NOs: 2 to 121 relate to human (Homo sapiens) mRNA sequences.

### Disease/Conditions

The invention relates to an inhibitor suitable for use, or for use, in treatment of a disease related to a disorder of haemostasis, such as haemophilia.

Haemophilia, or hemophilia is a mostly inherited genetic disorder that impairs the body's ability to make blood clots, a process needed to stop bleeding. This results in subjects bleeding for a longer time after an injury, easy bruising, and an increased risk of bleeding inside joints or the brain. Subjects with a mild case of the disease may have symptoms only after an accident or during surgery. Bleeding into a joint can result in permanent damage while bleeding in the brain can result in long term headaches, seizures, or a decreased level of consciousness.

There are two main types of haemophilia: haemophilia A, which occurs due to low amounts of clotting factor VIII, and haemophilia B, which occurs due to low levels of clotting factor IX. They are typically inherited from one's parents through an X chromosome carrying a nonfunctional gene. Rarely a new mutation may occur during early development or haemophilia may develop later in life due to antibodies forming against a clotting factor. Other types include haemophilia C, which occurs due to low levels of factor XI, Von Willebrand disease, which occurs due to low levels of a substance called von Willebrand factor, and parahaemophilia, which occurs due to low levels of factor V. Haemophilia A, B, and C prevent the intrinsic pathway from functioning properly; this clotting pathway is necessary when there is damage to the endothelium of a blood vessel. Acquired haemophilia is associated with cancers, autoimmune disorders, and pregnancy. Diagnosis is by testing the blood for its ability to clot and its levels of clotting factors.

In certain embodiments, the inhibitor of the present invention is suitable for treatment, or for treatment of haemophilia A, B and/or C. In certain embodiments, the inhibitor of the present invention is suitable for treatment, or for treatment of haemophilia A and/or B. In certain embodiments, the inhibitor of the present invention is suitable for treatment, or for treatment of acquired haemophilia. In certain embodiments, the inhibitor of the present invention is suitable for treatment, or for treatment of Willebrand disease. In certain embodiments, the inhibitor of the present invention is suitable for treatment, or for treatment of parahaemophilia.

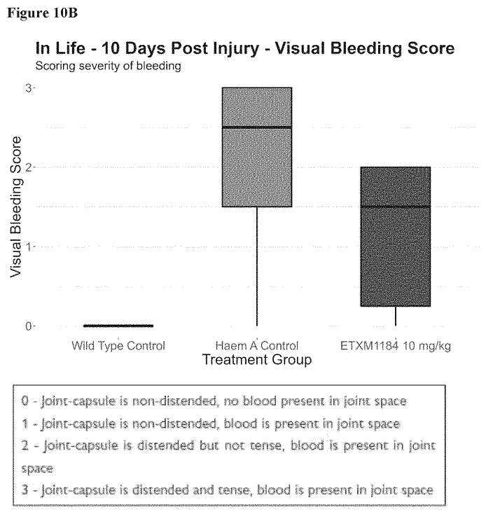

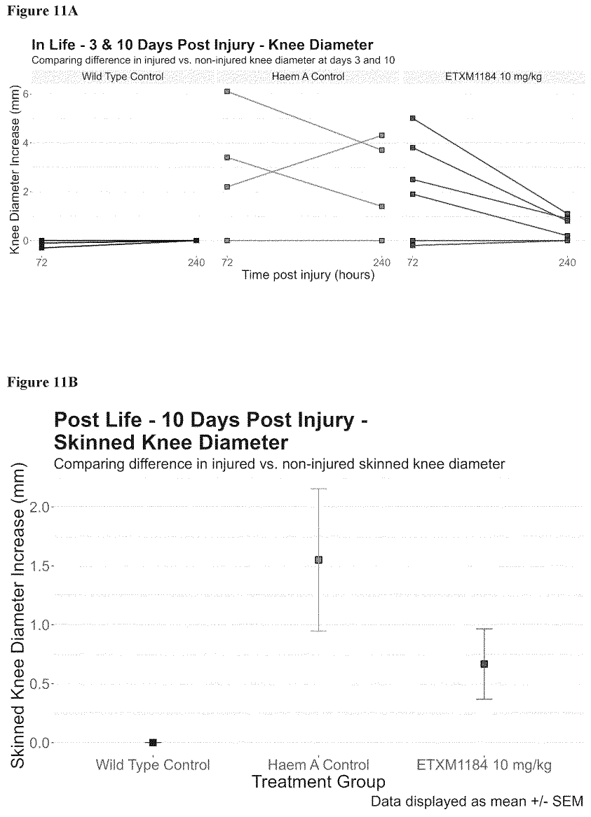

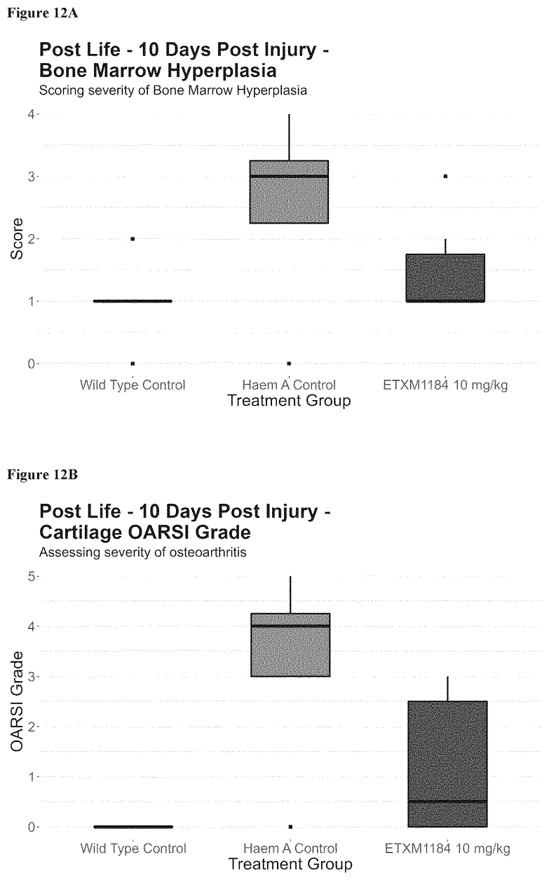

Without wishing to being bound by theory, treatment with the inhibitor of the invention results in a boost of clotting factor levels such that bleeding can be reduced or prevented, as demonstrated herein in FIG. 10. Thus, in a preferred embodiment, treatment with the inhibitor of the invention reduces or prevents bleeding episodes in a subject suffering from haemophilia. In another preferred embodiment, treatment with the inhibitor of the invention reduces or prevents bleeding into a joint of a subject suffering from haemophilia. In certain embodiments, treatment with the inhibitor of the invention reduces or prevents bleeding into a muscle or into the brain of a subject suffering from haemophilia.

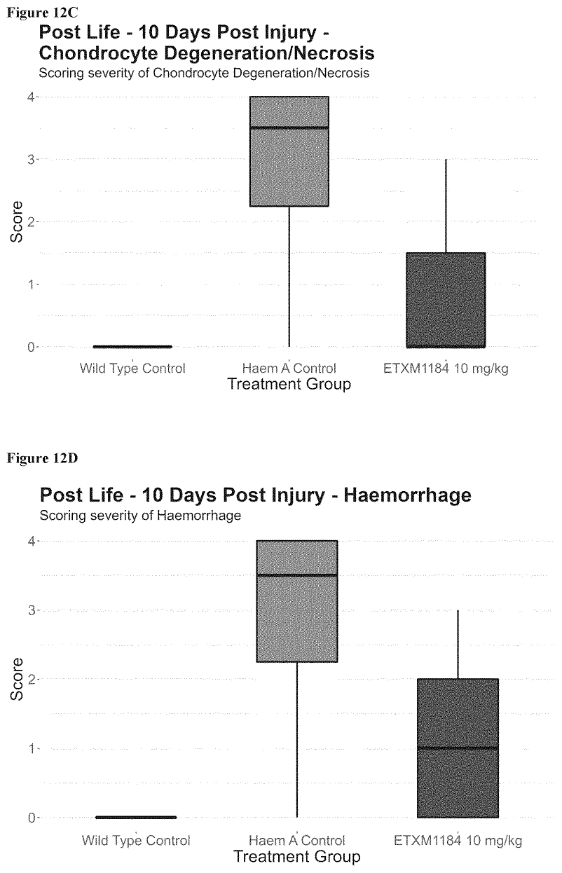

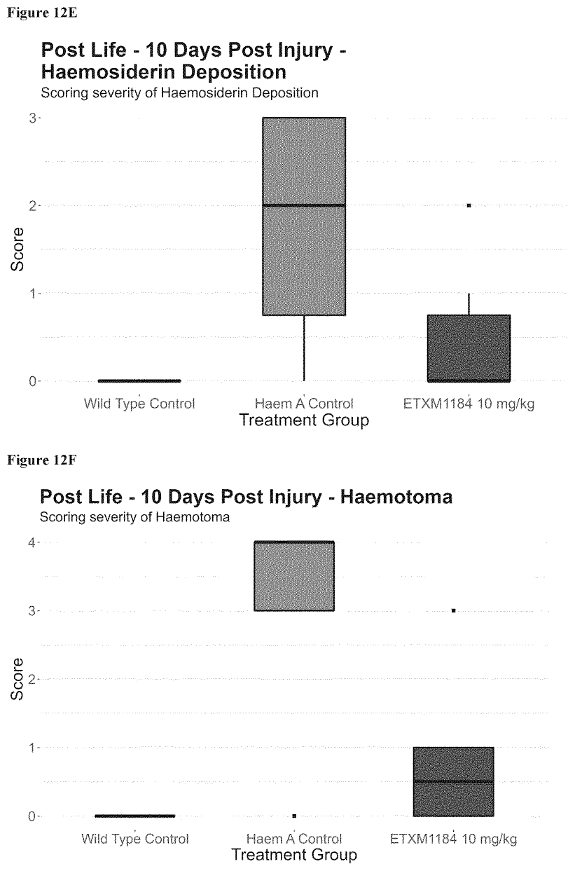

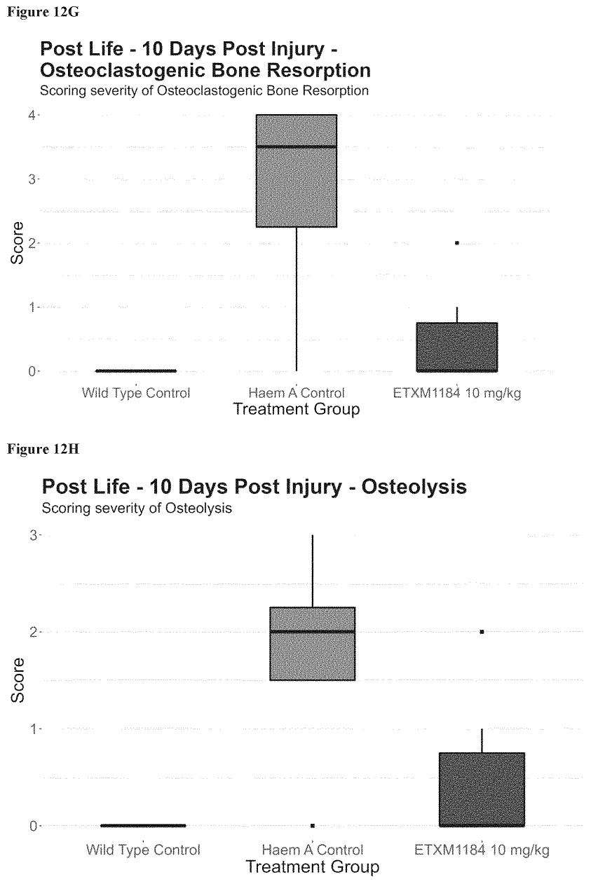

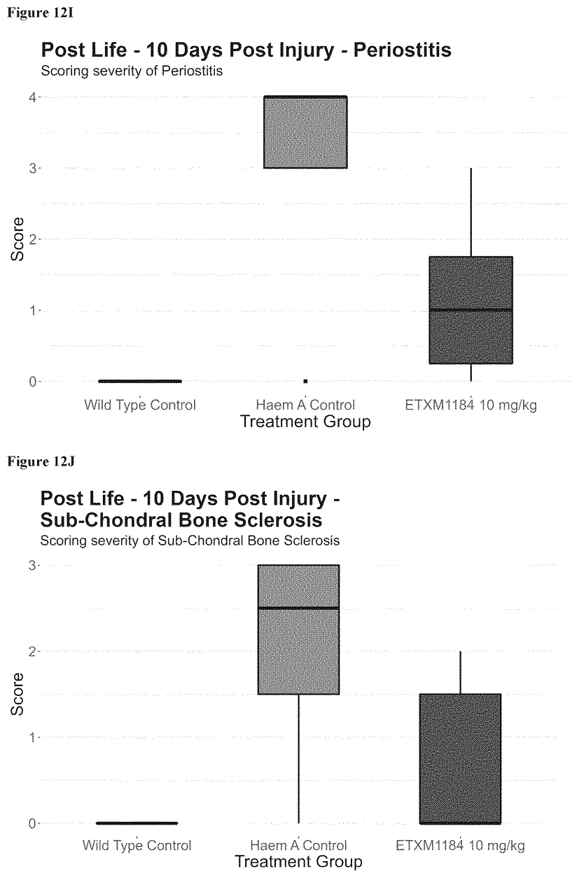

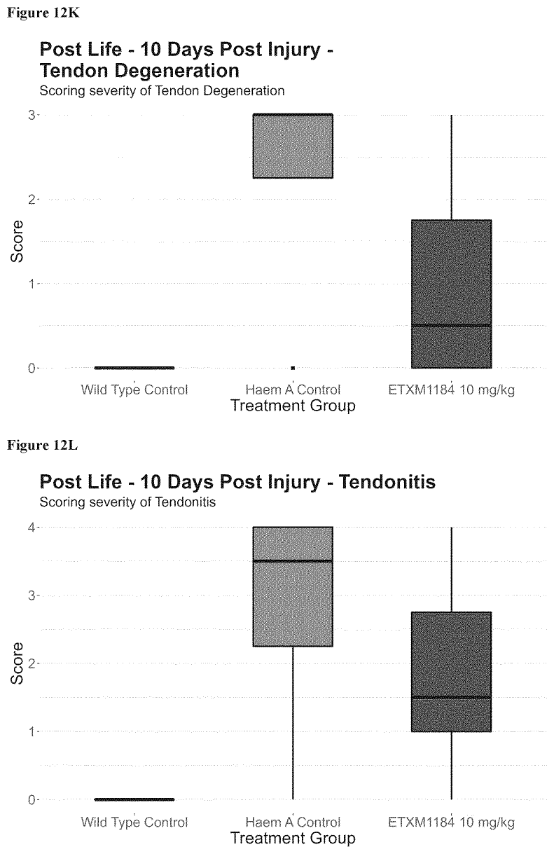

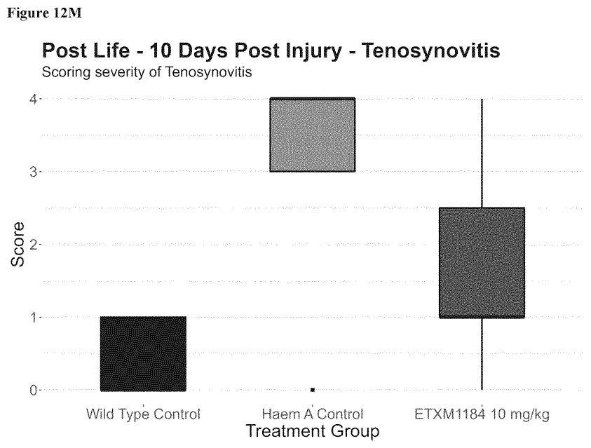

Alternatively or in addition, treatment of a subject, preferably a subject having a disorder of haemostasis, such as haemophilia, with the inhibitor of the invention may result in one or more of more of the following: In certain embodiments, treatment of a subject, preferably a subject having a disorder of haemostasis, such as haemophilia, with the inhibitor of the invention results in reduced bone marrow hyperplasia. As shown in FIG. 12A, treatment of Haem A mice with an inhibitor of the invention significantly reduced bone marrow hyperplasia in said mice.

In certain embodiments, treatment of a subject, preferably a subject having a disorder of haemostasis, such as haemophilia, with the inhibitor of the invention results in reduced osteoarthritis. As shown in FIG. 12B, treatment of Haem A mice with an inhibitor of the invention significantly reduced osteoarthritis in said mice.

In certain embodiments, treatment of a subject, preferably a subject having a disorder of haemostasis, such as haemophilia, with the inhibitor of the invention results in reduced chondrocyte degeneration/necrosis. As shown in FIG. 12C, treatment of Haem A mice with an inhibitor of the invention significantly reduced chondrocyte degeneration/necrosis in said mice.

In certain embodiments, treatment of a subject, preferably a subject having a disorder of haemostasis, such as haemophilia, with the inhibitor of the invention results in reduced haemorrhage. As shown in FIG. 12D, treatment of Haem A mice with an inhibitor of the invention significantly reduced haemorrhage in said mice.

In certain embodiments, treatment of a subject, preferably a subject having a disorder of haemostasis, such as haemophilia, with the inhibitor of the invention results in reduced haemosiderin deposition. As shown in FIG. 12E, treatment of Haem A mice with an inhibitor of the invention significantly reduced haemosiderin deposition in said mice.

In certain embodiments, treatment of a subject, preferably a subject having a disorder of haemostasis, such as haemophilia, with the inhibitor of the invention results in reduced occurrence of haematoma. As shown in FIG. 12F, treatment of Haem A mice with an inhibitor of the invention significantly reduced haematoma in said mice.

In certain embodiments, treatment of a subject, preferably a subject having a disorder of haemostasis, such as haemophilia, with the inhibitor of the invention results in reduced osteoclastogenic bone resorption. As shown in FIG. 12G, treatment of Haem A mice with an inhibitor of the invention significantly reduced osteoclastogenic bone resorption in said mice.

In certain embodiments, treatment of a subject, preferably a subject having a disorder of haemostasis, such as haemophilia, with the inhibitor of the invention results in reduced osteolysis. As shown in FIG. 12H, treatment of Haem A mice with an inhibitor of the invention significantly reduced osteolysis in said mice.

In certain embodiments, treatment of a subject, preferably a subject having a disorder of haemostasis, such as haemophilia, with the inhibitor of the invention results in reduced periostitis. As shown in FIG. 12I, treatment of Haem A mice with an inhibitor of the invention significantly reduced periostitis in said mice.

In certain embodiments, treatment of a subject, preferably a subject having a disorder of haemostasis, such as haemophilia, with the inhibitor of the invention results in reduced sub-chondral bone sclerosis. As shown in FIG. 12J, treatment of Haem A mice with an inhibitor of the invention significantly reduced sub-chondral bone sclerosis in said mice.

In certain embodiments, treatment of a subject, preferably a subject having a disorder of haemostasis, such as haemophilia, with the inhibitor of the invention results in reduced tendon degeneration. As shown in FIG. 12K, treatment of Haem A mice with an inhibitor of the invention significantly reduced tendon degeneration in said mice.

In certain embodiments, treatment of a subject, preferably a subject having a disorder of haemostasis, such as haemophilia, with the inhibitor of the invention results in reduced tendonitis. As shown in FIG. 12L, treatment of Haem A mice with an inhibitor of the invention significantly reduced tendonitis in said mice.

In certain embodiments, treatment of a subject, preferably a subject having a disorder of haemostasis, such as haemophilia, with the inhibitor of the invention results in reduced tenosynovitis. As shown in FIG. 12M, treatment of Haem A mice with an inhibitor of the invention significantly reduced tenosynovitis in said mice.

Thus, in a particular embodiment, the invention relates to an inhibitor suitable for use, or for use, in treatment of haemophilia, wherein the treatment of haemophilia is characterized by reduced bleeding and one or more of: reduced bone marrow hyperplasia, reduced osteoarthritis, reduced chondrocyte degeneration/necrosis, reduced haemorrhage, reduced haemosiderin deposition, reduced haematoma, reduced osteoclastogenic bone resorption, reduced osteolysis, reduced periostitis, reduced sub-chondral bone sclerosis, reduced tendon degeneration, reduced tendonitis, and/or reduced tenosynovitis.

The term “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to treating a disease in said human or other vertebrate. The timing and purpose of said treatment may vary from one individual to another, according to the subject's health. Thus, said treatment may be prophylactic, palliative, symptomatic and/or curative.

### Inhibitors

Inhibitors of the invention include nucleic acids such as siRNAs, antibodies and antigen binding fragments thereof, e.g., monoclonal antibodies, polypeptides, antibody-drug conjugates, and small molecules. Preferred are nucleic acids such as siRNA.

Certain preferred features of inhibitors of the invention, where these are oligonucelosides such as siRNA, are given below.

In certain embodiments, the nucleic acid comprises a first strand comprising a sequence that is at least partially complementary to a portion of RNA transcribed from the ZPI gene (SEQ ID NO:1). In a preferred embodiment, the nucleic acid comprises a first strand comprising a sequence that is at least partially complementary to a ZPI mRNA (NM_016186.3).

In certain embodiments, the nucleic acid for inhibiting expression of ZPI comprises a duplex region that comprises a first strand and a second strand that is at least partially complementary to the first strand, wherein said first strand is:
(i) at least partially complementary to a portion of RNA transcribed from the ZPI gene, and
(ii) comprises at least 17 contiguous nucleosides differing by 0 or 1 nucleosides from any one of SEQ ID NO:122-241.

In certain embodiments, the first strand comprises nucleosides 2-18 of any one of the sequences set forth in SEQ ID NO:122-241.

In certain embodiments, the first strand comprises any one of SEQ ID NO:122-241.

In certain embodiments, the nucleic acid for inhibiting expression of ZPI comprises a duplex region that comprises a first strand and a second strand that is at least partially complementary to the first strand, wherein said first strand is:
(i) at least partially complementary to a portion of RNA transcribed from the ZPI gene, and
(ii) comprises at least 21 contiguous nucleosides differing by 0 or 1 nucleosides from any one of SEQ ID NO:122-241.

In certain embodiments, the first strand comprises nucleosides 2-22 of any one of the sequences set forth in SEQ ID NO:122-241.

In certain embodiments, the second strand comprises a nucleoside sequence of at least 17 contiguous nucleosides differing by 0 or 1 nucleosides from any one of SEQ ID NO:242-361; wherein the second strand has a region of at least 85% complementarity over the 17 contiguous nucleosides to the first strand.

In certain embodiments, the second strand comprises a nucleoside sequence of at least 19 contiguous nucleosides differing by 0 or 1 nucleosides from any one of SEQ ID NO:242-361; wherein the second strand has a region of at least 85% complementarity over the 19 contiguous nucleosides to the first strand.

In certain embodiments, the second strand comprises a nucleoside sequence of at least 21 contiguous nucleosides differing by 0 or 1 nucleosides from any one of SEQ ID NO:242-361; wherein the second strand has a region of at least 85% complementarity over the 21 contiguous nucleosides to the first strand.

In certain embodiments, the second strand comprises any one of SEQ ID NO:242-361.

In certain embodiments, the nucleic acid comprises a first strand that comprises, consists of, or consists essentially of a nucleoside sequence differing by 0 or 1 nucleosides from any one of SEQ ID NO:122-241;
and a second strand that comprises, consists of, or consists essentially of a nucleoside sequence differing by 0 or 1 nucleosides from any one of SEQ ID NO:242-361.

It is preferred herein that the duplex region is formed between a first (antisense) strand and a complementary second (sense) strand. Exemplary pairs of complementary antisense and sense strands are listed in Table 2 below: Table 2:

TABLE 2

| | First (Antisense) Strand | | Second (Sense) Strand | |
|---|---|---|---|---|
| | Base Sequence 5′→3′ | | Base Sequence 5′→3′ | Corresponding |
| SEQ ID | (Shown as an Unmodified | SEQ ID | (Shown as an Unmodified | positions on |
| NO (AS) | Nucleotide Sequence) | NO (SS) | Nucleotide Sequence) | NM_016186.3 |
| SEQ ID | AAAUCCUUGUGGAUGAAGG | SEQ ID | UGCCUUCAUCCACAAGG | 991-1012 |
| NO: 122 | CAAA | NO: 242 | AUUU | |
| SEQ ID | GCCUCAUGGAGAUCUUUCG | SEQ ID | UGCGAAAGAUCUCCAUG | 756-777 |
| NO: 123 | CAGC | NO: 243 | AGGC | |
| SEQ ID | UUCUCCAUGAGGACCACCA | SEQ ID | GCUGGUGGUCCUCAUGG | 1390-1411 |
| NO: 124 | GCAU | NO: 244 | AGAA | |
| SEQ ID | UGCCUCAUGGAGAUCUUUC | SEQ ID | GCGAAAGAUCUCCAUGA | 757-778 |
| NO: 125 | GCAG | NO: 245 | GGCA | |
| SEQ ID | AGCAGCUCAUGCAUCUCAU | SEQ ID | GUAUGAGAUGCAUGAGC | 1531-1552 |
| NO: 126 | ACUU | NO: 246 | UGCU | |
| SEQ ID | UCAGGAUUAAUCUCAUCAA | SEQ ID | GUUUGAUGAGAUUAAUC | 1156-1177 |
| NO: 127 | ACAG | NO: 247 | CUGA | |
| SEQ ID | UUGGUUUCAGGAUUAAUCU | SEQ ID | UGAGAUUAAUCCUGAAA | 1162-1183 |
| NO: 128 | CAUC | NO: 248 | CCAA | |
| SEQ ID | GUUUCAGGAUUAAUCUCAU | SEQ ID | UGAUGAGAUUAAUCCUG | 1159-1180 |
| NO: 129 | CAAA | NO: 249 | AAAC | |
| SEQ ID | UGGUUUCAGGAUUAAUCUC | SEQ ID | AUGAGAUUAAUCCUGAA | 1161-1182 |
| NO: 130 | AUCA | NO: 250 | ACCA | |
| SEQ ID | GGUUUCAGGAUUAAUCUCA | SEQ ID | GAUGAGAUUAAUCCUGA | 1160-1181 |
| NO: 131 | UCAA | NO: 251 | AACC | |
| SEQ ID | AGGAUUAAUCUCAUCAAAC | SEQ ID | CUGUUUGAUGAGAUUAA | 1154-1175 |
| NO: 132 | AGUU | NO: 252 | UCCU | |
| SEQ ID | UCCUUGUGGAUGAAGGCAA | SEQ ID | UUUUGCCUUCAUCCACA | 988-1009 |
| NO: 133 | AACU | NO: 253 | AGGA | |
| SEQ ID | GUGCCUCAUGGAGAUCUUU | SEQ ID | CGAAAGAUCUCCAUGAG | 758-779 |
| NO: 134 | CGCA | NO: 254 | GCAC | |
| SEQ ID | UCUCCAUGAGGACCACCAG | SEQ ID | UGCUGGUGGUCCUCAUG | 1389-1410 |
| NO: 135 | CAUG | NO: 255 | GAGA | |
| SEQ ID | CAAAAUCCUUGUGGAUGAA | SEQ ID | CCUUCAUCCACAAGGAU | 993-1014 |
| NO: 136 | GGCA | NO: 256 | UUUG | |
| SEQ ID | CGUGCCUCAUGGAGAUCUU | SEQ ID | GAAAGAUCUCCAUGAGG | 759-780 |
| NO: 137 | UCGC | NO: 257 | CACG | |
| SEQ ID | AUCAAAAUCCUUGUGGAUG | SEQ ID | UUCAUCCACAAGGAUUU | 995-1016 |
| NO: 138 | AAGG | NO: 258 | UGAU | |
| SEQ ID | UUCAGGAUUAAUCUCAUCA | SEQ ID | UUUGAUGAGAUUAAUCC | 1157-1178 |
| NO: 139 | AACA | NO: 259 | UGAA | |
| SEQ ID | AAUCCUUGUGGAUGAAGGC | SEQ ID | UUGCCUUCAUCCACAAG | 990-1011 |
| NO: 140 | AAAA | NO: 260 | GAUU | |
| SEQ ID | CCAUCGUGCCUCAUGGAGA | SEQ ID | GAUCUCCAUGAGGCACG | 763-784 |
| NO: 141 | UCUU | NO: 261 | AUGG | |
| SEQ ID | UCCAUGAGGACCACCAGCA | SEQ ID | CAUGCUGGUGGUCCUCA | 1387-1408 |
| NO: 142 | UGGU | NO: 262 | UGGA | |
| SEQ ID | AUCCUUGUGGAUGAAGGCA | SEQ ID | UUUGCCUUCAUCCACAA | 989-1010 |
| NO: 143 | AAAC | NO: 263 | GGAU | |
| SEQ ID | AAAAUCCUUGUGGAUGAAG | SEQ ID | GCCUUCAUCCACAAGGA | 992-1013 |
| NO: 144 | GCAA | NO: 264 | UUUU | |
| SEQ ID | CAUGGUGGCAUUUCCUUGG | SEQ ID | UACCAAGGAAAUGCCAC | 1370-1391 |
| NO: 145 | UAGG | NO: 265 | CAUG | |
| SEQ ID | UUUCAGGAUUAAUCUCAUC | SEQ ID | UUGAUGAGAUUAAUCCU | 1158-1179 |
| NO: 146 | AAAC | NO: 266 | GAAA | |
| SEQ ID | UCAAAAUCCUUGUGGAUGA | SEQ ID | CUUCAUCCACAAGGAUU | 994-1015 |
| NO: 147 | AGGC | NO: 267 | UUGA | |
| SEQ ID | UCGUGCCUCAUGGAGAUCU | SEQ ID | AAAGAUCUCCAUGAGGC | 760-781 |
| NO: 148 | UUCG | NO: 268 | ACGA | |
| SEQ ID | CAGGAUUAAUCUCAUCAAA | SEQ ID | UGUUUGAUGAGAUUAAU | 1155-1176 |
| NO: 149 | CAGU | NO: 269 | CCUG | |
| SEQ ID | CUCCAUGAGGACCACCAGC | SEQ ID | AUGCUGGUGGUCCUCAU | 1388-1409 |
| NO: 150 | AUGG | NO: 270 | GGAG | |
| SEQ ID | CCUUGUGGAUGAAGGCAAA | SEQ ID | GUUUUGCCUUCAUCCAC | 987-1008 |
| NO: 151 | ACUC | NO: 271 | AAGG | |
| SEQ ID | UGAGGACCACCAGCAUGGU | SEQ ID | CCACCAUGCUGGUGGUC | 1383-1404 |
| NO: 152 | GGCA | NO: 272 | CUCA | |
| SEQ ID | AUGAGGACCACCAGCAUGG | SEQ ID | CACCAUGCUGGUGGUCC | 1384-1405 |
| NO: 153 | UGGC | NO: 273 | UCAU | |
| SEQ ID | AUCGUGCCUCAUGGAGAUC | SEQ ID | AAGAUCUCCAUGAGGCA | 761-782 |
| NO: 154 | UUUC | NO: 274 | CGAU | |
| SEQ ID | CUUGUGGAUGAAGGCAAAA | SEQ ID | AGUUUUGCCUUCAUCCA | 986-1007 |
| NO: 155 | CUCC | NO: 275 | CAAG | |
| SEQ ID | CAUGAGGACCACCAGCAUG | SEQ ID | ACCAUGCUGGUGGUCCU | 1385-1406 |
| NO: 156 | GUGG | NO: 276 | CAUG | |
| SEQ ID | CAUCGUGCCUCAUGGAGAU | SEQ ID | AGAUCUCCAUGAGGCAC | 762-783 |
| NO: 157 | CUUU | NO: 277 | GAUG | |
| SEQ ID | CCAUGAGGACCACCAGCAU | SEQ ID | CCAUGCUGGUGGUCCUC | 1386-1407 |
| NO: 158 | GGUG | NO: 278 | AUGG | |
| SEQ ID | UUCUUGUCAAAGGUGGAGG | SEQ ID | UGCCUCCACCUUUGACA | 1321-1342 |
| NO: 159 | CAAA | NO: 279 | AGAA | |
| SEQ ID | AAUUCUUGUCAAAGGUGGA | SEQ ID | CCUCCACCUUUGACAAG | 1323-1344 |
| NO: 160 | GGCA | NO: 280 | AAUU | |
| SEQ ID | UACAUCAUGGGCACCUUAA | SEQ ID | CAUUAAGGUGCCCAUGA | 1285-1306 |
| NO: 161 | UGGU | NO: 281 | UGUA | |
| SEQ ID | GGCAUUUCCUUGGUAGGGC | SEQ ID | CUGCCCUACCAAGGAAA | 1364-1385 |
| NO: 162 | AGUU | NO: 282 | UGCC | |
| SEQ ID | UUUCCUUGGUAGGGCAGUU | SEQ ID | CAAACUGCCCUACCAAG | 1360-1381 |
| NO: 163 | UGAG | NO: 283 | GAAA | |
| SEQ ID | UUCCUUGGUAGGGCAGUUU | SEQ ID | UCAAACUGCCCUACCAA | 1359-1380 |
| NO: 164 | GAGG | NO: 284 | GGAA | |
| SEQ ID | AAAUUCUUGUCAAAGGUGG | SEQ ID | CUCCACCUUUGACAAGA | 1324-1345 |
| NO: 165 | AGGC | NO: 285 | AUUU | |
| SEQ ID | UUGUCCAGGUGGAAAGUGU | SEQ ID | CGACACUUUCCACCUGG | 1255-1276 |
| NO: 166 | CGAC | NO: 286 | ACAA | |
| SEQ ID | GUACUUGUCCAGGUGGAAA | SEQ ID | ACUUUCCACCUGGACAA | 1259-1280 |
| NO: 167 | GUGU | NO: 287 | GUAC | |
| SEQ ID | AUUUCCUUGGUAGGGCAGU | SEQ ID | AAACUGCCCUACCAAGG | 1361-1382 |
| NO: 168 | UUGA | NO: 288 | AAAU | |
| SEQ ID | GCAUUUCCUUGGUAGGGCA | SEQ ID | ACUGCCCUACCAAGGAA | 1363-1384 |
| NO: 169 | GUUU | NO: 289 | AUGC | |
| SEQ ID | UACUUGUCCAGGUGGAAAG | SEQ ID | CACUUUCCACCUGGACA | 1258-1279 |
| NO: 170 | UGUC | NO: 290 | AGUA | |
| SEQ ID | ACAUCAUGGGCACCUUAAU | SEQ ID | CCAUUAAGGUGCCCAUG | 1284-1305 |
| NO: 171 | GGUC | NO: 291 | AUGU | |
| SEQ ID | UCUUGUCAAAGGUGGAGGC | SEQ ID | UUGCCUCCACCUUUGAC | 1320-1341 |
| NO: 172 | AAAC | NO: 292 | AAGA | |
| SEQ ID | UUGUACUUGUCCAGGUGGA | SEQ ID | UUUCCACCUGGACAAGU | 1261-1282 |
| NO: 173 | AAGU | NO: 293 | ACAA | |
| SEQ ID | CUUGGUAGGGCAGUUUGAG | SEQ ID | UCCUCAAACUGCCCUAC | 1356-1377 |
| NO: 174 | GACA | NO: 294 | CAAG | |
| SEQ ID | CAUUUCCUUGGUAGGGCAG | SEQ ID | AACUGCCCUACCAAGGA | 1362-1383 |
| NO: 175 | UUUG | NO: 295 | AAUG | |
| SEQ ID | CCUUGGUAGGGCAGUUUGA | SEQ ID | CCUCAAACUGCCCUACC | 1357-1378 |
| NO: 176 | GGAC | NO: 296 | AAGG | |
| SEQ ID | UUGGUAGGGCAGUUUGAGG | SEQ ID | GUCCUCAAACUGCCCUA | 1355-1376 |
| NO: 177 | ACAU | NO: 297 | CCAA | |
| SEQ ID | CUUGUCCAGGUGGAAAGUG | SEQ ID | GACACUUUCCACCUGGA | 1256-1277 |
| NO: 178 | UCGA | NO: 298 | CAAG | |
| SEQ ID | CUUGUCAAAGGUGGAGGCA | SEQ ID | UUUGCCUCCACCUUUGA | 1319-1340 |
| NO: 179 | AACU | NO: 299 | CAAG | |
| SEQ ID | ACUUGUCCAGGUGGAAAGU | SEQ ID | ACACUUUCCACCUGGAC | 1257-1278 |
| NO: 180 | GUCG | NO: 300 | AAGU | |
| SEQ ID | CUUGUACUUGUCCAGGUGG | SEQ ID | UUCCACCUGGACAAGUA | 1262-1283 |
| NO: 181 | AAAG | NO: 301 | CAAG | |
| SEQ ID | UGUACUUGUCCAGGUGGAA | SEQ ID | CUUUCCACCUGGACAAG | 1260-1281 |
| NO: 182 | AGUG | NO: 302 | UACA | |
| SEQ ID | UCCUUGGUAGGGCAGUUUG | SEQ ID | CUCAAACUGCCCUACCA | 1358-1379 |
| NO: 183 | AGGA | NO: 303 | AGGA | |
| SEQ ID | UUCCCUUUGAACAAGAUGU | SEQ ID | UUACAUCUUGUUCAAAG | 1198-1219 |
| NO: 184 | AAUC | NO: 304 | GGAA | |
| SEQ ID | AGGACCACCAGCAUGGUGG | SEQ ID | UGCCACCAUGCUGGUGG | 1381-1402 |
| NO: 185 | CAUU | NO: 305 | UCCU | |
| SEQ ID | UAUCAAAAUACCUCUUGGA | SEQ ID | UAUCCAAGAGGUAUUUU | 1038-1059 |
| NO: 186 | UAAA | NO: 306 | GAUA | |
| SEQ ID | ACCACCAGCAUGGUGGCAU | SEQ ID | AAAUGCCACCAUGCUGG | 1378-1399 |
| NO: 187 | UUCC | NO: 307 | UGGU | |
| SEQ ID | UCCCUUUGAACAAGAUGUA | SEQ ID | AUUACAUCUUGUUCAAA | 1197-1218 |
| NO: 188 | AUCC | NO: 308 | GGGA | |
| SEQ ID | UUGCCAUCGUGCCUCAUGG | SEQ ID | CUCCAUGAGGCACGAUG | 766-787 |
| NO: 189 | AGAU | NO: 309 | GCAA | |
| SEQ ID | UUUGAUGACAGGAGGCAUG | SEQ ID | UCCAUGCCUCCUGUCAU | 1721-1742 |
| NO: 190 | GAAU | NO: 310 | CAAA | |
| SEQ ID | UGAUGACAGGAGGCAUGGA | SEQ ID | AUUCCAUGCCUCCUGUC | 1719-1740 |
| NO: 191 | AUAA | NO: 311 | AUCA | |
| SEQ ID | CAGCAUGGUGGCAUUUCCU | SEQ ID | CAAGGAAAUGCCACCAU | 1373-1394 |
| NO: 192 | UGGU | NO: 312 | GCUG | |
| SEQ ID | UUUUCUCCAUGAGGACCAC | SEQ ID | UGGUGGUCCUCAUGGAG | 1392-1413 |
| NO: 193 | CAGC | NO: 313 | AAAA | |
| SEQ ID | CCAUUUCCCUUUGAACAAG | SEQ ID | AUCUUGUUCAAAGGGAA | 1202-1223 |
| NO: 194 | AUGU | NO: 314 | AUGG | |
| SEQ ID | CCAGCAUGGUGGCAUUUCC | SEQ ID | AAGGAAAUGCCACCAUG | 1374-1395 |
| NO: 195 | UUGG | NO: 315 | CUGG | |
| SEQ ID | AUUCUUGUCAAAGGUGGAG | SEQ ID | GCCUCCACCUUUGACAA | 1322-1343 |
| NO: 196 | GCAA | NO: 316 | GAAU | |
| SEQ ID | UUGUGGAUGAAGGCAAAAC | SEQ ID | GAGUUUUGCCUUCAUCC | 985-1006 |
| NO: 197 | UCCC | NO: 317 | ACAA | |
| SEQ ID | CUCAUGGAGAUCUUUCGCA | SEQ ID | GCUGCGAAAGAUCUCCA | 754-775 |
| NO: 198 | GCAG | NO: 318 | UGAG | |
| SEQ ID | UGUCAAAGGUGGAGGCAAA | SEQ ID | AGUUUGCCUCCACCUUU | 1317-1338 |
| NO: 199 | CUUG | NO: 319 | GACA | |
| SEQ ID | UUGUCAAAGGUGGAGGCAA | SEQ ID | GUUUGCCUCCACCUUUG | 1318-1339 |
| NO: 200 | ACUU | NO: 320 | ACAA | |
| SEQ ID | CAUUUCCCUUUGAACAAGA | SEQ ID | CAUCUUGUUCAAAGGGA | 1201-1222 |
| NO: 201 | UGUA | NO: 321 | AAUG | |
| SEQ ID | UGUGGAUGAAGGCAAAACU | SEQ ID | GGAGUUUUGCCUUCAUC | 984-1005 |
| NO: 202 | CCCC | NO: 322 | CACA | |
| SEQ ID | CCUCAUGGAGAUCUUUCGC | SEQ ID | CUGCGAAAGAUCUCCAU | 755-776 |
| NO: 203 | AGCA | NO: 323 | GAGG | |
| SEQ ID | CUUUGAUGACAGGAGGCAU | SEQ ID | CCAUGCCUCCUGUCAUC | 1722-1743 |
| NO: 204 | GGAA | NO: 324 | AAAG | |
| SEQ ID | UCACCACCCUGCCCAGAAA | SEQ ID | UGUUUCUGGGCAGGGUG | 1794-1815 |
| NO: 205 | CAGA | NO: 325 | GUGA | |
| SEQ ID | UGGAGAUCUUUCGCAGCAG | SEQ ID | GCCUGCUGCGAAAGAUC | 750-771 |
| NO: 206 | GCUG | NO: 326 | UCCA | |
| SEQ ID | GAUUAAUCUCAUCAAACAG | SEQ ID | AACUGUUUGAUGAGAUU | 1152-1173 |
| NO: 207 | UUUG | NO: 327 | AAUC | |
| SEQ ID | CACUUUGAUGACAGGAGGC | SEQ ID | AUGCCUCCUGUCAUCAA | 1724-1745 |
| NO: 208 | AUGG | NO: 328 | AGUG | |
| SEQ ID | GCAGCUCAUGCAUCUCAUA | SEQ ID | AGUAUGAGAUGCAUGAG | 1530-1551 |
| NO: 209 | CUUC | NO: 329 | CUGC | |
| SEQ ID | CAGCUCAUGCAUCUCAUAC | SEQ ID | AAGUAUGAGAUGCAUGA | 1529-1550 |
| NO: 210 | UUCU | NO: 330 | GCUG | |
| SEQ ID | UGGUAGGGCAGUUUGAGGA | SEQ ID | UGUCCUCAAACUGCCCU | 1354-1375 |
| NO: 211 | CAUG | NO: 331 | ACCA | |
| SEQ ID | GGAUUAAUCUCAUCAAACA | SEQ ID | ACUGUUUGAUGAGAUUA | 1153-1174 |
| NO: 212 | GUUU | NO: 332 | AUCC | |
| SEQ ID | AUCAUGGGCACCUUAAUGG | SEQ ID | GACCAUUAAGGUGCCCA | 1282-1303 |
| NO: 213 | UCUU | NO: 333 | UGAU | |
| SEQ ID | CAUCAUGGGCACCUUAAUG | SEQ ID | ACCAUUAAGGUGCCCAU | 1283-1304 |
| NO: 214 | GUCU | NO: 334 | GAUG | |
| SEQ ID | UUCACCACCCUGCCCAGAA | SEQ ID | GUUUCUGGGCAGGGUGG | 1795-1816 |
| NO: 215 | ACAG | NO: 335 | UGAA | |
| SEQ ID | CCACCCUGCCCAGAAACAG | SEQ ID | UUCUGUUUCUGGGCAGG | 1791-1812 |
| NO: 216 | AAGC | NO: 336 | GUGG | |
| SEQ ID | GCUUGAACUUCGGAAAGAA | SEQ ID | UUUUCUUUCCGAAGUUC | 1500-1521 |
| NO: 217 | AACU | NO: 337 | AAGC | |
| SEQ ID | AGCUUGAACUUCGGAAAGA | SEQ ID | UUUCUUUCCGAAGUUCA | 1501-1522 |
| NO: 218 | AAAC | NO: 338 | AGCU | |
| SEQ ID | ACCACCCUGCCCAGAAACA | SEQ ID | UCUGUUUCUGGGCAGGG | 1792-1813 |
| NO: 219 | GAAG | NO: 339 | UGGU | |
| SEQ ID | GGUAGGGCAGUUUGAGGAC | SEQ ID | AUGUCCUCAAACUGCCC | 1353-1374 |
| NO: 220 | AUGA | NO: 340 | UACC | |
| SEQ ID | UGUCCAGGUGGAAAGUGUC | SEQ ID | UCGACACUUUCCACCUG | 1254-1275 |
| NO: 221 | GACU | NO: 341 | GACA | |
| SEQ ID | AAAUCCUUGUGGAUGAAGG | SEQ ID | CCUUCAUCCACAAGGAU | 995-1014 |
| NO: 222 | | NO: 342 | UU | |
| SEQ ID | GCCUCAUGGAGAUCUUUCG | SEQ ID | CGAAAGAUCUCCAUGAG | 760-779 |
| NO: 223 | | NO: 343 | GC | |
| SEQ ID | UUCUCCAUGAGGACCACCA | SEQ ID | UGGUGGUCCUCAUGGAG | 1394-1413 |
| NO: 224 | | NO: 344 | AA | |
| SEQ ID | UGCCUCAUGGAGAUCUUUC | SEQ ID | GAAAGAUCUCCAUGAGG | 761-780 |
| NO: 225 | | NO: 345 | CA | |
| SEQ ID | AGCAGCUCAUGCAUCUCAU | SEQ ID | AUGAGAUGCAUGAGCUG | 1535-1554 |
| NO: 226 | | NO: 346 | CU | |
| SEQ ID | UCAGGAUUAAUCUCAUCAA | SEQ ID | UUGAUGAGAUUAAUCCU | 1160-1179 |
| NO: 227 | | NO: 347 | GA | |
| SEQ ID | UUGGUUUCAGGAUUAAUCU | SEQ ID | AGAUUAAUCCUGAAACC | 1166-1185 |
| NO: 228 | | NO: 348 | AA | |
| SEQ ID | GUUUCAGGAUUAAUCUCAU | SEQ ID | AUGAGAUUAAUCCUGAA | 1163-1182 |
| NO: 229 | | NO: 349 | AC | |
| SEQ ID | UGGUUUCAGGAUUAAUCUC | SEQ ID | GAGAUUAAUCCUGAAAC | 1165-1184 |
| NO: 230 | | NO: 350 | CA | |
| SEQ ID | GGUUUCAGGAUUAAUCUCA | SEQ ID | UGAGAUUAAUCCUGAAA | 1164-1183 |
| NO: 231 | | NO: 351 | CC | |
| SEQ ID | AGGAUUAAUCUCAUCAAAC | SEQ ID | GUUUGAUGAGAUUAAUC | 1158-1177 |
| NO: 232 | | NO: 352 | CU | |
| SEQ ID | UCCUUGUGGAUGAAGGCAA | SEQ ID | UUGCCUUCAUCCACAAG | 992-1011 |
| NO: 233 | | NO: 353 | GA | |
| SEQ ID | GUGCCUCAUGGAGAUCUUU | SEQ ID | AAAGAUCUCCAUGAGGC | 762-781 |
| NO: 234 | | NO: 354 | AC | |
| SEQ ID | UCUCCAUGAGGACCACCAG | SEQ ID | CUGGUGGUCCUCAUGGA | 1393-1412 |
| NO: 235 | | NO: 355 | GA | |
| SEQ ID | CAAAAUCCUUGUGGAUGAA | SEQ ID | UUCAUCCACAAGGAUUU | 997-1016 |
| NO: 236 | | NO: 356 | UG | |
| SEQ ID | CGUGCCUCAUGGAGAUCUU | SEQ ID | AAGAUCUCCAUGAGGCA | 763-782 |
| NO: 237 | | NO: 357 | CG | |
| SEQ ID | AUCAAAAUCCUUGUGGAUG | SEQ ID | CAUCCACAAGGAUUUUG | 999-1018 |
| NO: 238 | | NO: 358 | AU | |
| SEQ ID | UUCAGGAUUAAUCUCAUCA | SEQ ID | UGAUGAGAUUAAUCCUG | 1161-1180 |
| NO: 239 | | NO: 359 | AA | |
| SEQ ID | AAUCCUUGUGGAUGAAGGC | SEQ ID | GCCUUCAUCCACAAGGA | 994-1013 |
| NO: 240 | | NO: 360 | UU | |
| SEQ ID | CCAUCGUGCCUCAUGGAGA | SEQ ID | UCUCCAUGAGGCACGAU | 767-786 |
| NO: 241 | | NO: 361 | GG | |
| SEQ ID | AAAGUCCUUGUGGAUGAAG | SEQ ID | GCCUUCAUCCACAAGGA | NA |
| NO: 787 | GCAA | NO: 791 | CUUU | |
| SEQ ID | AACUUCUUGUCAAAGGUGG | SEQ ID | CUCCACCUUUGACAAGA | NA |
| NO: 788 | AGGC | NO: 792 | AGUU | |
| SEQ ID | UGUGGAUGAAGGCAAAGCU | SEQ ID | GUAGCUUUGCCUUCAUC | NA |
| NO: 789 | ACCC | NO: 793 | CACA | |

In a particular embodiment, the invention relates to a nucleic acid comprising first and second strands that comprise, consist of, or consist essentially of a nucleoside sequence differing by 0 or 1 nucleosides from any one of the following first and second sequences:

| Unmodified first strand | Unmodified second strand |
|---|---|
| SEQ ID NO: 148 | SEQ ID NO: 268 |
| SEQ ID NO: 145 | SEQ ID NO: 265 |
| SEQ ID NO: 144 | SEQ ID NO: 264 |
| SEQ ID NO: 165 | SEQ ID NO: 285 |
| SEQ ID NO: 202 | SEQ ID NO: 322 |

In certain embodiments, the nucleic acid for inhibiting expression of ZPI comprises a duplex region that comprises a first strand and a second strand that is at least partially complementary to the first strand, wherein said first strand is:
(i) at least partially complementary to a portion of RNA transcribed from the ZPI gene, and
(ii) comprises at least 17 contiguous nucleosides differing by 0 or 1 nucleosides from any one of SEQ ID NO: 362-561, 762-771, 782-786 or 795-797.

In certain embodiments, the first strand comprises nucleosides 2-18 of any one of the sequences set forth in SEQ TD NO: 362-561, 762-771, 782-786 or 795-797.

In certain embodiments, the nucleic acid for inhibiting expression of ZPI comprises a duplex region that comprises a first strand and a second strand that is at least partially complementary to the first strand, wherein said first strand is:
(i) at least partially complementary to a portion of RNA transcribed from the ZPI gene, and
(ii) comprises at least 21 contiguous nucleosides differing by 0 or 1 nucleosides from any one of SEQ ID NO: 362-561, 762-771, 782-786 or 795-797.

In certain embodiments, the first strand comprises nucleosides 2-22 ofany one of the sequences set forth in SEQ TD NO: 362-561, 762-771, 782-786 or 795-797.

In certain embodiments, the first strand comprises any one of SEQ TD NO:362-561, 762-771, 782-786 or 795-797.

The modification pattern of the nucleic acids as set forth in SEQ TD NO:362-561, 762-771, 782-786 or 795-797 is summarized in Table 3 below:

TABLE 3

| | | | Underlying | |
|---|---|---|---|---|
| | | | Base | |
| | | | Sequence | |
| | | | 5′ → 3′ | |
| Anti- | | SEQ ID | (Shown as an | SEQ ID |
| sense | Modified First | NO | Unmodified | NO |
| strand | (Antisense) Strand | (AS - | Nucleotide | (AS - |
| ID | 5′ → 3′ | mod) | Sequence) | unmod) |
| ETXS632 | AmsAfsAmUmCmCfUmUfGfUmGmGmA | SEQ ID | AAAUCCUUGUGGAU | SEQ ID |
| | mUfGmAfAmGmGmCmAmsAmsAm | NO: 362 | GAAGGCAAA | NO: 122 |
| ETXS634 | GmsCfsCmUmCmAfUmGfGfAmGmAmU | SEQ ID | GCCUCAUGGAGAUC | SEQ ID |
| | mCfUmUfUmCmGmCmAmsGmsCm | NO: 363 | UUUCGCAGC | NO: 123 |
| ETXS636 | UmsUfsCmUmCmCfAmUfGfAmGmGmA | SEQ ID | UUCUCCAUGAGGAC | SEQ ID |
| | mCfCmAfCmCmAmGmCmsAmsUm | NO: 364 | CACCAGCAU | NO: 124 |
| ETXS638 | UmsGfsCmCmUmCfAmUfGfGmAmGmA | SEQ ID | UGCCUCAUGGAGAU | SEQ ID |
| | mUfCmUfUmUmCmGmCmsAmsGm | NO: 365 | CUUUCGCAG | NO: 125 |
| ETXS640 | AmsGfsCmAmGmCfUmCfAfUmGmCmA | SEQ ID | AGCAGCUCAUGCAU | SEQ ID |
| | mUfCmUfCmAmUmAmCmsUmsUm | NO: 366 | CUCAUACUU | NO: 126 |
| ETXS642 | UmsCfsAmGmGmAfUmUfAfAmUmCmU | SEQ ID | UCAGGAUUAAUCUC | SEQ ID |
| | mCfAmUfCmAmAmAmCmsAmsGm | NO: 367 | AUCAAACAG | NO: 127 |
| ETXS644 | UmsUfsGmGmUmUfUmCfAfGmGmAmU | SEQ ID | UUGGUUUCAGGAUU | SEQ ID |
| | mUfAmAfUmCmUmCmAmsUmsCm | NO: 368 | AAUCUCAUC | NO: 128 |
| ETXS646 | GmsUfsUmUmCmAfGmGfAfUmUmAmA | SEQ ID | GUUUCAGGAUUAAU | SEQ ID |
| | mUfCmUfCmAmUmCmAmsAmsAm | NO: 369 | CUCAUCAAA | NO: 129 |
| ETXS648 | UmsGfsGmUmUmUfCmAfGfGmAmUmU | SEQ ID | UGGUUUCAGGAUUA | SEQ ID |
| | mAfAmUfCmUmCmAmUmsCmsAm | NO: 370 | AUCUCAUCA | NO: 130 |
| ETXS650 | GmsGfsUmUmUmCfAmGfGfAmUmUmA | SEQ ID | GGUUUCAGGAUUAA | SEQ ID |
| | mAfUmCfUmCmAmUmCmsAmsAm | NO: 371 | UCUCAUCAA | NO: 131 |
| ETXS652 | AmsGfsGmAmUmUfAmAfUfCmUmCmA | SEQ ID | AGGAUUAAUCUCAU | SEQ ID |
| | mUfCmAfAmAmCmAmGmsUmsUm | NO: 372 | CAAACAGUU | NO: 132 |
| ETXS654 | UmsCfsCmUmUmGfUmGfGfAmUmGmA | SEQ ID | UCCUUGUGGAUGAA | SEQ ID |
| | mAfGmGfCmAmAmAmAmsCmsUm | NO: 373 | GGCAAAACU | NO: 133 |
| ETXS656 | GmsUfsGmCmCmUfCmAfUfGmGmAmG | SEQ ID | GUGCCUCAUGGAGA | SEQ ID |
| | mAfUmCfUmUmUmCmGmsCmsAm | NO: 374 | UCUUUCGCA | NO: 134 |
| ETXS658 | UmsCfsUmCmCmAfUmGfAfGmGmAmC | SEQ ID | UCUCCAUGAGGACC | SEQ ID |
| | mCfAmCfCmAmGmCmAmsUmsGm | NO: 375 | ACCAGCAUG | NO: 135 |
| ETXS660 | CmsAfsAmAmAmUfCmCfUfUmGmUmG | SEQ ID | CAAAAUCCUUGUGG | SEQ ID |
| | mGfAmUfGmAmAmGmGmsCmsAm | NO: 376 | AUGAAGGCA | NO: 136 |
| ETXS662 | CmsGfsUmGmCmCfUmCfAfUmGmGmA | SEQ ID | CGUGCCUCAUGGAG | SEQ ID |
| | mGfAmUfCmUmUmUmCmsGmsCm | NO: 377 | AUCUUUCGC | NO: 137 |
| ETXS664 | AmsUfsCmAmAmAfAmUfCfCmUmUmG | SEQ ID | AUCAAAAUCCUUGU | SEQ ID |
| | mUfGmGfAmUmGmAmAmsGmsGm | NO: 378 | GGAUGAAGG | NO: 138 |
| ETXS666 | UmsUfsCmAmGmGfAmUfUfAmAmUmC | SEQ ID | UUCAGGAUUAAUCU | SEQ ID |
| | mUfCmAfUmCmAmAmAmsCmsAm | NO: 379 | CAUCAAACA | NO: 139 |
| ETXS668 | AmsAfsUmCmCmUfUmGfUfGmGmAmU | SEQ ID | AAUCCUUGUGGAUG | SEQ ID |
| | mGfAmAfGmGmCmAmAmsAmsAm | NO: 380 | AAGGCAAAA | NO: 140 |
| ETXS670 | CmsCfsAmUmCmGfUmGfCfCmUmCmA | SEQ ID | CCAUCGUGCCUCAU | SEQ ID |
| | mUfGmGfAmGmAmUmCmsUmsUm | NO: 381 | GGAGAUCUU | NO: 141 |
| ETXS672 | UmsCfsCmAmUmGfAmGfGfAmCmCmA | SEQ ID | UCCAUGAGGACCAC | SEQ ID |
| | mCfCmAfGmCmAmUmGmsGmsUm | NO: 382 | CAGCAUGGU | NO: 142 |
| ETXS674 | AmsUfsCmCmUmUfGmUfGfGmAmUmG | SEQ ID | AUCCUUGUGGAUGA | SEQ ID |
| | mAfAmGfGmCmAmAmAmsAmsCm | NO: 383 | AGGCAAAAC | NO: 143 |
| ETXS676 | AmsAfsAmAmUmCfCmUfUfGmUmGmG | SEQ ID | AAAAUCCUUGUGGA | SEQ ID |
| | mAfUmGfAmAmGmGmCmsAmsAm | NO: 384 | UGAAGGCAA | NO: 144 |
| ETXS678 | CmsAfsUmGmGmUfGmGfCfAmUmUmU | SEQ ID | CAUGGUGGCAUUUC | SEQ ID |
| | mCfCmUfUmGmGmUmAmsGmsGm | NO: 385 | CUUGGUAGG | NO: 145 |
| ETXS680 | UmsUfsUmCmAmGfGmAfUfUmAmAmU | SEQ ID | UUUCAGGAUUAAUC | SEQ ID |
| | mCfUmCfAmUmCmAmAmsAmsCm | NO: 386 | UCAUCAAAC | NO: 146 |
| ETXS682 | UmsCfsAmAmAmAfUmCfCfUmUmGmU | SEQ ID | UCAAAAUCCUUGUG | SEQ ID |
| | mGfGmAfUmGmAmAmGmsGmsCm | NO: 387 | GAUGAAGGC | NO: 147 |
| ETXS684 | UmsCfsGmUmGmCfCmUfCfAmUmGmG | SEQ ID | UCGUGCCUCAUGGA | SEQ ID |
| | mAfGmAfUmCmUmUmUmsCmsGm | NO: 388 | GAUCUUUCG | NO: 148 |
| ETXS686 | CmsAfsGmGmAmUfUmAfAfUmCmUmC | SEQ ID | CAGGAUUAAUCUCA | SEQ ID |
| | mAfUmCfAmAmAmCmAmsGmsUm | NO: 389 | UCAAACAGU | NO: 149 |
| ETXS688 | CmsUfsCmCmAmUfGmAfGfGmAmCmC | SEQ ID | CUCCAUGAGGACCA | SEQ ID |
| | mAfCmCfAmGmCmAmUmsGmsGm | NO: 390 | CCAGCAUGG | NO: 150 |
| ETXS690 | CmsCfsUmUmGmUfGmGfAfUmGmAmA | SEQ ID | CCUUGUGGAUGAAG | SEQ ID |
| | mGfGmCfAmAmAmAmCmsUmsCm | NO: 391 | GCAAAACUC | NO: 151 |
| ETXS692 | UmsGfsAmGmGmAfCmCfAfCmCmAmG | SEQ ID | UGAGGACCACCAGC | SEQ ID |
| | mCfAmUfGmGmUmGmGmsCmsAm | NO: 392 | AUGGUGGCA | NO: 152 |
| ETXS694 | AmsUfsGmAmGmGfAmCfCfAmCmCmA | SEQ ID | AUGAGGACCACCAG | SEQ ID |
| | mGfCmAfUmGmGmUmGmsGmsCm | NO: 393 | CAUGGUGGC | NO: 153 |
| ETXS696 | AmsUfsCmGmUmGfCmCfUfCmAmUmG | SEQ ID | AUCGUGCCUCAUGG | SEQ ID |
| | mGfAmGfAmUmCmUmUmsUmsCm | NO: 394 | AGAUCUUUC | NO: 154 |
| ETXS698 | CmsUfsUmGmUmGfGmAfUfGmAmAmG | SEQ ID | CUUGUGGAUGAAGG | SEQ ID |
| | mGfCmAfAmAmAmCmUmsCmsCm | NO: 395 | CAAAACUCC | NO: 155 |
| ETXS700 | CmsAfsUmGmAmGfGmAfCfCmAmCmC | SEQ ID | CAUGAGGACCACCA | SEQ ID |
| | mAfGmCfAmUmGmGmUmsGmsGm | NO: 396 | GCAUGGUGG | NO: 156 |
| ETXS702 | CmsAfsUmCmGmUfGmCfCfUmCmAmU | SEQ ID | CAUCGUGCCUCAUG | SEQ ID |
| | mGfGmAfGmAmUmCmUmsUmsUm | NO: 397 | GAGAUCUUU | NO: 157 |
| ETXS704 | CmsCfsAmUmGmAfGmGfAfCmCmAmC | SEQ ID | CCAUGAGGACCACC | SEQ ID |
| | mCfAmGfCmAmUmGmGmsUmsGm | NO: 398 | AGCAUGGUG | NO: 158 |
| ETXS706 | UmsUfsCmUmUmGfUmCfAfAmAmGmG | SEQ ID | UUCUUGUCAAAGGU | SEQ ID |
| | mUfGmGfAmGmGmCmAmsAmsAm | NO: 399 | GGAGGCAAA | NO: 159 |
| ETXS708 | AmsAfsUmUmCmUfUmGfUfCmAmAmA | SEQ ID | AAUUCUUGUCAAAG | SEQ ID |
| | mGfGmUfGmGmAmGmGmsCmsAm | NO: 400 | GUGGAGGCA | NO: 160 |
| ETXS710 | UmsAfsCmAmUmCfAmUfGfGmGmCmA | SEQ ID | UACAUCAUGGGCAC | SEQ ID |
| | mCfCmUfUmAmAmUmGmsGmsUm | NO: 401 | CUUAAUGGU | NO: 161 |
| ETXS712 | GmsGfsCmAmUmUfUmCfCfUmUmGmG | SEQ ID | GGCAUUUCCUUGGU | SEQ ID |
| | mUfAmGfGmGmCmAmGmsUmsUm | NO: 402 | AGGGCAGUU | NO: 162 |
| ETXS714 | UmsUfsUmCmCmUfUmGfGfUmAmGmG | SEQ ID | UUUCCUUGGUAGGG | SEQ ID |
| | mGfCmAfGmUmUmUmGmsAmsGm | NO: 403 | CAGUUUGAG | NO: 163 |
| ETXS716 | UmsUfsCmCmUmUfGmGfUfAmGmGmG | SEQ ID | UUCCUUGGUAGGGC | SEQ ID |
| | mCfAmGfUmUmUmGmAmsGmsGm | NO: 404 | AGUUUGAGG | NO: 164 |
| ETXS718 | AmsAfsAmUmUmCfUmUfGfUmCmAmA | SEQ ID | AAAUUCUUGUCAAA | SEQ ID |
| | mAfGmGfUmGmGmAmGmsGmsCm | NO: 405 | GGUGGAGGC | NO: 165 |
| ETXS720 | UmsUfsGmUmCmCfAmGfGfUmGmGmA | SEQ ID | UUGUCCAGGUGGAA | SEQ ID |
| | mAfAmGfUmGmUmCmGmsAmsCm | NO: 406 | AGUGUCGAC | NO: 166 |
| ETXS722 | GmsUfsAmCmUmUfGmUfCfCmAmGmG | SEQ ID | GUACUUGUCCAGGU | SEQ ID |
| | mUfGmGfAmAmAmGmUmsGmsUm | NO: 407 | GGAAAGUGU | NO: 167 |
| ETXS724 | AmsUfsUmUmCmCfUmUfGfGmUmAmG | SEQ ID | AUUUCCUUGGUAGG | SEQ ID |
| | mGfGmCfAmGmUmUmUmsGmsAm | NO: 408 | GCAGUUUGA | NO: 168 |
| ETXS726 | GmsCfsAmUmUmUfCmCfUfUmGmGmU | SEQ ID | GCAUUUCCUUGGUA | SEQ ID |
| | mAfGmGfGmCmAmGmUmsUmsUm | NO: 409 | GGGCAGUUU | NO: 169 |
| ETXS728 | UmsAfsCmUmUmGfUmCfCfAmGmGmU | SEQ ID | UACUUGUCCAGGUG | SEQ ID |
| | mGfGmAfAmAmGmUmGmsUmsCm | NO: 410 | GAAAGUGUC | NO: 170 |
| ETXS730 | AmsCfsAmUmCmAfUmGfGfGmCmAmC | SEQ ID | ACAUCAUGGGCACC | SEQ ID |
| | mCfUmUfAmAmUmGmGmsUmsCm | NO: 411 | UUAAUGGUC | NO: 171 |
| ETXS732 | UmsCfsUmUmGmUfCmAfAfAmGmGmU | SEQ ID | UCUUGUCAAAGGUG | SEQ ID |
| | mGfGmAfGmGmCmAmAmsAmsCm | NO: 412 | GAGGCAAAC | NO: 172 |
| ETXS734 | UmsUfsGmUmAmCfUmUfGfUmCmCmA | SEQ ID | UUGUACUUGUCCAG | SEQ ID |
| | mGfGmUfGmGmAmAmAmsGmsUm | NO: 413 | GUGGAAAGU | NO: 173 |
| ETXS736 | CmsUfsUmGmGmUfAmGfGfGmCmAmG | SEQ ID | CUUGGUAGGGCAGU | SEQ ID |
| | mUfUmUfGmAmGmGmAmsCmsAm | NO: 414 | UUGAGGACA | NO: 174 |
| ETXS738 | CmsAfsUmUmUmCfCmUfUfGmGmUmA | SEQ ID | CAUUUCCUUGGUAG | SEQ ID |
| | mGfGmGfCmAmGmUmUmsUmsGm | NO: 415 | GGCAGUUUG | NO: 175 |
| ETXS740 | CmsCfsUmUmGmGfUmAfGfGmGmCmA | SEQ ID | CCUUGGUAGGGCAG | SEQ ID |
| | mGfUmUfUmGmAmGmGmsAmsCm | NO: 416 | UUUGAGGAC | NO: 176 |
| ETXS742 | UmsUfsGmGmUmAfGmGfGfCmAmGmU | SEQ ID | UUGGUAGGGCAGUU | SEQ ID |
| | mUfUmGfAmGmGmAmCmsAmsUm | NO: 417 | UGAGGACAU | NO: 177 |
| ETXS744 | CmsUfsUmGmUmCfCmAfGfGmUmGmG | SEQ ID | CUUGUCCAGGUGGA | SEQ ID |
| | mAfAmAfGmUmGmUmCmsGmsAm | NO: 418 | AAGUGUCGA | NO: 178 |
| ETXS746 | CmsUfsUmGmUmCfAmAfAfGmGmUmG | SEQ ID | CUUGUCAAAGGUGG | SEQ ID |
| | mGfAmGfGmCmAmAmAmsCmsUm | NO: 419 | AGGCAAACU | NO: 179 |
| ETXS748 | AmsCfsUmUmGmUfCmCfAfGmGmUmG | SEQ ID | ACUUGUCCAGGUGG | SEQ ID |
| | mGfAmAfAmGmUmGmUmsCmsGm | NO: 420 | AAAGUGUCG | NO: 180 |
| ETXS750 | CmsUfsUmGmUmAfCmUfUfGmUmCmC | SEQ ID | CUUGUACUUGUCCA | SEQ ID |
| | mAfGmGfUmGmGmAmAmsAmsGm | NO: 421 | GGUGGAAAG | NO: 181 |
| ETXS752 | UmsGfsUmAmCmUfUmGfUfCmCmAmG | SEQ ID | UGUACUUGUCCAGG | SEQ ID |
| | mGfUmGfGmAmAmAmGmsUmsGm | NO: 422 | UGGAAAGUG | NO: 182 |
| ETXS754 | UmsCfsCmUmUmGfGmUfAfGmGmGmC | SEQ ID | UCCUUGGUAGGGCA | SEQ ID |
| | mAfGmUfUmUmGmAmGmsGmsAm | NO: 423 | GUUUGAGGA | NO: 183 |
| ETXS756 | UmsUfsCmCmCmUfUmUfGfAmAmCmA | SEQ ID | UUCCCUUUGAACAA | SEQ ID |
| | mAfGmAfUmGmUmAmAmsUmsCm | NO: 424 | GAUGUAAUC | NO: 184 |
| ETXS758 | AmsGfsGmAmCmCfAmCfCfAmGmCmA | SEQ ID | AGGACCACCAGCAU | SEQ ID |
| | mUfGmGfUmGmGmCmAmsUmsUm | NO: 425 | GGUGGCAUU | NO: 185 |
| ETXS760 | UmsAfsUmCmAmAfAmAfUfAmCmCmU | SEQ ID | UAUCAAAAUACCUC | SEQ ID |
| | mCfUmUfGmGmAmUmAmsAmsAm | NO: 426 | UUGGAUAAA | NO: 186 |
| ETXS762 | AmsCfsCmAmCmCfAmGfCfAmUmGmG | SEQ ID | ACCACCAGCAUGGU | SEQ ID |
| | mUfGmGfCmAmUmUmUmsCmsCm | NO: 427 | GGCAUUUCC | NO: 187 |
| ETXS764 | UmsCfsCmCmUmUfUmGfAfAmCmAmA | SEQ ID | UCCCUUUGAACAAG | SEQ ID |
| | mGfAmUfGmUmAmAmUmsCmsCm | NO: 428 | AUGUAAUCC | NO: 188 |
| ETXS766 | UmsUfsGmCmCmAfUmCfGfUmGmCmC | SEQ ID | UUGCCAUCGUGCCU | SEQ ID |
| | mUfCmAfUmGmGmAmGmsAmsUm | NO: 429 | CAUGGAGAU | NO: 189 |
| ETXS768 | UmsUfsUmGmAmUfGmAfCfAmGmGmA | SEQ ID | UUUGAUGACAGGAG | SEQ ID |
| | mGfGmCfAmUmGmGmAmsAmsUm | NO: 430 | GCAUGGAAU | NO: 190 |
| ETXS770 | UmsGfsAmUmGmAfCmAfGfGmAmGmG | SEQ ID | UGAUGACAGGAGGC | SEQ ID |
| | mCfAmUfGmGmAmAmUmsAmsAm | NO: 431 | AUGGAAUAA | NO: 191 |
| ETXS772 | CmsAfsGmCmAmUfGmGfUfGmGmCmA | SEQ ID | CAGCAUGGUGGCAU | SEQ ID |
| | mUfUmUfCmCmUmUmGmsGmsUm | NO: 432 | UUCCUUGGU | NO: 192 |
| ETXS774 | UmsUfsUmUmCmUfCmCfAfUmGmAmG | SEQ ID | UUUUCUCCAUGAGG | SEQ ID |
| | mGfAmCfCmAmCmCmAmsGmsCm | NO: 433 | ACCACCAGC | NO: 193 |
| ETXS776 | CmsCfsAmUmUmUfCmCfCfUmUmUmG | SEQ ID | CCAUUUCCCUUUGA | SEQ ID |
| | mAfAmCfAmAmGmAmUmsGmsUm | NO: 434 | ACAAGAUGU | NO: 194 |
| ETXS778 | CmsCfsAmGmCmAfUmGfGfUmGmGmC | SEQ ID | CCAGCAUGGUGGCA | SEQ ID |
| | mAfUmUfUmCmCmUmUmsGmsGm | NO: 435 | UUUCCUUGG | NO: 195 |
| ETXS780 | AmsUfsUmCmUmUfGmUfCfAmAmAmG | SEQ ID | AUUCUUGUCAAAGG | SEQ ID |
| | mGfUmGfGmAmGmGmCmsAmsAm | NO: 436 | UGGAGGCAA | NO: 196 |
| ETXS782 | UmsUfsGmUmGmGfAmUfGfAmAmGmG | SEQ ID | UUGUGGAUGAAGGC | SEQ ID |
| | mCfAmAfAmAmCmUmCmsCmsCm | NO: 437 | AAAACUCCC | NO: 197 |
| ETXS784 | CmsUfsCmAmUmGfGmAfGfAmUmCmU | SEQ ID | CUCAUGGAGAUCUU | SEQ ID |
| | mUfUmCfGmCmAmGmCmsAmsGm | NO: 438 | UCGCAGCAG | NO: 198 |
| ETXS786 | UmsGfsUmCmAmAfAmGfGfUmGmGmA | SEQ ID | UGUCAAAGGUGGAG | SEQ ID |
| | mGfGmCfAmAmAmCmUmsUmsGm | NO: 439 | GCAAACUUG | NO: 199 |
| ETXS788 | UmsUfsGmUmCmAfAmAfGfGmUmGmG | SEQ ID | UUGUCAAAGGUGGA | SEQ ID |
| | mAfGmGfCmAmAmAmCmsUmsUm | NO: 440 | GGCAAACUU | NO: 200 |
| ETXS790 | CmsAfsUmUmUmCfCmCfUfUmUmGmA | SEQ ID | CAUUUCCCUUUGAA | SEQ ID |
| | mAfCmAfAmGmAmUmGmsUmsAm | NO: 441 | CAAGAUGUA | NO: 201 |
| ETXS792 | UmsGfsUmGmGmAfUmGfAfAmGmGmC | SEQ ID | UGUGGAUGAAGGCA | SEQ ID |
| | mAfAmAfAmCmUmCmCmsCmsCm | NO: 442 | AAACUCCCC | NO: 202 |
| ETXS794 | CmsCfsUmCmAmUfGmGfAfGmAmUmC | SEQ ID | CCUCAUGGAGAUCU | SEQ ID |
| | mUfUmUfCmGmCmAmGmsCmsAm | NO: 443 | UUCGCAGCA | NO: 203 |
| ETXS796 | CmsUfsUmUmGmAfUmGfAfCmAmGmG | SEQ ID | CUUUGAUGACAGGA | SEQ ID |
| | mAfGmGfCmAmUmGmGmsAmsAm | NO: 444 | GGCAUGGAA | NO: 204 |
| ETXS798 | UmsCfsAmCmCmAfCmCfCfUmGmCmC | SEQ ID | UCACCACCCUGCCC | SEQ ID |
| | mCfAmGfAmAmAmCmAmsGmsAm | NO: 445 | AGAAACAGA | NO: 205 |
| ETXS800 | UmsGfsGmAmGmAfUmCfUfUmUmCmG | SEQ ID | UGGAGAUCUUUCGC | SEQ ID |
| | mCfAmGfCmAmGmGmCmsUmsGm | NO: 446 | AGCAGGCUG | NO: 206 |
| ETXS802 | GmsAfsUmUmAmAfUmCfUfCmAmUmC | SEQ ID | GAUUAAUCUCAUCA | SEQ ID |
| | mAfAmAfCmAmGmUmUmsUmsGm | NO: 447 | AACAGUUUG | NO: 207 |
| ETXS804 | CmsAfsCmUmUmUfGmAfUfGmAmCmA | SEQ ID | CACUUUGAUGACAG | SEQ ID |
| | mGfGmAfGmGmCmAmUmsGmsGm | NO: 448 | GAGGCAUGG | NO: 208 |
| ETXS806 | GmsCfsAmGmCmUfCmAfUfGmCmAmU | SEQ ID | GCAGCUCAUGCAUC | SEQ ID |
| | mCfUmCfAmUmAmCmUmsUmsCm | NO: 449 | UCAUACUUC | NO: 209 |
| ETXS808 | CmsAfsGmCmUmCfAmUfGfCmAmUmC | SEQ ID | CAGCUCAUGCAUCU | SEQ ID |
| | mUfCmAfUmAmCmUmUmsCmsUm | NO: 450 | CAUACUUCU | NO: 210 |
| ETXS810 | UmsGfsGmUmAmGfGmGfCfAmGmUmU | SEQ ID | UGGUAGGGCAGUUU | SEQ ID |
| | mUfGmAfGmGmAmCmAmsUmsGm | NO: 451 | GAGGACAUG | NO: 211 |
| ETXS812 | GmsGfsAmUmUmAfAmUfCfUmCmAmU | SEQ ID | GGAUUAAUCUCAUC | SEQ ID |
| | mCfAmAfAmCmAmGmUmsUmsUm | NO: 452 | AAACAGUUU | NO: 212 |
| ETXS814 | AmsUfsCmAmUmGfGmGfCfAmCmCmU | SEQ ID | AUCAUGGGCACCUU | SEQ ID |
| | mUfAmAfUmGmGmUmCmsUmsUm | NO: 453 | AAUGGUCUU | NO: 213 |
| ETXS816 | CmsAfsUmCmAmUfGmGfGfCmAmCmC | SEQ ID | CAUCAUGGGCACCU | SEQ ID |
| | mUfUmAfAmUmGmGmUmsCmsUm | NO: 454 | UAAUGGUCU | NO: 214 |
| ETXS818 | UmsUfsCmAmCmCfAmCfCfCmUmGmC | SEQ ID | UUCACCACCCUGCC | SEQ ID |
| | mCfCmAfGmAmAmAmCmsAmsGm | NO: 455 | CAGAAACAG | NO: 215 |
| ETXS820 | CmsCfsAmCmCmCfUmGfCfCmCmAmG | SEQ ID | CCACCCUGCCCAGA | SEQ ID |
| | mAfAmAfCmAmGmAmAmsGmsCm | NO: 456 | AACAGAAGC | NO: 216 |
| ETXS822 | GmsCfsUmUmGmAfAmCfUfUmCmGmG | SEQ ID | GCUUGAACUUCGGA | SEQ ID |
| | mAfAmAfGmAmAmAmAmsCmsUm | NO: 457 | AAGAAAACU | NO: 217 |
| ETXS824 | AmsGfsCmUmUmGfAmAfCfUmUmCmG | SEQ ID | AGCUUGAACUUCGG | SEQ ID |
| | mGfAmAfAmGmAmAmAmsAmsCm | NO: 458 | AAAGAAAAC | NO: 218 |
| ETXS826 | AmsCfsCmAmCmCfCmUfGfCmCmCmA | SEQ ID | ACCACCCUGCCCAG | SEQ ID |
| | mGfAmAfAmCmAmGmAmsAmsGm | NO: 459 | AAACAGAAG | NO: 219 |
| ETXS828 | GmsGfsUmAmGmGfGmCfAfGmUmUmU | SEQ ID | GGUAGGGCAGUUUG | SEQ ID |
| | mGfAmGfGmAmCmAmUmsGmsAm | NO: 460 | AGGACAUGA | NO: 220 |
| ETXS830 | UmsGfsUmCmCmAfGmGfUfGmGmAmA | SEQ ID | UGUCCAGGUGGAAA | SEQ ID |
| | mAfGmUfGmUmCmGmAmsCmsUm | NO: 461 | GUGUCGACU | NO: 221 |
| ETXS832 | AmsAfsAmUfCmCfUmUfGmUfGmGfA | SEQ ID | AAAUCCUUGUGGAU | SEQ ID |
| | mUfGmAfAmsGfsGm | NO: 462 | GAAGG | NO: 222 |
| ETXS834 | GmsCfsCmUfCmAfUmGfGmAfGmAfU | SEQ ID | GCCUCAUGGAGAUC | SEQ ID |
| | mCfUmUfUmsCfsGm | NO: 463 | UUUCG | NO: 223 |
| ETXS836 | UmsUfsCmUfCmCfAmUfGmAfGmGfA | SEQ ID | UUCUCCAUGAGGAC | SEQ ID |
| | mCfCmAfCmsCfsAm | NO: 464 | CACCA | NO: 224 |
| ETXS838 | UmsGfsCmCfUmCfAmUfGmGfAmGfA | SEQ ID | UGCCUCAUGGAGAU | SEQ ID |
| | mUfCmUfUmsUfsCm | NO: 465 | CUUUC | NO: 225 |
| ETXS840 | AmsGfsCmAfGmCfUmCfAmUfGmCfA | SEQ ID | AGCAGCUCAUGCAU | SEQ ID |
| | mUfCmUfCmsAfsUm | NO: 466 | CUCAU | NO: 226 |
| ETXS842 | UmsCfsAmGfGmAfUmUfAmAfUmCfU | SEQ ID | UCAGGAUUAAUCUC | SEQ ID |
| | mCfAmUfCmsAfsAm | NO: 467 | AUCAA | NO: 227 |
| ETXS844 | UmsUfsGmGfUmUfUmCfAmGfGmAfU | SEQ ID | UUGGUUUCAGGAUU | SEQ ID |
| | mUfAmAfUmsCfsUm | NO: 468 | AAUCU | NO: 228 |
| ETXS846 | GmsUfsUmUfCmAfGmGfAmUfUmAfA | SEQ ID | GUUUCAGGAUUAAU | SEQ ID |
| | mUfCmUfCmsAfsUm | NO: 469 | CUCAU | NO: 229 |
| ETXS848 | UmsGfsGmUfUmUfCmAfGmGfAmUfU | SEQ ID | UGGUUUCAGGAUUA | SEQ ID |
| | mAfAmUfCmsUfsCm | NO: 470 | AUCUC | NO: 230 |
| ETXS850 | GmsGfsUmUfUmCfAmGfGmAfUmUfA | SEQ ID | GGUUUCAGGAUUAA | SEQ ID |
| | mAfUmCfUmsCfsAm | NO: 471 | UCUCA | NO: 231 |
| ETXS852 | AmsGfsGmAfUmUfAmAfUmCfUmCfA | SEQ ID | AGGAUUAAUCUCAU | SEQ ID |
| | mUfCmAfAmsAfsCm | NO: 472 | CAAAC | NO: 232 |
| ETXS854 | UmsCfsCmUfUmGfUmGfGmAfUmGfA | SEQ ID | UCCUUGUGGAUGAA | SEQ ID |
| | mAfGmGfCmsAfsAm | NO: 473 | GGCAA | NO: 233 |
| ETXS856 | GmsUfsGmCfCmUfCmAfUmGfGmAfG | SEQ ID | GUGCCUCAUGGAGA | SEQ ID |
| | mAfUmCfUmsUfsUm | NO: 474 | UCUUU | NO: 234 |
| ETXS858 | UmsCfsUmCfCmAfUmGfAmGfGmAfC | SEQ ID | UCUCCAUGAGGACC | SEQ ID |
| | mCfAmCfCmsAfsGm | NO: 475 | ACCAG | NO: 235 |
| ETXS860 | CmsAfsAmAfAmUfCmCfUmUfGmUfG | SEQ ID | CAAAAUCCUUGUGG | SEQ ID |
| | mGfAmUfGmsAfsAm | NO: 476 | AUGAA | NO: 236 |
| ETXS862 | CmsGfsUmGfCmCfUmCfAmUfGmGfA | SEQ ID | CGUGCCUCAUGGAG | SEQ ID |
| | mGfAmUfCmsUfsUm | NO: 477 | AUCUU | NO: 237 |
| ETXS864 | AmsUfsCmAfAmAfAmUfCmCfUmUfG | SEQ ID | AUCAAAAUCCUUGU | SEQ ID |
| | mUfGmGfAmsUfsGm | NO: 478 | GGAUG | NO: 238 |
| ETXS866 | UmsUfsCmAfGmGfAmUfUmAfAmUfC | SEQ ID | UUCAGGAUUAAUCU | SEQ ID |
| | mUfCmAfUmsCfsAm | NO: 479 | CAUCA | NO: 239 |
| ETXS868 | AmsAfsUmCfCmUfUmGfUmGfGmAfU | SEQ ID | AAUCCUUGUGGAUG | SEQ ID |
| | mGfAmAfGmsGfsCm | NO: 480 | AAGGC | NO: 240 |
| ETXS870 | CmsCfsAmUfCmGfUmGfCmCfUmCfA | SEQ ID | CCAUCGUGCCUCAU | SEQ ID |
| | mUfGmGfAmsGfsAm | NO: 481 | GGAGA | NO: 241 |
| ETXS872 | AmsAfsAmUfCmCfUmUfGfUmGmGmA | SEQ ID | AAAUCCUUGUGGAU | SEQ ID |
| | mUfGmAfAmGmGmCmAmsAmsAm | NO: 482 | GAAGGCAAA | NO: 122 |
| ETXS874 | GmsCfsCmUfCmAfUmGfGfAmGmAmU | SEQ ID | GCCUCAUGGAGAUC | SEQ ID |
| | mCfUmUfUmCmGmCmAmsGmsCm | NO: 483 | UUUCGCAGC | NO: 123 |
| ETXS876 | UmsUfsCmUfCmCfAmUfGfAmGmGmA | SEQ ID | UUCUCCAUGAGGAC | SEQ ID |
| | mCfCmAfCmCmAmGmCmsAmsUm | NO: 484 | CACCAGCAU | NO: 124 |
| ETXS878 | UmsGfsCmCfUmCfAmUfGfGmAmGmA | SEQ ID | UGCCUCAUGGAGAU | SEQ ID |
| | mUfCmUfUmUmCmGmCmsAmsGm | NO: 485 | CUUUCGCAG | NO: 125 |
| ETXS880 | AmsGfsCmAfGmCfUmCfAfUmGmCmA | SEQ ID | AGCAGCUCAUGCAU | SEQ ID |
| | mUfCmUfCmAmUmAmCmsUmsUm | NO: 486 | CUCAUACUU | NO: 126 |
| ETXS882 | UmsCfsAmGfGmAfUmUfAfAmUmCmU | SEQ ID | UCAGGAUUAAUCUC | SEQ ID |
| | mCfAmUfCmAmAmAmCmsAmsGm | NO: 487 | AUCAAACAG | NO: 127 |
| ETXS884 | UmsUfsGmGfUmUfUmCfAfGmGmAmU | SEQ ID | UUGGUUUCAGGAUU | SEQ ID |
| | mUfAmAfUmCmUmCmAmsUmsCm | NO: 488 | AAUCUCAUC | NO: 128 |
| ETXS886 | GmsUfsUmUfCmAfGmGfAfUmUmAmA | SEQ ID | GUUUCAGGAUUAAU | SEQ ID |
| | mUfCmUfCmAmUmCmAmsAmsAm | NO: 489 | CUCAUCAAA | NO: 129 |
| ETXS888 | UmsGfsGmUfUmUfCmAfGfGmAmUmU | SEQ ID | UGGUUUCAGGAUUA | SEQ ID |
| | mAfAmUfCmUmCmAmUmsCmsAm | NO: 490 | AUCUCAUCA | NO: 130 |
| ETXS890 | GmsGfsUmUfUmCfAmGfGfAmUmUmA | SEQ ID | GGUUUCAGGAUUAA | SEQ ID |
| | mAfUmCfUmCmAmUmCmsAmsAm | NO: 491 | UCUCAUCAA | NO: 131 |
| ETXS892 | AmsGfsGmAfUmUfAmAfUfCmUmCmA | SEQ ID | AGGAUUAAUCUCAU | SEQ ID |
| | mUfCmAfAmAmCmAmGmsUmsUm | NO: 492 | CAAACAGUU | NO: 132 |
| ETXS894 | UmsCfsCmUfUmGfUmGfGfAmUmGmA | SEQ ID | UCCUUGUGGAUGAA | SEQ ID |
| | mAfGmGfCmAmAmAmAmsCmsUm | NO: 493 | GGCAAAACU | NO: 133 |
| ETXS896 | GmsUfsGmCfCmUfCmAfUfGmGmAmG | SEQ ID | GUGCCUCAUGGAGA | SEQ ID |
| | mAfUmCfUmUmUmCmGmsCmsAm | NO: 494 | UCUUUCGCA | NO: 134 |
| ETXS898 | UmsCfsUmCfCmAfUmGfAfGmGmAmC | SEQ ID | UCUCCAUGAGGACC | SEQ ID |
| | mCfAmCfCmAmGmCmAmsUmsGm | NO: 495 | ACCAGCAUG | NO: 135 |
| ETXS900 | CmsAfsAmAfAmUfCmCfUfUmGmUmG | SEQ ID | CAAAAUCCUUGUGG | SEQ ID |
| | mGfAmUfGmAmAmGmGmsCmsAm | NO: 496 | AUGAAGGCA | NO: 136 |
| ETXS902 | CmsGfsUmGfCmCfUmCfAfUmGmGmA | SEQ ID | CGUGCCUCAUGGAG | SEQ ID |
| | mGfAmUfCmUmUmUmCmsGmsCm | NO: 497 | AUCUUUCGC | NO: 137 |
| ETXS904 | AmsUfsCmAfAmAfAmUfCfCmUmUmG | SEQ ID | AUCAAAAUCCUUGU | SEQ ID |
| | mUfGmGfAmUmGmAmAmsGmsGm | NO: 498 | GGAUGAAGG | NO: 138 |
| ETXS906 | UmsUfsCmAfGmGfAmUfUfAmAmUmC | SEQ ID | UUCAGGAUUAAUCU | SEQ ID |
| | mUfCmAfUmCmAmAmAmsCmsAm | NO: 499 | CAUCAAACA | NO: 139 |
| ETXS908 | AmsAfsUmCfCmUfUmGfUfGmGmAmU | SEQ ID | AAUCCUUGUGGAUG | SEQ ID |
| | mGfAmAfGmGmCmAmAmsAmsAm | NO: 500 | AAGGCAAAA | NO: 140 |
| ETXS910 | CmsCfsAmUfCmGfUmGfCfCmUmCmA | SEQ ID | CCAUCGUGCCUCAU | SEQ ID |
| | mUfGmGfAmGmAmUmCmsUmsUm | NO: 501 | GGAGAUCUU | NO: 141 |
| ETXS912 | AmsAfsAmUfCmCfUmUfGfUmGmGmA | SEQ ID | AAAUCCUUGUGGAU | SEQ ID |
| | mUfGmAfAmGmGmCmAmsAmsAm | NO: 502 | GAAGGCAAA | NO: 122 |
| ETXS914 | GmsCfsCmUfCmAfUmGfGfAmGmAmU | SEQ ID | GCCUCAUGGAGAUC | SEQ ID |
| | mCfUmUfUmCmGmCmAmsGmsCm | NO: 503 | UUUCGCAGC | NO: 123 |
| ETXS916 | UmsUfsCmUfCmCfAmUfGfAmGmGmA | SEQ ID | UUCUCCAUGAGGAC | SEQ ID |
| | mCfCmAfCmCmAmGmCmsAmsUm | NO: 504 | CACCAGCAU | NO: 124 |
| ETXS918 | UmsGfsCmCfUmCfAmUfGfGmAmGmA | SEQ ID | UGCCUCAUGGAGAU | SEQ ID |
| | mUfCmUfUmUmCmGmCmsAmsGm | NO: 505 | CUUUCGCAG | NO: 125 |
| ETXS920 | AmsGfsCmAfGmCfUmCfAfUmGmCmA | SEQ ID | AGCAGCUCAUGCAU | SEQ ID |
| | mUfCmUfCmAmUmAmCmsUmsUm | NO: 506 | CUCAUACUU | NO: 126 |
| ETXS922 | UmsCfsAmGfGmAfUmUfAfAmUmCmU | SEQ ID | UCAGGAUUAAUCUC | SEQ ID |
| | mCfAmUfCmAmAmAmCmsAmsGm | NO: 507 | AUCAAACAG | NO: 127 |
| ETXS924 | UmsUfsGmGfUmUfUmCfAfGmGmAmU | SEQ ID | UUGGUUUCAGGAUU | SEQ ID |
| | mUfAmAfUmCmUmCmAmsUmsCm | NO: 508 | AAUCUCAUC | NO: 128 |
| ETXS926 | GmsUfsUmUfCmAfGmGfAfUmUmAmA | SEQ ID | GUUUCAGGAUUAAU | SEQ ID |
| | mUfCmUfCmAmUmCmAmsAmsAm | NO: 509 | CUCAUCAAA | NO: 129 |
| ETXS928 | UmsGfsGmUfUmUfCmAfGfGmAmUmU | SEQ ID | UGGUUUCAGGAUUA | SEQ ID |
| | mAfAmUfCmUmCmAmUmsCmsAm | NO: 510 | AUCUCAUCA | NO: 130 |
| ETXS930 | GmsGfsUmUfUmCfAmGfGfAmUmUmA | SEQ ID | GGUUUCAGGAUUAA | SEQ ID |
| | mAfUmCfUmCmAmUmCmsAmsAm | NO: 511 | UCUCAUCAA | NO: 131 |
| ETXS932 | AmsGfsGmAfUmUfAmAfUfCmUmCmA | SEQ ID | AGGAUUAAUCUCAU | SEQ ID |
| | mUfCmAfAmAmCmAmGmsUmsUm | NO: 512 | CAAACAGUU | NO: 132 |
| ETXS934 | UmsCfsCmUfUmGfUmGfGfAmUmGmA | SEQ ID | UCCUUGUGGAUGAA | SEQ ID |
| | mAfGmGfCmAmAmAmAmsCmsUm | NO: 513 | GGCAAAACU | NO: 133 |
| ETXS936 | GmsUfsGmCfCmUfCmAfUfGmGmAmG | SEQ ID | GUGCCUCAUGGAGA | SEQ ID |
| | mAfUmCfUmUmUmCmGmsCmsAm | NO: 514 | UCUUUCGCA | NO: 134 |
| ETXS938 | UmsCfsUmCfCmAfUmGfAfGmGmAmC | SEQ ID | UCUCCAUGAGGACC | SEQ ID |
| | mCfAmCfCmAmGmCmAmsUmsGm | NO: 515 | ACCAGCAUG | NO: 135 |
| ETXS940 | CmsAfsAmAfAmUfCmCfUfUmGmUmG | SEQ ID | CAAAAUCCUUGUGG | SEQ ID |
| | mGfAmUfGmAmAmGmGmsCmsAm | NO: 516 | AUGAAGGCA | NO: 136 |
| ETXS942 | CmsGfsUmGfCmCfUmCfAfUmGmGmA | SEQ ID | CGUGCCUCAUGGAG | SEQ ID |
| | mGfAmUfCmUmUmUmCmsGmsCm | NO: 517 | AUCUUUCGC | NO: 137 |
| ETXS944 | AmsUfsCmAfAmAfAmUfCfCmUmUmG | SEQ ID | AUCAAAAUCCUUGU | SEQ ID |
| | mUfGmGfAmUmGmAmAmsGmsGm | NO: 518 | GGAUGAAGG | NO: 138 |
| ETXS946 | UmsUfsCmAfGmGfAmUfUfAmAmUmC | SEQ ID | UUCAGGAUUAAUCU | SEQ ID |
| | mUfCmAfUmCmAmAmAmsCmsAm | NO: 519 | CAUCAAACA | NO: 139 |
| ETXS948 | AmsAfsUmCfCmUfUmGfUfGmGmAmU | SEQ ID | AAUCCUUGUGGAUG | SEQ ID |
| | mGfAmAfGmGmCmAmAmsAmsAm | NO: 520 | AAGGCAAAA | NO: 140 |
| ETXS950 | CmsCfsAmUfCmGfUmGfCfCmUmCmA | SEQ ID | CCAUCGUGCCUCAU | SEQ ID |
| | mUfGmGfAmGmAmUmCmsUmsUm | NO: 521 | GGAGAUCUU | NO: 141 |
| ETXS952 | AmsAfsAmUfCmCfUmUfGfUmGmGmA | SEQ ID | AAAUCCUUGUGGAU | SEQ ID |
| | mUfGmAfAmGmGmCmAmsAmsAm | NO: 522 | GAAGGCAAA | NO: 122 |
| ETXS954 | GmsCfsCmUfCmAfUmGfGfAmGmAmU | SEQ ID | GCCUCAUGGAGAUC | SEQ ID |
| | mCfUmUfUmCmGmCmAmsGmsCm | NO: 523 | UUUCGCAGC | NO: 123 |
| ETXS956 | UmsUfsCmUfCmCfAmUfGfAmGmGmA | SEQ ID | UUCUCCAUGAGGAC | SEQ ID |
| | mCfCmAfCmCmAmGmCmsAmsUm | NO: 524 | CACCAGCAU | NO: 124 |
| ETXS958 | UmsGfsCmCfUmCfAmUfGfGmAmGmA | SEQ ID | UGCCUCAUGGAGAU | SEQ ID |
| | mUfCmUfUmUmCmGmCmsAmsGm | NO: 525 | CUUUCGCAG | NO: 125 |
| ETXS960 | AmsGfsCmAfGmCfUmCfAfUmGmCmA | SEQ ID | AGCAGCUCAUGCAU | SEQ ID |
| | mUfCmUfCmAmUmAmCmsUmsUm | NO: 526 | CUCAUACUU | NO: 126 |
| ETXS962 | UmsCfsAmGfGmAfUmUfAfAmUmCmU | SEQ ID | UCAGGAUUAAUCUC | SEQ ID |
| | mCfAmUfCmAmAmAmCmsAmsGm | NO: 527 | AUCAAACAG | NO: 127 |
| ETXS964 | UmsUfsGmGfUmUfUmCfAfGmGmAmU | SEQ ID | UUGGUUUCAGGAUU | SEQ ID |
| | mUfAmAfUmCmUmCmAmsUmsCm | NO: 528 | AAUCUCAUC | NO: 128 |
| ETXS966 | GmsUfsUmUfCmAfGmGfAfUmUmAmA | SEQ ID | GUUUCAGGAUUAAU | SEQ ID |
| | mUfCmUfCmAmUmCmAmsAmsAm | NO: 529 | CUCAUCAAA | NO: 129 |
| ETXS968 | UmsGfsGmUfUmUfCmAfGfGmAmUmU | SEQ ID | UGGUUUCAGGAUUA | SEQ ID |
| | mAfAmUfCmUmCmAmUmsCmsAm | NO: 530 | AUCUCAUCA | NO: 130 |
| ETXS970 | GmsGfsUmUfUmCfAmGfGfAmUmUmA | SEQ ID | GGUUUCAGGAUUAA | SEQ ID |
| | mAfUmCfUmCmAmUmCmsAmsAm | NO: 531 | UCUCAUCAA | NO: 131 |
| ETXS972 | AmsGfsGmAfUmUfAmAfUfCmUmCmA | SEQ ID | AGGAUUAAUCUCAU | SEQ ID |
| | mUfCmAfAmAmCmAmGmsUmsUm | NO: 532 | CAAACAGUU | NO: 132 |
| ETXS974 | UmsCfsCmUfUmGfUmGfGfAmUmGmA | SEQ ID | UCCUUGUGGAUGAA | SEQ ID |
| | mAfGmGfCmAmAmAmAmsCmsUm | NO: 533 | GGCAAAACU | NO: 133 |
| ETXS976 | GmsUfsGmCfCmUfCmAfUfGmGmAmG | SEQ ID | GUGCCUCAUGGAGA | SEQ ID |
| | mAfUmCfUmUmUmCmGmsCmsAm | NO: 534 | UCUUUCGCA | NO: 134 |
| ETXS978 | UmsCfsUmCfCmAfUmGfAfGmGmAmC | SEQ ID | UCUCCAUGAGGACC | SEQ ID |
| | mCfAmCfCmAmGmCmAmsUmsGm | NO: 535 | ACCAGCAUG | NO: 135 |
| ETXS980 | CmsAfsAmAfAmUfCmCfUfUmGmUmG | SEQ ID | CAAAAUCCUUGUGG | SEQ ID |
| | mGfAmUfGmAmAmGmGmsCmsAm | NO: 536 | AUGAAGGCA | NO: 136 |
| ETXS982 | CmsGfsUmGfCmCfUmCfAfUmGmGmA | SEQ ID | CGUGCCUCAUGGAG | SEQ ID |
| | mGfAmUfCmUmUmUmCmsGmsCm | NO: 537 | AUCUUUCGC | NO: 137 |
| ETXS984 | AmsUfsCmAfAmAfAmUfCfCmUmUmG | SEQ ID | AUCAAAAUCCUUGU | SEQ ID |
| | mUfGmGfAmUmGmAmAmsGmsGm | NO: 538 | GGAUGAAGG | NO: 138 |
| ETXS986 | UmsUfsCmAfGmGfAmUfUfAmAmUmC | SEQ ID | UUCAGGAUUAAUCU | SEQ ID |
| | mUfCmAfUmCmAmAmAmsCmsAm | NO: 539 | CAUCAAACA | NO: 139 |
| ETXS988 | AmsAfsUmCfCmUfUmGfUfGmGmAmU | SEQ ID | AAUCCUUGUGGAUG | SEQ ID |
| | mGfAmAfGmGmCmAmAmsAmsAm | NO: 540 | AAGGCAAAA | NO: 140 |
| ETXS990 | CmsCfsAmUfCmGfUmGfCfCmUmCmA | SEQ ID | CCAUCGUGCCUCAU | SEQ ID |
| | mUfGmGfAmGmAmUmCmsUmsUm | NO: 541 | GGAGAUCUU | NO: 141 |
| ETXS992 | AmsAfsAmUfCmCfUmUmGmUmGmGmA | SEQ ID | AAAUCCUUGUGGAU | SEQ ID |
| | mUfGmAfAmGmGmCmAmsAmsAm | NO: 542 | GAAGGCAAA | NO: 122 |
| ETXS994 | GmsCfsCmUfCmAfUmGmGmAmGmAmU | SEQ ID | GCCUCAUGGAGAUC | SEQ ID |
| | mCfUmUfUmCmGmCmAmsGmsCm | NO: 543 | UUUCGCAGC | NO: 123 |
| ETXS996 | UmsUfsCmUfCmCfAmUmGmAmGmGmA | SEQ ID | UUCUCCAUGAGGAC | SEQ ID |
| | mCfCmAfCmCmAmGmCmsAmsUm | NO: 544 | CACCAGCAU | NO: 124 |
| ETXS998 | UmsGfsCmCfUmCfAmUmGmGmAmGmA | SEQ ID | UGCCUCAUGGAGAU | SEQ ID |
| | mUfCmUfUmUmCmGmCmsAmsGm | NO: 545 | CUUUCGCAG | NO: 125 |
| ETXS1000 | AmsGfsCmAfGmCfUmCmAmUmGmCmA | SEQ ID | AGCAGCUCAUGCAU | SEQ ID |
| | mUfCmUfCmAmUmAmCmsUmsUm | NO: 546 | CUCAUACUU | NO: 126 |
| ETXS1002 | UmsCfsAmGfGmAfUmUmAmAmUmCmU | SEQ ID | UCAGGAUUAAUCUC | SEQ ID |
| | mCfAmUfCmAmAmAmCmsAmsGm | NO: 547 | AUCAAACAG | NO: 127 |
| ETXS1004 | UmsUfsGmGfUmUfUmCmAmGmGmAmU | SEQ ID | UUGGUUUCAGGAUU | SEQ ID |
| | mUfAmAfUmCmUmCmAmsUmsCm | NO: 548 | AAUCUCAUC | NO: 128 |
| ETXS1006 | GmsUfsUmUfCmAfGmGmAmUmUmAmA | SEQ ID | GUUUCAGGAUUAAU | SEQ ID |
| | mUfCmUfCmAmUmCmAmsAmsAm | NO: 549 | CUCAUCAAA | NO: 129 |
| ETXS1008 | UmsGfsGmUfUmUfCmAmGmGmAmUmU | SEQ ID | UGGUUUCAGGAUUA | SEQ ID |
| | mAfAmUfCmUmCmAmUmsCmsAm | NO: 550 | AUCUCAUCA | NO: 130 |
| ETXS1010 | GmsGfsUmUfUmCfAmGmGmAmUmUmA | SEQ ID | GGUUUCAGGAUUAA | SEQ ID |
| | mAfUmCfUmCmAmUmCmsAmsAm | NO: 551 | UCUCAUCAA | NO: 131 |
| ETXS1012 | AmsGfsGmAfUmUfAmAmUmCmUmCmA | SEQ ID | AGGAUUAAUCUCAU | SEQ ID |
| | mUfCmAfAmAmCmAmGmsUmsUm | NO: 552 | CAAACAGUU | NO: 132 |
| ETXS1014 | UmsCfsCmUfUmGfUmGmGmAmUmGmA | SEQ ID | UCCUUGUGGAUGAA | SEQ ID |
| | mAfGmGfCmAmAmAmAmsCmsUm | NO: 553 | GGCAAAACU | NO: 133 |
| ETXS1016 | GmsUfsGmCfCmUfCmAmUmGmGmAmG | SEQ ID | GUGCCUCAUGGAGA | SEQ ID |
| | mAfUmCfUmUmUmCmGmsCmsAm | NO: 554 | UCUUUCGCA | NO: 134 |
| ETXS1018 | UmsCfsUmCfCmAfUmGmAmGmGmAmC | SEQ ID | UCUCCAUGAGGACC | SEQ ID |
| | mCfAmCfCmAmGmCmAmsUmsGm | NO: 555 | ACCAGCAUG | NO: 135 |
| ETXS1020 | CmsAfsAmAfAmUfCmCmUmUmGmUmG | SEQ ID | CAAAAUCCUUGUGG | SEQ ID |
| | mGfAmUfGmAmAmGmGmsCmsAm | NO: 556 | AUGAAGGCA | NO: 136 |
| ETXS1022 | CmsGfsUmGfCmCfUmCmAmUmGmGmA | SEQ ID | CGUGCCUCAUGGAG | SEQ ID |
| | mGfAmUfCmUmUmUmCmsGmsCm | NO: 557 | AUCUUUCGC | NO: 137 |
| ETXS1024 | AmsUfsCmAfAmAfAmUmCmCmUmUmG | SEQ ID | AUCAAAAUCCUUGU | SEQ ID |
| | mUfGmGfAmUmGmAmAmsGmsGm | NO: 558 | GGAUGAAGG | NO: 138 |
| ETXS1026 | UmsUfsCmAfGmGfAmUmUmAmAmUmC | SEQ ID | UUCAGGAUUAAUCU | SEQ ID |
| | mUfCmAfUmCmAmAmAmsCmsAm | NO: 559 | CAUCAAACA | NO: 139 |
| ETXS1028 | AmsAfsUmCfCmUfUmGmUmGmGmAmU | SEQ ID | AAUCCUUGUGGAUG | SEQ ID |
| | mGfAmAfGmGmCmAmAmsAmsAm | NO: 560 | AAGGCAAAA | NO: 140 |
| ETXS1030 | CmsCfsAmUfCmGfUmGmCmCmUmCmA | SEQ ID | CCAUCGUGCCUCAU | SEQ ID |
| | mUfGmGfAmGmAmUmCmsUmsUm | NO: 561 | GGAGAUCUU | NO: 141 |
| ETXS1032 | AmsAfsAmAfUmCfCmUmUmGmUmGmG | SEQ ID | AAAAUCCUUGUGGA | SEQ ID |
| | mAfUmGfAmAmGmGmCmsAmsAm | NO: 762 | UGAAGGCAA | NO: 144 |
| ETXS1034 | AmsAfsAmAmUmCfCmUfUmGmUmGmG | SEQ ID | AAAAUCCUUGUGGA | SEQ ID |
| | mAfUmGfAmAmGmGmCmsAmsAm | NO: 763 | UGAAGGCAA | NO: 144 |
| ETXS1036 | CmsAfsUmGfGmUfGmGmCmAmUmUmU | SEQ ID | CAUGGUGGCAUUUC | SEQ ID |
| | mCfCmUfUmGmGmUmAmsGmsGm | NO: 764 | CUUGGUAGG | NO: 145 |
| ETXS1038 | CmsAfsUmGmGmUfGmGfCmAmUmUmU | SEQ ID | CAUGGUGGCAUUUC | SEQ ID |
| | mCfCmUfUmGmGmUmAmsGmsGm | NO: 765 | CUUGGUAGG | NO: 145 |
| ETXS1040 | UmsCfsGmUfGmCfCmUmCmAmUmGmG | SEQ ID | UCGUGCCUCAUGGA | SEQ ID |
| | mAfGmAfUmCmUmUmUmsCmsGm | NO: 766 | GAUCUUUCG | NO: 148 |
| ETXS1042 | UmsCfsGmUmGmCfCmUfCmAmUmGmG | SEQ ID | UCGUGCCUCAUGGA | SEQ ID |
| | mAfGmAfUmCmUmUmUmsCmsGm | NO: 767 | GAUCUUUCG | NO: 148 |
| ETXS1044 | AmsAfsAmUfUmCfUmUmGmUmCmAmA | SEQ ID | AAAUUCUUGUCAAA | SEQ ID |
| | mAfGmGfUmGmGmAmGmsGmsCm | NO: 768 | GGUGGAGGC | NO: 165 |
| ETXS1046 | AmsAfsAmUmUmCfUmUfGmUmCmAmA | SEQ ID | AAAUUCUUGUCAAA | SEQ ID |
| | mAfGmGfUmGmGmAmGmsGmsCm | NO: 769 | GGUGGAGGC | NO: 165 |
| ETXS1048 | UmsGfsUmGfGmAfUmGmAmAmGmGmC | SEQ ID | UGUGGAUGAAGGCA | SEQ ID |
| | mAfAmAfAmCmUmCmCmsCmsCm | NO: 770 | AAACUCCCC | NO: 202 |
| ETXS1050 | UmsGfsUmGmGmAfUmGfAmAmGmGmC | SEQ ID | UGUGGAUGAAGGCA | SEQ ID |
| | mAfAmAfAmCmUmCmCmsCmsCm | NO: 771 | AAACUCCCC | NO: 202 |
| ETXS1051 | AmsAfsAmAmUmCfCmUmUmGmUmGmG | SEQ ID | AAAAUCCUUGUGGA | SEQ ID |
| | mAfUmGfAmAfGmGmCmsAmsAm | NO: 782 | UGAAGGCAA | NO: 144 |
| ETXS1052 | CmsAfsUmGmGmUfGmGmCmAmUmUmU | SEQ ID | CAUGGUGGCAUUUC | SEQ ID |
| | mCfCmUfUmGfGmUmAmsGmsGm | NO: 783 | CUUGGUAGG | NO: 145 |
| ETXS1053 | UmsCfsGmUmGmCfCmUmCmAmUmGmG | SEQ ID | UCGUGCCUCAUGGA | SEQ ID |
| | mAfGmAfUmCfUmUmUmsCmsGm | NO: 784 | GAUCUUUCG | NO: 148 |
| ETXS1054 | AmsAfsAmUmUmCfUmUmGmUmCmAmA | SEQ ID | AAAUUCUUGUCAAA | SEQ ID |
| | mAfGmGfUmGfGmAmGmsGmsCm | NO: 785 | GGUGGAGGC | NO: 165 |
| ETXS1055 | UmsGfsUmGmGmAfUmGmAmAmGmGmC | SEQ ID | UGUGGAUGAAGGCA | SEQ ID |
| | mAfAmAfAmCfUmCmCmsCmsCm | NO: 786 | AAACUCCCC | NO: 202 |
| ETXS2128 | AmsAfsAmGmUmCfCmUfUfGmUmGmG | SEQ ID | AAAGUCCUUGUGGA | SEQ ID |
| | mAfUmGfAmAmGmGmCmsAmsAm | NO: 795 | UGAAGGCAA | NO: 787 |
| ETXS2144 | AmsAfsCmUmUmCfUmUfGfUmCmAmA | SEQ ID | AACUUCUUGUCAAA | SEQ ID |
| | mAfGmGfUmGmGmAmGmsGmsCm | NO: 796 | GGUGGAGGC | NO: 788 |
| ETXS2152 | UmsGfsUmGmGmAfUmGfAfAmGmGmC | SEQ ID | UGUGGAUGAAGGCA | SEQ ID |
| | mAfAmAfGmCmUmAmCmsCmsCm | NO: 797 | AAGCUACCC | NO: 789 |

In certain embodiments, the second strand comprises a nucleoside sequence of at least 17 contiguous nucleosides differing by 0 or 1 nucleosides from any one of SEQ TD NO:562-761, 772-781 or 798-800; wherein the second strand has a region of at least 8500 complementarity over the 17 contiguous nucleosides to the first strand.

In certain embodiments, the second strand comprises a nucleoside sequence of at least 19 contiguous nucleosides differing by 0 or 1 nucleosides from any one of SEQ TD NO:562-761, 772-781 or 798-800; wherein the second strand has a region of at least 85% o complementarity over the 19 contiguous nucleosides to the first strand.

In certain embodiments, the second strand comprises a nucleoside sequence of at least 21 contiguous nucleosides differing by 0 or 1 nucleosides from any one of SEQ TD NO:562-761, 772-781 or 798-800; wherein the second strand has a region of at least 85% o complementarity over the 21 contiguous nucleosides to the first strand.

In certain embodiments, the second strand comprises any one of SEQ ID NO:562-761, 772-781 or 798-800.

The modification pattern of the nucleic acids as set forth in SEQ TD NO: 562-761, 772-781 or 798-800 is summarized in Table 4 below:

TABLE 4

| | | | Underlying | |
|---|---|---|---|---|
| | | | Base | |
| | | | Sequence | |
| | | | 5′ → 3′ | |
| | | SEQ ID | (Shown as an | SEQ ID |
| Sense | Modified Second | NO | Unmodified | NO |
| strand | (Sense) Strand | (SS - | Nucleotide | (SS - |
| ID | 5′ → 3′ | mod) | Sequence) | unmod) |
| ETXS631 | UmsGmsCmCmUmUmCfAmUfCfCfAmCmA | SEQ ID | UGCCUUCAUCCACA | SEQ ID |
| | mAmGmGmAmUmUmUm | NO: 562 | AGGAUUU | NO: 242 |
| ETXS633 | UmsGmsCmGmAmAmAfGmAfUfCfUmCmC | SEQ ID | UGCGAAAGAUCUCC | SEQ ID |
| | mAmUmGmAmGmGmCm | NO: 563 | AUGAGGC | NO: 243 |
| ETXS635 | GmsCmsUmGmGmUmGfGmUfCfCfUmCmA | SEQ ID | GCUGGUGGUCCUCA | SEQ ID |
| | mUmGmGmAmGmAmAm | NO: 564 | UGGAGAA | NO: 244 |
| ETXS637 | GmsCmsGmAmAmAmGfAmUfCfUfCmCmA | SEQ ID | GCGAAAGAUCUCCA | SEQ ID |
| | mUmGmAmGmGmCmAm | NO: 565 | UGAGGCA | NO: 245 |
| ETXS639 | GmsUmsAmUmGmAmGfAmUfGfCfAmUmG | SEQ ID | GUAUGAGAUGCAUG | SEQ ID |
| | mAmGmCmUmGmCmUm | NO: 566 | AGCUGCU | NO: 246 |
| ETXS641 | GmsUmsUmUmGmAmUfGmAfGfAfUmUmA | SEQ ID | GUUUGAUGAGAUUA | SEQ ID |
| | mAmUmCmCmUmGmAm | NO: 567 | AUCCUGA | NO: 247 |
| ETXS643 | UmsGmsAmGmAmUmUfAmAfUfCfCmUmG | SEQ ID | UGAGAUUAAUCCUG | SEQ ID |
| | mAmAmAmCmCmAmAm | NO: 568 | AAACCAA | NO: 248 |
| ETXS645 | UmsGmsAmUmGmAmGfAmUfUfAfAmUmC | SEQ ID | UGAUGAGAUUAAUC | SEQ ID |
| | mCmUmGmAmAmAmCm | NO: 569 | CUGAAAC | NO: 249 |
| ETXS647 | AmsUmsGmAmGmAmUfUmAfAfUfCmCmU | SEQ ID | AUGAGAUUAAUCCU | SEQ ID |
| | mGmAmAmAmCmCmAm | NO: 570 | GAAACCA | NO: 250 |
| ETXS649 | GmsAmsUmGmAmGmAfUmUfAfAfUmCmC | SEQ ID | GAUGAGAUUAAUCC | SEQ ID |
| | mUmGmAmAmAmCmCm | NO: 571 | UGAAACC | NO: 251 |
| ETXS651 | CmsUmsGmUmUmUmGfAmUfGfAfGmAmU | SEQ ID | CUGUUUGAUGAGAU | SEQ ID |
| | mUmAmAmUmCmCmUm | NO: 572 | UAAUCCU | NO: 252 |
| ETXS653 | UmsUmsUmUmGmCmCfUmUfCfAfUmCmC | SEQ ID | UUUUGCCUUCAUCC | SEQ ID |
| | mAmCmAmAmGmGmAm | NO: 573 | ACAAGGA | NO: 253 |
| ETXS655 | CmsGmsAmAmAmGmAfUmCfUfCfCmAmU | SEQ ID | CGAAAGAUCUCCAU | SEQ ID |
| | mGmAmGmGmCmAmCm | NO: 574 | GAGGCAC | NO: 254 |
| ETXS657 | UmsGmsCmUmGmGmUfGmGfUfCfCmUmC | SEQ ID | UGCUGGUGGUCCUC | SEQ ID |
| | mAmUmGmGmAmGmAm | NO: 575 | AUGGAGA | NO: 255 |
| ETXS659 | CmsCmsUmUmCmAmUfCmCfAfCfAmAmG | SEQ ID | CCUUCAUCCACAAG | SEQ ID |
| | mGmAmUmUmUmUmGm | NO: 576 | GAUUUUG | NO: 256 |
| ETXS661 | GmsAmsAmAmGmAmUfCmUfCfCfAmUmG | SEQ ID | GAAAGAUCUCCAUG | SEQ ID |
| | mAmGmGmCmAmCmGm | NO: 577 | AGGCACG | NO: 257 |
| ETXS663 | UmsUmsCmAmUmCmCfAmCfAfAfGmGmA | SEQ ID | UUCAUCCACAAGGA | SEQ ID |
| | mUmUmUmUmGmAmUm | NO: 578 | UUUUGAU | NO: 258 |
| ETXS665 | UmsUmsUmGmAmUmGfAmGfAfUfUmAmA | SEQ ID | UUUGAUGAGAUUAA | SEQ ID |
| | mUmCmCmUmGmAmAm | NO: 579 | UCCUGAA | NO: 259 |
| ETXS667 | UmsUmsGmCmCmUmUfCmAfUfCfCmAmC | SEQ ID | UUGCCUUCAUCCAC | SEQ ID |
| | mAmAmGmGmAmUmUm | NO: 580 | AAGGAUU | NO: 260 |
| ETXS669 | GmsAmsUmCmUmCmCfAmUfGfAfGmGmC | SEQ ID | GAUCUCCAUGAGGC | SEQ ID |
| | mAmCmGmAmUmGmGm | NO: 581 | ACGAUGG | NO: 261 |
| ETXS671 | CmsAmsUmGmCmUmGfGmUfGfGfUmCmC | SEQ ID | CAUGCUGGUGGUCC | SEQ ID |
| | mUmCmAmUmGmGmAm | NO: 582 | UCAUGGA | NO: 262 |
| ETXS673 | UmsUmsUmGmCmCmUfUmCfAfUfCmCmA | SEQ ID | UUUGCCUUCAUCCA | SEQ ID |
| | mCmAmAmGmGmAmUm | NO: 583 | CAAGGAU | NO: 263 |
| ETXS675 | GmsCmsCmUmUmCmAfUmCfCfAfCmAmA | SEQ ID | GCCUUCAUCCACAA | SEQ ID |
| | mGmGmAmUmUmUmUm | NO: 584 | GGAUUUU | NO: 264 |
| ETXS677 | UmsAmsCmCmAmAmGfGmAfAfAfUmGmC | SEQ ID | UACCAAGGAAAUGC | SEQ ID |
| | mCmAmCmCmAmUmGm | NO: 585 | CACCAUG | NO: 265 |
| ETXS679 | UmsUmsGmAmUmGmAfGmAfUfUfAmAmU | SEQ ID | UUGAUGAGAUUAAU | SEQ ID |
| | mCmCmUmGmAmAmAm | NO: 586 | CCUGAAA | NO: 266 |
| ETXS681 | CmsUmsUmCmAmUmCfCmAfCfAfAmGmG | SEQ ID | CUUCAUCCACAAGG | SEQ ID |
| | mAmUmUmUmUmGmAm | NO: 587 | AUUUUGA | NO: 267 |
| ETXS683 | AmsAmsAmGmAmUmCfUmCfCfAfUmGmA | SEQ ID | AAAGAUCUCCAUGA | SEQ ID |
| | mGmGmCmAmCmGmAm | NO: 588 | GGCACGA | NO: 268 |
| ETXS685 | UmsGmsUmUmUmGmAfUmGfAfGfAmUmU | SEQ ID | UGUUUGAUGAGAUU | SEQ ID |
| | mAmAmUmCmCmUmGm | NO: 589 | AAUCCUG | NO: 269 |
| ETXS687 | AmsUmsGmCmUmGmGfUmGfGfUfCmCmU | SEQ ID | AUGCUGGUGGUCCU | SEQ ID |
| | mCmAmUmGmGmAmGm | NO: 590 | CAUGGAG | NO: 270 |
| ETXS689 | GmsUmsUmUmUmGmCfCmUfUfCfAmUmC | SEQ ID | GUUUUGCCUUCAUC | SEQ ID |
| | mCmAmCmAmAmGmGm | NO: 591 | CACAAGG | NO: 271 |
| ETXS691 | CmsCmsAmCmCmAmUfGmCfUfGfGmUmG | SEQ ID | CCACCAUGCUGGUG | SEQ ID |
| | mGmUmCmCmUmCmAm | NO: 592 | GUCCUCA | NO: 272 |
| ETXS693 | CmsAmsCmCmAmUmGfCmUfGfGfUmGmG | SEQ ID | CACCAUGCUGGUGG | SEQ ID |
| | mUmCmCmUmCmAmUm | NO: 593 | UCCUCAU | NO: 273 |
| ETXS695 | AmsAmsGmAmUmCmUfCmCfAfUfGmAmG | SEQ ID | AAGAUCUCCAUGAG | SEQ ID |
| | mGmCmAmCmGmAmUm | NO: 594 | GCACGAU | NO: 274 |
| ETXS697 | AmsGmsUmUmUmUmGfCmCfUfUfCmAmU | SEQ ID | AGUUUUGCCUUCAU | SEQ ID |
| | mCmCmAmCmAmAmGm | NO: 595 | CCACAAG | NO: 275 |
| ETXS699 | AmsCmsCmAmUmGmCfUmGfGfUfGmGmU | SEQ ID | ACCAUGCUGGUGGU | SEQ ID |
| | mCmCmUmCmAmUmGm | NO: 596 | CCUCAUG | NO: 276 |
| ETXS701 | AmsGmsAmUmCmUmCfCmAfUfGfAmGmG | SEQ ID | AGAUCUCCAUGAGG | SEQ ID |
| | mCmAmCmGmAmUmGm | NO: 597 | CACGAUG | NO: 277 |
| ETXS703 | CmsCmsAmUmGmCmUfGmGfUfGfGmUmC | SEQ ID | CCAUGCUGGUGGUC | SEQ ID |
| | mCmUmCmAmUmGmGm | NO: 598 | CUCAUGG | NO: 278 |
| ETXS705 | UmsGmsCmCmUmCmCfAmCfCfUfUmUmG | SEQ ID | UGCCUCCACCUUUG | SEQ ID |
| | mAmCmAmAmGmAmAm | NO: 599 | ACAAGAA | NO: 279 |
| ETXS707 | CmsCmsUmCmCmAmCfCmUfUfUfGmAmC | SEQ ID | CCUCCACCUUUGAC | SEQ ID |
| | mAmAmGmAmAmUmUm | NO: 600 | AAGAAUU | NO: 280 |
| ETXS709 | CmsAmsUmUmAmAmGfGmUfGfCfCmCmA | SEQ ID | CAUUAAGGUGCCCA | SEQ ID |
| | mUmGmAmUmGmUmAm | NO: 601 | UGAUGUA | NO: 281 |
| ETXS711 | CmsUmsGmCmCmCmUfAmCfCfAfAmGmG | SEQ ID | CUGCCCUACCAAGG | SEQ ID |
| | mAmAmAmUmGmCmCm | NO: 602 | AAAUGCC | NO: 282 |
| ETXS713 | CmsAmsAmAmCmUmGfCmCfCfUfAmCmC | SEQ ID | CAAACUGCCCUACC | SEQ ID |
| | mAmAmGmGmAmAmAm | NO: 603 | AAGGAAA | NO: 283 |
| ETXS715 | UmsCmsAmAmAmCmUfGmCfCfCfUmAmC | SEQ ID | UCAAACUGCCCUAC | SEQ ID |
| | mCmAmAmGmGmAmAm | NO: 604 | CAAGGAA | NO: 284 |
| ETXS717 | CmsUmsCmCmAmCmCfUmUfUfGfAmCmA | SEQ ID | CUCCACCUUUGACA | SEQ ID |
| | mAmGmAmAmUmUmUm | NO: 605 | AGAAUUU | NO: 285 |
| ETXS719 | CmsGmsAmCmAmCmUfUmUfCfCfAmCmC | SEQ ID | CGACACUUUCCACC | SEQ ID |
| | mUmGmGmAmCmAmAm | NO: 606 | UGGACAA | NO: 286 |
| ETXS721 | AmsCmsUmUmUmCmCfAmCfCfUfGmGmA | SEQ ID | ACUUUCCACCUGGA | SEQ ID |
| | mCmAmAmGmUmAmCm | NO: 607 | CAAGUAC | NO: 287 |
| ETXS723 | AmsAmsAmCmUmGmCfCmCfUfAfCmCmA | SEQ ID | AAACUGCCCUACCA | SEQ ID |
| | mAmGmGmAmAmAmUm | NO: 608 | AGGAAAU | NO: 288 |
| ETXS725 | AmsCmsUmGmCmCmCfUmAfCfCfAmAmG | SEQ ID | ACUGCCCUACCAAG | SEQ ID |
| | mGmAmAmAmUmGmCm | NO: 609 | GAAAUGC | NO: 289 |
| ETXS727 | CmsAmsCmUmUmUmCfCmAfCfCfUmGmG | SEQ ID | CACUUUCCACCUGG | SEQ ID |
| | mAmCmAmAmGmUmAm | NO: 610 | ACAAGUA | NO: 290 |
| ETXS729 | CmsCmsAmUmUmAmAfGmGfUfGfCmCmC | SEQ ID | CCAUUAAGGUGCCC | SEQ ID |
| | mAmUmGmAmUmGmUm | NO: 611 | AUGAUGU | NO: 291 |
| ETXS731 | UmsUmsGmCmCmUmCfCmAfCfCfUmUmU | SEQ ID | UUGCCUCCACCUUU | SEQ ID |
| | mGmAmCmAmAmGmAm | NO: 612 | GACAAGA | NO: 292 |
| ETXS733 | UmsUmsUmCmCmAmCfCmUfGfGfAmCmA | SEQ ID | UUUCCACCUGGACA | SEQ ID |
| | mAmGmUmAmCmAmAm | NO: 613 | AGUACAA | NO: 293 |
| ETXS735 | UmsCmsCmUmCmAmAfAmCfUfGfCmCmC | SEQ ID | UCCUCAAACUGCCC | SEQ ID |
| | mUmAmCmCmAmAmGm | NO: 614 | UACCAAG | NO: 294 |
| ETXS737 | AmsAmsCmUmGmCmCfCmUfAfCfCmAmA | SEQ ID | AACUGCCCUACCAA | SEQ ID |
| | mGmGmAmAmAmUmGm | NO: 615 | GGAAAUG | NO: 295 |
| ETXS739 | CmsCmsUmCmAmAmAfCmUfGfCfCmCmU | SEQ ID | CCUCAAACUGCCCU | SEQ ID |
| | mAmCmCmAmAmGmGm | NO: 616 | ACCAAGG | NO: 296 |
| ETXS741 | GmsUmsCmCmUmCmAfAmAfCfUfGmCmC | SEQ ID | GUCCUCAAACUGCC | SEQ ID |
| | mCmUmAmCmCmAmAm | NO: 617 | CUACCAA | NO: 297 |
| ETXS743 | GmsAmsCmAmCmUmUfUmCfCfAfCmCmU | SEQ ID | GACACUUUCCACCU | SEQ ID |
| | mGmGmAmCmAmAmGm | NO: 618 | GGACAAG | NO: 298 |
| ETXS745 | UmsUmsUmGmCmCmUfCmCfAfCfCmUmU | SEQ ID | UUUGCCUCCACCUU | SEQ ID |
| | mUmGmAmCmAmAmGm | NO: 619 | UGACAAG | NO: 299 |
| ETXS747 | AmsCmsAmCmUmUmUfCmCfAfCfCmUmG | SEQ ID | ACACUUUCCACCUG | SEQ ID |
| | mGmAmCmAmAmGmUm | NO: 620 | GACAAGU | NO: 300 |
| ETXS749 | UmsUmsCmCmAmCmCfUmGfGfAfCmAmA | SEQ ID | UUCCACCUGGACAA | SEQ ID |
| | mGmUmAmCmAmAmGm | NO: 621 | GUACAAG | NO: 301 |
| ETXS751 | CmsUmsUmUmCmCmAfCmCfUfGfGmAmC | SEQ ID | CUUUCCACCUGGAC | SEQ ID |
| | mAmAmGmUmAmCmAm | NO: 622 | AAGUACA | NO: 302 |
| ETXS753 | CmsUmsCmAmAmAmCfUmGfCfCfCmUmA | SEQ ID | CUCAAACUGCCCUA | SEQ ID |
| | mCmCmAmAmGmGmAm | NO: 623 | CCAAGGA | NO: 303 |
| ETXS755 | UmsUmsAmCmAmUmCfUmUfGfUfUmCmA | SEQ ID | UUACAUCUUGUUCA | SEQ ID |
| | mAmAmGmGmGmAmAm | NO: 624 | AAGGGAA | NO: 304 |
| ETXS757 | UmsGmsCmCmAmCmCfAmUfGfCfUmGmG | SEQ ID | UGCCACCAUGCUGG | SEQ ID |
| | mUmGmGmUmCmCmUm | NO: 625 | UGGUCCU | NO: 305 |
| ETXS759 | UmsAmsUmCmCmAmAfGmAfGfGfUmAmU | SEQ ID | UAUCCAAGAGGUAU | SEQ ID |
| | mUmUmUmGmAmUmAm | NO: 626 | UUUGAUA | NO: 306 |
| ETXS761 | AmsAmsAmUmGmCmCfAmCfCfAfUmGmC | SEQ ID | AAAUGCCACCAUGC | SEQ ID |
| | mUmGmGmUmGmGmUm | NO: 627 | UGGUGGU | NO: 307 |
| ETXS763 | AmsUmsUmAmCmAmUfCmUfUfGfUmUmC | SEQ ID | AUUACAUCUUGUUC | SEQ ID |
| | mAmAmAmGmGmGmAm | NO: 628 | AAAGGGA | NO: 308 |
| ETXS765 | CmsUmsCmCmAmUmGfAmGfGfCfAmCmG | SEQ ID | CUCCAUGAGGCACG | SEQ ID |
| | mAmUmGmGmCmAmAm | NO: 629 | AUGGCAA | NO: 309 |
| ETXS767 | UmsCmsCmAmUmGmCfCmUfCfCfUmGmU | SEQ ID | UCCAUGCCUCCUGU | SEQ ID |
| | mCmAmUmCmAmAmAm | NO: 630 | CAUCAAA | NO: 310 |
| ETXS769 | AmsUmsUmCmCmAmUfGmCfCfUfCmCmU | SEQ ID | AUUCCAUGCCUCCU | SEQ ID |
| | mGmUmCmAmUmCmAm | NO: 631 | GUCAUCA | NO: 311 |
| ETXS771 | CmsAmsAmGmGmAmAfAmUfGfCfCmAmC | SEQ ID | CAAGGAAAUGCCAC | SEQ ID |
| | mCmAmUmGmCmUmGm | NO: 632 | CAUGCUG | NO: 312 |
| ETXS773 | UmsGmsGmUmGmGmUfCmCfUfCfAmUmG | SEQ ID | UGGUGGUCCUCAUG | SEQ ID |
| | mGmAmGmAmAmAmAm | NO: 633 | GAGAAAA | NO: 313 |
| ETXS775 | AmsUmsCmUmUmGmUfUmCfAfAfAmGmG | SEQ ID | AUCUUGUUCAAAGG | SEQ ID |
| | mGmAmAmAmUmGmGm | NO: 634 | GAAAUGG | NO: 314 |
| ETXS777 | AmsAmsGmGmAmAmAfUmGfCfCfAmCmC | SEQ ID | AAGGAAAUGCCACC | SEQ ID |
| | mAmUmGmCmUmGmGm | NO: 635 | AUGCUGG | NO: 315 |
| ETXS779 | GmsCmsCmUmCmCmAfCmCfUfUfUmGmA | SEQ ID | GCCUCCACCUUUGA | SEQ ID |
| | mCmAmAmGmAmAmUm | NO: 636 | CAAGAAU | NO: 316 |
| ETXS781 | GmsAmsGmUmUmUmUfGmCfCfUfUmCmA | SEQ ID | GAGUUUUGCCUUCA | SEQ ID |
| | mUmCmCmAmCmAmAm | NO: 637 | UCCACAA | NO: 317 |
| ETXS783 | GmsCmsUmGmCmGmAfAmAfGfAfUmCmU | SEQ ID | GCUGCGAAAGAUCU | SEQ ID |
| | mCmCmAmUmGmAmGm | NO: 638 | CCAUGAG | NO: 318 |
| ETXS785 | AmsGmsUmUmUmGmCfCmUfCfCfAmCmC | SEQ ID | AGUUUGCCUCCACC | SEQ ID |
| | mUmUmUmGmAmCmAm | NO: 639 | UUUGACA | NO: 319 |
| ETXS787 | GmsUmsUmUmGmCmCfUmCfCfAfCmCmU | SEQ ID | GUUUGCCUCCACCU | SEQ ID |
| | mUmUmGmAmCmAmAm | NO: 640 | UUGACAA | NO: 320 |
| ETXS789 | CmsAmsUmCmUmUmGfUmUfCfAfAmAmG | SEQ ID | CAUCUUGUUCAAAG | SEQ ID |
| | mGmGmAmAmAmUmGm | NO: 641 | GGAAAUG | NO: 321 |
| ETXS791 | GmsGmsAmGmUmUmUfUmGfCfCfUmUmC | SEQ ID | GGAGUUUUGCCUUC | SEQ ID |
| | mAmUmCmCmAmCmAm | NO: 642 | AUCCACA | NO: 322 |
| ETXS793 | CmsUmsGmCmGmAmAfAmGfAfUfCmUmC | SEQ ID | CUGCGAAAGAUCUC | SEQ ID |
| | mCmAmUmGmAmGmGm | NO: 643 | CAUGAGG | NO: 323 |
| ETXS795 | CmsCmsAmUmGmCmCfUmCfCfUfGmUmC | SEQ ID | CCAUGCCUCCUGUC | SEQ ID |
| | mAmUmCmAmAmAmGm | NO: 644 | AUCAAAG | NO: 324 |
| ETXS797 | UmsGmsUmUmUmCmUfGmGfGfCfAmGmG | SEQ ID | UGUUUCUGGGCAGG | SEQ ID |
| | mGmUmGmGmUmGmAm | NO: 645 | GUGGUGA | NO: 325 |
| ETXS799 | GmsCmsCmUmGmCmUfGmCfGfAfAmAmG | SEQ ID | GCCUGCUGCGAAAG | SEQ ID |
| | mAmUmCmUmCmCmAm | NO: 646 | AUCUCCA | NO: 326 |
| ETXS801 | AmsAmsCmUmGmUmUfUmGfAfUfGmAmG | SEQ ID | AACUGUUUGAUGAG | SEQ ID |
| | mAmUmUmAmAmUmCm | NO: 647 | AUUAAUC | NO: 327 |
| ETXS803 | AmsUmsGmCmCmUmCfCmUfGfUfCmAmU | SEQ ID | AUGCCUCCUGUCAU | SEQ ID |
| | mCmAmAmAmGmUmGm | NO: 648 | CAAAGUG | NO: 328 |
| ETXS805 | AmsGmsUmAmUmGmAfGmAfUfGfCmAmU | SEQ ID | AGUAUGAGAUGCAU | SEQ ID |
| | mGmAmGmCmUmGmCm | NO: 649 | GAGCUGC | NO: 329 |
| ETXS807 | AmsAmsGmUmAmUmGfAmGfAfUfGmCmA | SEQ ID | AAGUAUGAGAUGCA | SEQ ID |
| | mUmGmAmGmCmUmGm | NO: 650 | UGAGCUG | NO: 330 |
| ETXS809 | UmsGmsUmCmCmUmCfAmAfAfCfUmGmC | SEQ ID | UGUCCUCAAACUGC | SEQ ID |
| | mCmCmUmAmCmCmAm | NO: 651 | CCUACCA | NO: 331 |
| ETXS811 | AmsCmsUmGmUmUmUfGmAfUfGfAmGmA | SEQ ID | ACUGUUUGAUGAGA | SEQ ID |
| | mUmUmAmAmUmCmCm | NO: 652 | UUAAUCC | NO: 332 |
| ETXS813 | GmsAmsCmCmAmUmUfAmAfGfGfUmGmC | SEQ ID | GACCAUUAAGGUGC | SEQ ID |
| | mCmCmAmUmGmAmUm | NO: 653 | CCAUGAU | NO: 333 |
| ETXS815 | AmsCmsCmAmUmUmAfAmGfGfUfGmCmC | SEQ ID | ACCAUUAAGGUGCC | SEQ ID |
| | mCmAmUmGmAmUmGm | NO: 654 | CAUGAUG | NO: 334 |
| ETXS817 | GmsUmsUmUmCmUmGfGmGfCfAfGmGmG | SEQ ID | GUUUCUGGGCAGGG | SEQ ID |
| | mUmGmGmUmGmAmAm | NO: 655 | UGGUGAA | NO: 335 |
| ETXS819 | UmsUmsCmUmGmUmUfUmCfUfGfGmGmC | SEQ ID | UUCUGUUUCUGGGC | SEQ ID |
| | mAmGmGmGmUmGmGm | NO: 656 | AGGGUGG | NO: 336 |
| ETXS821 | UmsUmsUmUmCmUmUfUmCfCfGfAmAmG | SEQ ID | UUUUCUUUCCGAAG | SEQ ID |
| | mUmUmCmAmAmGmCm | NO: 657 | UUCAAGC | NO: 337 |
| ETXS823 | UmsUmsUmCmUmUmUfCmCfGfAfAmGmU | SEQ ID | UUUCUUUCCGAAGU | SEQ ID |
| | mUmCmAmAmGmCmUm | NO: 658 | UCAAGCU | NO: 338 |
| ETXS825 | UmsCmsUmGmUmUmUfCmUfGfGfGmCmA | SEQ ID | UCUGUUUCUGGGCA | SEQ ID |
| | mGmGmGmUmGmGmUm | NO: 659 | GGGUGGU | NO: 339 |
| ETXS827 | AmsUmsGmUmCmCmUfCmAfAfAfCmUmG | SEQ ID | AUGUCCUCAAACUG | SEQ ID |
| | mCmCmCmUmAmCmCm | NO: 660 | CCCUACC | NO: 340 |
| ETXS829 | UmsCmsGmAmCmAmCfUmUfUfCfCmAmC | SEQ ID | UCGACACUUUCCAC | SEQ ID |
| | mCmUmGmGmAmCmAm | NO: 661 | CUGGACA | NO: 341 |
| ETXS831 | CfsCmsUfUmCfAmUfCmCfAmCfAmAfG | SEQ ID | CCUUCAUCCACAAG | SEQ ID |
| | mGfAmUfUmUf | NO: 662 | GAUUU | NO: 342 |
| ETXS833 | CfsGmsAfAmAfGmAfUmCfUmCfCmAfU | SEQ ID | CGAAAGAUCUCCAU | SEQ ID |
| | mGfAmGfGmCf | NO: 663 | GAGGC | NO: 343 |
| ETXS835 | UfsGmsGfUmGfGmUfCmCfUmCfAmUfG | SEQ ID | UGGUGGUCCUCAUG | SEQ ID |
| | mGfAmGfAmAf | NO: 664 | GAGAA | NO: 344 |
| ETXS837 | GfsAmsAfAmGfAmUfCmUfCmCfAmUfG | SEQ ID | GAAAGAUCUCCAUG | SEQ ID |
| | mAfGmGfCmAf | NO: 665 | AGGCA | NO: 345 |
| ETXS839 | AfsUmsGfAmGfAmUfGmCfAmUfGmAfG | SEQ ID | AUGAGAUGCAUGAG | SEQ ID |
| | mCfUmGfCmUf | NO: 666 | CUGCU | NO: 346 |
| ETXS841 | UfsUmsGfAmUfGmAfGmAfUmUfAmAfU | SEQ ID | UUGAUGAGAUUAAU | SEQ ID |
| | mCfCmUfGmAf | NO: 667 | CCUGA | NO: 347 |
| ETXS843 | AfsGmsAfUmUfAmAfUmCfCmUfGmAfA | SEQ ID | AGAUUAAUCCUGAA | SEQ ID |
| | mAfCmCfAmAf | NO: 668 | ACCAA | NO: 348 |
| ETXS845 | AfsUmsGfAmGfAmUfUmAfAmUfCmCfU | SEQ ID | AUGAGAUUAAUCCU | SEQ ID |
| | mGfAmAfAmCf | NO: 669 | GAAAC | NO: 349 |
| ETXS847 | GfsAmsGfAmUfUmAfAmUfCmCfUmGfA | SEQ ID | GAGAUUAAUCCUGA | SEQ ID |
| | mAfAmCfCmAf | NO: 670 | AACCA | NO: 350 |
| ETXS849 | UfsGmsAfGmAfUmUfAmAfUmCfCmUfG | SEQ ID | UGAGAUUAAUCCUG | SEQ ID |
| | mAfAmAfCmCf | NO: 671 | AAACC | NO: 351 |
| ETXS851 | GfsUmsUfUmGfAmUfGmAfGmAfUmUfA | SEQ ID | GUUUGAUGAGAUUA | SEQ ID |
| | mAfUmCfCmUf | NO: 672 | AUCCU | NO: 352 |
| ETXS853 | UfsUmsGfCmCfUmUfCmAfUmCfCmAfC | SEQ ID | UUGCCUUCAUCCAC | SEQ ID |
| | mAfAmGfGmAf | NO: 673 | AAGGA | NO: 353 |
| ETXS855 | AfsAmsAfGmAfUmCfUmCfCmAfUmGfA | SEQ ID | AAAGAUCUCCAUGA | SEQ ID |
| | mGfGmCfAmCf | NO: 674 | GGCAC | NO: 354 |
| ETXS857 | CfsUmsGfGmUfGmGfUmCfCmUfCmAfU | SEQ ID | CUGGUGGUCCUCAU | SEQ ID |
| | mGfGmAfGmAf | NO: 675 | GGAGA | NO: 355 |
| ETXS859 | UfsUmsCfAmUfCmCfAmCfAmAfGmGfA | SEQ ID | UUCAUCCACAAGGA | SEQ ID |
| | mUfUmUfUmGf | NO: 676 | UUUUG | NO: 356 |
| ETXS861 | AfsAmsGfAmUfCmUfCmCfAmUfGmAfG | SEQ ID | AAGAUCUCCAUGAG | SEQ ID |
| | mGfCmAfCmGf | NO: 677 | GCACG | NO: 357 |
| ETXS863 | CfsAmsUfCmCfAmCfAmAfGmGfAmUfU | SEQ ID | CAUCCACAAGGAUU | SEQ ID |
| | mUfUmGfAmUf | NO: 678 | UUGAU | NO: 358 |
| ETXS865 | UfsGmsAfUmGfAmGfAmUfUmAfAmUfC | SEQ ID | UGAUGAGAUUAAUC | SEQ ID |
| | mCfUmGfAmAf | NO: 679 | CUGAA | NO: 359 |
| ETXS867 | GfsCmsCfUmUfCmAfUmCfCmAfCmAfA | SEQ ID | GCCUUCAUCCACAA | SEQ ID |
| | mGfGmAfUmUf | NO: 680 | GGAUU | NO: 360 |
| ETXS869 | UfsCmsUfCmCfAmUfGmAfGmGfCmAfC | SEQ ID | UCUCCAUGAGGCAC | SEQ ID |
| | mGfAmUfGmGf | NO: 681 | GAUGG | NO: 361 |
| ETXS871 | UmsGmsCmCmUmUmCfAfUfCfCfAmCmA | SEQ ID | UGCCUUCAUCCACA | SEQ ID |
| | mAmGmGmAmUfUmUm | NO: 682 | AGGAUUU | NO: 242 |
| ETXS873 | UmsGmsCmGmAmAmAfGfAfUfCfUmCmC | SEQ ID | UGCGAAAGAUCUCC | SEQ ID |
| | mAmUmGmAmGfGmCm | NO: 683 | AUGAGGC | NO: 243 |
| ETXS875 | GmsCmsUmGmGmUmGfGfUfCfCfUmCmA | SEQ ID | GCUGGUGGUCCUCA | SEQ ID |
| | mUmGmGmAmGfAmAm | NO: 684 | UGGAGAA | NO: 244 |
| ETXS877 | GmsCmsGmAmAmAmGfAfUfCfUfCmCmA | SEQ ID | GCGAAAGAUCUCCA | SEQ ID |
| | mUmGmAmGmGfCmAm | NO: 685 | UGAGGCA | NO: 245 |
| ETXS879 | GmsUmsAmUmGmAmGfAfUfGfCfAmUmG | SEQ ID | GUAUGAGAUGCAUG | SEQ ID |
| | mAmGmCmUmGfCmUm | NO: 686 | AGCUGCU | NO: 246 |
| ETXS881 | GmsUmsUmUmGmAmUfGfAfGfAfUmUmA | SEQ ID | GUUUGAUGAGAUUA | SEQ ID |
| | mAmUmCmCmUfGmAm | NO: 687 | AUCCUGA | NO: 247 |
| ETXS883 | UmsGmsAmGmAmUmUfAfAfUfCfCmUmG | SEQ ID | UGAGAUUAAUCCUG | SEQ ID |
| | mAmAmAmCmCfAmAm | NO: 688 | AAACCAA | NO: 248 |
| ETXS885 | UmsGmsAmUmGmAmGfAfUfUfAfAmUmC | SEQ ID | UGAUGAGAUUAAUC | SEQ ID |
| | mCmUmGmAmAfAmCm | NO: 689 | CUGAAAC | NO: 249 |
| ETXS887 | AmsUmsGmAmGmAmUfUfAfAfUfCmCmU | SEQ ID | AUGAGAUUAAUCCU | SEQ ID |
| | mGmAmAmAmCfCmAm | NO: 690 | GAAACCA | NO: 250 |
| ETXS889 | GmsAmsUmGmAmGmAfUfUfAfAfUmCmC | SEQ ID | GAUGAGAUUAAUCC | SEQ ID |
| | mUmGmAmAmAfCmCm | NO: 691 | UGAAACC | NO: 251 |
| ETXS891 | CmsUmsGmUmUmUmGfAfUfGfAfGmAmU | SEQ ID | CUGUUUGAUGAGAU | SEQ ID |
| | mUmAmAmUmCfCmUm | NO: 692 | UAAUCCU | NO: 252 |
| ETXS893 | UmsUmsUmUmGmCmCfUfUfCfAfUmCmC | SEQ ID | UUUUGCCUUCAUCC | SEQ ID |
| | mAmCmAmAmGfGmAm | NO: 693 | ACAAGGA | NO: 253 |
| ETXS895 | CmsGmsAmAmAmGmAfUfCfUfCfCmAmU | SEQ ID | CGAAAGAUCUCCAU | SEQ ID |
| | mGmAmGmGmCfAmCm | NO: 694 | GAGGCAC | NO: 254 |
| ETXS897 | UmsGmsCmUmGmGmUfGfGfUfCfCmUmC | SEQ ID | UGCUGGUGGUCCUC | SEQ ID |
| | mAmUmGmGmAfGmAm | NO: 695 | AUGGAGA | NO: 255 |
| ETXS899 | CmsCmsUmUmCmAmUfCfCfAfCfAmAmG | SEQ ID | CCUUCAUCCACAAG | SEQ ID |
| | mGmAmUmUmUfUmGm | NO: 696 | GAUUUUG | NO: 256 |
| ETXS901 | GmsAmsAmAmGmAmUfCfUfCfCfAmUmG | SEQ ID | GAAAGAUCUCCAUG | SEQ ID |
| | mAmGmGmCmAfCmGm | NO: 697 | AGGCACG | NO: 257 |
| ETXS903 | UmsUmsCmAmUmCmCfAfCfAfAfGmGmA | SEQ ID | UUCAUCCACAAGGA | SEQ ID |
| | mUmUmUmUmGfAmUm | NO: 698 | UUUUGAU | NO: 258 |
| ETXS905 | UmsUmsUmGmAmUmGfAfGfAfUfUmAmA | SEQ ID | UUUGAUGAGAUUAA | SEQ ID |
| | mUmCmCmUmGfAmAm | NO: 699 | UCCUGAA | NO: 259 |
| ETXS907 | UmsUmsGmCmCmUmUfCfAfUfCfCmAmC | SEQ ID | UUGCCUUCAUCCAC | SEQ ID |
| | mAmAmGmGmAfUmUm | NO: 700 | AAGGAUU | NO: 260 |
| ETXS909 | GmsAmsUmCmUmCmCfAfUfGfAfGmGmC | SEQ ID | GAUCUCCAUGAGGC | SEQ ID |
| | mAmCmGmAmUfGmGm | NO: 701 | ACGAUGG | NO: 261 |
| ETXS911 | UmsGmsCmCmUmUfCfAmUfCfCfAfCmA | SEQ ID | UGCCUUCAUCCACA | SEQ ID |
| | mAmGmGmAmUmUmUm | NO: 702 | AGGAUUU | NO: 242 |
| ETXS913 | UmsGmsCmGmAmAfAfGmAfUfCfUfCmC | SEQ ID | UGCGAAAGAUCUCC | SEQ ID |
| | mAmUmGmAmGmGmCm | NO: 703 | AUGAGGC | NO: 243 |
| ETXS915 | GmsCmsUmGmGmUfGfGmUfCfCfUfCmA | SEQ ID | GCUGGUGGUCCUCA | SEQ ID |
| | mUmGmGmAmGmAmAm | NO: 704 | UGGAGAA | NO: 244 |
| ETXS917 | GmsCmsGmAmAmAfGfAmUfCfUfCfCmA | SEQ ID | GCGAAAGAUCUCCA | SEQ ID |
| | mUmGmAmGmGmCmAm | NO: 705 | UGAGGCA | NO: 245 |
| ETXS919 | GmsUmsAmUmGmAfGfAmUfGfCfAfUmG | SEQ ID | GUAUGAGAUGCAUG | SEQ ID |
| | mAmGmCmUmGmCmUm | NO: 706 | AGCUGCU | NO: 246 |
| ETXS921 | GmsUmsUmUmGmAfUfGmAfGfAfUfUmA | SEQ ID | GUUUGAUGAGAUUA | SEQ ID |
| | mAmUmCmCmUmGmAm | NO: 707 | AUCCUGA | NO: 247 |
| ETXS923 | UmsGmsAmGmAmUfUfAmAfUfCfCfUmG | SEQ ID | UGAGAUUAAUCCUG | SEQ ID |
| | mAmAmAmCmCmAmAm | NO: 708 | AAACCAA | NO: 248 |
| ETXS925 | UmsGmsAmUmGmAfGfAmUfUfAfAfUmC | SEQ ID | UGAUGAGAUUAAUC | SEQ ID |
| | mCmUmGmAmAmAmCm | NO: 709 | CUGAAAC | NO: 249 |
| ETXS927 | AmsUmsGmAmGmAfUfUmAfAfUfCfCmU | SEQ ID | AUGAGAUUAAUCCU | SEQ ID |
| | mGmAmAmAmCmCmAm | NO: 710 | GAAACCA | NO: 250 |
| ETXS929 | GmsAmsUmGmAmGfAfUmUfAfAfUfCmC | SEQ ID | GAUGAGAUUAAUCC | SEQ ID |
| | mUmGmAmAmAmCmCm | NO: 711 | UGAAACC | NO: 251 |
| ETXS931 | CmsUmsGmUmUmUfGfAmUfGfAfGfAmU | SEQ ID | CUGUUUGAUGAGAU | SEQ ID |
| | mUmAmAmUmCmCmUm | NO: 712 | UAAUCCU | NO: 252 |
| ETXS933 | UmsUmsUmUmGmCfCfUmUfCfAfUfCmC | SEQ ID | UUUUGCCUUCAUCC | SEQ ID |
| | mAmCmAmAmGmGmAm | NO: 713 | ACAAGGA | NO: 253 |
| ETXS935 | CmsGmsAmAmAmGfAfUmCfUfCfCfAmU | SEQ ID | CGAAAGAUCUCCAU | SEQ ID |
| | mGmAmGmGmCmAmCm | NO: 714 | GAGGCAC | NO: 254 |
| ETXS937 | UmsGmsCmUmGmGfUfGmGfUfCfCfUmC | SEQ ID | UGCUGGUGGUCCUC | SEQ ID |
| | mAmUmGmGmAmGmAm | NO: 715 | AUGGAGA | NO: 255 |
| ETXS939 | CmsCmsUmUmCmAfUfCmCfAfCfAfAmG | SEQ ID | CCUUCAUCCACAAG | SEQ ID |
| | mGmAmUmUmUmUmGm | NO: 716 | GAUUUUG | NO: 256 |
| ETXS941 | GmsAmsAmAmGmAfUfCmUfCfCfAfUmG | SEQ ID | GAAAGAUCUCCAUG | SEQ ID |
| | mAmGmGmCmAmCmGm | NO: 717 | AGGCACG | NO: 257 |
| ETXS943 | UmsUmsCmAmUmCfCfAmCfAfAfGfGmA | SEQ ID | UUCAUCCACAAGGA | SEQ ID |
| | mUmUmUmUmGmAmUm | NO: 718 | UUUUGAU | NO: 258 |
| ETXS945 | UmsUmsUmGmAmUfGfAmGfAfUfUfAmA | SEQ ID | UUUGAUGAGAUUAA | SEQ ID |
| | mUmCmCmUmGmAmAm | NO: 719 | UCCUGAA | NO: 259 |
| ETXS947 | UmsUmsGmCmCmUfUfCmAfUfCfCfAmC | SEQ ID | UUGCCUUCAUCCAC | SEQ ID |
| | mAmAmGmGmAmUmUm | NO: 720 | AAGGAUU | NO: 260 |
| ETXS949 | GmsAmsUmCmUmCfCfAmUfGfAfGfGmC | SEQ ID | GAUCUCCAUGAGGC | SEQ ID |
| | mAmCmGmAmUmGmGm | NO: 721 | ACGAUGG | NO: 261 |
| ETXS951 | UmsGmsCmCmUmUmCfAmUfCfCfAfCmA | SEQ ID | UGCCUUCAUCCACA | SEQ ID |
| | mAmGmGmAmUmUmUm | NO: 722 | AGGAUUU | NO: 242 |
| ETXS953 | UmsGmsCmGmAmAmAfGmAfUfCfUfCmC | SEQ ID | UGCGAAAGAUCUCC | SEQ ID |
| | mAmUmGmAmGmGmCm | NO: 723 | AUGAGGC | NO: 243 |
| ETXS955 | GmsCmsUmGmGmUmGfGmUfCfCfUfCmA | SEQ ID | GCUGGUGGUCCUCA | SEQ ID |
| | mUmGmGmAmGmAmAm | NO: 724 | UGGAGAA | NO: 244 |
| ETXS957 | GmsCmsGmAmAmAmGfAmUfCfUfCfCmA | SEQ ID | GCGAAAGAUCUCCA | SEQ ID |
| | mUmGmAmGmGmCmAm | NO: 725 | UGAGGCA | NO: 245 |
| ETXS959 | GmsUmsAmUmGmAmGfAmUfGfCfAfUmG | SEQ ID | GUAUGAGAUGCAUG | SEQ ID |
| | mAmGmCmUmGmCmUm | NO: 726 | AGCUGCU | NO: 246 |
| ETXS961 | GmsUmsUmUmGmAmUfGmAfGfAfUfUmA | SEQ ID | GUUUGAUGAGAUUA | SEQ ID |
| | mAmUmCmCmUmGmAm | NO: 727 | AUCCUGA | NO: 247 |
| ETXS963 | UmsGmsAmGmAmUmUfAmAfUfCfCfUmG | SEQ ID | UGAGAUUAAUCCUG | SEQ ID |
| | mAmAmAmCmCmAmAm | NO: 728 | AAACCAA | NO: 248 |
| ETXS965 | UmsGmsAmUmGmAmGfAmUfUfAfAfUmC | SEQ ID | UGAUGAGAUUAAUC | SEQ ID |
| | mCmUmGmAmAmAmCm | NO: 729 | CUGAAAC | NO: 249 |
| ETXS967 | AmsUmsGmAmGmAmUfUmAfAfUfCfCmU | SEQ ID | AUGAGAUUAAUCCU | SEQ ID |
| | mGmAmAmAmCmCmAm | NO: 730 | GAAACCA | NO: 250 |
| ETXS969 | GmsAmsUmGmAmGmAfUmUfAfAfUfCmC | SEQ ID | GAUGAGAUUAAUCC | SEQ ID |
| | mUmGmAmAmAmCmCm | NO: 731 | UGAAACC | NO: 251 |
| ETXS971 | CmsUmsGmUmUmUmGfAmUfGfAfGfAmU | SEQ ID | CUGUUUGAUGAGAU | SEQ ID |
| | mUmAmAmUmCmCmUm | NO: 732 | UAAUCCU | NO: 252 |
| ETXS973 | UmsUmsUmUmGmCmCfUmUfCfAfUfCmC | SEQ ID | UUUUGCCUUCAUCC | SEQ ID |
| | mAmCmAmAmGmGmAm | NO: 733 | ACAAGGA | NO: 253 |
| ETXS975 | CmsGmsAmAmAmGmAfUmCfUfCfCfAmU | SEQ ID | CGAAAGAUCUCCAU | SEQ ID |
| | mGmAmGmGmCmAmCm | NO: 734 | GAGGCAC | NO: 254 |
| ETXS977 | UmsGmsCmUmGmGmUfGmGfUfCfCfUmC | SEQ ID | UGCUGGUGGUCCUC | SEQ ID |
| | mAmUmGmGmAmGmAm | NO: 735 | AUGGAGA | NO: 255 |
| ETXS979 | CmsCmsUmUmCmAmUfCmCfAfCfAfAmG | SEQ ID | CCUUCAUCCACAAG | SEQ ID |
| | mGmAmUmUmUmUmGm | NO: 736 | GAUUUUG | NO: 256 |
| ETXS981 | GmsAmsAmAmGmAmUfCmUfCfCfAfUmG | SEQ ID | GAAAGAUCUCCAUG | SEQ ID |
| | mAmGmGmCmAmCmGm | NO: 737 | AGGCACG | NO: 257 |
| ETXS983 | UmsUmsCmAmUmCmCfAmCfAfAfGfGmA | SEQ ID | UUCAUCCACAAGGA | SEQ ID |
| | mUmUmUmUmGmAmUm | NO: 738 | UUUUGAU | NO: 258 |
| ETXS985 | UmsUmsUmGmAmUmGfAmGfAfUfUfAmA | SEQ ID | UUUGAUGAGAUUAA | SEQ ID |
| | mUmCmCmUmGmAmAm | NO: 739 | UCCUGAA | NO: 259 |
| ETXS987 | UmsUmsGmCmCmUmUfCmAfUfCfCfAmC | SEQ ID | UUGCCUUCAUCCAC | SEQ ID |
| | mAmAmGmGmAmUmUm | NO: 740 | AAGGAUU | NO: 260 |
| ETXS989 | GmsAmsUmCmUmCmCfAmUfGfAfGfGmC | SEQ ID | GAUCUCCAUGAGGC | SEQ ID |
| | mAmCmGmAmUmGmGm | NO: 741 | ACGAUGG | NO: 261 |
| ETXS991 | UmsGmsCmCmUmUmCfAmUfCfCfAfCmA | SEQ ID | UGCCUUCAUCCACA | SEQ ID |
| | mAmGmGmAmUmUmUm | NO: 742 | AGGAUUU | NO: 242 |
| ETXS993 | UmsGmsCmGmAmAmAfGmAfUfCfUfCmC | SEQ ID | UGCGAAAGAUCUCC | SEQ ID |
| | mAmUmGmAmGmGmCm | NO: 743 | AUGAGGC | NO: 243 |
| ETXS995 | GmsCmsUmGmGmUmGfGmUfCfCfUfCmA | SEQ ID | GCUGGUGGUCCUCA | SEQ ID |
| | mUmGmGmAmGmAmAm | NO: 744 | UGGAGAA | NO: 244 |
| ETXS997 | GmsCmsGmAmAmAmGfAmUfCfUfCfCmA | SEQ ID | GCGAAAGAUCUCCA | SEQ ID |
| | mUmGmAmGmGmCmAm | NO: 745 | UGAGGCA | NO: 245 |
| ETXS999 | GmsUmsAmUmGmAmGfAmUfGfCfAfUmG | SEQ ID | GUAUGAGAUGCAUG | SEQ ID |
| | mAmGmCmUmGmCmUm | NO: 746 | AGCUGCU | NO: 246 |
| ETXS1001 | GmsUmsUmUmGmAmUfGmAfGfAfUfUmA | SEQ ID | GUUUGAUGAGAUUA | SEQ ID |
| | mAmUmCmCmUmGmAm | NO: 747 | AUCCUGA | NO: 247 |
| ETXS1003 | UmsGmsAmGmAmUmUfAmAfUfCfCfUmG | SEQ ID | UGAGAUUAAUCCUG | SEQ ID |
| | mAmAmAmCmCmAmAm | NO: 748 | AAACCAA | NO: 248 |
| ETXS1005 | UmsGmsAmUmGmAmGfAmUfUfAfAfUmC | SEQ ID | UGAUGAGAUUAAUC | SEQ ID |
| | mCmUmGmAmAmAmCm | NO: 749 | CUGAAAC | NO: 249 |
| ETXS1007 | AmsUmsGmAmGmAmUfUmAfAfUfCfCmU | SEQ ID | AUGAGAUUAAUCCU | SEQ ID |
| | mGmAmAmAmCmCmAm | NO: 750 | GAAACCA | NO: 250 |
| ETXS1009 | GmsAmsUmGmAmGmAfUmUfAfAfUfCmC | SEQ ID | GAUGAGAUUAAUCC | SEQ ID |
| | mUmGmAmAmAmCmCm | NO: 751 | UGAAACC | NO: 251 |
| ETXS1011 | CmsUmsGmUmUmUmGfAmUfGfAfGfAmU | SEQ ID | CUGUUUGAUGAGAU | SEQ ID |
| | mUmAmAmUmCmCmUm | NO: 752 | UAAUCCU | NO: 252 |
| ETXS1013 | UmsUmsUmUmGmCmCfUmUfCfAfUfCmC | SEQ ID | UUUUGCCUUCAUCC | SEQ ID |
| | mAmCmAmAmGmGmAm | NO: 753 | ACAAGGA | NO: 253 |
| ETXS1015 | CmsGmsAmAmAmGmAfUmCfUfCfCfAmU | SEQ ID | CGAAAGAUCUCCAU | SEQ ID |
| | mGmAmGmGmCmAmCm | NO: 754 | GAGGCAC | NO: 254 |
| ETXS1017 | UmsGmsCmUmGmGmUfGmGfUfCfCfUmC | SEQ ID | UGCUGGUGGUCCUC | SEQ ID |
| | mAmUmGmGmAmGmAm | NO: 755 | AUGGAGA | NO: 255 |
| ETXS1019 | CmsCmsUmUmCmAmUfCmCfAfCfAfAmG | SEQ ID | CCUUCAUCCACAAG | SEQ ID |
| | mGmAmUmUmUmUmGm | NO: 756 | GAUUUUG | NO: 256 |
| ETXS1021 | GmsAmsAmAmGmAmUfCmUfCfCfAfUmG | SEQ ID | GAAAGAUCUCCAUG | SEQ ID |
| | mAmGmGmCmAmCmGm | NO: 757 | AGGCACG | NO: 257 |
| ETXS1023 | UmsUmsCmAmUmCmCfAmCfAfAfGfGmA | SEQ ID | UUCAUCCACAAGGA | SEQ ID |
| | mUmUmUmUmGmAmUm | NO: 758 | UUUUGAU | NO: 258 |
| ETXS1025 | UmsUmsUmGmAmUmGfAmGfAfUfUfAmA | SEQ ID | UUUGAUGAGAUUAA | SEQ ID |
| | mUmCmCmUmGmAmAm | NO: 759 | UCCUGAA | NO: 259 |
| ETXS1027 | UmsUmsGmCmCmUmUfCmAfUfCfCfAmC | SEQ ID | UUGCCUUCAUCCAC | SEQ ID |
| | mAmAmGmGmAmUmUm | NO: 760 | AAGGAUU | NO: 260 |
| ETXS1029 | GmsAmsUmCmUmCmCfAmUfGfAfGfGmC | SEQ ID | GAUCUCCAUGAGGC | SEQ ID |
| | mAmCmGmAmUmGmGm | NO: 761 | ACGAUGG | NO: 261 |
| ETXS1031 | iaiaGmsCmsCmUmUmCmAfUmCfCfAfC | SEQ ID | GCCUUCAUCCACAA | SEQ ID |
| | fAmAmGmGmAmUmUmUmUm | NO: 772 | GGAUUUU | NO: 264 |
| ETXS1033 | iaiaGmsCmsCmUmUmCmAmUmCfCfAfC | SEQ ID | GCCUUCAUCCACAA | SEQ ID |
| | mAmAmGmGmAmUmUmUmUm | NO: 773 | GGAUUUU | NO: 264 |
| ETXS1035 | iaiaUmsAmsCmCmAmAmGfGmAfAfAfU | SEQ ID | UACCAAGGAAAUGC | SEQ ID |
| | fGmCmCmAmCmCmAmUmGm | NO: 774 | CACCAUG | NO: 265 |
| ETXS1037 | iaiaUmsAmsCmCmAmAmGmGmAfAfAfU | SEQ ID | UACCAAGGAAAUGC | SEQ ID |
| | mGmCmCmAmCmCmAmUmGm | NO: 775 | CACCAUG | NO: 265 |
| ETXS1039 | iaiaAmsAmsAmGmAmUmCfUmCfCfAfU | SEQ ID | AAAGAUCUCCAUGA | SEQ ID |
| | fGmAmGmGmCmAmCmGmAm | NO: 776 | GGCACGA | NO: 268 |
| ETXS1041 | iaiaAmsAmsAmGmAmUmCmUmCfCfAfU | SEQ ID | AAAGAUCUCCAUGA | SEQ ID |
| | mGmAmGmGmCmAmCmGmAm | NO: 777 | GGCACGA | NO: 268 |
| ETXS1043 | iaiaCmsUmsCmCmAmCmCfUmUfUfGfA | SEQ ID | CUCCACCUUUGACA | SEQ ID |
| | fCmAmAmGmAmAmUmUmUm | NO: 778 | AGAAUUU | NO: 285 |
| ETXS1045 | iaiaCmsUmsCmCmAmCmCmUmUfUfGfA | SEQ ID | CUCCACCUUUGACA | SEQ ID |
| | mCmAmAmGmAmAmUmUmUm | NO: 779 | AGAAUUU | NO: 285 |
| ETXS1047 | iaiaGmsGmsAmGmUmUmUfUmGfCfCfU | SEQ ID | GGAGUUUUGCCUUC | SEQ ID |
| | fUmCmAmUmCmCmAmCmAm | NO: 780 | AUCCACA | NO: 322 |
| ETXS1049 | iaiaGmsGmsAmGmUmUmUmUmGfCfCfU | SEQ ID | GGAGUUUUGCCUUC | SEQ ID |
| | mUmCmAmUmCmCmAmCmAm | NO: 781 | AUCCACA | NO: 322 |
| ETXS2127 | GmsCmsCmUmUmCmAfUmCfCfAfCmAmA | SEQ ID | GCCUUCAUCCACAA | SEQ ID |
| | mGmGmAmCmUmUmUm | NO: 798 | GGACUUU | NO: 791 |
| ETXS2143 | CmsUmsCmCmAmCmCfUmUfUfGfAmCmA | SEQ ID | CUCCACCUUUGACA | SEQ ID |
| | mAmGmAmAmGmUmUm | NO: 799 | AGAAGUU | NO: 792 |
| ETXS2151 | GmsUmsAmGmCmUmUfUmGfCfCfUmUmC | SEQ ID | GUAGCUUUGCCUUC | SEQ ID |
| | mAmUmCmCmAmCmAm | NO: 800 | AUCCACA | NO: 793 |

As used herein, and in particular in Tables 3 and 4, the following abbreviations are used for modified nucleosides:

Am stands for 2′-O-methyl-adenosine, Cm stands for 2′-O-methyl-cytidine, Gm stands for 2′-O-methyl-guanosine, Um stands for 2′-O-methyl-uridine, Af stands for 2′-Fluoro-adenosine, Cf stands for 2′-Fluoro-cytidine, Gf stands for 2′-Fluoro-guanosine and Uf stands for 2′-Fluoro-uridine.

Furthermore, the letter “s” is used as abbreviation for a phosphorothioate linkage between two consecutive (modified) nucleosides. For example, the abbreviation “AmsAm” is used for two consecutive 2′-O-methyl-adenosine nucleosides that are linked via a 3′5′ phosphorothioate linkage. No abbreviation is used for nucleosides that are linked via a standard 3′5′ phosphodiester linkage. For example, the abbreviation “AmAm” is used for two consecutive 2′-O-methyl-adenosine nucleosides that are linked via a 3′5′ phosphodiester linkage.

In certain embodiments, the nucleic acid comprises a first strand that comprises, consists of, or consists essentially ofa (modified) nucleoside sequence differing by 0 or 1 nucleosides from any one of SEQ ID NO:362-561, 762-771, 782-786 or 795-797;
and a second strand that comprises, consists of, or consists essentially of a (modified) nucleoside sequence differing by 0 or 1 nucleosides from any one of SEQ ID NO:562-761, 772-781 or 798-800.

Preferred combinations of complementary modified antisense (first) and sense (second) strands are listed below in Table 5:

TABLE 5

| Duplex ID | First (Antisense) strand ID | Second (Sense) strand ID |
|---|---|---|
| ETXM316 | ETXS632 (SEQ ID NO: 362) | ETXS631 (SEQ ID NO: 562) |
| ETXM317 | ETXS634 (SEQ ID NO: 363) | ETXS633 (SEQ ID NO: 563) |
| ETXM318 | ETXS636 (SEQ ID NO: 364) | ETXS635 (SEQ ID NO: 564) |
| ETXM319 | ETXS638 (SEQ ID NO: 365) | ETXS637 (SEQ ID NO: 565) |
| ETXM320 | ETXS640 (SEQ ID NO: 366) | ETXS639 (SEQ ID NO: 566) |
| ETXM321 | ETXS642 (SEQ ID NO: 367) | ETXS641 (SEQ ID NO: 567) |
| ETXM322 | ETXS644 (SEQ ID NO: 368) | ETXS643 (SEQ ID NO: 568) |
| ETXM323 | ETXS646 (SEQ ID NO: 369) | ETXS645 (SEQ ID NO: 569) |
| ETXM324 | ETXS648 (SEQ ID NO: 370) | ETXS647 (SEQ ID NO: 570) |
| ETXM325 | ETXS650 (SEQ ID NO: 371) | ETXS649 (SEQ ID NO: 571) |
| ETXM326 | ETXS652 (SEQ ID NO: 372) | ETXS651 (SEQ ID NO: 572) |
| ETXM327 | ETXS654 (SEQ ID NO: 373) | ETXS653 (SEQ ID NO: 573) |
| ETXM328 | ETXS656 (SEQ ID NO: 374) | ETXS655 (SEQ ID NO: 574) |
| ETXM329 | ETXS658 (SEQ ID NO: 375) | ETXS657 (SEQ ID NO: 575) |
| ETXM330 | ETXS660 (SEQ ID NO: 376) | ETXS659 (SEQ ID NO: 576) |
| ETXM331 | ETXS662 (SEQ ID NO: 377) | ETXS661 (SEQ ID NO: 577) |
| ETXM332 | ETXS664 (SEQ ID NO: 378) | ETXS663 (SEQ ID NO: 578) |
| ETXM333 | ETXS666 (SEQ ID NO: 379) | ETXS665 (SEQ ID NO: 579) |
| ETXM334 | ETXS668 (SEQ ID NO: 380) | ETXS667 (SEQ ID NO: 580) |
| ETXM335 | ETXS670 (SEQ ID NO: 381) | ETXS669 (SEQ ID NO: 581) |
| ETXM336 | ETXS672 (SEQ ID NO: 382) | ETXS671 (SEQ ID NO: 582) |
| ETXM337 | ETXS674 (SEQ ID NO: 383) | ETXS673 (SEQ ID NO: 583) |
| ETXM338 | ETXS676 (SEQ ID NO: 384) | ETXS675 (SEQ ID NO: 584) |
| ETXM339 | ETXS678 (SEQ ID NO: 385) | ETXS677 (SEQ ID NO: 585) |
| ETXM340 | ETXS680 (SEQ ID NO: 386) | ETXS679 (SEQ ID NO: 586) |
| ETXM341 | ETXS682 (SEQ ID NO: 387) | ETXS681 (SEQ ID NO: 587) |
| ETXM342 | ETXS684 (SEQ ID NO: 388) | ETXS683 (SEQ ID NO: 588) |
| ETXM343 | ETXS686 (SEQ ID NO: 389) | ETXS685 (SEQ ID NO: 589) |
| ETXM344 | ETXS688 (SEQ ID NO: 390) | ETXS687 (SEQ ID NO: 590) |
| ETXM345 | ETXS690 (SEQ ID NO: 391) | ETXS689 (SEQ ID NO: 591) |
| ETXM346 | ETXS692 (SEQ ID NO: 392) | ETXS691 (SEQ ID NO: 592) |
| ETXM347 | ETXS694 (SEQ ID NO: 393) | ETXS693 (SEQ ID NO: 593) |
| ETXM348 | ETXS696 (SEQ ID NO: 394) | ETXS695 (SEQ ID NO: 594) |
| ETXM349 | ETXS698 (SEQ ID NO: 395) | ETXS697 (SEQ ID NO: 595) |
| ETXM350 | ETXS700 (SEQ ID NO: 396) | ETXS699 (SEQ ID NO: 596) |
| ETXM351 | ETXS702 (SEQ ID NO: 397) | ETXS701 (SEQ ID NO: 597) |
| ETXM352 | ETXS704 (SEQ ID NO: 398) | ETXS703 (SEQ ID NO: 598) |
| ETXM353 | ETXS706 (SEQ ID NO: 399) | ETXS705 (SEQ ID NO: 599) |
| ETXM354 | ETXS708 (SEQ ID NO: 400) | ETXS707 (SEQ ID NO: 600) |
| ETXM355 | ETXS710 (SEQ ID NO: 401) | ETXS709 (SEQ ID NO: 601) |
| ETXM356 | ETXS712 (SEQ ID NO: 402) | ETXS711 (SEQ ID NO: 602) |
| ETXM357 | ETXS714 (SEQ ID NO: 403) | ETXS713 (SEQ ID NO: 603) |
| ETXM358 | ETXS716 (SEQ ID NO: 404) | ETXS715 (SEQ ID NO: 604) |
| ETXM359 | ETXS718 (SEQ ID NO: 405) | ETXS717 (SEQ ID NO: 605) |
| ETXM360 | ETXS720 (SEQ ID NO: 406) | ETXS719 (SEQ ID NO: 606) |
| ETXM361 | ETXS722 (SEQ ID NO: 407) | ETXS721 (SEQ ID NO: 607) |
| ETXM362 | ETXS724 (SEQ ID NO: 408) | ETXS723 (SEQ ID NO: 608) |
| ETXM363 | ETXS726 (SEQ ID NO: 409) | ETXS725 (SEQ ID NO: 609) |
| ETXM364 | ETXS728 (SEQ ID NO: 410) | ETXS727 (SEQ ID NO: 610) |
| ETXM365 | ETXS730 (SEQ ID NO: 411) | ETXS729 (SEQ ID NO: 611) |
| ETXM366 | ETXS732 (SEQ ID NO: 412) | ETXS731 (SEQ ID NO: 612) |
| ETXM367 | ETXS734 (SEQ ID NO: 413) | ETXS733 (SEQ ID NO: 613) |
| ETXM368 | ETXS736 (SEQ ID NO: 414) | ETXS735 (SEQ ID NO: 614) |
| ETXM369 | ETXS738 (SEQ ID NO: 415) | ETXS737 (SEQ ID NO: 615) |
| ETXM370 | ETXS740 (SEQ ID NO: 416) | ETXS739 (SEQ ID NO: 616) |
| ETXM371 | ETXS742 (SEQ ID NO: 417) | ETXS741 (SEQ ID NO: 617) |
| ETXM372 | ETXS744 (SEQ ID NO: 418) | ETXS743 (SEQ ID NO: 618) |
| ETXM373 | ETXS746 (SEQ ID NO: 419) | ETXS745 (SEQ ID NO: 619) |
| ETXM374 | ETXS748 (SEQ ID NO: 420) | ETXS747 (SEQ ID NO: 620) |
| ETXM375 | ETXS750 (SEQ ID NO: 421) | ETXS749 (SEQ ID NO: 621) |
| ETXM376 | ETXS752 (SEQ ID NO: 422) | ETXS751 (SEQ ID NO: 622) |
| ETXM377 | ETXS754 (SEQ ID NO: 423) | ETXS753 (SEQ ID NO: 623) |
| ETXM378 | ETXS756 (SEQ ID NO: 424) | ETXS755 (SEQ ID NO: 624) |
| ETXM379 | ETXS758 (SEQ ID NO: 425) | ETXS757 (SEQ ID NO: 625) |
| ETXM380 | ETXS760 (SEQ ID NO: 426) | ETXS759 (SEQ ID NO: 626) |
| ETXM381 | ETXS762 (SEQ ID NO: 427) | ETXS761 (SEQ ID NO: 627) |
| ETXM382 | ETXS764 (SEQ ID NO: 428) | ETXS763 (SEQ ID NO: 628) |
| ETXM383 | ETXS766 (SEQ ID NO: 429) | ETXS765 (SEQ ID NO: 629) |
| ETXM384 | ETXS768 (SEQ ID NO: 430) | ETXS767 (SEQ ID NO: 630) |
| ETXM385 | ETXS770 (SEQ ID NO: 431) | ETXS769 (SEQ ID NO: 631) |
| ETXM386 | ETXS772 (SEQ ID NO: 432) | ETXS771 (SEQ ID NO: 632) |
| ETXM387 | ETXS774 (SEQ ID NO: 433) | ETXS773 (SEQ ID NO: 633) |
| ETXM388 | ETXS776 (SEQ ID NO: 434) | ETXS775 (SEQ ID NO: 634) |
| ETXM389 | ETXS778 (SEQ ID NO: 435) | ETXS777 (SEQ ID NO: 635) |
| ETXM390 | ETXS780 (SEQ ID NO: 436) | ETXS779 (SEQ ID NO: 636) |
| ETXM391 | ETXS782 (SEQ ID NO: 437) | ETXS781 (SEQ ID NO: 637) |
| ETXM392 | ETXS784 (SEQ ID NO: 438) | ETXS783 (SEQ ID NO: 638) |
| ETXM393 | ETXS786 (SEQ ID NO: 439) | ETXS785 (SEQ ID NO: 639) |
| ETXM394 | ETXS788 (SEQ ID NO: 440) | ETXS787 (SEQ ID NO: 640) |
| ETXM395 | ETXS790 (SEQ ID NO: 441) | ETXS789 (SEQ ID NO: 641) |
| ETXM396 | ETXS792 (SEQ ID NO: 442) | ETXS791 (SEQ ID NO: 642) |
| ETXM397 | ETXS794 (SEQ ID NO: 443) | ETXS793 (SEQ ID NO: 643) |
| ETXM398 | ETXS796 (SEQ ID NO: 444) | ETXS795 (SEQ ID NO: 644) |
| ETXM399 | ETXS798 (SEQ ID NO: 445) | ETXS797 (SEQ ID NO: 645) |
| ETXM400 | ETXS800 (SEQ ID NO: 446) | ETXS799 (SEQ ID NO: 646) |
| ETXM401 | ETXS802 (SEQ ID NO: 447) | ETXS801 (SEQ ID NO: 647) |
| ETXM402 | ETXS804 (SEQ ID NO: 448) | ETXS803 (SEQ ID NO: 648) |
| ETXM403 | ETXS806 (SEQ ID NO: 449) | ETXS805 (SEQ ID NO: 649) |
| ETXM404 | ETXS808 (SEQ ID NO: 450) | ETXS807 (SEQ ID NO: 650) |
| ETXM405 | ETXS810 (SEQ ID NO: 451) | ETXS809 (SEQ ID NO: 651) |
| ETXM406 | ETXS812 (SEQ ID NO: 452) | ETXS811 (SEQ ID NO: 652) |
| ETXM407 | ETXS814 (SEQ ID NO: 453) | ETXS813 (SEQ ID NO: 653) |
| ETXM408 | ETXS816 (SEQ ID NO: 454) | ETXS815 (SEQ ID NO: 654) |
| ETXM409 | ETXS818 (SEQ ID NO: 455) | ETXS817 (SEQ ID NO: 655) |
| ETXM410 | ETXS820 (SEQ ID NO: 456) | ETXS819 (SEQ ID NO: 656) |
| ETXM411 | ETXS822 (SEQ ID NO: 457) | ETXS821 (SEQ ID NO: 657) |
| ETXM412 | ETXS824 (SEQ ID NO: 458) | ETXS823 (SEQ ID NO: 658) |
| ETXM413 | ETXS826 (SEQ ID NO: 459) | ETXS825 (SEQ ID NO: 659) |
| ETXM414 | ETXS828 (SEQ ID NO: 460) | ETXS827 (SEQ ID NO: 660) |
| ETXM415 | ETXS830 (SEQ ID NO: 461) | ETXS829 (SEQ ID NO: 661) |
| ETXM416 | ETXS832 (SEQ ID NO: 462) | ETXS831 (SEQ ID NO: 662) |
| ETXM417 | ETXS834 (SEQ ID NO: 463) | ETXS833 (SEQ ID NO: 663) |
| ETXM418 | ETXS836 (SEQ ID NO: 464) | ETXS835 (SEQ ID NO: 664) |
| ETXM419 | ETXS838 (SEQ ID NO: 465) | ETXS837 (SEQ ID NO: 665) |
| ETXM420 | ETXS840 (SEQ ID NO: 466) | ETXS839 (SEQ ID NO: 666) |
| ETXM421 | ETXS842 (SEQ ID NO: 467) | ETXS841 (SEQ ID NO: 667) |
| ETXM422 | ETXS844 (SEQ ID NO: 468) | ETXS843 (SEQ ID NO: 668) |
| ETXM423 | ETXS846 (SEQ ID NO: 469) | ETXS845 (SEQ ID NO: 669) |
| ETXM424 | ETXS848 (SEQ ID NO: 470) | ETXS847 (SEQ ID NO: 670) |
| ETXM425 | ETXS850 (SEQ ID NO: 471) | ETXS849 (SEQ ID NO: 671) |
| ETXM426 | ETXS852 (SEQ ID NO: 472) | ETXS851 (SEQ ID NO: 672) |
| ETXM427 | ETXS854 (SEQ ID NO: 473) | ETXS853 (SEQ ID NO: 673) |
| ETXM428 | ETXS856 (SEQ ID NO: 474) | ETXS855 (SEQ ID NO: 674) |
| ETXM429 | ETXS858 (SEQ ID NO: 475) | ETXS857 (SEQ ID NO: 675) |
| ETXM430 | ETXS860 (SEQ ID NO: 476) | ETXS859 (SEQ ID NO: 676) |
| ETXM431 | ETXS862 (SEQ ID NO: 477) | ETXS861 (SEQ ID NO: 677) |
| ETXM432 | ETXS864 (SEQ ID NO: 478) | ETXS863 (SEQ ID NO: 678) |
| ETXM433 | ETXS866 (SEQ ID NO: 479) | ETXS865 (SEQ ID NO: 679) |
| ETXM434 | ETXS868 (SEQ ID NO: 480) | ETXS867 (SEQ ID NO: 680) |
| ETXM435 | ETXS870 (SEQ ID NO: 481) | ETXS869 (SEQ ID NO: 681) |
| ETXM436 | ETXS872 (SEQ ID NO: 482) | ETXS871 (SEQ ID NO: 682) |
| ETXM437 | ETXS874 (SEQ ID NO: 483) | ETXS873 (SEQ ID NO: 683) |
| ETXM438 | ETXS876 (SEQ ID NO: 484) | ETXS875 (SEQ ID NO: 684) |
| ETXM439 | ETXS878 (SEQ ID NO: 485) | ETXS877 (SEQ ID NO: 685) |
| ETXM440 | ETXS880 (SEQ ID NO: 486) | ETXS879 (SEQ ID NO: 686) |
| ETXM441 | ETXS882 (SEQ ID NO: 487) | ETXS881 (SEQ ID NO: 687) |
| ETXM442 | ETXS884 (SEQ ID NO: 488) | ETXS883 (SEQ ID NO: 688) |
| ETXM443 | ETXS886 (SEQ ID NO: 489) | ETXS885 (SEQ ID NO: 689) |
| ETXM444 | ETXS888 (SEQ ID NO: 490) | ETXS887 (SEQ ID NO: 690) |
| ETXM445 | ETXS890 (SEQ ID NO: 491) | ETXS889 (SEQ ID NO: 691) |
| ETXM446 | ETXS892 (SEQ ID NO: 492) | ETXS891 (SEQ ID NO: 692) |
| ETXM447 | ETXS894 (SEQ ID NO: 493) | ETXS893 (SEQ ID NO: 693) |
| ETXM448 | ETXS896 (SEQ ID NO: 494) | ETXS895 (SEQ ID NO: 694) |
| ETXM449 | ETXS898 (SEQ ID NO: 495) | ETXS897 (SEQ ID NO: 695) |
| ETXM450 | ETXS900 (SEQ ID NO: 496) | ETXS899 (SEQ ID NO: 696) |
| ETXM451 | ETXS902 (SEQ ID NO: 497) | ETXS901 (SEQ ID NO: 697) |
| ETXM452 | ETXS904 (SEQ ID NO: 498) | ETXS903 (SEQ ID NO: 698) |
| ETXM453 | ETXS906 (SEQ ID NO: 499) | ETXS905 (SEQ ID NO: 699) |
| ETXM454 | ETXS908 (SEQ ID NO: 500) | ETXS907 (SEQ ID NO: 700) |
| ETXM455 | ETXS910 (SEQ ID NO: 501) | ETXS909 (SEQ ID NO: 701) |
| ETXM456 | ETXS912 (SEQ ID NO: 502) | ETXS911 (SEQ ID NO: 702) |
| ETXM457 | ETXS914 (SEQ ID NO: 503) | ETXS913 (SEQ ID NO: 703) |
| ETXM458 | ETXS916 (SEQ ID NO: 504) | ETXS915 (SEQ ID NO: 704) |
| ETXM459 | ETXS918 (SEQ ID NO: 505) | ETXS917 (SEQ ID NO: 705) |
| ETXM460 | ETXS920 (SEQ ID NO: 506) | ETXS919 (SEQ ID NO: 706) |
| ETXM461 | ETXS922 (SEQ ID NO: 507) | ETXS921 (SEQ ID NO: 707) |
| ETXM462 | ETXS924 (SEQ ID NO: 508) | ETXS923 (SEQ ID NO: 708) |
| ETXM463 | ETXS926 (SEQ ID NO: 509) | ETXS925 (SEQ ID NO: 709) |
| ETXM464 | ETXS928 (SEQ ID NO: 510) | ETXS927 (SEQ ID NO: 710) |
| ETXM465 | ETXS930 (SEQ ID NO: 511) | ETXS929 (SEQ ID NO: 711) |
| ETXM466 | ETXS932 (SEQ ID NO: 512) | ETXS931 (SEQ ID NO: 712) |
| ETXM467 | ETXS934 (SEQ ID NO: 513) | ETXS933 (SEQ ID NO: 713) |
| ETXM468 | ETXS936 (SEQ ID NO: 514) | ETXS935 (SEQ ID NO: 714) |
| ETXM469 | ETXS938 (SEQ ID NO: 515) | ETXS937 (SEQ ID NO: 715) |
| ETXM470 | ETXS940 (SEQ ID NO: 516) | ETXS939 (SEQ ID NO: 716) |
| ETXM471 | ETXS942 (SEQ ID NO: 517) | ETXS941 (SEQ ID NO: 717) |
| ETXM472 | ETXS944 (SEQ ID NO: 518) | ETXS943 (SEQ ID NO: 718) |
| ETXM473 | ETXS946 (SEQ ID NO: 519) | ETXS945 (SEQ ID NO: 719) |
| ETXM474 | ETXS948 (SEQ ID NO: 520) | ETXS947 (SEQ ID NO: 720) |
| ETXM475 | ETXS950 (SEQ ID NO: 521) | ETXS949 (SEQ ID NO: 721) |
| ETXM476 | ETXS952 (SEQ ID NO: 522) | ETXS951 (SEQ ID NO: 722) |
| ETXM477 | ETXS954 (SEQ ID NO: 523) | ETXS953 (SEQ ID NO: 723) |
| ETXM478 | ETXS956 (SEQ ID NO: 524) | ETXS955 (SEQ ID NO: 724) |
| ETXM479 | ETXS958 (SEQ ID NO: 525) | ETXS957 (SEQ ID NO: 725) |
| ETXM480 | ETXS960 (SEQ ID NO: 526) | ETXS959 (SEQ ID NO: 726) |
| ETXM481 | ETXS962 (SEQ ID NO: 527) | ETXS961 (SEQ ID NO: 727) |
| ETXM482 | ETXS964 (SEQ ID NO: 528) | ETXS963 (SEQ ID NO: 728) |
| ETXM483 | ETXS966 (SEQ ID NO: 529) | ETXS965 (SEQ ID NO: 729) |
| ETXM484 | ETXS968 (SEQ ID NO: 530) | ETXS967 (SEQ ID NO: 730) |
| ETXM485 | ETXS970 (SEQ ID NO: 531) | ETXS969 (SEQ ID NO: 731) |
| ETXM486 | ETXS972 (SEQ ID NO: 532) | ETXS971 (SEQ ID NO: 732) |
| ETXM487 | ETXS974 (SEQ ID NO: 533) | ETXS973 (SEQ ID NO: 733) |
| ETXM488 | ETXS976 (SEQ ID NO: 534) | ETXS975 (SEQ ID NO: 734) |
| ETXM489 | ETXS978 (SEQ ID NO: 535) | ETXS977 (SEQ ID NO: 735) |
| ETXM490 | ETXS980 (SEQ ID NO: 536) | ETXS979 (SEQ ID NO: 736) |
| ETXM491 | ETXS982 (SEQ ID NO: 537) | ETXS981 (SEQ ID NO: 737) |
| ETXM492 | ETXS984 (SEQ ID NO: 538) | ETXS983 (SEQ ID NO: 738) |
| ETXM493 | ETXS986 (SEQ ID NO: 539) | ETXS985 (SEQ ID NO: 739) |
| ETXM494 | ETXS988 (SEQ ID NO: 540) | ETXS987 (SEQ ID NO: 740) |
| ETXM495 | ETXS990 (SEQ ID NO: 541) | ETXS989 (SEQ ID NO: 741) |
| ETXM496 | ETXS992 (SEQ ID NO: 542) | ETXS991 (SEQ ID NO: 742) |
| ETXM497 | ETXS994 (SEQ ID NO: 543) | ETXS993 (SEQ ID NO: 743) |
| ETXM498 | ETXS996 (SEQ ID NO: 544) | ETXS995 (SEQ ID NO: 744) |
| ETXM499 | ETXS998 (SEQ ID NO: 545) | ETXS997 (SEQ ID NO: 745) |
| ETXM500 | ETXS1000 (SEQ ID NO: 546) | ETXS999 (SEQ ID NO: 746) |
| ETXM501 | ETXS1002 (SEQ ID NO: 547) | ETXS1001 (SEQ ID NO: 747) |
| ETXM502 | ETXS1004 (SEQ ID NO: 548) | ETXS1003 (SEQ ID NO: 748) |
| ETXM503 | ETXS1006 (SEQ ID NO: 549) | ETXS1005 (SEQ ID NO: 749) |
| ETXM504 | ETXS1008 (SEQ ID NO: 550) | ETXS1007 (SEQ ID NO: 750) |
| ETXM505 | ETXS1010 (SEQ ID NO: 551) | ETXS1009 (SEQ ID NO: 751) |
| ETXM506 | ETXS1012 (SEQ ID NO: 552) | ETXS1011 (SEQ ID NO: 752) |
| ETXM507 | ETXS1014 (SEQ ID NO: 553) | ETXS1013 (SEQ ID NO: 753) |
| ETXM508 | ETXS1016 (SEQ ID NO: 554) | ETXS1015 (SEQ ID NO: 754) |
| ETXM509 | ETXS1018(SEQ ID NO: 555) | ETXS1017 (SEQ ID NO: 755) |
| ETXM510 | ETXS1020 (SEQ ID NO: 556) | ETXS1019 (SEQ ID NO: 756) |
| ETXM511 | ETXS1022 (SEQ ID NO: 557) | ETXS1021 (SEQ ID NO: 757) |
| ETXM512 | ETXS1024 (SEQ ID NO: 558) | ETXS1023 (SEQ ID NO: 758) |
| ETXM513 | ETXS1026 (SEQ ID NO: 559) | ETXS1025 (SEQ ID NO: 759) |
| ETXM514 | ETXS1028 (SEQ ID NO: 560) | ETXS1027 (SEQ ID NO: 760) |
| ETXM515 | ETXS1030 (SEQ ID NO: 561) | ETXS1029 (SEQ ID NO: 761) |
| ETXM1064 | ETXS2128 (SEQ ID NO: 795) | ETXS2127 (SEQ ID NO: 798) |
| ETXM1072 | ETXS2144 (SEQ ID NO: 796) | ETXS2143 (SEQ ID NO: 799) |
| ETXM1076 | ETXS2152 (SEQ ID NO: 797) | ETXS2151 (SEQ ID NO: 800) |
| ETXM1180 | ETXS1032 (SEQ ID NO: 762) | ETXS1031 (SEQ ID NO: 772) |
| ETXM1181 | ETXS1034 (SEQ ID NO: 763) | ETXS1033 (SEQ ID NO: 773) |
| ETXM1184 | ETXS1036 (SEQ ID NO: 764) | ETXS1035 (SEQ ID NO: 774) |
| ETXM1185 | ETXS1038 (SEQ ID NO: 765) | ETXS1037 (SEQ ID NO: 775) |
| ETXM1157 | ETXS1040 (SEQ ID NO: 766) | ETXS1039 (SEQ ID NO: 776) |
| ETXM1162 | ETXS1042 (SEQ ID NO: 767) | ETXS1041 (SEQ ID NO: 777) |
| ETXM1188 | ETXS1044 (SEQ ID NO: 768) | ETXS1043 (SEQ ID NO: 778) |
| ETXM1189 | ETXS1046 (SEQ ID NO: 769) | ETXS1045 (SEQ ID NO: 779) |
| ETXM1192 | ETXS1048 (SEQ ID NO: 770) | ETXS1047 (SEQ ID NO: 780) |
| ETXM1193 | ETXS1050 (SEQ ID NO: 771) | ETXS1049 (SEQ ID NO: 781) |
| ETXM1194 | ETXS1051 (SEQ ID NO: 782) | ETXS1033 (SEQ ID NO: 773) |
| ETXM1195 | ETXS1052 (SEQ ID NO: 783) | ETXS1037 (SEQ ID NO: 775) |
| ETXM1196 | ETXS1053 (SEQ ID NO: 784) | ETXS1041 (SEQ ID NO: 777) |
| ETXM1197 | ETXS1054 (SEQ ID NO: 785) | ETXS1045 (SEQ ID NO: 779) |
| ETXM1198 | ETXS1055 (SEQ ID NO: 786) | ETXS1049 (SEQ ID NO: 781) |

In a particularly preferred embodiment, the invention relates to a nucleic acid comprising first and second strands that comprise, consist of, or consist essentially of a nucleoside sequence differing by 0 or 1 nucleosides from any one of the following first and second sequences:

| Duplex ID | Modified first strand | Modified second strand |
|---|---|---|
| ETXM342 | SEQ ID NO: 388 | SEQ ID NO: 588 |
| ETXM339 | SEQ ID NO: 385 | SEQ ID NO: 585 |
| ETXM338 | SEQ ID NO: 384 | SEQ ID NO: 584 |
| ETXM359 | SEQ ID NO: 405 | SEQ ID NO: 605 |
| ETXM396 | SEQ ID NO: 442 | SEQ ID NO: 642 |

In an even more preferred embodiment, the invention relates to a nucleic acid comprising first and second strands that comprise, consist of, or consist essentially of a nucleoside sequence differing by 0 or 1 nucleosides from any one of the following first and second sequences:

| Duplex ID | Modified first strand | Modified second strand |
|---|---|---|
| ETXM1180 | SEQ ID NO: 762 | SEQ ID NO: 772 |
| ETXM1181 | SEQ ID NO: 763 | SEQ ID NO: 773 |
| ETXM1184 | SEQ ID NO: 764 | SEQ ID NO: 774 |
| ETXM1185 | SEQ ID NO: 765 | SEQ ID NO: 775 |
| ETXM1157 | SEQ ID NO: 766 | SEQ ID NO: 776 |
| ETXM1162 | SEQ ID NO: 767 | SEQ ID NO: 777 |
| ETXM1188 | SEQ ID NO: 768 | SEQ ID NO: 778 |
| ETXM1189 | SEQ ID NO: 769 | SEQ ID NO: 779 |
| ETXM1192 | SEQ ID NO: 770 | SEQ ID NO: 780 |
| ETXM1193 | SEQ ID NO: 771 | SEQ ID NO: 781 |
| ETXM1194 | SEQ ID NO: 782 | SEQ ID NO: 773 |
| ETXM1195 | SEQ ID NO: 783 | SEQ ID NO: 775 |
| ETXM1196 | SEQ ID NO: 784 | SEQ ID NO: 777 |
| ETXM1197 | SEQ ID NO: 785 | SEQ ID NO: 779 |
| ETXM1198 | SEQ ID NO: 786 | SEQ ID NO: 781 |

In case of ambiguity between the sequences in this specification and the sequences in the attached sequence listing, the sequences provided herein are considered to be the correct sequences.

### Abasic Nucleotides

In certain embodiments, there are 1, e.g. 2, e.g. 3, e.g. 4 or more abasic nucleosides present in nucleic acids according to the invention. Abasic nucleosides are modified nucleosides because they lack the base normally seen at position 1 of the sugar moiety. Typically, there will be a hydrogen at position 1 of the sugar moiety of the abasic nucleosides present in a nucleic acid according to the present invention.

The abasic nucleosides are in the terminal region of the second strand, preferably located within the terminal 5 nucleosides of the end of the strand. The terminal region may be the terminal 5 nucleosides, which includes abasic nucleosides.

The second strand may comprise, as preferred features (which are all specifically contemplated in combination unless mutually exclusive):
2, or more than 2, abasic nucleosides in a terminal region of the second strand; and/or
2, or more than 2, abasic nucleosides in either the 5′ or 3′ terminal region of the second strand; and/or
2, or more than 2, abasic nucleosides in either the 5′ or 3′ terminal region of the second strand, wherein the abasic nucleosides are present in an overhang as herein described; and/or
2, or more than 2, consecutive abasic nucleosides in a terminal region of the second strand, wherein preferably one such abasic nucleosides is a terminal nucleosides; and/or
2, or more than 2, consecutive abasic nucleosides in either the 5′ or 3′ terminal region of the second strand, wherein preferably one such abasic nucleosides is a terminal nucleosides in either the 5′ or 3′ terminal region of the second strand; and/or
a reversed internucleoside linkage connects at least one abasic nucleoside to an adjacent basic nucleoside in a terminal region of the second strand; and/or
a reversed internucleoside linkage connects at least one abasic nucleoside to an adjacent basic nucleoside in either the 5′ or 3′ terminal region of the second strand; and/or
an abasic nucleoside as the penultimate nucleoside which is connected via the reversed linkage to the nucleoside which is not the terminal nucleoside (called the antepenultimate nucleoside herein); and/or
abasic nucleosides as the 2 terminal nucleosides connected via a 5′-3′ linkage when reading the strand in the direction towards the terminus comprising the terminal nucleosides;
abasic nucleosides as the 2 terminal nucleosides connected via a 3′-5′ linkage when reading the strand in the direction towards the terminus comprising the terminal nucleosides;
abasic nucleosides as the terminal 2 positions, wherein the penultimate nucleoside is connected via the reversed linkage to the antepenultimate nucleoside, and wherein the reversed linkage is a 5-5′ reversed linkage or a 3′-3′ reversed linkage;
abasic nucleosides as the terminal 2 positions, wherein the penultimate nucleoside is connected via the reversed linkage to the antepenultimate nucleoside, and wherein either
(1) the reversed linkage is a 5-5′ reversed linkage and the linkage between the terminal and penultimate abasic nucleosides is 3′5′ when reading towards the terminus comprising the terminal and penultimate abasic nucleosides; or
(2) the reversed linkage is a 3-3′ reversed linkage and the linkage between the terminal and penultimate abasic nucleosides is 5′3′ when reading towards the terminus comprising the terminal and penultimate abasic nucleosides.

Preferably there is an abasic nucleoside at the terminus of the second strand.

Preferably there are 2 or at least 2 abasic nucleosides in the terminal region of the second strand, preferably at the terminal and penultimate positions.

Preferably 2 or more abasic nucleosides are consecutive, for example all abasic nucleosides may be consecutive. For example, the terminal 1 or terminal 2 or terminal 3 or terminal 4 nucleotides may be abasic nucleosides.

An abasic nucleoside may also be linked to an adjacent nucleoside through a 5′-3′ phosphodiester linkage or reversed linkage unless there is only 1 abasic nucleoside at the terminus, in which case it will have a reversed linkage to the adjacent nucleoside.

A reversed linkage (which may also be referred to as an inverted linkage, which is also seen in the art), comprises either a 5′-5′, a 3-′3′, a 3′-2′ or a 2′-3′ phosphodiester linkage between the adjacent sugar moieties of the nucleosides.

Abasic nucleosides which are not terminal will have 2 phosphodiester bonds, one with each adjacent nucleoside, and these may be a reversed linkage or may be a 5′-3 phosphodiester bond or may be one of each.

A preferred embodiment comprises 2 abasic nucleosides at the terminal and penultimate positions of the second strand, and wherein the reversed internucleoside linkage is located between the penultimate (abasic) nucleoside and the antepenultimate nucleoside.

Preferably there are 2 abasic nucleosides at the terminal and penultimate positions of the second strand and the penultimate nucleoside is linked to the antepenultimate nucleoside through a reversed internucleoside linkage and is linked to the terminal nucleoside through a 5′-3′ or 3′-5′ phosphodiester linkage (reading in the direction of the terminus of the molecule).

Different preferred features are as follows:

The reversed internucleoside linkage is a 3′-3′ reversed linkage. The reversed internucleoside linkage is at a terminal region which is distal to the 5′ terminal phosphate of the second strand.

The reversed internucleoside linkage is a 5′-5′ reversed linkage. The reversed internucleoside linkage is at a terminal region which is distal to the 3′ terminal hydroxide of the second strand.

In certain embodiments, the second strand comprises 2 consecutive abasic nucleosides in the 5′ terminal region of the second strand, wherein one such abasic nucleoside is a terminal nucleoside at the 5′ terminal region of the second strand and the other abasic nucleoside is a penultimate nucleoside at the 5′ terminal region of the second strand, wherein: (a) said penultimate abasic nucleoside is connected to an adjacent first basic nucleoside in an adjacent 5′ near terminal region through a reversed internucleoside linkage; and (b) the reversed linkage is a 5-5′ reversed linkage; and (c) the linkage between the terminal and penultimate abasic nucleosides is 3′5′ when reading towards the terminus comprising the terminal and penultimate abasic nucleosides. More typically, (i) the first strand and the second strand each has a length of 23 nucleosides; (ii) two phosphorothioate internucleoside linkages are respectively between three consecutive positions in said 5′ near terminal region of the second strand, wherein a first phosphorothioate internucleoside linkage is present between said adjacent first basic nucleoside of (a) and an adjacent second basic nucleoside in said 5′ near terminal region of the second strand, and a second phosphorothioate internucleoside linkage is present between said adjacent second basic nucleoside and an adjacent third basic nucleoside in said 5′ near terminal region of the second strand; (iii) two phosphorothioate internucleoside linkages are respectively between three consecutive positions in both 5′ and 3′ terminal regions of the first strand, whereby a terminal nucleoside respectively at each of the 5′ and 3′ terminal regions of said first strand is each attached to a respective 5′ and 3′ adjacent penultimate nucleoside by a phosphorothioate internucleoside linkage, and each first 5′ and 3′ penultimate nucleoside is attached to a respective 5′ and 3′ adjacent antepenultimate nucleoside by a phosphorothioate internucleoside linkage; and (iv) the second strand of the nucleic acid is conjugated directly or indirectly to one or more ligand moieties at the 3′ terminal region of the second strand.

Alternatively the second strand comprises 2 consecutive abasic nucleosides preferably in an overhang in the 3′ terminal region of the second strand, wherein one such abasic nucleoside is a terminal nucleoside at the 3′ terminal region of the second strand and the other abasic nucleoside is a penultimate nucleoside at the 3′ terminal region of the second strand, wherein: (a) said penultimate abasic nucleoside is connected to an adjacent first basic nucleoside in an adjacent 3′ near terminal region through a reversed internucleoside linkage; and (b) the reversed linkage is a 3-3′ reversed linkage; and (c) the linkage between the terminal and penultimate abasic nucleosides is 5′-3′ when reading towards the terminus comprising the terminal and penultimate abasic nucleosides. More typically, (i) the first strand and the second strand each has a length of 23 nucleosides; (ii) two phosphorothioate internucleoside linkages are respectively between three consecutive positions in said 3′ near terminal region of the second strand, wherein a first phosphorothioate internucleoside linkage is present between said adjacent first basic nucleoside of (a) and an adjacent second basic nucleoside in said 3′ near terminal region of the second strand, and a second phosphorothioate internucleoside linkage is present between said adjacent second basic nucleoside and an adjacent third basic nucleoside in said 3′ near terminal region of the second strand; (iii) two phosphorothioate internucleoside linkages are respectively between three consecutive positions in both 5′ and 3′ terminal regions of the first strand, whereby a terminal nucleoside respectively at each of the 5′ and 3′ terminal regions of said first strand is each attached to a respective 5′ and 3′ adjacent penultimate nucleoside by a phosphorothioate internucleoside linkage, and each first 5′ and 3′ penultimate nucleoside is attached to a respective 5′ and 3′ adjacent antepenultimate nucleoside by a phosphorothioate internucleoside linkage; and (iv) the second strand of the nucleic acid is conjugated directly or indirectly to one or more ligand moieties at the 5′ terminal region of the second strand.

Examples of the structures are as follows (where the specific RNA nucleosides shown are not limiting and could be any RNA nucleoside):
A A 3′-3′ reversed bond (and also showing the 5′-3 direction of the last phosphodiester bond between the two abasic molecules reading towards the terminus of the molecule)

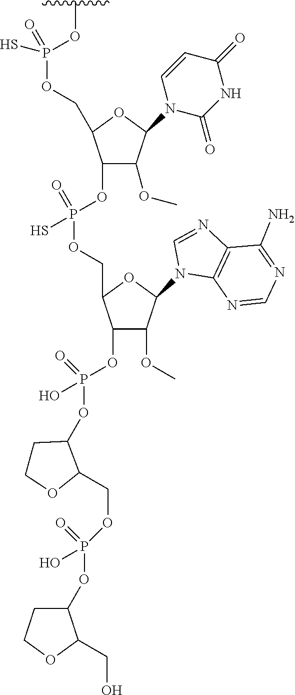 [SMILES: *OP(=O)(S)OCC1O[C@@H](n2ccc(=O)[nH]c2=O)C(OC)C1OP(=O)(S)OCC1O[C@@H](n2cnc3c(N)ncnc32)C(OC)C1OP(=O)(O)OC1CCOC1COP(=O)(O)OC1CCOC1CO]

B Illustrating a 5′-5′ reversed bond (and also showing the 3′-5′ direction of the last phosphodiester bond between the two abasic molecules reading towards the terminus of the molecule)

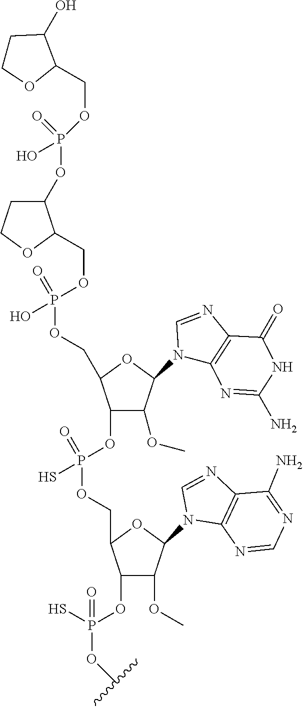 [SMILES: *OP(=O)(S)OC1C(COP(=O)(S)OC2C(COP(=O)(O)OCC3OCCC3OP(=O)(O)OCC3OCCC3O)O[C@@H](n3cnc4c(=O)[nH]c(N)nc43)C2OC)O[C@@H](n2cnc3c(N)ncnc32)C1OC]

The abasic nucleoside or abasic nucleosides present in the nucleic acid are provided in the presence of a reversed internucleoside linkage or linkages, namely a 5′-5′ or a 3′-3′ reversed internucleoside linkage. A reversed linkage occurs as a result of a change of orientation of an adjacent nucleoside sugar, such that the sugar will have a 3′-5′ orientation as opposed to the conventional 5′-3′ orientation (with reference to the numbering of ring atoms on the nucleoside sugars). The abasic nucleoside or nucleosides as present in the nucleic acids of the invention preferably include such inverted nucleoside sugars.

In the case of a terminal nucleoside having an inverted orientation, then this will result in an “inverted” end configuration for the overall nucleic acid. Whilst certain structures drawn and referenced herein are represented using conventional 5′-3′ direction (with reference to the numbering of ring atoms on the nucleoside sugars), it will be appreciated that the presence of a terminal nucleoside having a change of orientation and a proximal 3′-3′ reversed linkage, will result in a nucleic acid having an overall 5′-5′ end structure (i.e. the conventional 3′ end nucleoside becomes a 5′ end nucleoside). Alternatively, it will be appreciated that the presence of a terminal nucleoside having a change of orientation and a proximal 5′-5′ reversed linkage will result in a nucleic acid with an overall 3′-3′ end structure.

The proximal 3′-3′ or 5′-5′ reversed linkage as herein described, may comprise the reversed linkage being directly adjacent/attached to a terminal nucleoside having an inverted orientation, such as a single terminal nucleoside having an inverted orientation. Alternatively, the proximal 3′-3′ or 5′-5′ reversed linkage as herein described, may comprise the reversed linkage being adjacent 2, or more than 2, nucleosides having an inverted orientation, such as 2, or more than 2, terminal region nucleosides having an inverted orientation, such as the terminal and penultimate nucleosides. In this way, the reversed linkage may be attached to a penultimate nucleoside having an inverted orientation. While a skilled addressee will appreciate that inverted orientations as described above can result in nucleic acid molecules having overall 3′-3′ or 5′-5′ end structures as described herein, it will also be appreciated that with the presence of one or more additional reversed linkages and/or nucleosides having an inverted orientation, then the overall nucleic acid may have 3′-5′ end structures corresponding to the conventionally positioned 5′/3′ ends.

In one aspect the nucleic acid may have a 3′-3′ reversed linkage, and the terminal sugar moiety may comprise a 5′ OH rather than a 5′ phosphate group at the 5′ position of that terminal sugar.

A skilled person would therefore clearly understand that 5′-5′, 3′-3′ and 3′-5′ (reading in the direction of that terminus) end variants of the more conventional 5′-3′ structures (with reference to the numbering of ring atoms on the end nucleoside sugars) drawn herein are included in the scope of the disclosure, where a reversed linkage or linkages is/are present.

In the situation of e.g. a reversed internucleoside linkage and/or one or more nucleosides having an inverted orientation creating an inverted end, and where the relative position of a linkage (e.g. to a linker) or the location of an internal feature (such as a modified nucleoside) is defined relative to the 5′ or 3′ end of the nucleic acid, then the 5′ or 3′ end is the conventional 5′ or 3′ end which would have existed had a reversed linkage not been in place, and wherein the conventional 5′ or 3′ end is determined by consideration of the directionality of the majority of the internal nucleoside linkages and/or nucleoside orientation within the nucleic acid. It is possible to tell from these internal bonds and/or nucleoside orientation which ends of the nucleic acid would constitute the conventional 5′ and 3′ ends (with reference to the numbering of ring atoms on the end nucleoside sugars) of the molecule absent the reversed linkage.

For example, in the structure shown below there are abasic residues in the first 2 positions located at the “5′” end. Where the terminal nucleoside has an inverted orientation then the “5′” end indicated in the diagram below, which is the conventional 5′ end, can in fact comprise a 3′ OH in view of the inverted nucleoside at the terminal position. Nevertheless the majority of the molecule will comprise conventional internucleoside linkages that run from the 3′ OH of the sugar to the 5′ phosphate of the next sugar, when reading in the standard 5′ [PO4] to 3′ [OH] direction of a nucleic acid molecule (with reference to the numbering of ring atoms on the nucleoside sugars), which can be used to determine the conventional 5′ and 3′ ends that would be found absent the inverted end configuration.

A 5′ A-A-Me-Me-Me-Me-Me-Me-F-Me-F—F—F-Me-Me-Me-Me-Me-Me-Me-Me-Me-Me 3′

The reversed bond is preferably located at the end of the nucleic acid e.g. RNA which is distal to a ligand moiety, such as a GalNAc containing portion, of the molecule.

GalNAc-siRNA constructs with a 5′-GalNAc on the sense strand can have a reversed linkage on the opposite end of the sense strand.

GalNAc-siRNA constructs with a 3′-GalNAc on the sense strand can have a reversed linkage on the opposite end of the sense strand.

### Nucleic Acid Lengths

In one aspect the i) the first strand of the nucleic acid has a length in the range of 17 to 30 nucleosides, preferably 19 to 25 nucleosides, more preferably 19 or 23 nucleosides; and/or ii) the second strand of the nucleic acid has a length in the range of 17 to 30 nucleosides, preferably 19 to 25 nucleosides, more preferably 19 or 21 nucleosides.

Typically, the duplex region of the nucleic acid is between 17 and 30 nucleosides in length, more preferably is 19 or 21 nucleosides in length. Similarly, the region of complementarity between the first strand and the portion of RNA transcribed from the ZPI gene is between 17 and 30 nucleosides in length. Generally, the duplex structure of the nucleic acid e.g. an iRNA is about 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

Similarly, the region of complementarity of an antisense sequence to a target sequence and/or the region of complementarity of an antisense sequence to a sense sequence is about 15 to 30 nucleosides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleosides in length. Ranges and lengths intermediate to the above recited ranges and lengths are also contemplated to be part of the invention.

In certain preferred embodiments, the region of complementarity of an antisense sequence to a target sequence and/or the region of complementarity of an antisense sequence to a sense sequence is at least 17 nucleosides in length. For example, the region of complementarity between the antisense strand and the target is 19 to 21 nucleosides in length, for example, the region of complementarity is 21 nucleosides in length.

In preferred embodiments, each strand is no more than 30 nucleosides in length.

In certain preferred embodiments, the duplex structure of the nucleic acid e.g. an siRNA is 19 or 21 base pairs in length. In particularly preferred embodiment, the duplex may have one of the following structures:
e.g., ETXM316-ETXM415, ETXM436-ETXM515 and ETXM1064-ETXM1198:

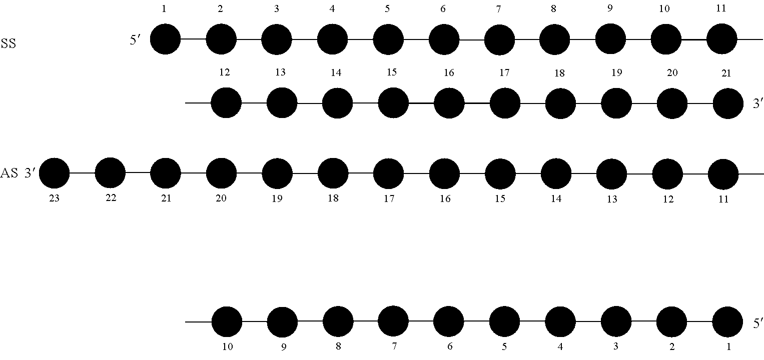

e.g., ETXM416-ETXM435:

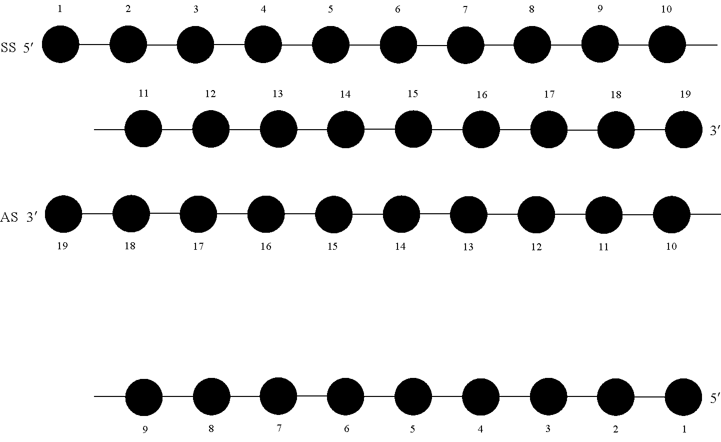

A nucleic acid e.g. a dsRNA as described herein can further include one or more single-stranded nucleoside overhangs e.g., 1-4, 2-4, 1-3, 2-3, 1, 2, 3, or 4 nucleosides. A nucleoside overhang can comprise or consist of a nucleoside/nucleoside analog, including a deoxynucleoside/nucleoside. The overhang(s) can be on the sense strand, the antisense strand, or any combination thereof. Furthermore, the nucleoside(s) of an overhang can be present on the 5′-end, 3′-end, or both ends of an antisense or sense strand of a nucleic acid e.g. a dsRNA.

In certain preferred embodiments, at least one strand comprises a 3′ overhang of at least 1 nucleoside, e.g., at least one strand comprises a 3′ overhang of at least 2 nucleosides. The overhang is suitably on the antisense/guide strand and/or the sense/passenger strand.

### Nucleic Acid Modifications

In certain embodiments, the nucleic acid e.g. an RNA of the invention e.g., a dsiRNA, does not comprise further modifications, e.g., chemical modifications or conjugations known in the art and described herein.

In other preferred embodiments, the nucleic acid e.g. RNA of the invention, e.g., a dsiRNA, is further chemically modified to enhance stability or other beneficial characteristics.

In certain embodiments of the invention, substantially all of the nucleosides are modified.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

Modifications include, for example, end modifications, e.g., 5′-end modifications (phosphorylation, conjugation, inverted linkages) or 3′-end modifications (conjugation, DNA nucleosides within an RNA, or RNA nucleosides within a DNA, inverted linkages, etc.); base modifications, e.g., replacement with stabilizing bases, destabilizing bases, or bases that base pair with an expanded repertoire of partners, conjugated bases; sugar modifications (e.g., at the 2′-position or 4′-position) or replacement of the sugar; or backbone modifications, including modification or replacement of the phosphodiester linkages.

Specific examples of nucleic acids such as siRNA compounds useful in the embodiments described herein include, but are not limited to RNAs containing modified backbones or no natural internucleoside linkages. Nucleic acids such as RNAs having modified backbones include, among others, those that do not have a phosphorus atom in the backbone. For the purposes of this specification, and as sometimes referenced in the art, modified nucleic acids e.g. RNAs that do not have a phosphorus atom in their internucleoside backbone can also be considered to be oligonucleosides. In some embodiments, a modified nucleic acid e.g. an siRNA will have a phosphorus atom in its internucleoside backbone.

Modified nucleic acid e.g. RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 5′-3′ or 5-2′. Various salts, mixed salts and free acid forms are also included.

Modified nucleic acids e.g. RNAs can also contain one or more substituted sugar moieties. The nucleic acids e.g. siRNAs, e.g., dsiRNAs, featured herein can include one of the following at the 2′-position: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted. 2′ O-methyl and 2′-F are preferred modifications.

In certain preferred embodiments, the nucleic acid comprises at least one modified nucleoside.

The nucleic acid of the invention may comprise one or more modified nucleosides on the first strand and/or the second strand.

In some embodiments, substantially all of the nucleosides of the sense strand and all of the nucleosides of the antisense strand comprise a modification.

In some embodiments, all of the nucleosides of the sense strand and substantially all of the nucleosides of the antisense strand comprise a modification.

In some embodiments, all of the nucleosides of the sense strand and all of the nucleosides of the antisense strand comprise a modification.

In one embodiment, at least one of the modified nucleosides is selected from the group consisting of a deoxy-nucleoside, a 3′-terminal deoxy-thymine (dT) nucleoside, a 2′-O-methyl modified nucleoside (also called herein 2′-Me, where Me is a methoxy), a 2′-fluoro modified nucleoside, a 2′-deoxy-modified nucleoside, a locked nucleoside, an unlocked nucleoside, a conformationally restricted nucleoside, a constrained ethyl nucleoside, an abasic nucleoside, a 2′-amino-modified nucleoside, a 2′-O-allyl-modified nucleoside, 2′-C-alkyl-modified nucleoside, 2′-hydroxly-modified nucleoside, a 2′-methoxyethyl modified nucleoside, a 2′-O-alkyl-modified nucleoside, a morpholino nucleoside, a phosphoramidate, a non-natural base comprising nucleoside, a tetrahydropyran modified nucleoside, a 1,5-anhydrohexitol modified nucleoside, a cyclohexenyl modified nucleoside, a nucleoside comprising a phosphorothioate group, a nucleoside comprising a methylphosphonate group, a nucleoside comprising a 5′-phosphate, and a nucleoside comprising a 5′-phosphate mimic. In another embodiment, the modified nucleosides comprise a short sequence of 3′-terminal deoxy-thymine nucleosides (dT).

Modifications on the nucleosides may preferably be selected from the group including, but not limited to, LNA, HNA, CeNA, 2-methoxyethyl, 2′-O-alkyl, 2-O-allyl, 2′-C-allyl, 2′-fluoro, 2′-deoxy, 2′-hydroxyl, and combinations thereof. In another embodiment, the modifications on the nucleosides are 2-O-methyl (“2-Me”) or 2′-fluoro modifications.

One preferred modification is a modification at the 2′-OH group of the ribose sugar, optionally selected from 2′-Me or 2′-F modifications.

Preferred nucleic acid comprise one or more nucleosides on the first strand and/or the second strand which are modified, to form modified nucleosides, as follows:

A nucleic acid wherein the modification is a modification at the 2′-OH group of the ribose sugar, optionally selected from 2′-Me or 2′-F modifications.

A nucleic acid wherein the first strand comprises a 2′-F modification at any of position 2, position 6, position 14, or any combination thereof, counting from position 1 of said first strand.

A nucleic acid wherein the second strand comprises a 2′-F modification at any of position 7, position 9, position 11, or any combination thereof, counting from position 1 of said second strand.

A nucleic acid wherein the second strand comprises a 2′-F modification at position 7 and/or 9, and/or 11, and/or 13, counting from position 1 of said second strand.

A nucleic acid wherein the second strand comprises a 2′-F modification at position 7 and 9 and 11 counting from position 1 of said second strand.

A nucleic acid wherein the first and second strand each comprise 2′-Me and 2′-F modifications.

A nucleic which comprises at least one thermally destabilizing modification, suitably at one or more of positions 1 to 9 of the first strand counting from position 1 of the first strand, and/or at one or more of positions on the second strand aligned with positions 1 to 9 of the first strand, wherein the destabilizing modification is selected from a modified unlocked nucleic acid (UNA) and a glycol nucleic acid (GNA), preferably a glycol nucleic acid. A nucleic acid wherein the nucleic acid comprises 3 or more 2′-F modifications at positions 7 to 13 of the second strand, such as 4, 5, 6 or 7 2′-F modifications at positions 7 to 13 of the second strand, counting from position 1 of said second strand.

A nucleic acid wherein said second strand comprises at least 3, such as 4, 5 or 6, 2′-Me modifications at positions 1 to 6 of the second strand, counting from position 1 of said second strand.

A nucleic acid wherein said first strand comprises at least 5 2′-Me consecutive modifications at the 3′ terminal region, preferably including the terminal nucleoside at the 3′ terminal region, or at least within 1 or 2 nucleosides from the terminal nucleoside at the 3′ terminal region.

A nucleic acid wherein said first strand comprises 7 2′-Me consecutive modifications at the 3′ terminal region, preferably including the terminal nucleoside at the 3′ terminal region.

A nucleic acid which comprises at least one thermally destabilizing modification at position 7 of the first strand, counting from position 1 of the first strand.

A nucleic acid which is an siRNA oligonucleoside, wherein the siRNA oligonucleoside comprises at least 3 2′-F modifications at positions 6 to 12 of the second strand, counting from position 1 of said second strand.

A nucleic acid which is an siRNA oligonucleoside, wherein said second strand comprises at least 3 2′-Me modifications at positions 1 to 6 of the second strand, counting from position 1 of said second strand.

A nucleic acid which is an siRNA oligonucleoside, wherein each of the first and second strands comprises an alternating modification pattern, preferably a fully alternating modification pattern along the entire length of each of the first and second strands, wherein the nucleosides of the first strand are modified by (i) 2′Me modifications on the odd numbered nucleosides counting from position 1 of the first strand, and (ii) 2′F modifications on the even numbered nucleosides counting from position 1 of the first strand, and nucleosides of the second strand are modified by (i) 2′F modifications on the odd numbered nucleosides counting from position 1 of the second strand, and (ii) 2′Me modifications on the even numbered nucleosides counting from position 1 of the second strand. Typically, such fully alternating modification patterns are present in a blunt ended oligonucleoside, wherein each of the first and second strands are 19 or 23 nucleosides in length.

Position 1 of the first or the second strand is the nucleoside which is the closest to the end of the nucleic acid (ignoring any abasic nucleosides) and that is joined to an adjacent nucleoside (at Position 2) via a 3′ to 5′ internal bond, with reference to the bonds between the sugar moieties of the backbone, and reading in a direction away from that end of the molecule.

It can therefore be seen that “position 1 of the sense strand” is the 5′ most nucleoside (not including abasic nucleosides) at the conventional 5′ end of the sense strand. Typically, the nucleoside at this position 1 of the sense strand will be equivalent to the 5′ nucleoside of the selected target nucleic acid sequence, and more generally the sense strand will have equivalent nucleosides to those of the target nucleic acid sequence starting from this position 1 of the sense strand, whilst also allowing for acceptable mismatches between the sequences.

As used herein, “position 1 of the antisense strand” is the 5′ most nucleoside (not including abasic nucleosides) at the conventional 5′ end of the antisense strand. As hereinbefore described, there will be a region of complementarity between the sense and antisense strands, and in this way the antisense strand will also have a region of complementarity to the target nucleic acid sequence as referred to above.

In certain embodiments, the nucleic acid e.g. RNAi agent further comprises at least one phosphorothioate or methylphosphonate internucleoside linkage. For example the phosphorothioate or methylphosphonate internucleoside linkage can be at the 3′-terminus or in the terminal region of one strand, i.e., the sense strand or the antisense strand; or at the ends of both strands, the sense strand and the antisense strand.

In certain embodiments, the phosphorothioate or methylphosphonate internucleoside linkage is at the 5′terminus or in the terminal region of one strand, i.e., the sense strand or the antisense strand; or at the ends of both strands, the sense strand and the antisense strand.

In certain embodiments, a phosphorothioate or a methylphosphonate internucleoside linkage is at both the 5′- and 3′-terminus or in the terminal region of one strand, i.e., the sense strand or the antisense strand; or at the ends of both strands, the sense strand and the antisense strand.

Any nucleic acid may comprise one or more phosphorothioate (PS) modifications within the nucleic acid, such as at least two PS internucleoside bonds at the ends of a strand.

At least one of the oligoribonucleoside strands preferably comprises at least two consecutive phosphorothioate modifications in the last 3 nucleosides of the oligonucleoside.

The invention therefore also relates to: A nucleic acid disclosed herein which comprises phosphorothioate internucleoside linkages respectively between at least two or three consecutive positions, such as in a 5′ and/or 3′ terminal region and/or near terminal region of the second strand, whereby said near terminal region is preferably adjacent said terminal region wherein said one or more abasic nucleosides of said second strand is/are located.

A nucleic acid disclosed herein which comprises phosphorothioate internucleoside linkages respectively between at least two or three consecutive positions in a 5′ and/or 3′ terminal region of the first strand, whereby preferably the terminal position at the 5′ and/or 3′ terminal region of said first strand is attached to its adjacent position by a phosphorothioate internucleoside linkage.

The nucleic acid strand may be an RNA comprising a phosphorothioate internucleoside linkage between the three nucleosides contiguous with 2 terminally located abasic nucleosides.

A preferred nucleic acid is a double stranded RNA comprising 2 adjacent abasic nucleosides at the 5′ terminus of the second strand and a ligand moiety comprising one or more GalNAc ligand moieties at the opposite 3′ end of the second strand. Further preferred, the same nucleic acid may also comprise a phosphorothioate bond between nucleotides at positions 3-4 and 4-5 of the second strand, reading from the position 1 of the second strand. Further preferred, the same nucleic acid may also comprise a 2′ F modification at positions 7, 9 and 11 of the second strand.

Preferred modifications of nucleic acids having the structure

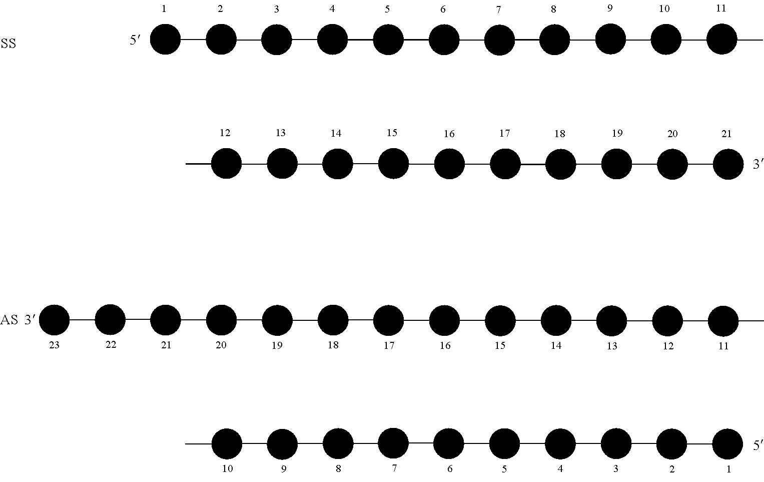

are as follows:

A nucleic acid wherein modified nucleosides of said second strand comprise a modification pattern according to any one of the following (5′-3′):

Me-Me-Me-Me-Me-Me-F—F—F—F—F-Me-Me-Me-Me-Me-Me-Me-F-Me-Me, or

Me-Me-Me-Me-Me-F—F-Me-F—F—F—F-Me-Me-Me-Me-Me-Me-Me-Me-Me, or

Me-Me-Me-Me-Me-Me-F-Me-F—F—F—F-Me-Me-Me-Me-Me-Me-Me-Me-Me, or

Me-Me-Me-Me-Me-Me-Me-Me-F—F—F-Me-Me-Me-Me-Me-Me-Me-Me-Me-Me, or

Me-Me-Me-Me-Me-Me-F-Me-F—F—F-Me-Me-Me-Me-Me-Me-Me-Me-Me-Me.

A nucleic acid wherein modified nucleosides of said second strand comprise a modification pattern according to any one of the following (5′-3′):

Me(s)Me(s)Me-Me-Me-Me-F—F—F—F—F-Me-Me-Me-Me-Me-Me-Me-F-Me-Me, or

Me(s)Me(s)Me-Me-Me-F—F-Me-F—F—F—F-Me-Me-Me-Me-Me-Me-Me-Me-Me, or

Me(s)Me(s)Me-Me-Me-Me-F-Me-F—F—F—F-Me-Me-Me-Me-Me-Me-Me-Me-Me, or

Me(s)Me(s)Me-Me-Me-Me-Me-Me-F—F—F-Me-Me-Me-Me-Me-Me-Me-Me-Me-Me, or

Me(s)Me(s)Me-Me-Me-Me-F-Me-F—F—F-Me-Me-Me-Me-Me-Me-Me-Me-Me-Me, or

Me-Me-Me-Me-Me-Me-F—F—F—F—F-Me-Me-Me-Me-Me-Me-Me-F(s)Me(s)Me, or

Me-Me-Me-Me-Me-F—F-Me-F—F—F—F-Me-Me-Me-Me-Me-Me-Me(s)Me(s)Me, or

Me-Me-Me-Me-Me-Me-F-Me-F—F—F—F-Me-Me-Me-Me-Me-Me-Me(s)Me(s)Me, or

Me-Me-Me-Me-Me-Me-Me-Me-F—F—F-Me-Me-Me-Me-Me-Me-Me-Me(s)Me(s)Me, or

Me-Me-Me-Me-Me-Me-F-Me-F—F—F-Me-Me-Me-Me-Me-Me-Me-Me(s)Me(s)Me,

wherein (s) is a phosphorothioate internucleoside linkage.

A nucleic acid wherein modified nucleosides of said second strand comprise a modification pattern according to any one of the following (5′-3′):

ia-ia-Me-Me-Me-Me-Me-Me-F—F—F—F—F-Me-Me-Me-Me-Me-Me-Me-F-Me-Me, or

ia-ia-Me-Me-Me-Me-Me-F—F-Me-F—F—F—F-Me-Me-Me-Me-Me-Me-Me-Me-Me, or

ia-ia-Me-Me-Me-Me-Me-Me-F-Me-F—F—F—F-Me-Me-Me-Me-Me-Me-Me-Me-Me, or

ia-ia-Me-Me-Me-Me-Me-Me-Me-Me-F—F—F-Me-Me-Me-Me-Me-Me-Me-Me-Me-Me, or

ia-ia-Me-Me-Me-Me-Me-Me-F-Me-F—F—F-Me-Me-Me-Me-Me-Me-Me-Me-Me-Me, or

Me-Me-Me-Me-Me-Me-F—F—F—F—F-Me-Me-Me-Me-Me-Me-Me-F-Me-Me-ia-ia, or

Me-Me-Me-Me-Me-F—F-Me-F—F—F—F-Me-Me-Me-Me-Me-Me-Me-Me-Me-ia-ia, or

Me-Me-Me-Me-Me-Me-F-Me-F—F—F—F-Me-Me-Me-Me-Me-Me-Me-Me-Me-ia-ia, or

Me-Me-Me-Me-Me-Me-Me-Me-F—F—F-Me-Me-Me-Me-Me-Me-Me-Me-Me-Me-ia-ia, or

Me-Me-Me-Me-Me-Me-F-Me-F—F—F-Me-Me-Me-Me-Me-Me-Me-Me-Me-Me-ia-ia,

wherein ia represents an inverted abasic nucleoside, and when the inverted abasic nucleosides as represented by ia-ia are present at the 3′ terminus of the second strand, said inverted abasic nucleosides are present in a 2 nucleoside overhang.

A nucleic acid wherein modified nucleosides of said second strand comprise a modification pattern according to any one of the following (5′-3′):

ia-ia-Me(s)Me(s)Me-Me-Me-Me-F—F—F—F—F-Me-Me-Me-Me-Me-Me-Me-F-Me-Me, or

ia-ia-Me(s)Me(s)Me-Me-Me-F—F-Me-F—F—F—F-Me-Me-Me-Me-Me-Me-Me-Me-Me, or

ia-ia-Me(s)Me(s)Me-Me-Me-Me-F-Me-F—F—F—F-Me-Me-Me-Me-Me-Me-Me-Me-Me, or

ia-ia-Me(s)Me(s)Me-Me-Me-Me-Me-Me-F—F—F-Me-Me-Me-Me-Me-Me-Me-Me-Me-Me, or

ia-ia-Me(s)Me(s)Me-Me-Me-Me-F-Me-F—F—F-Me-Me-Me-Me-Me-Me-Me-Me-Me-Me, or

Me-Me-Me-Me-Me-Me-F—F—F—F—F-Me-Me-Me-Me-Me-Me-Me-F(s)Me(s)Me-ia-ia, or

Me-Me-Me-Me-Me-F—F-Me-F—F—F—F-Me-Me-Me-Me-Me-Me-Me(s)Me(s)Me-ia-ia, or

Me-Me-Me-Me-Me-Me-F-Me-F—F—F—F-Me-Me-Me-Me-Me-Me-Me(s)Me(s)Me-ia-ia, or

Me-Me-Me-Me-Me-Me-Me-Me-F—F—F-Me-Me-Me-Me-Me-Me-Me-Me(s)Me(s)Me-ia-ia, or

Me-Me-Me-Me-Me-Me-F-Me-F—F—F-Me-Me-Me-Me-Me-Me-Me-Me(s)Me(s)Me-ia-ia,

wherein:

(s) is a phosphorothioate internucleoside linkage, ia represents an inverted abasic nucleoside, and when the inverted abasic nucleosides as represented by ia-ia are present at the 3′ terminus of the second strand, said inverted abasic nucleosides are present in a 2 nucleoside overhang.

A nucleic acid wherein modified nucleosides comprise any one of the following modification patterns:

Modification pattern 1: Second strand (5′-3′): Me-Me-Me-Me-Me-Me-F—F—F—F—F-Me-Me-Me-Me-Me-Me-Me-F-Me-Me, First strand (5′-3′): Me-F-Me-F-Me-F-Me-F— F-Me-Me-Me-Me-F-Me-F-Me-Me-Me-Me-Me-Me-Me

Or Modification pattern 2: Second strand (5′-3′): Me-Me-Me-Me-Me-F—F-Me-F—F—F—F-Me-Me-Me-Me-Me-Me-Me-Me-Me, First strand (5′-3′): Me-F-Me-F-Me—F-Me-F—F-Me-Me-Me-Me-F-Me-F-Me-Me-Me-Me-Me-Me-Me

Or Modification pattern 3: Second strand (5′-3′): Me-Me-Me-Me-Me-Me-F-Me-F—F—F—F-Me-Me-Me-Me-Me-Me-Me-Me-Me, First strand (5′-3′): Me-F-Me-F-Me-F-Me-F— F-Me-Me-Me-Me-F-Me-F-Me-Me-Me-Me-Me-Me-Me

Or Modification pattern 4: Second strand (5′-3′): Me-Me-Me-Me-Me-Me-F-Me-F—F—F—F-Me-Me-Me-Me-Me-Me-Me-Me-Me, First strand (5′-3′): Me-F-Me-F-Me-F-Me-Me-Me-Me-Me-Me-Me-F-Me-F-Me-Me-Me-Me-Me-Me-Me

Or Modification pattern 5: Second strand (5′-3′): Me-Me-Me-Me-Me-Me-Me-Me-F—F—F-Me-Me-Me-Me-Me-Me-Me-Me-Me-Me, First strand (5′-3′): Me-F-Me-Me-Me-F-Me-Me-F-Me-Me-Me-Me-F-Me-F-Me-Me-Me-Me-Me-Me-Me

Or Modification pattern 6: Second strand (5′-3′): Me-Me-Me-Me-Me-Me-F-Me-F—F—F-Me-Me-Me-Me-Me-Me-Me-Me-Me-Me, First strand (5′-3′): Me-F-Me-Me-Me-F-Me-Me-F-Me-Me-Me-Me-F-Me-F-Me-Me-Me-Me-Me-Me-Me.

A nucleic acid wherein modified nucleosides comprise any one of the following modification patterns:

Modification pattern 1: Second strand (5′-3′): Me(s)Me(s)Me-Me-Me-Me-F—F—F—F—F-Me-Me-Me-Me-Me-Me-Me-F-Me-Me, First strand (5′-3′): Me(s)F(s)Me-F-Me—F-Me-F—F-Me-Me-Me-Me-F-Me-F-Me-Me-Me-Me-Me(s)Me(s)Me

Or Modification pattern 2: Second strand (5′-3′): Me(s)Me(s)Me-Me-Me-F—F-Me-F—F—F—F-Me-Me-Me-Me-Me-Me-Me-Me-Me, First strand (5′-3′): Me(s)F(s)Me-F-Me-F-Me-F— F-Me-Me-Me-Me-F-Me-F-Me-Me-Me-Me-Me(s)Me(s)Me

Or Modification pattern 3: Second strand (5′-3′): Me(s)Me(s)Me-Me-Me-Me-F-Me-F—F—F—F-Me-Me-Me-Me-Me-Me-Me-Me-Me, First strand (5′-3′): Me(s)F(s)Me-F-Me-F-Me-F— F-Me-Me-Me-Me-F-Me-F-Me-Me-Me-Me-Me(s)Me(s)Me

Or Modification pattern 4: Second strand (5′-3′): Me(s)Me(s)Me-Me-Me-Me-F-Me-F—F—F— F-Me-Me-Me-Me-Me-Me-Me-Me-Me, First strand (5′-3′): Me(s)F(s)Me-F-Me-F-Me-Me-Me-Me-Me-Me-Me-F-Me-F-Me-Me-Me-Me-Me(s)Me(s)Me

Or Modification pattern 5: Second strand (5′-3′): Me(s)Me(s)Me-Me-Me-Me-Me-Me-F—F—F-Me-Me-Me-Me-Me-Me-Me-Me-Me-Me, First strand (5′-3′): Me(s)F(s)Me-Me-Me-F-Me-Me-F-Me-Me-Me-Me-F-Me-F-Me-Me-Me-Me-Me(s)Me(s)Me

Or Modification pattern 6: Second strand (5′-3′): Me(s)Me(s)Me-Me-Me-Me-F-Me-F—F—F-Me-Me-Me-Me-Me-Me-Me-Me-Me-Me, First strand (5′-3′): Me(s)F(s)Me-Me-Me-F-Me-Me-F-Me-Me-Me-Me-F-Me-F-Me-Me-Me-Me-Me(s)Me(s)Me

wherein (s) is a phosphorothioate internucleoside linkage.

A nucleic acid wherein modified nucleosides comprise any one of the following modification patterns:

Modification pattern 1: Second strand (5′-3′): Me-Me-Me-Me-Me-Me-F—F—F—F—F-Me-Me-Me-Me-Me-Me-Me-F(s)Me(s)Me, First strand (5′-3′): Me(s)F(s)Me-F-Me-F-Me-F— F-Me-Me-Me-Me-F-Me-F-Me-Me-Me-Me-Me(s)Me(s)Me

Or Modification pattern 2: Second strand (5′-3′): Me-Me-Me-Me-Me-F—F-Me-F—F—F—F-Me-Me-Me-Me-Me-Me-Me(s)Me(s)Me, First strand (5′-3′): Me(s)F(s)Me-F-Me-F-Me-F— F-Me-Me-Me-Me-F-Me-F-Me-Me-Me-Me-Me(s)Me(s)Me

Or Modification pattern 3: Second strand (5′-3′): Me-Me-Me-Me-Me-Me-F-Me-F—F—F—F-Me-Me-Me-Me-Me-Me-Me(s)Me(s)Me, First strand (5′-3′): Me(s)F(s)Me-F-Me-F-Me-F— F-Me-Me-Me-Me-F-Me-F-Me-Me-Me-Me-Me(s)Me(s)Me

Or Modification pattern 4: Second strand (5′-3′): Me-Me-Me-Me-Me-Me-F-Me-F—F—F— F-Me-Me-Me-Me-Me-Me-Me(s)Me(s)Me, First strand (5′-3′): Me(s)F(s)Me-F-Me-F-Me-Me-Me-Me-Me-Me-Me-F-Me-F-Me-Me-Me-Me-Me(s)Me(s)Me

Or Modification pattern 5: Second strand (5′-3′): Me-Me-Me-Me-Me-Me-Me-Me-F—F—F-Me-Me-Me-Me-Me-Me-Me-Me(s)Me(s)Me, First strand (5′-3′): Me(s)F(s)Me-Me-Me-F-Me-Me-F-Me-Me-Me-Me-F-Me-F-Me-Me-Me-Me-Me(s)Me(s)Me

Or Modification pattern 6: Second strand (5′-3′): Me-Me-Me-Me-Me-Me-F-Me-F—F—F-Me-Me-Me-Me-Me-Me-Me-Me(s)Me(s)Me, First strand (5′-3′): Me(s)F(s)Me-Me-Me-F-Me-Me-F-Me-Me-Me-Me-F-Me-F-Me-Me-Me-Me-Me(s)Me(s)Me

wherein (s) is a phosphorothioate internucleoside linkage.

A nucleic acid wherein modified nucleosides comprise any one of the following modification patterns:

Modification pattern 1: Second strand (5′-3′): ia-ia-Me-Me-Me-Me-Me-Me-F—F—F—F—F-Me-Me-Me-Me-Me-Me-Me-F-Me-Me, First strand (5′-3′): Me-F-Me-F-Me-F-Me-F—F-Me-Me-Me-Me-F-Me-F-Me-Me-Me-Me-Me-Me-Me

Or Modification pattern 2: Second strand (5′-3′): ia-ia-Me-Me-Me-Me-Me-F—F-Me-F—F—F—F-Me-Me-Me-Me-Me-Me-Me-Me-Me, First strand(5′-3′): Me-F-Me—F-Me-F-Me-F—F-Me-Me-Me-Me-F-Me-F-Me-Me-Me-Me-Me-Me-Me

Or Modification pattern 3: Second strand (5′-3′): ia-ia-Me-Me-Me-Me-Me-Me-F-Me-F—F—F—F-Me-Me-Me-Me-Me-Me-Me-Me-Me, First strand(5′-3′): Me-F-Me—F-Me-F-Me-F—F-Me-Me-Me-Me-F-Me-F-Me-Me-Me-Me-Me-Me-Me

Or Modification pattern 4: Second strand (5′-3′): ia-ia-Me-Me-Me-Me-Me-Me-F-Me-F—F—F—F-Me-Me-Me-Me-Me-Me-Me-Me-Me, First strand (5′-3′): Me-F-Me-F-Me-F-Me-Me-Me-Me-Me-Me-Me-F-Me-F-Me-Me-Me-Me-Me-Me-Me

Or Modification pattern 5: Second strand (5′-3′): ia-ia-Me-Me-Me-Me-Me-Me-Me-Me-F—F-Me-Me-Me-Me-Me-Me-Me-Me-Me-Me, First strand (5′-3′): Me-F-Me-Me-Me-F-Me-Me-F-Me-Me-Me-Me-F-Me-F-Me-Me-Me-Me-Me-Me-Me

Or Modification pattern 6: Second strand (5′-3′): ia-ia-Me-Me-Me-Me-Me-Me-F-Me-F—F—F-Me-Me-Me-Me-Me-Me-Me-Me, First strand (5′-3′): Me-F-Me-Me-Me-F-Me-Me-F-Me-Me-Me-Me-F-Me-F-Me-Me-Me-Me-Me-Me-Me,

wherein ia represents an inverted abasic nucleoside.

A nucleic acid wherein modified nucleosides comprise any one of the following modification patterns:

Modification pattern 1: Second strand (5′-3′): Me-Me-Me-Me-Me-Me-F—F—F—F—F-Me-Me-Me-Me-Me-Me-Me-F-Me-Me-ia-ia, First strand (5′-3′): Me-F-Me-F-Me-F-Me-F— F-Me-Me-Me-Me-F-Me-F-Me-Me-Me-Me-Me-Me-Me

Or Modification pattern 2: Second strand (5′-3′): Me-Me-Me-Me-Me-F—F-Me-F—F—F—F-Me-Me-Me-Me-Me-Me-Me-Me-Me-ia-ia, First strand (5′-3′): Me-F-Me—F-Me-F-Me-F—F-Me-Me-Me-Me-F-Me-F-Me-Me-Me-Me-Me-Me-Me

Or Modification pattern 3: Second strand (5′-3′): Me-Me-Me-Me-Me-Me-F-Me-F—F—F—F-Me-Me-Me-Me-Me-Me-Me-Me-Me-ia-ia, First strand (5′-3′): Me-F-Me-F-Me-F-Me-F— F-Me-Me-Me-Me-F-Me-F-Me-Me-Me-Me-Me-Me-Me

Or Modification pattern 4: Second strand (5′-3′): Me-Me-Me-Me-Me-Me-F-Me-F—F—F—F-Me-Me-Me-Me-Me-Me-Me-Me-Me-ia-ia, First strand (5′-3′): Me-F-Me-F-Me-F-Me-Me-Me-Me-Me-Me-Me-F-Me-F-Me-Me-Me-Me-Me-Me-Me

Or Modification pattern 5: Second strand (5′-3′): Me-Me-Me-Me-Me-Me-Me-Me-F—F—F-Me-Me-Me-Me-Me-Me-Me-Me-Me-Me-ia-ia, First strand (5′-3′): Me-F-Me-Me-Me-F-Me-Me-F-Me-Me-Me-Me-F-Me-F-Me-Me-Me-Me-Me-Me-Me

Or Modification pattern 6: Second strand (5′-3′): Me-Me-Me-Me-Me-Me-F-Me-F—F—F-Me-Me-Me-Me-Me-Me-Me-Me-Me-Me-ia-ia,—First strand (5′-3′): Me-F-Me-Me-Me-F-Me-Me-F-Me-Me-Me-Me-F-Me-F-Me-Me-Me-Me-Me-Me-Me,

wherein ia represents an inverted abasic nucleoside, and when the inverted abasic nucleosides as represented by ia-ia are present at the 3′ terminus of the second strand, said inverted abasic nucleosides are present in a 2 nucleoside overhang.

A nucleic acid wherein modified nucleosides comprise any one of the following modification patterns:

Modification pattern 1: Second strand (5′-3′): ia-ia-Me(s)Me(s)Me-Me-Me-Me-F—F—F—F— F-Me-Me-Me-Me-Me-Me-Me-F-Me-Me, First strand (5′-3′): Me(s)F(s)Me-F-Me-F-Me-F— F-Me-Me-Me-Me-F-Me-F-Me-Me-Me-Me-Me(s)Me(s)Me

Or Modification pattern 2: Second strand (5′-3′): ia-ia-Me(s)Me(s)Me-Me-Me-F—F-Me-F— F—F—F-Me-Me-Me-Me-Me-Me-Me-Me-Me, First strand (5′-3′): Me(s)F(s)Me-F-Me-F-Me-F—F-Me-Me-Me-Me-F-Me-F-Me-Me-Me-Me-Me(s)Me(s)Me

Or Modification pattern 3: Second strand (5′-3′): ia-ia-Me(s)Me(s)Me-Me-Me-Me-F-Me-F— F—F—F-Me-Me-Me-Me-Me-Me-Me-Me-Me, First strand (5′-3′): Me(s)F(s)Me-F-Me-F-Me-F—F-Me-Me-Me-Me-F-Me-F-Me-Me-Me-Me-Me(s)Me(s)Me

Or Modification pattern 4: Second strand (5′-3′): ia-ia-Me(s)Me(s)Me-Me-Me-Me-F-Me-F—F—F—F-Me-Me-Me-Me-Me-Me-Me-Me-Me, First strand (5′-3′): Me(s)F(s)Me-F-Me-F-Me-Me-Me-Me-Me-Me-Me-F-Me-F-Me-Me-Me-Me-Me(s)Me(s)Me

Or Modification pattern 5: Second strand (5′-3′): ia-ia-Me(s)Me(s)Me-Me-Me-Me-Me-Me-F— F—F-Me-Me-Me-Me-Me-Me-Me-Me-Me-Me, First strand (5′-3′): Me(s)F(s)Me-Me-Me-F-Me-Me-F-Me-Me-Me-Me-F-Me-F-Me-Me-Me-Me-Me(s)Me(s)Me

Or Modification pattern 6: Second strand (5′-3′): ia-ia-Me(s)Me(s)Me-Me-Me-Me-F-Me-F— F—F-Me-Me-Me-Me-Me-Me-Me-Me-Me-Me, First strand (5′-3′): Me(s)F(s)Me-Me-Me-F-Me-Me-F-Me-Me-Me-Me-F-Me-F-Me-Me-Me-Me-Me(s)Me(s)Me

wherein:

(s) is a phosphorothioate internucleoside linkage, ia represents an inverted abasic nucleoside.

A nucleic acid wherein modified nucleosides comprise any one of the following modification patterns:

Modification pattern 1: Second strand (5′-3′): Me-Me-Me-Me-Me-Me-F—F—F—F—F-Me-Me-Me-Me-Me-Me-Me-F(s)Me(s)Me-ia-ia, First strand (5′-3′): Me(s)F(s)Me-F-Me-F-Me-F— F-Me-Me-Me-Me-F-Me-F-Me-Me-Me-Me-Me(s)Me(s)Me

Or Modification pattern 2: Second strand (5′-3′): Me-Me-Me-Me-Me-F—F-Me-F—F—F—F-Me-Me-Me-Me-Me-Me-Me(s)Me(s)Me-ia-ia, First strand (5′-3′): Me(s)F(s)Me-F-Me-F-Me-F—F-Me-Me-Me-Me-F-Me-F-Me-Me-Me-Me-Me(s)Me(s)Me

Or Modification pattern 3: Second strand (5′-3′): Me-Me-Me-Me-Me-Me-F-Me-F—F—F—F-Me-Me-Me-Me-Me-Me-Me(s)Me(s)Me-ia-ia, First strand (5′-3′): Me(s)F(s)Me-F-Me-F-Me-F—F-Me-Me-Me-Me-F-Me-F-Me-Me-Me-Me-Me(s)Me(s)Me

Or Modification pattern 4: Second strand (5′-3′): Me-Me-Me-Me-Me-Me-F-Me-F—F—F— F-Me-Me-Me-Me-Me-Me-Me(s)Me(s)Me-ia-ia, First strand (5′-3′): Me(s)F(s)Me-F-Me-F-Me-Me-Me-Me-Me-Me-Me-F-Me-F-Me-Me-Me-Me-Me(s)Me(s)Me

Or Modification pattern 5: Second strand (5′-3′): Me-Me-Me-Me-Me-Me-Me-Me-F—F—F-Me-Me-Me-Me-Me-Me-Me-Me(s)Me(s)Me-ia-ia, First strand (5′-3′): Me(s)F(s)Me-Me-Me-F-Me-Me-F-Me-Me-Me-Me-F-Me-F-Me-Me-Me-Me-Me(s)Me(s)Me

Or Modification pattern 6: Second strand (5′-3′): Me-Me-Me-Me-Me-Me-F-Me-F—F—F-Me-Me-Me-Me-Me-Me-Me-Me(s)Me(s)Me-ia-ia, First strand (5′-3′): Me(s)F(s)Me-Me-Me-F-Me-Me-F-Me-Me-Me-Me-F-Me-F-Me-Me-Me-Me-Me(s)Me(s)Me

wherein: (s) is a phosphorothioate internucleoside linkage, ia represents an inverted abasic nucleoside, and when the inverted abasic nucleosides as represented by ia-ia are present at the 3′ terminus of the second strand, said inverted abasic nucleosides are present in a 2 nucleoside overhang.

A nucleic acid wherein the first strand comprises a 2′ sugar modification pattern wherein said modifications are selected at least from 2′Me and 2′F sugar modifications, provided that the overall number of 2′F sugar modifications in the first strand does not consist of four, or six, 2′F modifications.

A nucleic acid wherein the first strand comprises a 2′ sugar modification pattern wherein said modifications are selected at least from 2′Me and 2′F sugar modifications, wherein the overall number of 2′F sugar modifications in the first strand consists of three, five or seven 2′F modifications.

A nucleic acid wherein the first strand comprises a 2′ sugar modification pattern wherein said modifications are selected at least from 2′Me and 2′F sugar modifications, wherein the overall number of 2′F sugar modifications in the first strand consists of three 2′F modifications. A nucleic acid wherein the first strand comprises a 2′ sugar modification pattern wherein said modifications are selected at least from 2′Me and 2′F sugar modifications, wherein the overall number of 2′F sugar modifications in the first strand consists of five 2′F modifications.

A nucleic acid wherein the first strand comprises a 2′ sugar modification pattern as follows (5′-3′):

Me-F-Me-X₂-Me-F-(Me)₇-(F-Me)₂-X₃-Me-X₄-(Me)₃

wherein X₂, X₃ and X₄ are selected from 2′Me and 2′F sugar modifications, provided that for X₂, X₃ and X₄ at least one is a 2′F sugar modification, and the other two sugar modifications are 2′Me sugar modifications.

A nucleic acid wherein the first strand comprises a 2′ sugar modification pattern as follows (5′-3′):

Me-F-Me-X₂-Me-F-(Me)₇-(F-Me)₂-X₃-Me-X₄-(Me)₃

wherein X₂ is a 2′F sugar modification, and X₃ and X₄ are 2′Me sugar modifications.

A nucleic acid wherein the first strand comprises a 2′ sugar modification pattern as follows (5′-3′):

Me-F-Me-X₂-Me-F-(Me)₇-(F-Me)₂-X₃-Me-X₄-(Me)₃

wherein X₃ is a 2′F sugar modification, and X₂ and X₄ are 2′Me sugar modifications.

A nucleic acid wherein the first strand comprises a 2′ sugar modification pattern as follows (5′-3′):

Me-F-Me-X₂-Me-F-(Me)₇-(F-Me)₂-X₃-Me-X₄-(Me)₃

wherein X₄ is a 2′F sugar modification, and X₂ and X₃ are 2′Me sugar modifications.

A nucleic acid wherein the first strand comprises a 2′ sugar modification pattern wherein said modifications are selected at least from 2′Me and 2′F sugar modifications, wherein the overall number of 2′F sugar modifications in the first strand consists of seven 2′F modifications.

A nucleic acid wherein the first strand comprises a 2′ sugar modification pattern as follows (5′-3′):

Me-F-Me-X₂-Me-F-Me-(F)₂-(Me)₄-(F-Me)₂-X₃-Me-X₄-(Me)₃

wherein X₂, X₃ and X₄ are selected from 2′Me and 2′F sugar modifications, provided that for X₂, X₃ and X₄ at least one is a 2′F sugar modification, and the other two sugar modifications are 2′Me sugar modifications.

A nucleic acid wherein the first strand comprises a 2′ sugar modification pattern as follows (5′-3′):

Me-F-Me-X₂-Me-F-Me-(F)₂-(Me)₄-(F-Me)₂-X₃-Me-X₄-(Me)₃

wherein X₂ is a 2′F sugar modification, and X₃ and X₄ are 2′Me sugar modifications.

A nucleic acid wherein the first strand comprises a 2′ sugar modification pattern as follows (5′-3′):

Me-F-Me-X₂-Me-F-Me-(F)₂-(Me)₄-(F-Me)₂-X₃-Me-X₄-(Me)₃

wherein X₃ is a 2′F sugar modification, and X₂ and X₄ are 2′Me sugar modifications.

A nucleic acid wherein the first strand comprises a 2′ sugar modification pattern as follows (5′-3′):

Me-F-Me-X₂-Me-F-Me-(F)₂-(Me)₄-(F-Me)₂-X₃-Me-X₄-(Me)₃

wherein X₄ is a 2′F sugar modification, and X₂ and X₃ are 2′Me sugar modifications.

A nucleic acid wherein the first strand comprises a 2′ sugar modification pattern as follows (5′-3′):

Me-F-(Me)₃-X₁-(Me)₇-F-Me-F-(Me)₇

wherein X₁ is a thermally destabilising modification.

A nucleic acid wherein the first strand comprises a 2′ sugar modification pattern as follows (5′-3′):

Me-F-(Me)₃-X₁-Me-(F)₂-(Me)₄-F-Me-F-(Me)₇

wherein X₁ is a thermally destabilising modification.

A nucleic acid wherein the second strand comprises a 2′ sugar modification pattern as follows (5′-3′):

(Me)₈-(F)₃-(Me)₁₀.

A nucleic acid wherein the second strand comprises a 2′ sugar modification pattern as follows (5′-3′):

(Me)₈-(F)₃-(Me)₁₀, and

wherein the first strand comprises a 2′ sugar modification pattern wherein said modifications are selected at least from 2′Me and 2′F sugar modifications, provided that the overall number of 2′F sugar modifications in the first strand does not consist of four, or six, 2′F modifications.

A nucleic acid wherein the second strand comprises a 2′ sugar modification pattern as follows (5′-3′):

(Me)₈-(F)₃-(Me)₁₀, and

wherein the first strand comprises a 2′ sugar modification pattern wherein said modifications are selected at least from 2′Me and 2′F sugar modifications, wherein the overall number of 2′F sugar modifications in the first strand consists of three, five or seven 2′F modifications.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):

(Me)₈-(F)₃-(Me)₁₀, and

wherein nucleosides of said first strand comprise a 2′ sugar modification pattern as follows (5′-3′):

Me-F-(Me)₃-X₁-(Me)₇-F-Me-F-(Me)₇, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):

(Me)₈-(F)₃-(Me)₁₀, and

wherein nucleosides of said first strand comprise a 2′ sugar modification pattern as follows (5′-3′):

(Me-F)₃-(Me)₇-F-Me-F-(Me)₇.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):

(Me)₈-(F)₃-(Me)₁₀, and

wherein nucleosides of said first strand comprise a 2′ sugar modification pattern as follows (5′-3′):

Me-F-(Me)₃-F-(Me)₇-(F-Me)₂-F-(Me)₅.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):

(Me)₈-(F)₃-(Me)₁₀, and wherein nucleosides of said first strand comprise a 2′ sugar modification pattern as follows (5′-3′):

Me-F-(Me)₃-F-(Me)₇-F-Me-F-(Me)₃-F-(Me)₃.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):

(Me)₈-(F)₃-(Me)₁₀, and

wherein nucleosides of said first strand comprise a 2′ sugar modification pattern as follows (5′-3′):

Me-F-(Me)₃-X₁-Me-(F)₂-(Me)₄-F-Me-F-(Me)₇, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):

(Me)₈-(F)₃-(Me)₁₀, and

wherein nucleosides of said first strand comprise a 2′ sugar modification pattern as follows (5′-3′):

(Me-F)₃-Me-(F)₂-(Me)₄-(F-Me)₂-(Me)₆.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):

(Me)₈-(F)₃-(Me)₁₀, and

wherein nucleosides of said first strand comprise a 2′ sugar modification pattern as follows (5′-3′):

Me-F-(Me)₃-F-Me-(F)₂-(Me)₄-(F-Me)₂-F-(Me)₅.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):

(Me)₈-(F)₃-(Me)₁₀, and

wherein nucleosides of said first strand comprise a 2′ sugar modification pattern as follows (5′-3′):

Me-F-(Me)₃-F-Me-(F)₂-(Me)₄-(F-Me)₂-(Me)₂-F-(Me)₃.

A nucleic acid wherein the second strand comprises a 2′ sugar, and abasic modification pattern as follows (5′-3′):

ia-ia-(Me)₈-(F)₃-(Me)₁₀

wherein ia represents an inverted abasic nucleoside.

A nucleic acid wherein the second strand comprises a 2′ sugar, and abasic modification pattern as follows (5′-3′):

ia-ia-(Me)₈-(F)₃-(Me)₁₀, wherein ia represents an inverted abasic nucleoside; and

wherein the first strand comprises a 2′ sugar modification pattern wherein said modifications are selected at least from 2′Me and 2′F sugar modifications, provided that the overall number of 2′F sugar modifications in the first strand does not consist of four, or six, 2′F modifications.

A nucleic acid wherein the second strand comprises a 2′ sugar, and abasic modification pattern as follows (5′-3′):

ia-ia-(Me)₈-(F)₃-(Me)₁₀, wherein ia represents an inverted abasic nucleoside; and

wherein the first strand comprises a 2′ sugar modification pattern wherein said modifications are selected at least from 2′Me and 2′F sugar modifications, wherein the overall number of 2′F sugar modifications in the first strand consists of three, five or seven 2′F modifications.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-(Me)₈-(F)₃-(Me)₁₀, wherein ia represents an inverted abasic nucleoside; and

wherein nucleosides of said first strand comprise a 2′ sugar modification pattern as follows (5′-3′):

Me-F-(Me)₃-X₁-(Me)₇-F-Me-F-(Me)₇, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-(Me)₈-(F)₃-(Me)₁₀, wherein ia represents an inverted abasic nucleoside; and

wherein nucleosides of said first strand comprise a 2′ sugar modification pattern as follows (5′-3′):

(Me-F)₃-(Me)₇-F-Me-F-(Me)₇.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-(Me)₈-(F)₃-(Me)₁₀, wherein ia represents an inverted abasic nucleoside; and

wherein nucleosides of said first strand comprise a 2′ sugar modification pattern as follows (5′-3′):

Me-F-(Me)₃-F-(Me)₇-(F-Me)₂-F-(Me)₅.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-(Me)₈-(F)₃-(Me)₁₀, wherein ia represents an inverted abasic nucleoside; and wherein nucleosides of said first strand comprise a 2′ sugar modification pattern as follows (5′-3′):

Me-F-(Me)₃-F-(Me)₇-F-Me-F-(Me)₃-F-(Me)₃.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-(Me)₈-(F)₃-(Me)₁₀, wherein ia represents an inverted abasic nucleoside; and wherein nucleosides of said first strand comprise a 2′ sugar modification pattern as follows (5′-3′):

Me-F-(Me)₃-X₁-Me-(F)₂-(Me)₄-F-Me-F-(Me)₇,

wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-(Me)₈-(F)₃-(Me)₁₀, wherein ia represents an inverted abasic nucleoside; and

wherein nucleosides of said first strand comprise a 2′ sugar modification pattern as follows (5′-3′):

(Me-F)₃-Me-(F)₂-(Me)₄-(F-Me)₂-(Me)₆.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-(Me)₈-(F)₃-(Me)₁₀, wherein ia represents an inverted abasic nucleoside; and

wherein nucleosides of said first strand comprise a 2′ sugar modification pattern as follows (5′-3′):

Me-F-(Me)₃-F-Me-(F)₂-(Me)₄-(F-Me)₂-F-(Me)₅.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-(Me)₈-(F)₃-(Me)₁₀, wherein ia represents an inverted abasic nucleoside; and

wherein nucleosides of said first strand comprise a 2′ sugar modification pattern as follows (5′-3′):

Me-F-(Me)₃-F-Me-(F)₂-(Me)₄-(F-Me)₂-(Me)₂-F-(Me)₃.

A nucleic acid wherein the second strand comprises a 2′ sugar modification pattern as follows (5′-3′):

ia-ia-Me(s)Me(s) (Me)₆-(F)₃-(Me)₁₀,

wherein ia represents an inverted abasic nucleoside, and (s) represents a phosphorothioate linkage.

A nucleic acid wherein the second strand comprises a 2′ sugar modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage; and

wherein the first strand comprises a 2′ sugar modification pattern wherein said modifications are selected at least from 2′Me and 2′F sugar modifications, provided that the overall number of 2′F sugar modifications in the first strand does not consist of four, or six, 2′F modifications.

A nucleic acid wherein the second strand comprises a 2′ sugar modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage; and

wherein the first strand comprises a 2′ sugar modification pattern wherein said modifications are selected at least from 2′Me and 2′F sugar modifications, wherein the overall number of 2′F sugar modifications in the first strand consists of three, five or seven 2′F modifications.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage, and

wherein nucleosides of said first strand comprise a 2′ sugar modification pattern as follows (5′-3′):

Me(s)F(s)(Me)₃-X₁-(Me)₇-F-Me-F-(Me)₅(s)Me(s)Me, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage, and

wherein nucleosides of said first strand comprise a 2′ sugar modification pattern as follows (5′-3′):

Me(s)F(s)Me-F-Me-F-(Me)₇-F-Me-F-(Me)₅(s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage, and

wherein nucleosides of said first strand comprise a 2′ sugar modification pattern as follows (5′-3′):

Me(s)F(s)(Me)₃-F-(Me)₇-(F-Me)₂-F-(Me)₃(s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage, and

wherein nucleosides of said first strand comprise a 2′ sugar modification pattern as follows (5′-3′):

Me(s)F(s)(Me)₃-F-(Me)₇-F-Me-F-(Me)₃-F-Me(s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage, and

wherein nucleosides of said first strand comprise a 2′ sugar modification pattern as follows (5′-3′):

Me(s)F(s)(Me)₃-X₁-Me-(F)₂-(Me)₄-F-Me-F-(Me)₅(s)Me(s)Me, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage, and

wherein nucleosides of said first strand comprise a 2′ sugar modification pattern as follows (5′-3′):

Me(s)F(s)Me-F-Me-F-Me-(F)₂-(Me)₄-(F-Me)₂-(Me)₄(s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage, and

wherein nucleosides of said first strand comprise a 2′ sugar modification pattern as follows (5′-3′):

Me(s)F(s)(Me)₃-F-Me-(F)₂-(Me)₄-(F-Me)₂-F-(Me)₃(s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):

ia-ia-Me(s)Me(s) (Me)₆-(F)₃-(Me)₁₀, wherein ia represents an inverted abasic nucleoside, and (s) represents a phosphorothioate linkage, and

wherein nucleosides of said first strand comprise a 2′ sugar modification pattern as follows (5′-3′):

Me(s)F(s)(Me)₃-F-Me-(F)₂-(Me)₄-(F-Me)₂-(Me)₂-F-Me(s)Me(s)Me.

Preferred modifications are as follows:

Modification pattern 1:

Second strand (5′-3′): ia-ia-Me-Me-Me-Me-Me-Me-Me-Me-F—F—F-Me-Me-Me-Me-Me-Me-Me-Me-Me-Me,

First strand (5′-3′): Me-F-Me-Me-Me-X₁-Me-Me-Me-Me-Me-Me-Me-F-Me-F-Me-Me-Me-Me-Me-Me-Me, wherein X₁ is a thermally destabilising modification;

Or Modification pattern 2:

Second strand (5′-3′): ia-ia-Me-Me-Me-Me-Me-Me-Me-Me-F—F—F-Me-Me-Me-Me-Me-Me-Me-Me-Me-Me,

First strand (5′-3′): Me-F-Me-F-Me-F-Me-Me-Me-Me-Me-Me-Me-F-Me—F-Me-Me-Me-Me-Me-Me-Me;

Or Modification pattern 3:

Second strand (5′-3′): ia-ia-Me-Me-Me-Me-Me-Me-Me-Me-F—F—F-Me-Me-Me-Me-Me-Me-Me-Me-Me-Me,

First strand (5′-3′): Me-F-Me-Me-Me-F-Me-Me-Me-Me-Me-Me-Me-F-Me-F-Me-F-Me-Me-Me-Me-Me;

Or Modification pattern 4:

Second strand (5′-3′): ia-ia-Me-Me-Me-Me-Me-Me-Me-Me-F—F—F-Me-Me-Me-Me-Me-Me-Me-Me-Me-Me,

First strand (5′-3′): Me-F-Me-Me-Me-F-Me-Me-Me-Me-Me-Me-Me-F-Me-F-Me-Me-Me-F-Me-Me-Me;

Or Modification pattern 5:

Second strand (5′-3′): ia-ia-Me-Me-Me-Me-Me-Me-Me-Me-F—F—F-Me-Me-Me-Me-Me-Me-Me-Me-Me-Me,

First strand (5′-3′): Me-F-Me-Me-Me-X₁-Me-F—F-Me-Me-Me-Me-F-Me—F-Me-Me-Me-Me-Me-Me-Me, wherein X₁ is a thermally destabilising modification;

Or Modification pattern 6:

Second strand (5′-3′): ia-ia-Me-Me-Me-Me-Me-Me-Me-Me-F—F—F-Me-Me-Me-Me-Me-Me-Me-Me-Me-Me,

First strand (5′-3′): Me-F-Me-F-Me-F-Me-F—F-Me-Me-Me-Me-F-Me-F-Me-Me-Me-Me-Me-Me-Me;

Or Modification pattern 7:

Second strand (5′-3′): ia-ia-Me-Me-Me-Me-Me-Me-Me-Me-F—F—F-Me-Me-Me-Me-Me-Me-Me-Me-Me-Me,

First strand (5′-3′): Me-F-Me-Me-Me-F-Me-F—F-Me-Me-Me-Me-F-Me—F-Me-F-Me-Me-Me-Me-Me;

Or Modification pattern 8:

Second strand (5′-3′): ia-ia-Me-Me-Me-Me-Me-Me-Me-Me-F—F—F-Me-Me-Me-Me-Me-Me-Me-Me-Me-Me,

First strand (5′-3′): Me-F-Me-Me-Me-F-Me-F—F-Me-Me-Me-Me-F-Me—F-Me-Me-Me-F-Me-Me-Me.

Particularly preferred modifications are as follows:

Modification pattern 1:

Second strand (5′-3′): ia-ia-Me(s)Me(s)Me-Me-Me-Me-Me-Me-F—F—F-Me-Me-Me-Me-Me-Me-Me-Me-Me-Me,

First strand (5′-3′): Me(s)F(s)Me-Me-Me-X₁-Me-Me-Me-Me-Me-Me-Me-F-Me-F-Me-Me-Me-Me-Me(s)Me(s)Me, wherein X₁ is a thermally destabilising modification;

Or Modification pattern 2:

Second strand (5′-3′): ia-ia-Me(s)Me(s)Me-Me-Me-Me-Me-Me-F—F—F-Me-Me-Me-Me-Me-Me-Me-Me-Me-Me,

First strand (5′-3′): Me(s)F(s)Me-F-Me-F-Me-Me-Me-Me-Me-Me-Me-F-Me-F-Me-Me-Me-Me-Me(s)Me(s)Me;

Or Modification pattern 3:

Second strand (5′-3′): ia-ia-Me(s)Me(s)Me-Me-Me-Me-Me-Me-F—F—F-Me-Me-Me-Me-Me-Me-Me-Me-Me-Me,

First strand (5′-3′): Me(s)F(s)Me-Me-Me-F-Me-Me-Me-Me-Me-Me-Me-F-Me-F-Me-F-Me-Me-Me(s)Me(s)Me;

Or Modification pattern 4:

Second strand (5′-3′): ia-ia-Me(s)Me(s)Me-Me-Me-Me-Me-Me-F—F—F-Me-Me-Me-Me-Me-Me-Me-Me-Me-Me,

First strand (5′-3′): Me(s)F(s)Me-Me-Me-F-Me-Me-Me-Me-Me-Me-Me-F-Me-F-Me-Me-Me-F-Me(s)Me(s)Me;

Or Modification pattern 5:

Second strand (5′-3′): ia-ia-Me(s)Me(s)Me-Me-Me-Me-Me-Me-F—F—F-Me-Me-Me-Me-Me-Me-Me-Me-Me-Me,

First strand (5′-3′): Me(s)F(s)Me-Me-Me-X₁-Me-F—F-Me-Me-Me-Me-F-Me—F-Me-Me-Me-Me-Me(s)Me(s)Me, wherein X₁ is a thermally destabilising modification;

Or Modification pattern 6:

Second strand (5′-3′): ia-ia-Me(s)Me(s)Me-Me-Me-Me-Me-Me-F—F—F-Me-Me-Me-Me-Me-Me-Me-Me-Me-Me,

First strand (5′-3′): Me(s)F(s)Me-F-Me-F-Me-F—F-Me-Me-Me-Me-F-Me-F-Me-Me-Me-Me-Me(s)Me(s)Me;

Or Modification pattern 7:

Second strand (5′-3′): ia-ia-Me(s)Me(s)Me-Me-Me-Me-Me-Me-F—F—F-Me-Me-Me-Me-Me-Me-Me-Me-Me-Me,

First strand (5′-3′): Me(s)F(s)Me-Me-Me-F-Me-F—F-Me-Me-Me-Me-F-Me—F-Me-F-Me-Me-Me(s)Me(s)Me;

Or Modification pattern 8:

Second strand (5′-3′): ia-ia-Me(s)Me(s)Me-Me-Me-Me-Me-Me-F—F—F-Me-Me-Me-Me-Me-Me-Me-Me-Me-Me,

First strand (5′-3′): Me(s)F(s)Me-Me-Me-F-Me-F—F-Me-Me-Me-Me-F-Me—F-Me-Me-Me-F-Me(s)Me(s)Me;

wherein (s) is a phosphorothioate internucleoside linkage.

### Conjugation of Nucleic Acid to Ligand

Another modification of a nucleic acid e.g. RNA e.g. an siRNA of the invention involves linking the nucleic acid e.g. the siRNA to one or more ligand moieties e.g. to enhance the activity, cellular distribution, or cellular uptake of the nucleic acid e.g. siRNA e.g., into a cell.

In some embodiments, the ligand moiety described can be attached to a nucleic acid e.g. an siRNA oligonucleoside, via a linker that can be cleavable or non-cleavable. The term “linker” or “linking group” means an organic moiety that connects two parts of a compound, e.g., covalently attaches two parts of a compound.

The ligand can be attached to the 3′ or 5′ end of the sense strand.

The ligand is preferably conjugated to 3′ end of the sense strand of the nucleic acid e.g. an siRNA agent.

The invention therefore relates in a further aspect to a conjugate for inhibiting expression of a target e.g. a target gene, in a cell, said conjugate comprising a nucleic acid portion and one or more ligand moieties, said nucleic acid portion comprising a nucleic acid as disclosed herein.

In one aspect the second strand of the nucleic acid is conjugated directly or indirectly (e.g. via a linker) to the one or more ligand moiety(s), wherein said ligand moiety is typically present at a terminal region of the second strand, preferably at the 3′ terminal region thereof.

In certain embodiments, the ligand moiety comprises a GalNAc or GalNAc derivative attached to the nucleic acid e.g. dsiRNA through a linker.

Therefore, the invention relates to a conjugate wherein the ligand moiety comprises
i) one or more GalNAc ligands; and/or
ii) one or more GalNAc ligand derivatives; and/or
iii) one or more GalNAc ligands conjugated to said nucleic acid through a linker.

Said GalNAc ligand may be conjugated directly or indirectly to the 5′ or 3′ terminal region of the second strand of the nucleic acid, preferably at the 3′ terminal region thereof.

GalNAc ligands are well known in the art and described in, inter alia, EP3775207A1.

In some embodiments, the ligand moiety comprises one or more ligands.

In some embodiments, the ligand moiety comprises one or more carbohydrate ligands.

In some embodiments, the one or more carbohydrates can be a monosaccharide, disaccharide, trisaccharide, tetrasaccharide, oligosaccharide and/or polysaccharide.

In some embodiments, the one or more carbohydrates comprise one or more galactose moieties, one or more lactose moieties, one or more N-AcetylGalactosamine moieties, and/or one or more mannose moieties.

In some embodiments, the one or more carbohydrates comprise one or more N-Acetyl-Galactosamine moieties.

In some embodiments, the compounds as described anywhere herein comprise two or three N-AcetylGalactosamine moieties.

In some embodiments, the one or more ligands are attached in a linear configuration, or in a branched configuration, for example each configuration being respectively attached to a branch point in an overall linker.

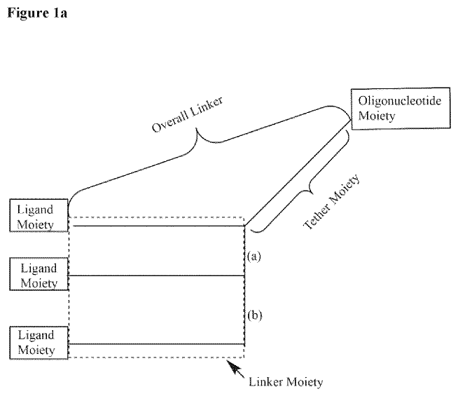

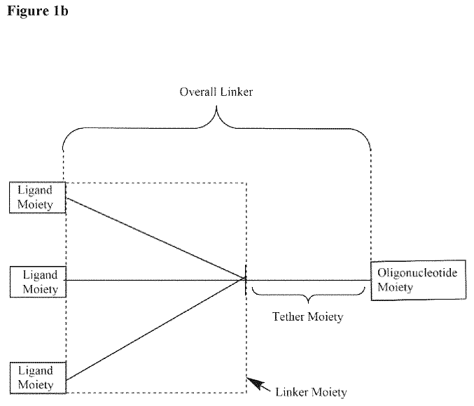

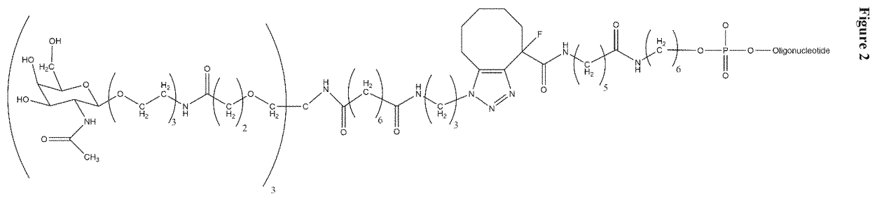

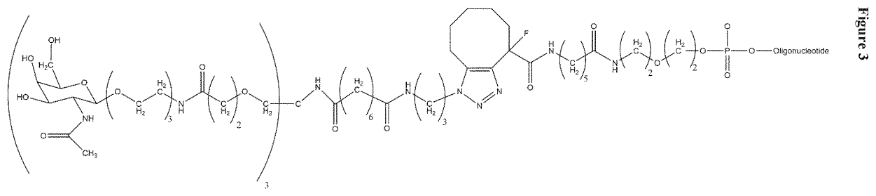

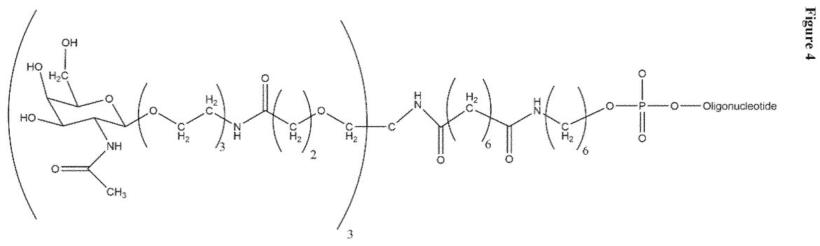

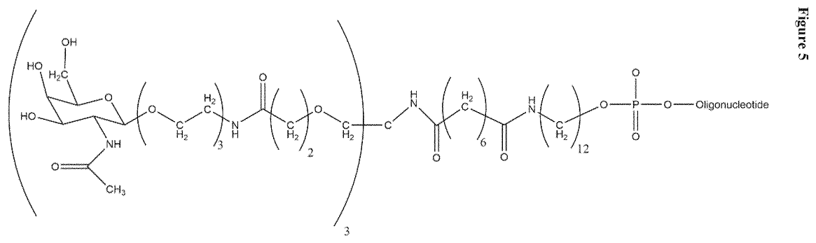

Exemplary linear configurations and Exemplary branched configurations are shown in FIGS. 1a and 1b:

In FIG. 1a, (linear), (a) and/or (b) can typically represent connecting bonds or groups, such as phosphate or phosphorothioate groups.

In FIG. 1b, (branched), in some embodiments, the one or more ligands are attached as a biantennary or triantennary branched configuration. Typically, a triantennary branched configuration can be preferred, such as an N-AcetylGalactosamine triantennary branched configuration.

### Linker

Exemplary compounds of the invention comprise a ‘linker moiety’, such as that as depicted in Formula (I), that is part of an overall ‘linker’.

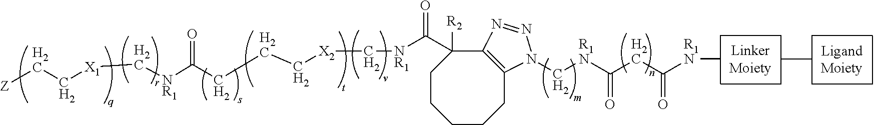

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.

As will be further understood in the art, exemplary compounds of the invention comprise an overall linker that is located between the oligonucleoside moiety and the ligand moiety of these compounds. The overall linker, thereby ‘links’ the oligonucleoside moiety and the ligand moiety to each other.

The overall linker is often notionally envisaged as comprising one or more linker building blocks. For example, there is a linker portion that is depicted as the ‘linker moiety’ as represented in Formula (I) positioned adjacent the ligand moiety and attaching the ligand moiety, typically via a branch point, directly or indirectly to the oligonucleoside moiety. The linker moiety as depicted in Formula (I) can also often be referred to as the ‘ligand arm or arms’ of the overall linker. There can also, but not always, be a further linker portion between the oligonucleoside moiety and the branch point, that is often referred to as the ‘tether moiety’ of the overall linker, ‘tethering’ the oligonucleoside moiety to the remainder of the conjugated compound. Such ‘ligand arms’ and/or ‘linker moieties’ and/or ‘tether moieties’ can be envisaged by reference to the linear and/or branched configurations as set out above.

As can be seen from the claims, and the reminder of the patent specification, the scope of the present invention extends to linear or branched configurations, and with no limitation as to the number of individual ligands that might be present. Furthermore, the addressee will also be aware that there are many structures that could be used as the linker moiety, based on the state of the art and the expertise of an oligonucleoside chemist.

The remainder of the overall linker (other than the linker moiety) as set out in the claims, and the remainder of the patent specification, is shown by its chemical constituents in Formula (I), which the inventors consider to be particularly unique to the current invention. In more general terms, however, these chemical constituents could be described as a ‘tether moiety’ as hereinbefore described, wherein the ‘tether moiety’ is that portion of the overall linker which comprises the group of atoms between Z, namely the oligonucleoside moiety, and the linker moiety as depicted in Formula (I).

### Tether Moiety of Formula I

In relation to Formula (I), the ‘tether moiety’ comprises the group of atoms between Z, namely the oligonucleoside moiety, and the linker moiety.

In some embodiments, R₁ is hydrogen at each occurrence. In some embodiments, R₁ is methyl.

In some embodiments, R₁ is ethyl.

In some embodiments, R₂ is hydroxy. In some embodiments, R₂ is halo. In some embodiments, R₂ is fluoro. In some embodiments, R₂ is chloro. In some embodiments, R₂ is bromo. In some embodiments, R₂ is iodo. In some embodiments, R₂ is nitro.

In some embodiments, X₁ is methylene. In some embodiments, X₁ is oxygen. In some embodiments, X₁ is sulfur.

In some embodiments, X₂ is methylene. In some embodiments, X₂ is oxygen. In some embodiments, X₂ is sulfur.

In some embodiments, m=3.

In some embodiments, n=6.

In some embodiments, X₁ is oxygen and X₂ is methylene. In some embodiments, both X₁ and X₂ are methylene.

In some embodiments, q=1, r=2, s=1, t=1, v=1. In some embodiments, q=1, r=3, s=1, t=1, v=1.

In some embodiments, R₁ is hydrogen at each occurrence, n=6, m=3, R₂ is fluoro, X₂ is methylene, v=1, t=1, s=1, X₁ is methylene, q=1 and r=2.

Thus, in some embodiments, exemplary compounds of the invention comprise the following structure:

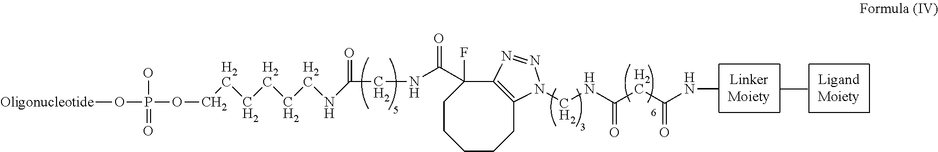

In some embodiments, R₁ is hydrogen at each occurrence, n=6, m=3, R₂ is fluoro, X₂ is methylene, y=1, t=1, s=1, X₁ is oxygen, q=1 and r=2.

Thus, in some embodiments, exemplary compounds of the invention comprise the following structure:

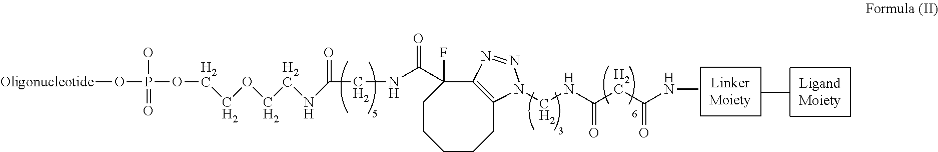

### Alternative Tether Moieties

During the synthesis of compounds of the present invention, alternative tether moiety structures may arise. In some embodiments, alternative tether moieties have a change of one or more atoms in the tether moiety of the overall linker compared to tether moieties described anywhere herein.

In some embodiments, the alternative tether moiety is a compound of Formula (I) as described anywhere herein, wherein R₂ is hydroxy.

In some embodiments, R₁ is hydrogen at each occurrence, n=6, m=3, R₂ is hydroxy, X₂ is methylene, v=1, t=1, s=1, X₁ is methylene, q=1 and r=2.

Thus, in some embodiments, compounds of the invention comprise the following structure:

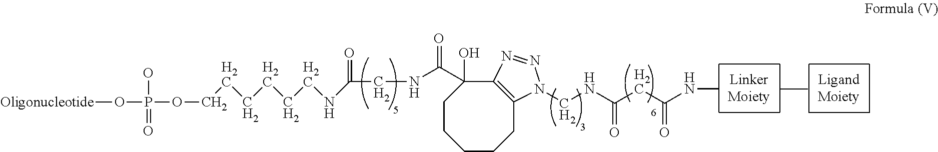

In some embodiments, R₁ is hydrogen at each occurrence, n=6, m=3, R₂ is hydroxy, X₂ is methylene, v=1, t=1, s=1, X₁ is oxygen, q=1 and r=2.

Thus, in some embodiments, compounds of the invention comprise the following structure:

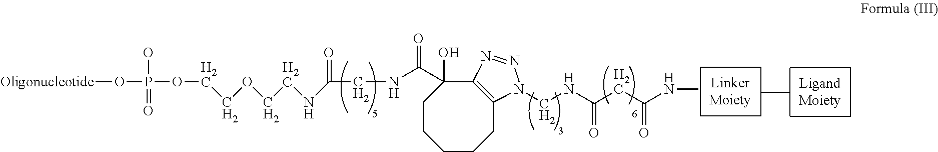

### Linker Moiety

In relation to Formula (I), the ‘linker moiety’ as depicted in Formula (I) comprises the group of atoms located between the tether moiety as described anywhere herein, and the ligand moiety as described anywhere herein.

In some embodiments:

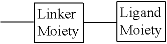

as depicted in Formula (I) as described anywhere herein is any of Formulae (VIa), (VIb) or (VIc), preferably Formula (VIa):

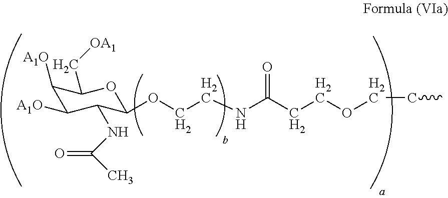

wherein:
A₁ is hydrogen, or a suitable hydroxy protecting group;
a is an integer of 2 or 3; and
b is an integer of 2 to 5; or

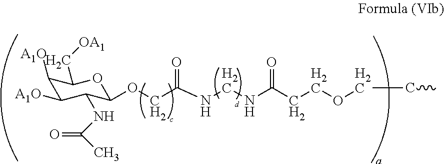

wherein:
A₁ is hydrogen, or a suitable hydroxy protecting group;
a is an integer of 2 or 3; and
c and d are independently integers of 1 to 6; or

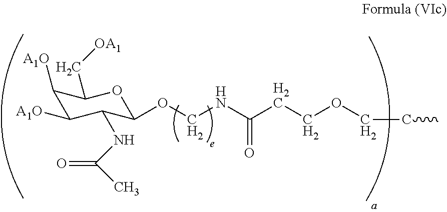

wherein:
A₁ is hydrogen, or a suitable hydroxy protecting group;
a is an integer of 2 or 3; and
e is an integer of 2 to 10.

In some embodiments, the moiety:

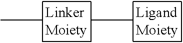

as depicted in Formula (I) is Formula (VIa):

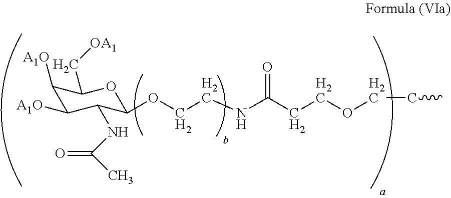

wherein:
A₁ is hydrogen, or a suitable hydroxy protecting group;
a is 3; and
b is an integer of 3.

In some embodiments, the moiety:

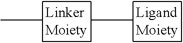

as depicted in Formula (I) as described anywhere herein is Formula (VII):

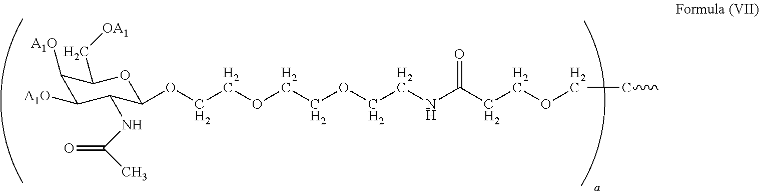

wherein:
A₁ is hydrogen;
a is an integer of 2 or 3, preferably 3.

Other exemplary compounds of the invention comprise a ‘linker moiety’, as depicted in Formula (I*), that is part of an overall ‘linker’.

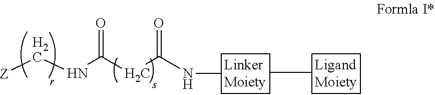

Where:
r and s are independently an integer selected from 1 to 16; and
Z is an oligonucleoside moiety.

As will be further understood in the art, exemplary compounds of the invention comprise an overall linker that is located between the oligonucleoside moiety and the ligand moiety of these compounds. The overall linker, thereby ‘links’ the oligonucleoside moiety and the ligand moiety to each other.

The overall linker is often notionally envisaged as comprising one or more linker building blocks. For example, there is a linker portion that is depicted as the ‘linker moiety’ as represented in Formula (I*) positioned adjacent the ligand moiety and attaching the ligand moiety, typically via a branch point, directly or indirectly to the oligonucleoside moiety. The linker moiety as depicted in Formula (I*) can also often be referred to as the ‘ligand arm or arms’ of the overall linker. There can also, but not always, be a further linker portion between the oligonucleoside moiety and the branch point, that is often referred to as the ‘tether moiety’ of the overall linker, ‘tethering’ the oligonucleoside moiety to the remainder of the conjugated compound. Such ‘ligand arms’ and/or ‘linker moieties’ and/or ‘tether moieties’ can be envisaged by reference to the linear and/or branched configurations as set out above.

As can be seen from the claims, and the reminder of the patent specification, the scope of the present invention extends to linear or branched configurations, and with no limitation as to the number of individual ligands that might be present. Furthermore, the addressee will also be aware that there are many structures that could be used as the linker moiety, based on the state of the art and the expertise of an oligonucleoside chemist.

The remainder of the overall linker (other than the linker moiety) as set out in the claims, and the remainder of the patent specification, is shown by its chemical constituents in Formula (I), which the inventors consider to be particularly unique to the current invention. In more general terms, however, these chemical constituents could be described as a ‘tether moiety’ as hereinbefore described, wherein the ‘tether moiety’ is that portion of the overall linker which comprises the group of atoms between Z, namely the oligonucleoside moiety, and the linker moiety as depicted in Formula (I).

### Tether Moiety

In relation to Formula (I*), the ‘tether moiety’ comprises the group of atoms between Z, namely the oligonucleoside moiety, and the linker moiety.

In some embodiments, s is an integer selected from 4 to 12. In some embodiments, s is 6.

In some embodiments, r is an integer selected from 4 to 14. In some embodiments, r is 6. In some embodiments, r is 12.

In some embodiments, r is 12 and s is 6.

Thus, in some embodiments, exemplary compounds of the invention comprise the following structure:

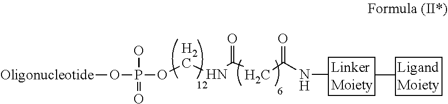

In some embodiments, r is 6 and s is 6.

Thus, in some embodiments, exemplary compounds of the invention comprise the following structure:

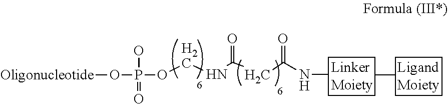

### Linker Moiety

In relation to Formula (I*), the ‘linker moiety’ as depicted in Formula (I) comprises the group of atoms located between the tether moiety as described anywhere herein, and the ligand moiety as described anywhere herein.

In some embodiments, the moiety:

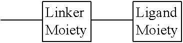

as depicted in Formula (I*) as described anywhere herein is any of Formulae (IV*), (V*) or (VI*), preferably Formula (IV*):

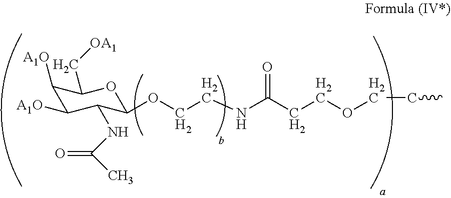

wherein:
A₁ is hydrogen, or a suitable hydroxy protecting group;
a is an integer of 2 or 3; and
b is an integer of 2 to 5; or

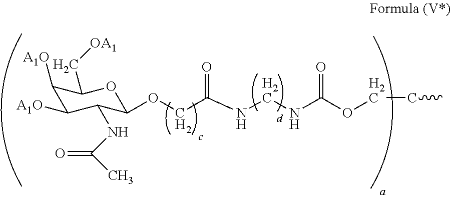

wherein:
A₁ is hydrogen, or a suitable hydroxy protecting group;
a is an integer of 2 or 3; and
c and d are independently integers of 1 to 6; or

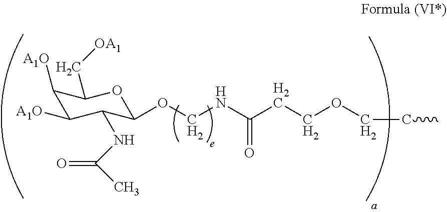

wherein:
A₁ is hydrogen, or a suitable hydroxy protecting group;
a is an integer of 2 or 3; and
e is an integer of 2 to 10.

In some embodiments, the moiety:

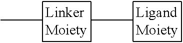

as depicted in Formula (I) is Formula (VIa*):

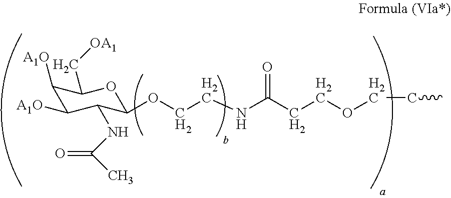

wherein:
A₁ is hydrogen, or a suitable hydroxy protecting group;
a is 3; and
b is an integer of 3.

In some embodiments, the moiety:

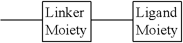

as depicted in Formula (I) as described anywhere herein is Formula (VII*):

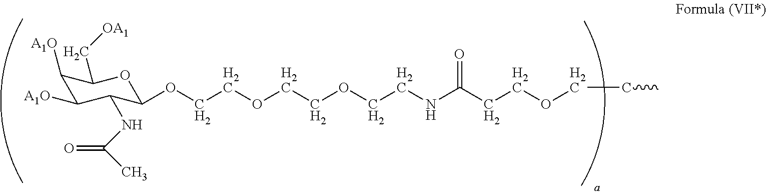

wherein:
A₁ is hydrogen;
a is an integer of 2 or 3.

In some embodiments, a=2. In some embodiments, a=3. In some embodiments, b=3.

### Vector and Cell

In one aspect, the invention provides a cell containing a nucleic acid, such as inhibitory RNA [RNAi] as described herein.

In one aspect, the invention provides a cell comprising a vector as described herein.

In one aspect the invention provides a vector comprising an oligonucleotide inhibitor, e.g. an iRNA e.g. siRNA.

### Pharmaceutically Acceptable Compositions

In one aspect, the invention provides a pharmaceutical composition for inhibiting expression of a target gene, the composition comprising an inhibitor such as an oligomer such as a nucleic acid as disclosed herein.

The pharmaceutically acceptable composition may comprise an excipient and or carrier.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or poly anhydrides; (22) bulking agents, such as polypeptides and amino acids (23) serum component, such as serum albumin, HDL and LDL; and (22) other non-toxic compatible substances employed in pharmaceutical formulations.

Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable excipients include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

In one embodiment, the nucleic acid or composition is administered in an unbuffered solution. In certain embodiments, the unbuffered solution is saline or water. In other embodiments, the nucleic acid e.g. RNAi agent is administered in a buffered solution. In such embodiments, the buffer solution can comprise acetate, citrate, prolamine, carbonate, or phosphate, or any combination thereof. For example, the buffer solution can be phosphate buffered saline (PBS).

### Dosages

The pharmaceutical compositions of the invention may be administered in dosages sufficient to inhibit expression of a gene or modify the expression or function of a target. In general, where the composition comprising a nucleic acid, a suitable dose of a nucleic acid e.g. an siRNA of the invention will be in the range of about 0.001 to about 200.0 milligrams per kilogram body weight of the recipient per day, generally in the range of about 1 to 50 mg per kilogram body weight per day. Typically, a suitable dose of a nucleic acid e.g. an siRNA of the invention will be in the range of about 0.1 mg/kg to about 5.0 mg/kg, e.g., about 0.3 mg/kg and about 3.0 mg/kg.

A repeat-dose regimen may include administration of a therapeutic amount of a nucleic acid e.g. siRNA on a regular basis, such as every other day or once a year. In certain embodiments, the nucleic acid e.g. siRNA is administered about once per month to about once per quarter (i.e., about once every three months).

In various embodiments, the nucleic acid e.g. siRNA agent is administered at a dose of about 0.01 mg/kg to about 10 mg/kg or about 0.5 mg/kg to about 50 mg/kg. In some embodiments, the nucleic acid e.g. siRNA agent is administered at a dose of about 10 mg/kg to about 30 mg/kg. In certain embodiments, the nucleic acid e.g. siRNA agent is administered at a dose selected from about 0.5 mg/kg 1 mg/kg, 1.5 mg/kg, 3 mg/kg, 5 mg/kg, 10 mg/kg, and 30 mg/kg. In certain embodiments, the nucleic acid e.g. siRNA agent is administered about once per week, once per month, once every other two months, or once a quarter (i.e., once every three months) at a dose of about 0.1 mg/kg to about 5.0 mg/kg. In certain embodiments, the nucleic acid e.g. siRNA agent is administered to the subject once a week. In certain embodiments, the nucleic acid e.g. siRNA agent is administered to the subject once a month. In certain embodiments, the nucleic acid e.g. siRNA agent is administered once per quarter (i.e., every three months).

After an initial treatment regimen, the treatments can be administered on a less frequent basis. For example, after administration weekly or biweekly for three months, administration can be repeated once per month, for six months, or a year; or longer.

The pharmaceutical composition can be administered once daily, or administered as two, three, or more sub-doses at appropriate intervals throughout the day or even using continuous infusion or delivery through a controlled release formulation. In that case, the nucleic acid e.g. siRNA contained in each sub-dose must be correspondingly smaller in order to achieve the total daily dosage. The dosage unit can also be compounded for delivery over several days, e.g., using a conventional sustained release formulation which provides sustained release of the nucleic acid e.g. siRNA over a several day period. Sustained release formulations are well known in the art and are particularly useful for delivery of agents at a particular site, such as could be used with the agents of the present invention. In this embodiment, the dosage unit contains a corresponding multiple of the daily dose.

In other embodiments, a single dose of the pharmaceutical compositions can be long lasting, such that subsequent doses are administered at not more than 3, 4, or 5 day intervals, or at not more than 1, 2, 3, or 4 week intervals. In some embodiments of the invention, a single dose of the pharmaceutical compositions of the invention is administered once per week. In other embodiments of the invention, a single dose of the pharmaceutical compositions of the invention is administered bimonthly. In certain embodiments, the siRNA is administered about once per month to about once per quarter (i.e., about once every three months), or even every 6 months or 12 months.

Estimates of effective dosages and in vivo half-lives for the individual nucleic acid e.g. siRNAs encompassed by the invention can be made using conventional methodologies or on the basis of in vivo testing using an appropriate animal model, as known in the art.

The pharmaceutical compositions of the present invention can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal, or intramuscular injection or infusion; subdermal, e.g., via an implanted device; or intracranial, e.g., by intraparenchymal, intrathecal or intraventricular administration. In certain preferred embodiments, the compositions are administered by intravenous infusion or injection. In certain embodiments, the compositions are administered by subcutaneous injection.

In one embodiment, the nucleic acid e.g. siRNA agent is administered to the subject subcutaneously.

The inhibitor e.g. nucleic acid e.g. siRNA can be delivered in a manner to target a particular tissue (e.g. in particular liver cells).

### Methods for Inhibiting Gene Expression or Inhibition of Target Expression or Function

The present invention also provides methods of inhibiting expression of a gene in a cell and methods for inhibiting expression and/or function of other target molecules. The methods include contacting a cell with a nucleic acid of the invention e.g. siRNA agent, such as double stranded siRNA in an amount effective to inhibit expression of the gene in the cell, thereby inhibiting expression of the gene in the cell. In a preferred embodiment, the gene is ZPI.

Contacting of a cell with the inhibitor e.g. the nucleic acid e.g. an siRNA, such as a double stranded siRNA agent, may be done in vitro or in vivo. Contacting a cell in vivo with the inhibitor nucleic acid e.g. siRNA includes contacting a cell or group of cells within a subject, e.g., a human subject, with the nucleic acid e.g. siRNA. Combinations of in vitro and in vivo methods of contacting a cell are also possible. Contacting a cell may be direct or indirect, as discussed above. Furthermore, contacting a cell may be accomplished via a targeting ligand moiety, including any ligand moiety described herein or known in the art. In preferred embodiments, the targeting ligand moiety is a carbohydrate moiety, e.g. a GalNAc3 ligand, or any other ligand moiety that directs the siRNA agent to a site of interest.

The term “inhibiting,” as used herein, is used interchangeably with “reducing,” “silencing,” “downregulating”, “suppressing”, and other similar terms, and includes any level of inhibition.

In some embodiments of the methods of the invention, expression or activity of a gene or an inhibition target is inhibited by at least 30%, 35%, 40%, 45%, 50%, 55%, 60%, 65%, 70%, 75%, 80%, 85%, 90%, or 95%, or to below the level of detection of the assay, preferably when determined by qPCR as described herein and/or when the siRNA is introduced into the target cell by transfection. In certain embodiments, the methods include a clinically relevant inhibition of expression of a target gene e.g. as demonstrated by a clinically relevant outcome after treatment of a subject with an agent to reduce the expression of the gene and/or activity of the target

In some embodiments, when transfected into the cells, the nucleic acid of the invention inhibits expression of the ZPI gene with an IC50 value lower than 2500 pM, 2400 pM, 2300 pM, 2200 pM, 2100 pM, 2000 pM, 1900 pM, 1800 pM, 1700 pM, 1600 pM, 1500 pM, 1400 pM, 1300 pM, 1200 pM, 1100 pM, 1000 pM, 900 pM, 800 pM, 700 pM, 600 pM, 500 pM, 400 pM, 300 pM, 200 pM or 100 pM, preferably when determined by qPCR, more preferably by reverse transcriptase (RT)-qPCR, as described herein.

In a preferred embodiment, when transfected into the cells, the nucleic acid of the invention inhibits expression of the ZPI gene with an IC50 value lower than 2500 pM. In a more preferred embodiment, when transfected into the cells, the nucleic acid of the invention inhibits expression of the ZPI gene with an IC50 value lower than 1000 pM. In an even more preferred embodiment, when transfected into the cells, the nucleic acid of the invention inhibits expression of the ZPI gene with an IC50 value lower than 500 pM. In a most preferred embodiment, when transfected into the cells, the nucleic acid of the invention inhibits expression of the ZPI gene with an IC50 value lower than 100 pM.

Inhibition of expression of the ZPI gene may be quantified by the following method:

Huh7 cells (human hepatocyte-derived cell line, obtained from JCRB Cell Bank) may be maintained in Dulbecco's Modified Eagle Medium (DMEM) supplemented with 10% FBS at 37° C. in an atmosphere of 5% CO2. Cells may then be transfected with siRNA duplexes targeting ZPI mRNA or a negative control siRNA (siRNA-control; sense strand 5′-UUCUCCGAACGUGUCACGUTT-3′ (SEQ ID NO:794), antisense strand 5′-ACGUGACACGUUCGGAGAATT-3′ (SEQ ID NO:790)) using 10×3-fold serial dilutions over a final duplex concentration range of 20 nM to 1 pM. Transfection may be carried out by adding 9.7 μL Opti-MEM (ThermoFisher) plus 0.3 μL Lipofectamine RNAiMAX (ThermoFisher) to 10 μL of each siRNA duplex. The mixture may be incubated at room temperature for 15 minutes before being added to 100 μL of complete growth medium containing 20,000 Huh7 cells. Cells may be incubated for 24 hours at 37° C./5% CO2 prior to total RNA purification using a RNeasy 96 Kit (Qiagen). Each duplex may be tested by transfection in duplicate wells in a single experiment.

cDNA synthesis may be performed using FastQuant RT (with gDNase) Kit (Tiangen). Real-time quantitative PCR (qPCR) may be performed on an ABI Prism 7900HT or ABI QuantStudio 7 with primers specific for human ZPI (Hs01547819_ml) and human GAPDH (Hs02786624_g1) using FastStart Universal Probe Master Kit (Roche).

qPCR may be performed in duplicate on cDNA derived from each well and the mean cycle threshold (Ct) calculated. Relative ZPI expression may be calculated from mean Ct values using the comparative Ct (ΔΔCt) method, normalised to GAPDH and relative to untreated cells. Maximum percent inhibition of ZPI expression and IC50 values may be calculated using a four parameter (variable slope) model using GraphPad Prism 9.

Alternatively or in addition, inhibition of expression of the ZPI gene may be characterized by a reduction of mean relative expression of the ZPI gene.

In some embodiments, when cells are transfected with 0.1 nM of the nucleic acid of the invention, the mean relative expression of ZPI is below 1, 0.9, 0.8, 0.7, 0.6, 0.5, or 0.4, preferably when determined by qPCR, more preferably by reverse transcriptase (RT)-qPCR, as described herein.

In some embodiments, when cells are transfected with 5 nM of the nucleic acid of the invention, the mean relative expression of ZPI is below 1, 0.9, 0.8, 0.7, 0.6, 0.5, 0.4 or 0.3, preferably when determined by qPCR, more preferably by reverse transcriptase (RT)-qPCR, as described herein.

Mean relative expression of the ZPI gene may be quantified by the following method:

Huh7 cells (human hepatocyte-derived cell line, obtained from JCRB Cell Bank) may be maintained in Dulbecco's Modified Eagle Medium (DMEM) supplemented with 10% FBS at 37° C. in at atmosphere of 5% CO₂. Cells may be transfected with siRNA duplexes targeting ZPI mRNA or a negative control siRNA (siRNA-control; sense strand 5′-UUCUCCGAACGUGUCACGUTT-3′ (SEQ ID NO:794), antisense strand 5′-ACGUGACACGUUCGGAGAATT-3′ (SEQ ID NO:790)) at a final duplex concentration of 5 nM and 0.1 nM. Transfection may be carried out by adding 9.7 μL Opti-MEM (ThermoFisher) plus 0.3 μL Lipofectamine RNAiMAX (ThermoFisher) to 10 μL of each siRNA duplex. The mixture may be incubated at room temperature for 15 minutes before being added to 100 μL of complete growth medium containing 20,000 Huh7 cells. Cells may be incubated for 24 hours at 37° C./5% CO₂ prior to total RNA purification using a RNeasy 96 Kit (Qiagen). Each duplex may be tested by transfection in duplicate wells in two independent experiments.

cDNA synthesis may be performed using FastQuant RT (with gDNase) Kit (Tiangen). Real-time quantitative PCR (qPCR) may be performed on an ABI Prism 7900HT or ABI QuantStudio 7 with primers specific for human ZPI (Hs01547819_m1) and human GAPDH (Hs02786624_g1) using FastStart Universal Probe Master Kit (Roche).

qPCR may be performed in duplicate on cDNA derived from each well and the mean Ct calculated. Relative ZPI expression may be calculated from mean Ct values using the comparative Ct (ΔΔCt) method, normalised to GAPDH and relative to untreated cells.

Inhibition of the expression of a gene may be manifested by a reduction of the amount of mRNA of the target gene of interest in comparison to a suitable control. Inhibition of the function of a target may be manifested by a reduction of the activity of the target in comparison to a suitable control.

In other embodiments, inhibition of the expression of a gene or other target may be assessed in terms of a reduction of a parameter that is functionally linked to gene expression, e.g, protein expression or signalling pathways.

Methods of Treating or Preventing Diseases Associated with Gene Expression/Expression of Function of a Target.

The present invention also provides methods of using nucleic acid e.g. an siRNA of the invention or a composition containing nucleic acid e.g. an siRNA of the invention to reduce or inhibit gene expression in a cell or reduce expression or function of a target. The methods include contacting the cell with a nucleic acid e.g. dsiRNA of the invention and maintaining the cell for a time sufficient to obtain degradation of the mRNA transcript of a gene, thereby inhibiting expression of the gene in the cell. Reduction in gene expression or function of a target can be assessed by any methods known in the art. In a preferred embodiment, the gene is ZPI.

In the methods of the invention the cell may be contacted in vitro or in vivo, i.e., the cell may be within a subject.

A cell suitable for treatment using the methods of the invention may be any cell that expresses a gene of interest or target of interest associated with disease.

The in vivo methods of the invention may include administering to a subject a composition containing a nucleic acid of the invention e.g. an iRNA, where the nucleic acid e.g. siRNA includes a nucleoside sequence that is complementary to at least a part of an RNA transcript of the gene of the mammal to be treated, or complementary to another nucleic acid the expression and/or function of which is associated with diseases.

The present invention further provides methods of treatment of a subject in need thereof. The treatment methods of the invention include administering a nucleic acid such as an siRNA of the invention to a subject, e.g., a subject that would benefit from a reduction or inhibition of the expression of a gene and/or expression and/or function of a target, in a therapeutically effective amount e.g. a nucleic acid such as an siRNA targeting a gene or a pharmaceutical composition comprising the nucleic acid targeting a gene.

A nucleic acid e.g. siRNA of the invention may be administered as a “free” nucleic acid or “free” siRNA, administered in the absence of a pharmaceutical composition. The naked nucleic acid may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution can be adjusted such that it is suitable for administering to a subject.

Alternatively, a nucleic acid e.g. siRNA of the invention may be administered as a pharmaceutical composition, such as a dsiRNA liposomal formulation.

In one embodiment, the method includes administering a composition featured herein such that expression of the target gene is decreased, such as for about 1, 2, 3, 4, 5, 6, 7, 8, 12, 16, 18, 24 hours, 28, 32, or about 36 hours. In one embodiment, expression of the target gene is decreased for an extended duration, e.g., at least about two, three, four days or more, e.g., about one week, two weeks, three weeks, or four weeks or longer, e.g., about 1 month, 2 months, or 3 months.

Subjects can be administered a therapeutic amount of nucleic acid e.g. siRNA, such as about 0.01 mg/kg to about 200 mg/kg.

The nucleic acid e.g. siRNA can be administered by intravenous infusion over a period of time, on a regular basis. In certain embodiments, after an initial treatment regimen, the treatments can be administered on a less frequent basis. Administration of the siRNA can reduce gene product levels of a target gene, e.g., in a cell or tissue of the patient by at least about 20%, 25%, 30%, 35%, 40%, 45%, 50%, 55%, 60%, 65%, 70%, 75%, 80%, 85%, 90%, or 95%, or below the level of detection of the assay method used. In certain embodiments, administration results in clinical stabilization or preferably clinically relevant reduction of at least one sign or symptom of a gene-associated disorder.

Alternatively, the nucleic acid e.g. siRNA can be administered subcutaneously, i.e., by subcutaneous injection. One or more injections may be used to deliver the desired daily dose of nucleic acid e.g. s iRNA to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of nucleic acid on a regular basis, such as every other day or to once a year. In certain embodiments, the nucleic acid is administered about once per month to about once per quarter (i.e., about once every three months).

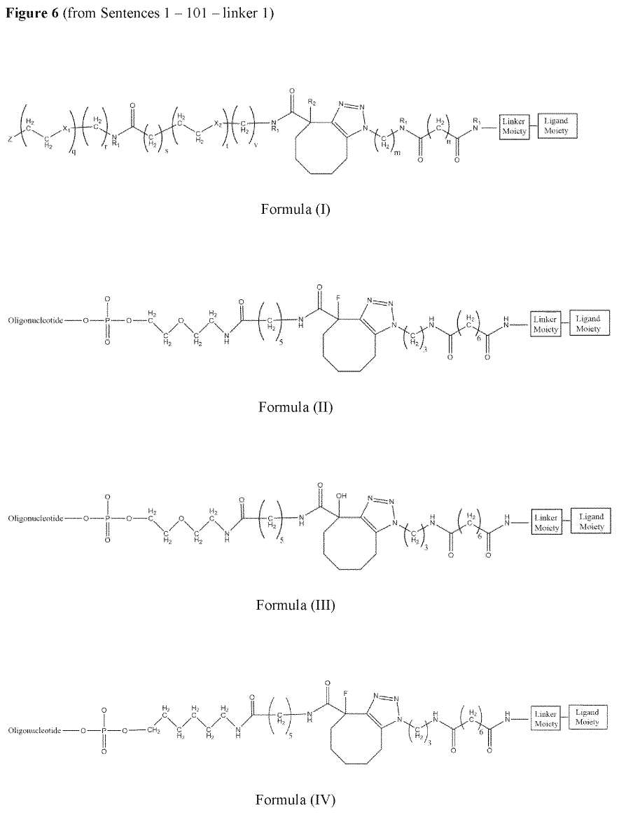

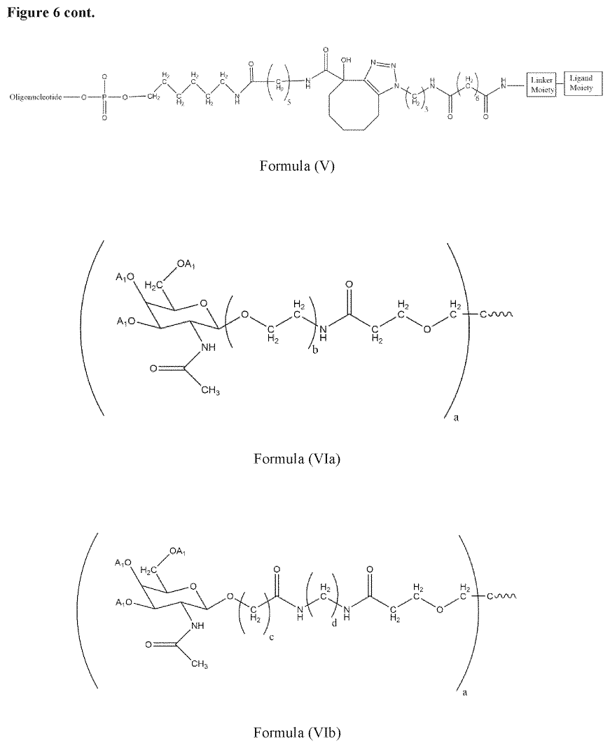

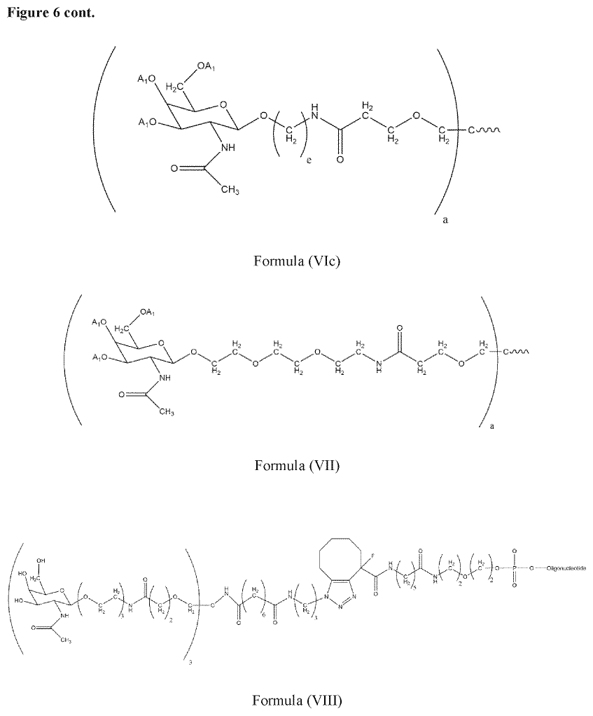

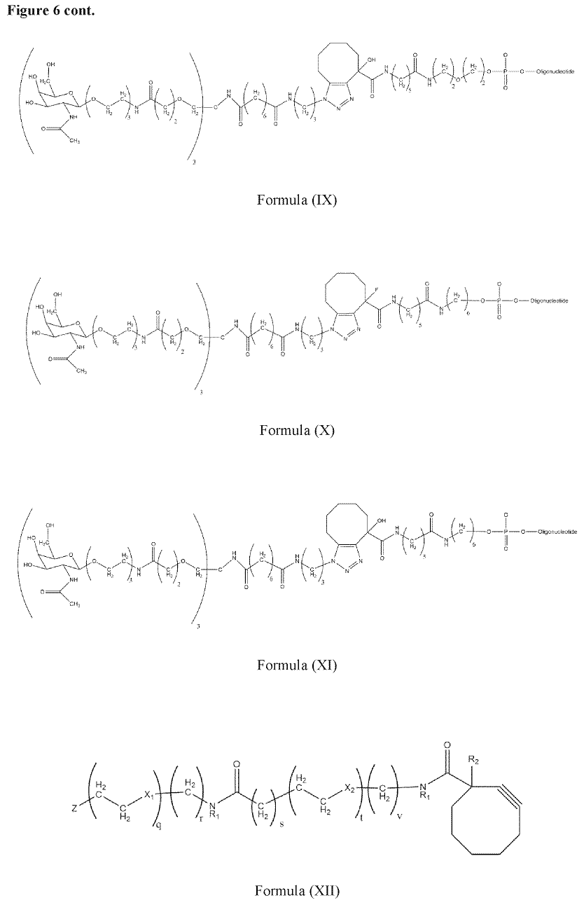

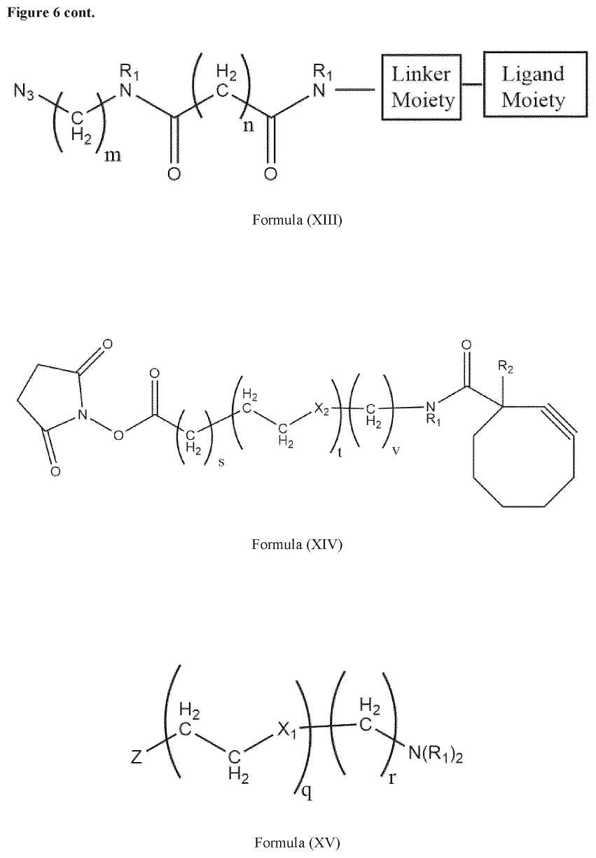

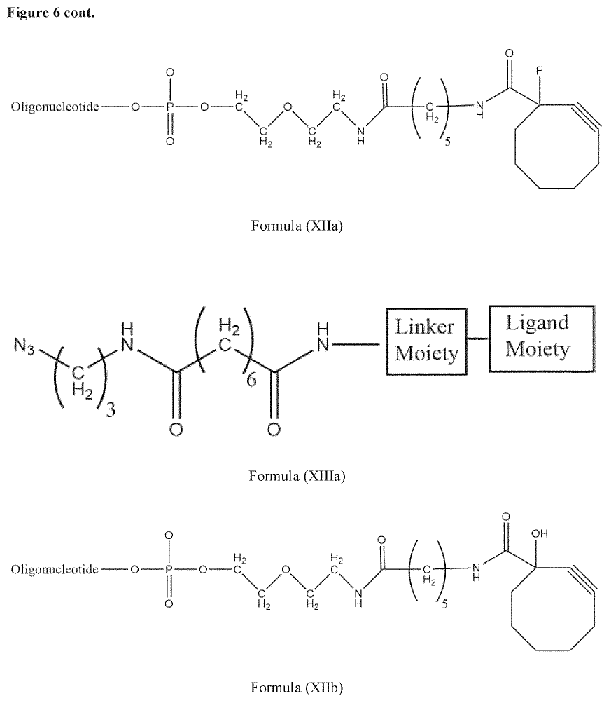

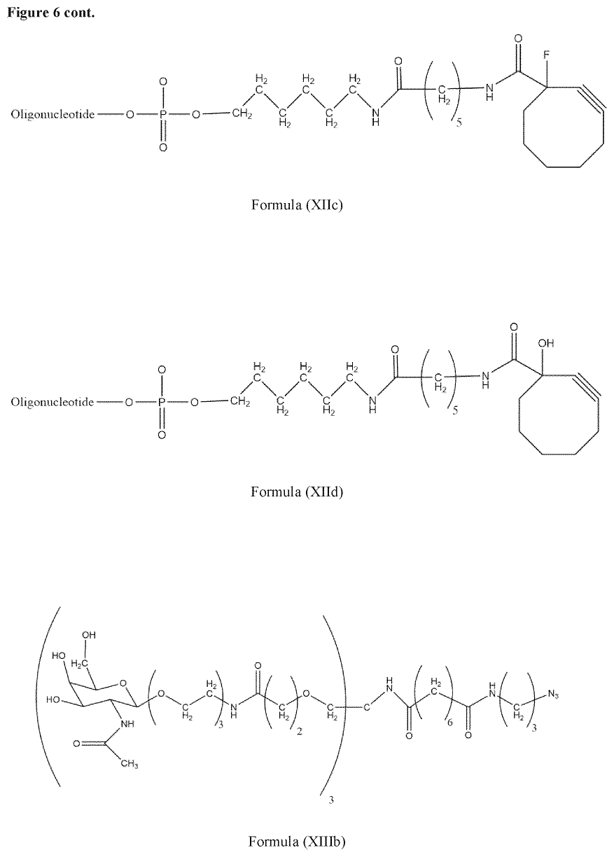

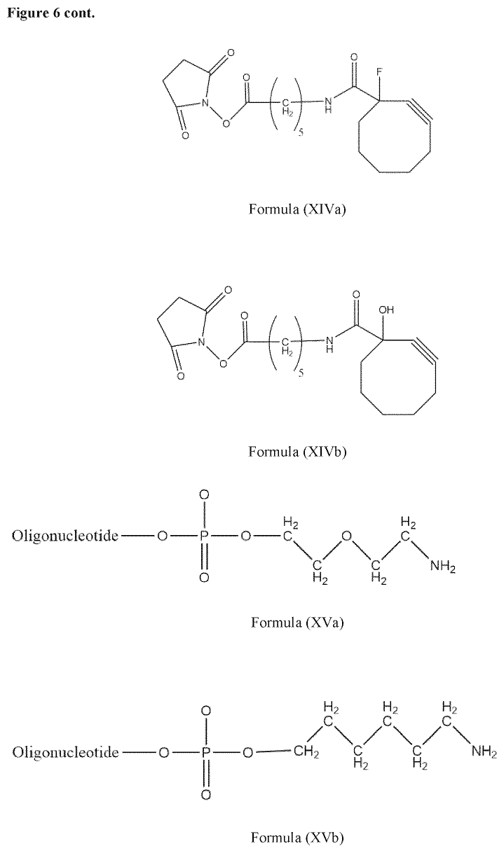

In one aspect the present invention may be applied in the compounds, processes, compositions or uses of the following Sentences numbered 1-101 (wherein reference to any Formula in the Sentences 1-101 refers only to those Formulas that are defined within Sentences 1-101. These formulae are reproduced in FIG. 6)
1. A compound comprising the following structure:

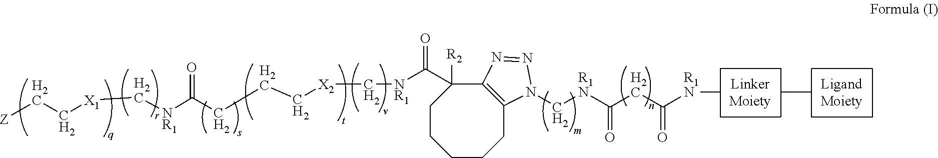

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
2. A compound according to Sentence 1, wherein R₁ is hydrogen at each occurrence.
3. A compound according to Sentence 1, wherein R₁ is methyl.
4. A compound according to Sentence 1, wherein R₁ is ethyl.
5. A compound according to any of Sentences 1 to 4, wherein R₂ is hydroxy.
6. A compound according to any of Sentences 1 to 4, wherein R₂ is halo.
7. A compound according to Sentence 6, wherein R₂ is fluoro.
8. A compound according to Sentence 6, wherein R₂ is chloro.
9. A compound according to Sentence 6, wherein R₂ is bromo.
10. A compound according to Sentence 6, wherein R₂ is iodo.
11. A compound according to Sentence 6, wherein R₂ is nitro.
12. A compound according to any of Sentences 1 to 11, wherein X₁ is methylene.
13. A compound according to any of Sentences 1 to 11, wherein X₁ is oxygen.
14. A compound according to any of Sentences 1 to 11, wherein X₁ is sulfur.
15. A compound according to any of Sentences 1 to 14, wherein X₂ is methylene.
16. A compound according to any of Sentences 1 to 15, wherein X₂ is oxygen.
17. A compound according to any of Sentences 1 to 16, wherein X₂ is sulfur.
18. A compound according to any of Sentences 1 to 17, wherein m=3.
19. A compound according to any of Sentences 1 to 18, wherein n=6.
20. A compound according to Sentences 13 and 15, wherein X₁ is oxygen and X₂ is methylene, and preferably wherein:
   q=1,
   r=2,
   s=1,
   t=1,
   v=1.
21. A compound according to Sentences 12 and 15, wherein both Xi and X₂ are methylene, and preferably wherein:
   q=1,
   r=3,
   s=1,
   t=1,
   v=1.
22. A compound according to any of Sentences 1 to 21, wherein Z is:

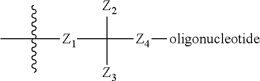

wherein:
Z₁, Z₂, Z₃, Z₄ are independently at each occurrence oxygen or sulfur; and
one the bonds between P and Z₂, and P and Z₃ is a single bond and the other bond is a double bond.
23. A compound according to Sentence 22, wherein said oligonucleoside is an RNA compound capable of modulating, preferably inhibiting, expression of a target gene.
24. A compound according to Sentence 23, wherein said RNA compound comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends.
25. A compound according to Sentence 24, wherein the RNA compound is attached at the 5′ end of its second strand to the adjacent phosphate.
26. A compound according to Sentence 24, wherein the RNA compound is attached at the 3′ end of its second strand to the adjacent phosphate.
27. A compound of Formula (II):

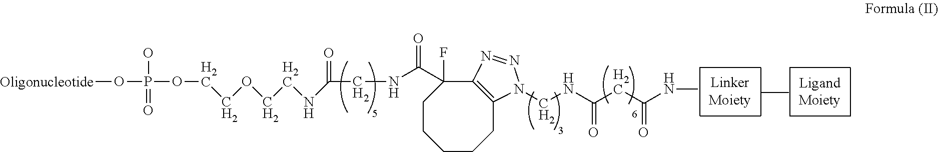

28. A compound of Formula (III):

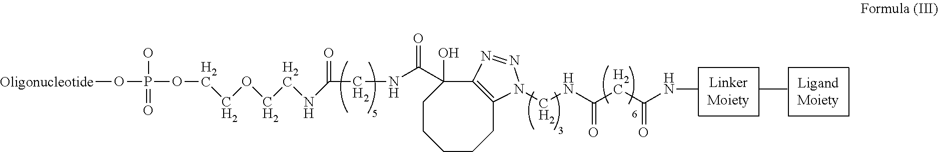

29. A compound according to Sentence 27 or 28, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
30. A composition comprising a compound of Formula (II) as defined in Sentence 27, and a compound of Formula (III) as defined in Sentence 28, optionally dependent on Sentence 29.
31. A composition according to Sentence 30, wherein said compound of Formula (III) as defined in Sentence 28 is present in an amount in the range of 10 to 15% by weight of said composition.
32. A compound of Formula (IV):

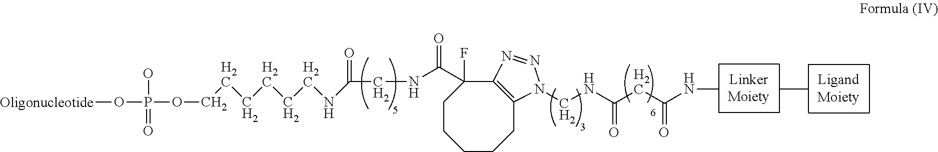

33. A compound of Formula (V):

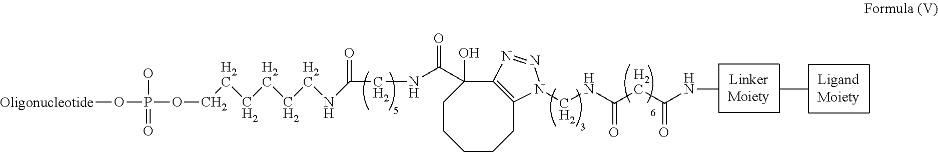

34. A compound according to Sentence 32 or 33, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
35. A composition comprising a compound of Formula (IV) as defined in Sentence 32, and a compound of Formula (V) as defined in Sentence 33, optionally dependent on Sentence 34.
36. A composition according to Sentence 35, wherein said compound of Formula (V) as defined in Sentence 33 is present in an amount in the range of 10 to 15% by weight of said composition.
37. A compound as defined in any of Sentences 1 to 29, or 32 to 34, wherein the oligonucleoside comprises an RNA duplex which further comprises one or more riboses modified at the 2′ position, preferably a plurality of riboses modified at the 2′ position.
38. A compound according to Sentence 37, wherein the modifications are chosen from 2′-O-methyl, 2′-deoxy-fluoro, and 2′-deoxy.
39. A compound according to any of Sentences 1 to 29, or 32 to 34, or 37 to 38, wherein the oligonucleoside further comprises one or more degradation protective moieties at one or more ends.
40. A compound according to Sentence 39, wherein said one or more degradation protective moieties are not present at the end of the oligonucleoside strand that carries the ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the strand that carries the ligand moieties.
41. A compound according to any of Sentences 1 to 29, or 32 to 34, or 37 to 40, wherein said ligand moiety as depicted in Formula (I) in Sentence 1 comprises one or more ligands.
42. A compound according to Sentence 41, wherein said ligand moiety as depicted in Formula (I) in Sentence 1 comprises one or more carbohydrate ligands.
43. A compound according to Sentence 42, wherein said one or more carbohydrates can be a monosaccharide, disaccharide, trisaccharide, tetrasaccharide, oligosaccharide or polysaccharide.
44. A compound according to Sentence 43, wherein said one or more carbohydrates comprise one or more galactose moieties, one or more lactose moieties, one or more N-AcetylGalactosamine moieties, and/or one or more mannose moieties.
45. A compound according to Sentence 44, wherein said one or more carbohydrates comprise one or more N-Acetyl-Galactosamine moieties.
46. A compound according to Sentence 45, which comprises two or three N-AcetylGalactosamine moieties.
47. A compound according to any of Sentences 41 to 46, wherein said one or more ligands are attached in a linear configuration, or in a branched configuration.
48. A compound according to Sentence 47, wherein said one or more ligands are attached as a biantennary or triantennary branched configuration.
49. A compound according to Sentences 46 to 48, wherein said moiety:

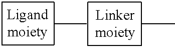

as depicted in Formula (I) in Sentence 1 is any of Formulae (VIa), (VIb) or (VIc), preferably Formula (VIa):

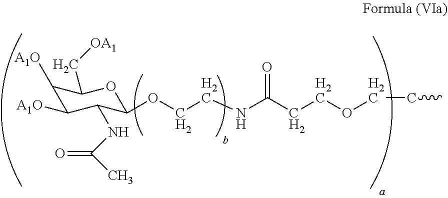

wherein:
A₁ is hydrogen, or a suitable hydroxy protecting group;
a is an integer of 2 or 3; and
b is an integer of 2 to 5; or

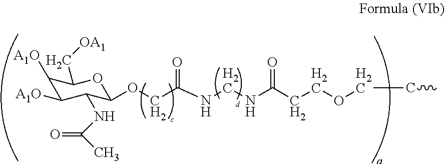

wherein:
A₁ is hydrogen, or a suitable hydroxy protecting group;
a is an integer of 2 or 3; and
c and d are independently integers of 1 to 6; or

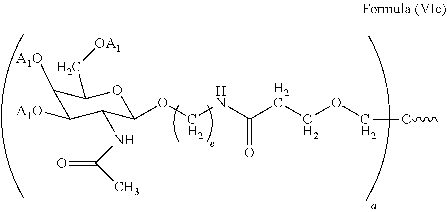

wherein:
A₁ is hydrogen, or a suitable hydroxy protecting group;
a is an integer of 2 or 3; and
e is an integer of 2 to 10.
50. A compound according to Sentences 46 to 48, wherein said moiety:

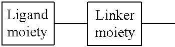

as depicted in Formula (I) in Sentence 1 is Formula (VII):

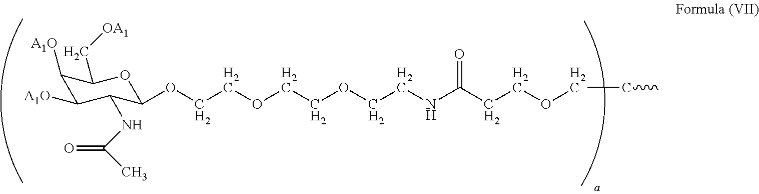

wherein:
A₁ is hydrogen;
a is an integer of 2 or 3.
51. A compound according to Sentence 49 or 50, wherein a=2.
52. A compound according to Sentence 49 or 50, wherein a=3.
53. A compound according to Sentence 49, wherein b=3.
54. A compound of Formula (VIII):

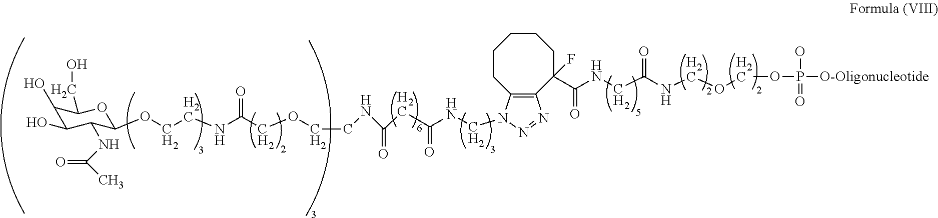

55. A compound of Formula (IX):

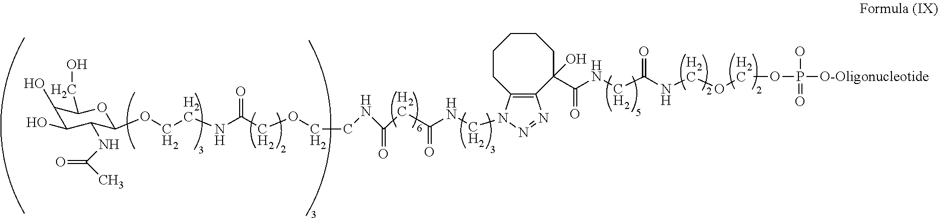

56. A compound according to Sentence 54 or 55, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
57. A composition comprising a compound of Formula (VIII) as defined in Sentence 54, and a compound of Formula (IX) as defined in Sentence 55, optionally dependent on Sentence 56.
58. A composition according to Sentence 57, wherein said compound of Formula (IX) as defined in Sentence 55 is present in an amount in the range of 10 to 15% by weight of said composition.
59. A compound of Formula (X):

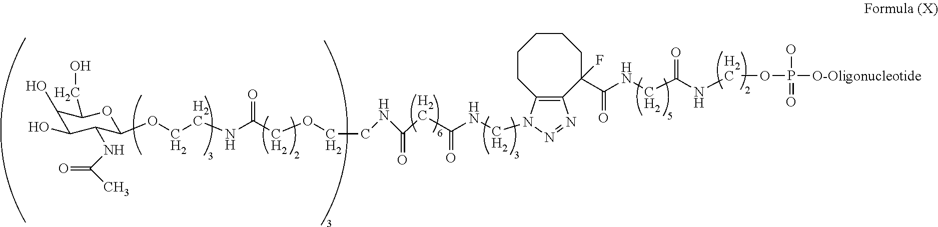

60. A compound of formula (XI):

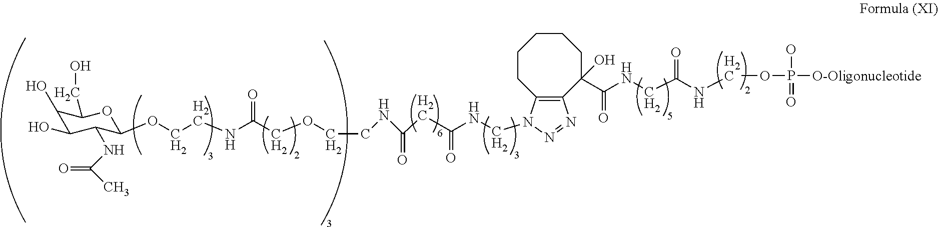

61. A compound according to Sentence 59 or 60, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
62. A composition comprising a compound of Formula (X) as defined in Sentence 59, and a compound of Formula (XI) as defined in Sentence 60, optionally dependent on Sentence 61.
63. A composition according to Sentence 62, wherein said compound of Formula (XI) as defined in Sentence 60 is present in an amount in the range of 10 to 15% by weight of said composition.
64. A compound as defined in any of Sentences 54 to 63, wherein the oligonucleoside comprises an RNA duplex which further comprises one or more riboses modified at the 2′ position, preferably a plurality of riboses modified at the 2′ position.
65. A compound according to Sentence 64, wherein the modifications are chosen from 2′-O-methyl, 2′-deoxy-fluoro, and 2′-deoxy.
66. A compound according to any of Sentences 54 to 65, wherein the oligonucleoside further comprises one or more degradation protective moieties at one or more ends.
67. A compound according to Sentence 66, wherein said one or more degradation protective moieties are not present at the end of the oligonucleoside strand that carries the ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the strand that carries the ligand moieties, as shown in any of Formulae (VIII), (IX), (X) or (XI) in any of Sentences 54, 55, 59 or 60.
68. A process of preparing a compound according to any of Sentences 1 to 29, 32 to 34, 37 to 56, 59 to 61, and 64 to 67, and/or a composition according to any of Sentences 30, 31, 35, 36, 57, 58, 62, 63, which comprises reacting compounds of Formulae (XII) and (XIII):

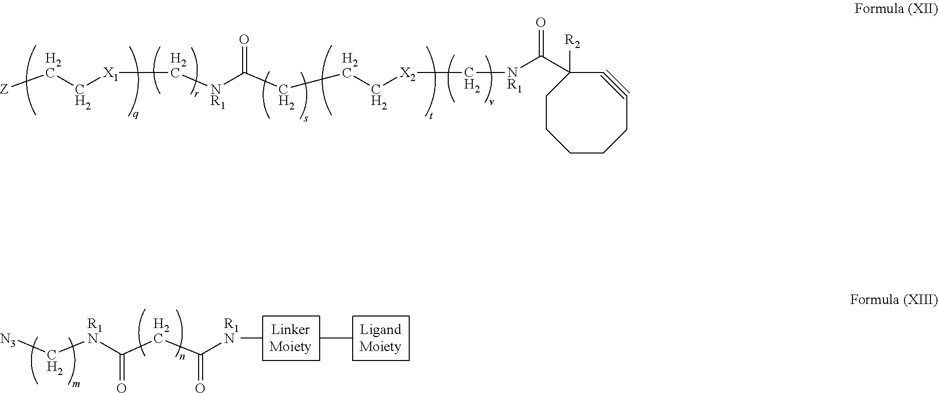

herein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety;
and where appropriate carrying out deprotection of the ligand and/or annealing of a second strand for the oligonucleoside moiety.
69. A process according to Sentence 68, wherein a compound of Formula (XII) is prepared by reacting compounds of Formulae (XIV) and (XV):

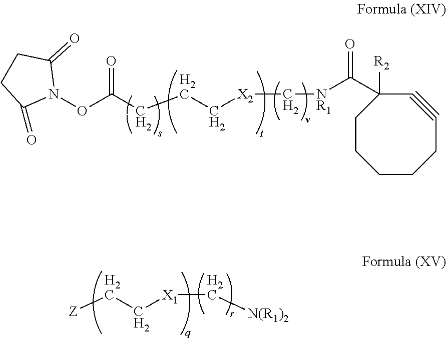

R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
70. A process according to Sentence 68, to prepare a compound according to any of Sentences 20, 25, 27, 29, 54, 56, and/or a composition according to any of Sentences 30, 31, 57, 58, wherein:
compound of Formula (XII) is Formula (XIIa):

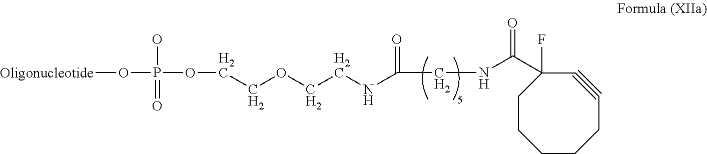

and compound of Formula (XIII) is Formula (XIIIa):

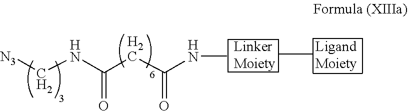

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
71. A process according to Sentence 68, to prepare a compound according to any of Sentences 20, 25, 28, 29, 55, 56, and/or a composition according to any of Sentences 30, 31, 57, 58, wherein:
compound of Formula (XII) is Formula (XIIb):

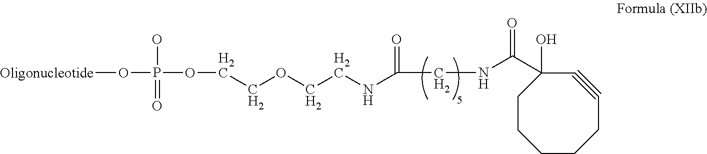

and compound of Formula (XIII) is Formula (XIIIa):

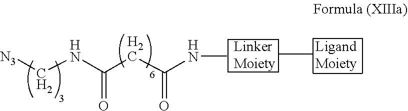

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
72. A process according to Sentence 68, to prepare a compound according to any of Sentences 21, 26, 32, 34, 59, 61, and/or a composition according to any of Sentences 35, 36, 62, 63, wherein:
compound of Formula (XII) is Formula (XIIc):

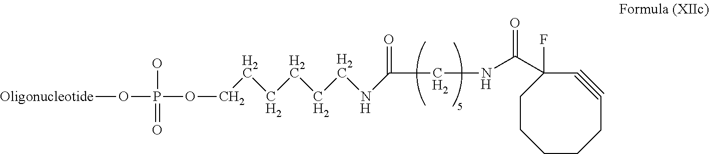

and compound of Formula (XIII) is Formula (XIIIa):

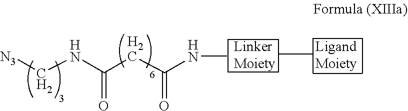

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
73. A process according to Sentence 68, to prepare a compound according to any of Sentences 21, 26, 33, 34, 60, 61, and/or a composition according to any of Sentences 35, 36, 62, 63, wherein:
compound of Formula (XII) is Formula (XIId):

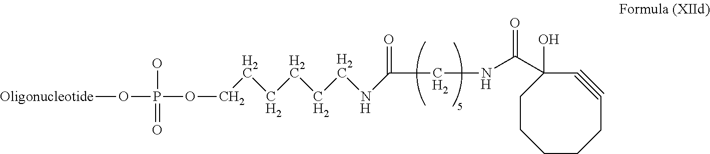

and compound of Formula (XIII) is Formula (XIIIa):

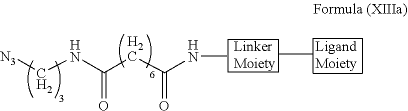

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
74. A process according to any of Sentences 70 to 73, wherein:
compound of Formula (XIIIa) is Formula (XIIIb):

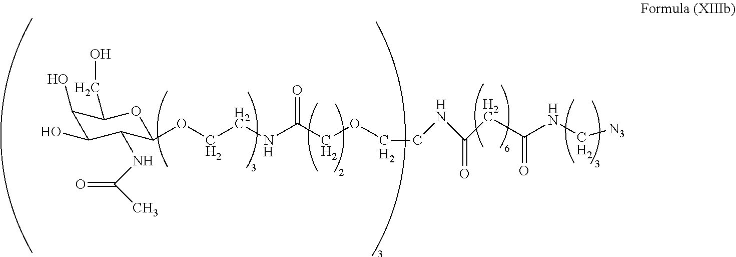

75. A process according to Sentences 69, as dependent on Sentences 70 to 73, wherein: compound of Formula (XIV) is either Formula (XIVa) or Formula (XIVb):

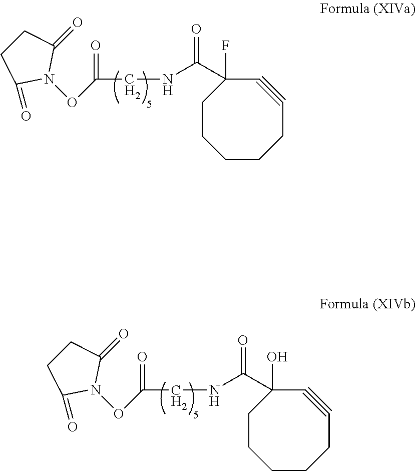

and compound of Formula (XV) is either Formula (XVa) or Formula (XIVb):

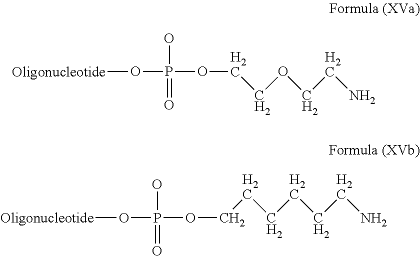

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein (i) said RNA duplex is attached at the 5′ end of its second strand to the adjacent phosphate in Formula (XVa), or (ii) said RNA duplex is attached at the 3′ end of its second strand to the adjacent phosphate in Formula (XVb).
76. A compound of Formula (XII):

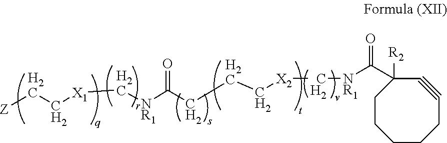

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
77. A compound of Formula (XIIa):

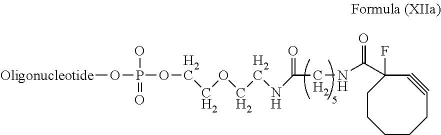

78. A compound of Formula (XIIb):

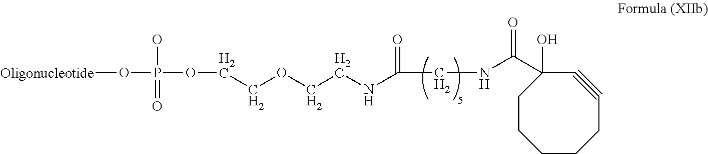

79. A compound of Formula (XIIc):

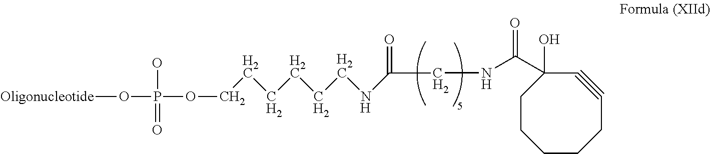

80. A compound of Formula (XIId):

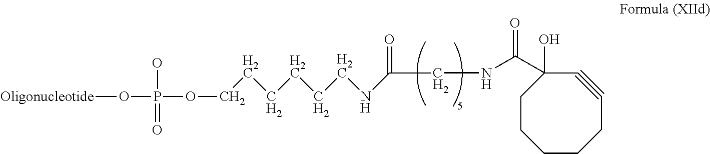

81. A compound of Formula (XIII):

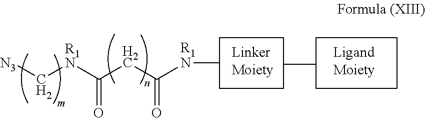

wherein:
R₁ at each occurrence is independently selected from the group consisting of hydrogen, methyl and ethyl;
m is an integer of from 1 to 6;
n is an integer of from 1 to 10.
82. A compound of Formula (XIIIa):

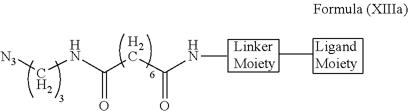

83. A compound of Formula (XIIIb):

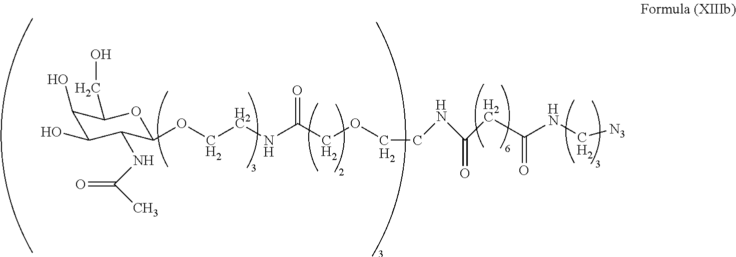

84. A compound of Formula (XIV):

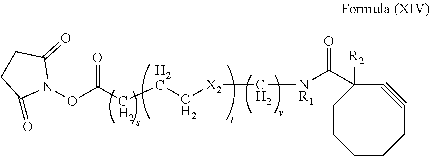

wherein:
R₁ is selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₂ is selected from the group consisting of methylene, oxygen and sulfur;
s, t, v are independently integers from 0 to 4, with the proviso that s, t and v cannot all be 0 at the same time.
85. A compound of Formula (XIVa):

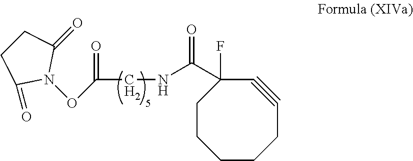

Formula (XIVa)
86. A compound of Formula (XIVb):

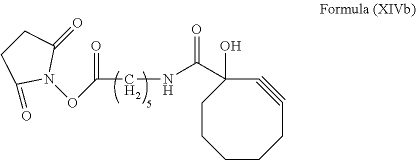

87. A compound of Formula (XV):

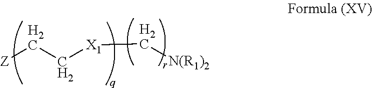

wherein:
R₁ at each occurrence is independently selected from the group consisting of hydrogen, methyl and ethyl;
X₁ is selected from the group consisting of methylene, oxygen and sulfur;
q and r are independently integers from 0 to 4, with the proviso that q and r cannot both be 0 at the same time;
Z is an oligonucleoside moiety.
88. A compound of Formula (XVa):

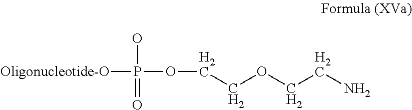

89. A compound of Formula (XVb):

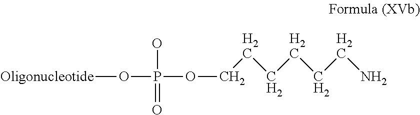

90. Use of a compound according to any of Sentences 76, 81 to 84, 87, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63.
91. Use of a compound according to Sentence 85, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63, wherein R₂═F.
92. Use of a compound according to Sentence 86, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63, wherein R₂═OH.
93. Use of a compound according to Sentence 77, for the preparation of a compound according to any of Sentences 20, 25, 27, 29, 54, 56, and/or a composition according to any of Sentences 30, 31, 57, 58.
94. Use of a compound according to Sentence 78, for the preparation of a compound according to any of Sentences 20, 25, 28, 29, 55, 56, and/or a composition according to any of Sentences 30, 31, 57, 58.
95. Use of a compound according to Sentence 79, for the preparation of a compound according to any of Sentences 21, 26, 32, 34, 59, 61, and/or a composition according to any of Sentences 35, 36, 62, 63.
96. Use of a compound according to Sentence 80, for the preparation of a compound according to any of Sentences 21, 26, 33, 34, 60, 61, and/or a composition according to any of Sentences 35, 36, 62, 63.
97. Use of a compound according to Sentence 88, for the preparation of a compound according to any of Sentences 20, 25, 27 to 29, 54 to 56, and/or a composition according to any of Sentences 30, 31, 57, 58.
98. Use of a compound according to Sentence 89, for the preparation of a compound according to any of Sentences 21, 26, 32 to 34, 59 to 61, and/or a composition according to any of Sentences 35, 36, 62, 63.
99. A compound or composition obtained, or obtainable by a process according to any of Sentences 68 to 75.
100. A pharmaceutical composition comprising of a compound according to any of Sentences 1 to 29, 32 to 34, 37 to 56, 59 to 61, and 64 to 67, and/or a composition according to any of Sentences 30, 31, 35, 36, 57, 58, 62 and 63, together with a pharmaceutically acceptable carrier, diluent or excipient.
   A compound according to any of Sentences 1 to 29, 32 to 34, 37 to 56, 59 to 61, and 64 to 67, and/or a composition according to any of Sentences 30, 31, 35, 36, 57, 58, 62 and 63, for use in therapy.

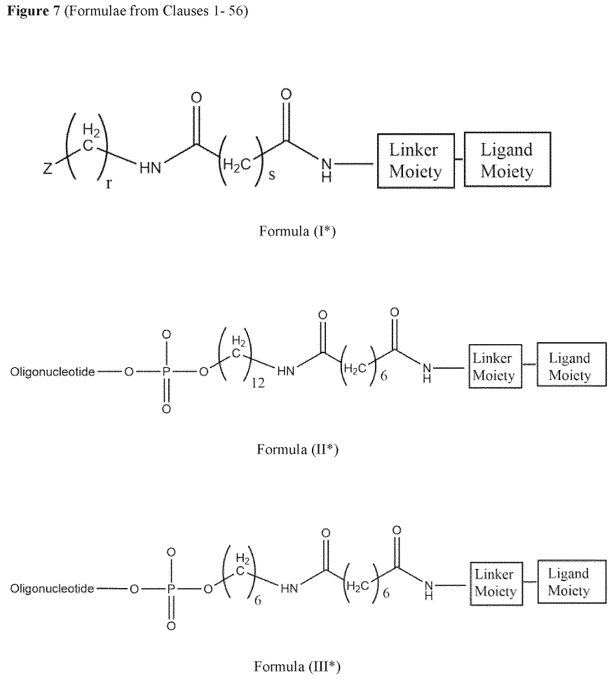

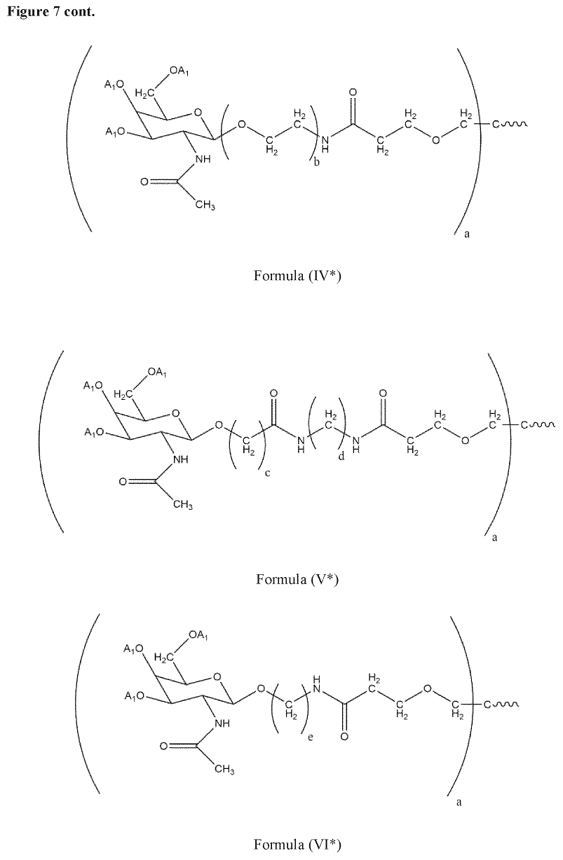

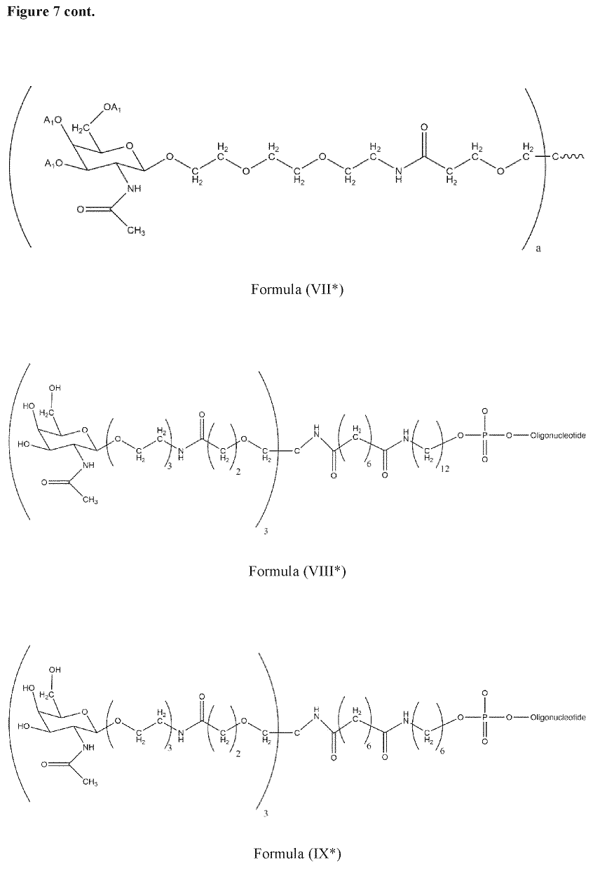

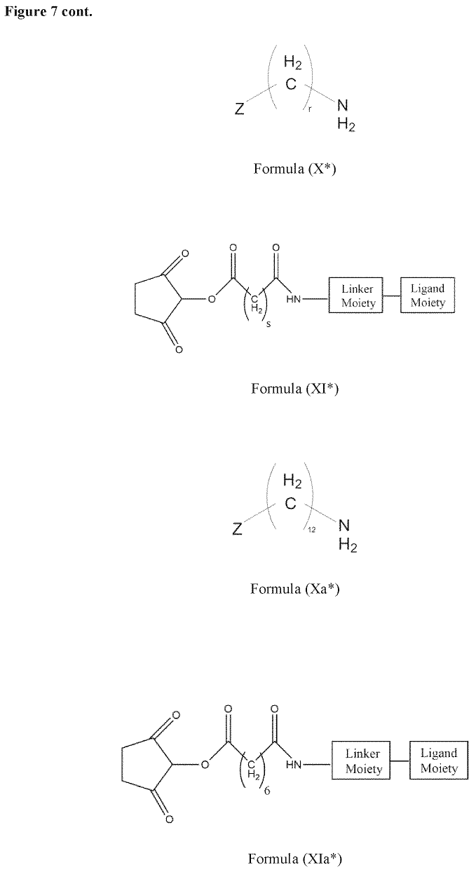

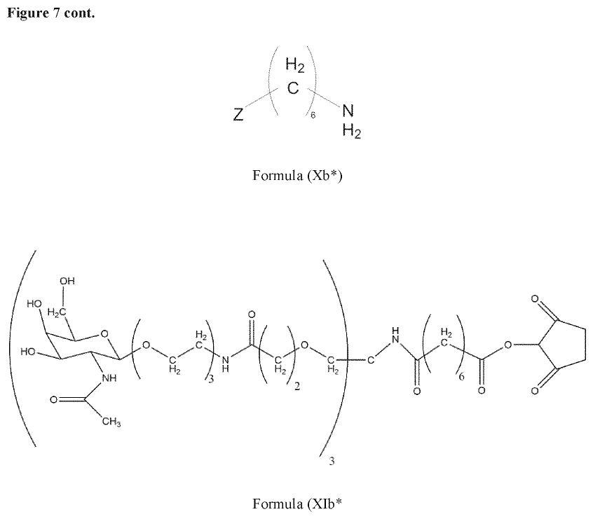

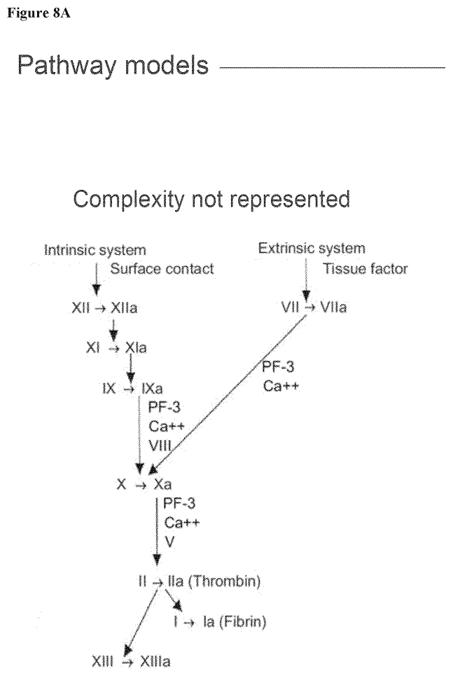

In another aspect the present invention may be applied in the compounds, processes, compositions or uses of the following Clauses numbered 1-56 (wherein reference to any Formula in the Clauses refers only to those Formulas that are defined within Clause 1-56. These formulae are reproduced in FIG. 7).
1. A compound comprising the following structure:

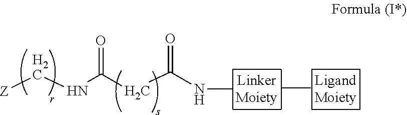

wherein:
r and s are independently an integer selected from 1 to 16; and
Z is an oligonucleoside moiety.
2. A compound according to Clause 1, wherein s is an integer selected from 4 to 12.
3. A compound according to Clause 2, wherein s is 6.
4. A compound according to any of Clauses 1 to 3, wherein r is an integer selected from 4 to 14.
5. A compound according to Clause 4, wherein r is 6.
6. A compound according to Clause 4, wherein r is 12.
7. A compound according to Clause 5, which is dependent on Clause 3.
8. A compound according to Clause 6, which is dependent on Clause 3.
9. A compound according to any of Clauses 1 to 8, wherein Z is:

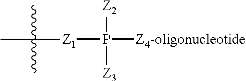

wherein:
Z₁, Z₂, Z₃, Z₄ are independently at each occurrence oxygen or sulfur; and
one the bonds between P and Z₂, and P and Z₃ is a single bond and the other bond is a double bond.
10. A compound according to any of Clauses 1 to 9, wherein said oligonucleoside is an RNA compound capable of modulating, preferably inhibiting, expression of a target gene.
11. A compound according to any of Clause 10, wherein said RNA compound comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends.
12. A compound according to Clause 11, preferably also dependent on Clauses 3 and 6, wherein the RNA compound is attached at the 5′ end of its second strand to the adjacent phosphate.
13. A compound according to Clause 11, preferably also dependent on Clauses 3 and 5, wherein the RNA compound is attached at the 3′ end of its second strand to the adjacent phosphate.
14. A compound of Formula (II*), preferably dependent on Clause 12:

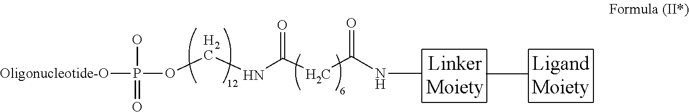

15. A compound of Formula (III*), preferably dependent on Clause 13:

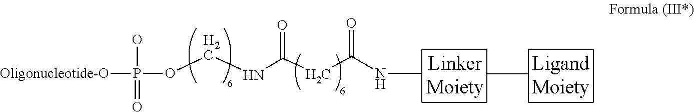

16. A compound as defined in any of Clauses 1 to 15, wherein the oligonucleoside comprises an RNA duplex which further comprises one or more riboses modified at the 2′ position, preferably a plurality of riboses modified at the 2′ position.
17. A compound according to Clause 16, wherein the modifications are chosen from 2′-O-methyl, 2′-deoxy-fluoro, and 2′-deoxy.
18. A compound according to any of Clauses 1 to 17, wherein the oligonucleoside further comprises one or more degradation protective moieties at one or more ends.
19. A compound according to Clause 18, wherein said one or more degradation protective moieties are not present at the end of the oligonucleoside strand that carries the linker/ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the same strand to the end that carries the linker/ligand moieties.
20. A compound according to any of Clauses 1 to 19, wherein said ligand moiety as depicted in Formula (I*) in Clause 1 comprises one or more ligands.
21. A compound according to Clause 20, wherein said ligand moiety as depicted in Formula (I*) in Clause 1 comprises one or more carbohydrate ligands.
22. A compound according to Clause 21, wherein said one or more carbohydrates can be a monosaccharide, disaccharide, trisaccharide, tetrasaccharide, oligosaccharide or polysaccharide.
23. A compound according to Clause 22, wherein said one or more carbohydrates comprise one or more galactose moieties, one or more lactose moieties, one or more N-AcetylGalactosamine moieties, and/or one or more mannose moieties.
24. A compound according to Clause 23, wherein said one or more carbohydrates comprise one or more N-Acetyl-Galactosamine moieties.
25. A compound according to Clause 24, which comprises two or three N-AcetylGalactosamine moieties.
26. A compound according to any of the preceding Clauses, wherein said one or more ligands are attached in a linear configuration, or in a branched configuration.
27. A compound according to Clause 26, wherein said one or more ligands are attached as a biantennary or triantennary branched configuration.
28. A compound according to Clauses 20 to 27, wherein said moiety:

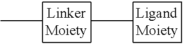

as depicted in Formula (I*) in Clause 1 is any of Formulae (IV*), (V*) or (VI*), preferably Formula (IV*):

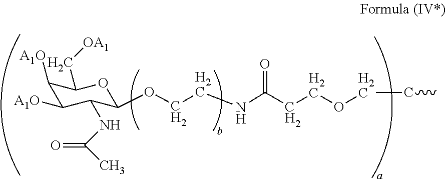

wherein:
A₁ is hydrogen, or a suitable hydroxy protecting group;
a is an integer of 2 or 3; and
b is an integer of 2 to 5; or

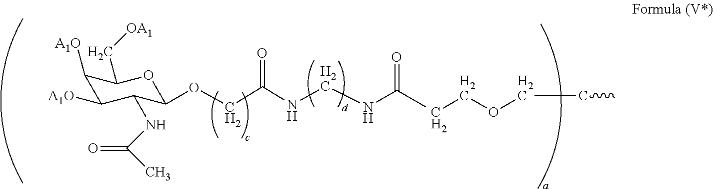

wherein:
A₁ is hydrogen, or a suitable hydroxy protecting group;
a is an integer of 2 or 3; and
c and d are independently integers of 1 to 6; or

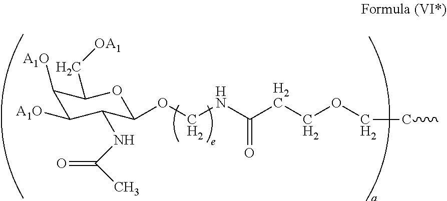

wherein:
A₁ is hydrogen, or a suitable hydroxy protecting group;
a is an integer of 2 or 3; and
e is an integer of 2 to 10.
29. A compound according to any of Clauses 1 to 28, wherein said moiety:

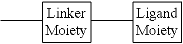

as depicted in Formula (I*) in Clause 1 is Formula (VII*):

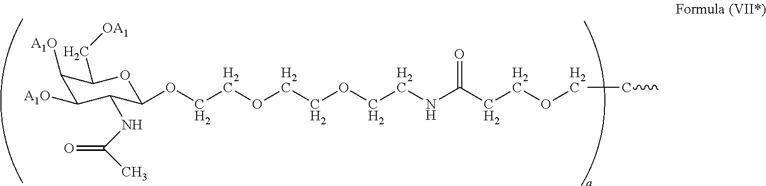

wherein:
A₁ is hydrogen;
a is an integer of 2 or 3.
30. A compound according to Clause 28 or 29, wherein a=2.
31. A compound according to Clause 28 or 29, wherein a=3.
32. A compound according to Clause 28, wherein b=3.
33. A compound of Formula (VIII*):

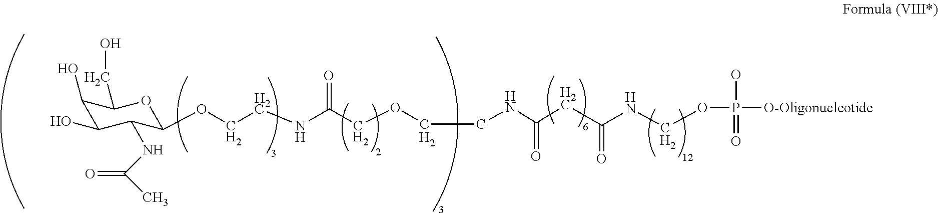

34. A compound of Formula (IX*):

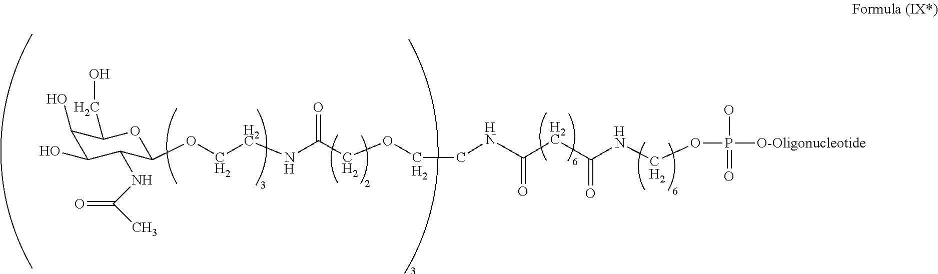

35. A compound according to Clause 33 or 34, wherein the oligonucleoside comprises an RNA duplex which further comprises one or more riboses modified at the 2′ position, preferably a plurality of riboses modified at the 2′ position.
36. A compound according to Clause 35, wherein the modifications are chosen from 2′-O-methyl, 2′-deoxy-fluoro, and 2′-deoxy.
37. A compound according to any of Clauses 33 to 36, wherein the oligonucleoside further comprises one or more degradation protective moieties at one or more ends.
38. A compound according to Clause 37, wherein said one or more degradation protective moieties are not present at the end of the oligonucleoside strand that carries the linker/ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the same strand to the end that carries the linker/ligand moieties.
39. A compound according to Clause 33, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
40. A compound according to Clause 34, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
41. A process of preparing a compound according to any of Clauses 1 to 40, which comprises reacting compounds of Formulae (X*) and (XI*):

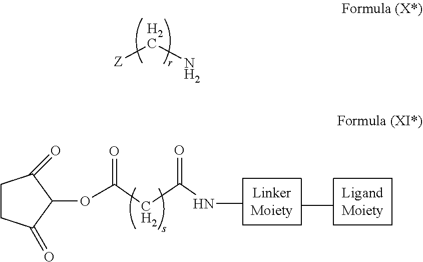

wherein:
r and s are independently an integer selected from 1 to 16; and
Z is an oligonucleoside moiety;
and where appropriate carrying out deprotection of the ligand and/or annealing of a second strand for the oligonucleoside.
42. A process according to Clause 41, to prepare a compound according to any of Clauses 6, 8 to 14, 16 to 33, and 35 to 40, wherein:
compound of Formula (X*) is Formula (Xa*):

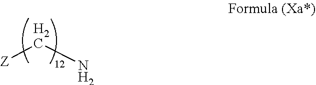

and compound of Formula (XI*) is Formula (XIa*):

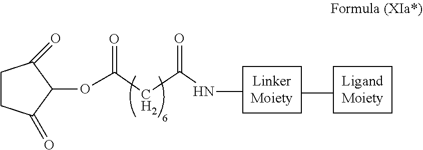

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
43. A process according to Clause 41, to prepare a compound according to any of Clauses 5, 7, 9 to 13, 15 to 32, and 34 to 40, wherein:
compound of Formula (X*) is Formula (Xb*):

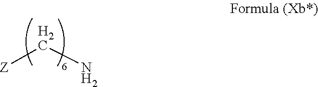

and compound of Formula (XI*) is Formula (XIa*):

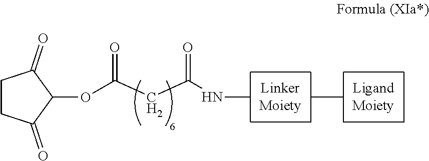

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
44. A process according to Clauses 42 or 43, wherein:
compound of Formula (XIa*) is Formula (XIb*):

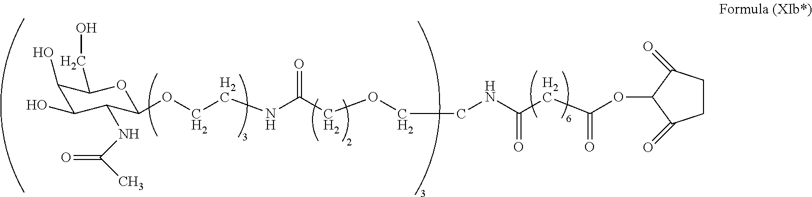

45. A compound of Formula (X*):

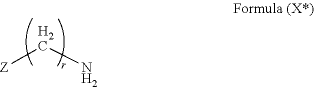

wherein:
r is independently an integer selected from 1 to 16; and
Z is an oligonucleoside moiety.
46. A compound of Formula (Xa*):

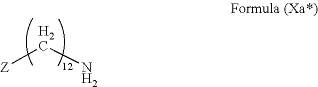

47. A compound of Formula (Xb*):

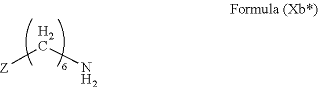

48. A compound of Formula (XI*):

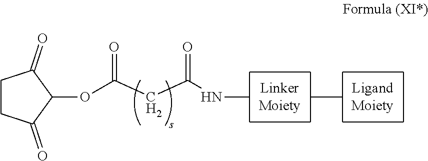

wherein:
s is independently an integer selected from 1 to 16; and
Z is an oligonucleoside moiety.
49. A compound of Formula (XIa*):

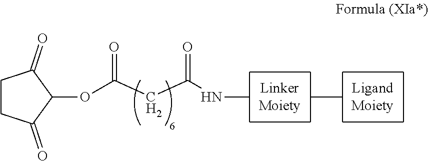

50. A compound of Formula (XIb*):

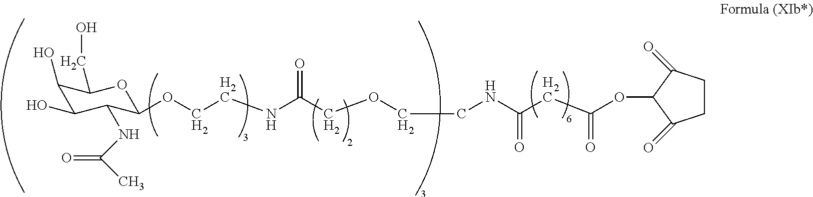

51. Use of a compound according to any of Clauses 45 and 48 to 50, for the preparation of a compound according to any of Clauses 1 to 40.
52. Use of a compound according to Clause 46, for the preparation of a compound according to any of Clauses 6, 8 to 14, 16 to 33, and 35 to 40.
53. Use of a compound according to Clause 47, for the preparation of a compound according to any of Clauses 5, 7, 9 to 13, 15 to 32, and 34 to 40.
54. A compound or composition obtained, or obtainable by a process according to any of Clauses 41 to 44.
55. A pharmaceutical composition comprising of a compound according to any of Clauses 1 to 40, together with a pharmaceutically acceptable carrier, diluent or excipient.
   A compound according to any of Clauses 1 to 40, for use in therapy.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in thet y and are to be included within the spirit and purview of this application and scope of the appended claims.

### Example 1-Target Identification

### BACKGROUND

All biological functions including haemostasis (the physiological control of bleeding) arise from the co-ordinated interaction of hundreds of interacting molecules—primarily proteins—and can be thought of as the emergent functional consequence of protein-protein interaction networks where each node in the network is a protein and each edge linking the proteins can represent a range of possible interaction types from complex formation to catalytic activation etc.

Historically such functions have been represented as simplistic linear pathways. As knowledge has grown however, it has become clear that pathways are more complex and that the minimum level of complexity that adequately represents the functional properties of a biological process, and can capture the characteristics of resilience to perturbation and robustness to random damage of individual components, is a network.

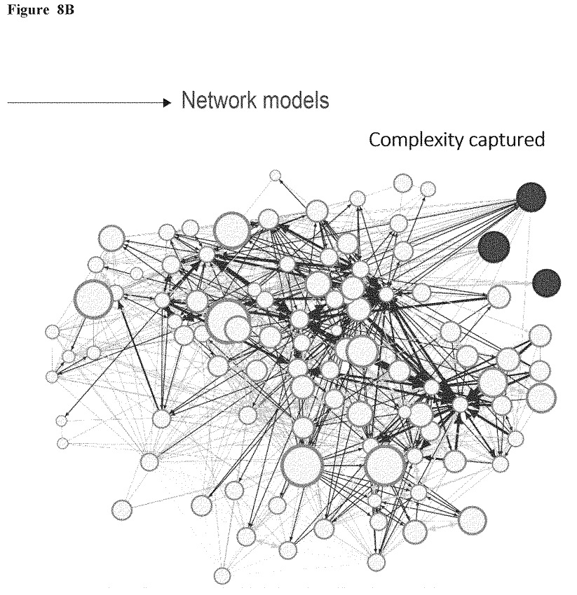

Networks subserving biological function may consist of several interacting canonical pathways as well as additional proteins that are primarily engaged when the canonical function is perturbed. It is therefore critical to develop approaches that can model this complexity in a meaningful and tractable way; and generate target hypotheses that take account of the inherent resistance to change that is a consequence of network robustness. An example of the additional complexity captured by this framework is shown in FIG. 8.

Oversimplification of biology and the failure to make rational drug target choices based on realistic models of biological processes has contributed to the poor success rate of drug discovery. Furthermore, there has been a lack of rigorous objective methods both for making network models of processes and for prioritising protein targets within these models. As a consequence target selection decisions are frequently made on an ad hoc basis based on preference or evidence unrelated to the functional model.

Haemostasis is a complex process that involves many cell types and proteins and which is tightly regulated by a system of pro and anti-haemostatic mechanisms. In haemophilia and in other bleeding disorders there are genetic failures of individual protein components that have varying degrees of impact on the ability of the haemostatic system to control bleeding. There is a large unmet need for well tolerated and safe therapies that improve haemostasis and are easy for patients to comply with.

### Process for Identifying Processes and Targets

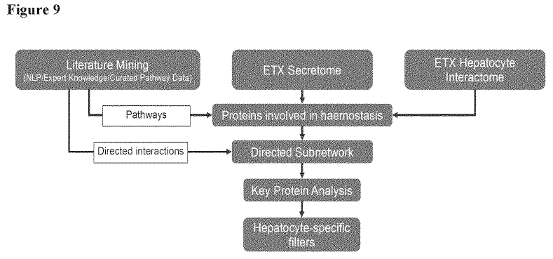

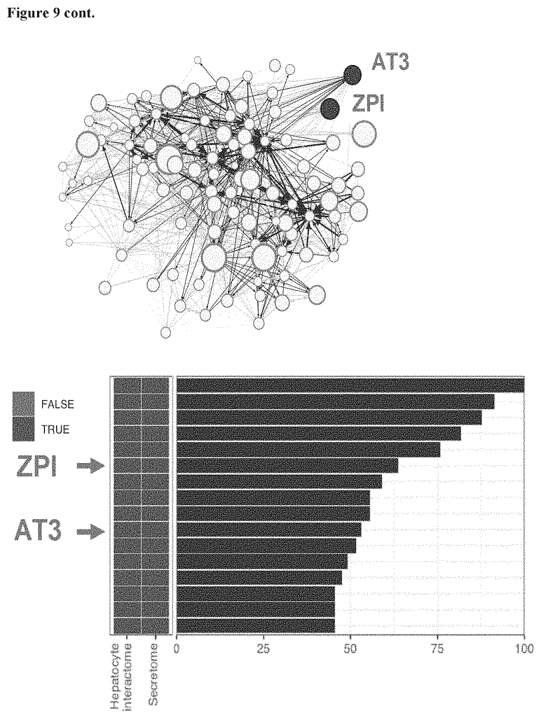

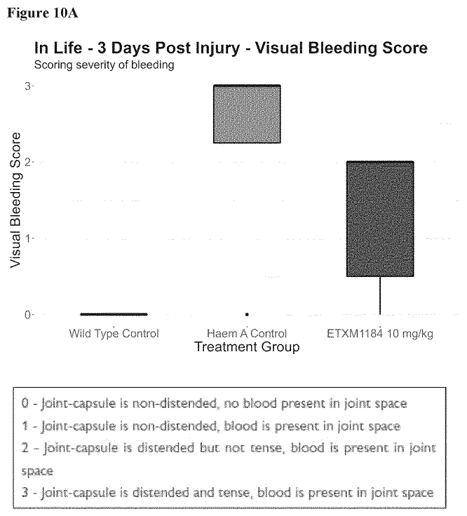

The inventors have used a network biology approach to create network models of haemostasis (FIG. 9). The network models are designed to capture all of the important proteins involved in the process as well as their connections and, importantly, the direction of information flow between pairs of proteins.

The inventors have analysed these network models using proprietary analytical methods. These methods use the directional information to capture key ‘target’ properties such as whether a protein is an integrator of information, a key conduit of information to other parts of the network, an influencer of key proteins and the extent to which an influencer is influenced or influences other proteins (based on absolute and relative number and direction of inputs and outputs). The directional information also enables hierarchical relationships between proteins to be imputed. Proteins higher in the hierarchy and with certain properties may be preferred over others with otherwise similar properties. The relative specificity and magnitude of each property relative to the others made it possible for the inventors to score and rank proteins in terms of their target suitability.

The ability to characterise the properties of these targets in terms of network relationships enables judgements to be made on the selectivity and magnitude of effect in the chosen context and hence the suitability of each for a given indication.

This enabled the inventors to identify targets that will provide improved control of bleeding in haemophilia and other bleeding disorders. The analyses used for this purpose are specifically tailored to find novel and non-obvious indirect potentiators of bleeding whose knockdown by GalNAc-siRNA in hepatocytes will be beneficial in haemophilia by suppressing production of a secreted protein that plays a regulatory role in haemostasis.

In particular, selected groups of haemostasis-related pathway protein sets were used to generate algorithmically a set of directed network models using an internal proprietary database of protein-protein relationships that includes ‘direction’ of interaction. This directional information was derived from a range of public and internal databases supplemented by imputed direction from natural language processing of text from scientific publications. The inventor's network construction algorithms used these sources of information to build ‘directed’ models of the key biological processes.

Proprietary analytical techniques were then applied to the network models to identify pharmacologically viable targets from within the networks whose knockdown will have a significant influence on the network and by extension on the biological function being modeled.

The algorithms make extensive use of directional information and hierarchical relationships to identify targets with a range of specific properties that will make them good siRNA targets. Targets were then further filtered by protein class and hepatocyte specific properties according to the therapeutic requirements.

The above workflow leveraging proprietary data resources and network node metrics has identified two clinically validated modulators of haemostasis that are being developed for treatment of bleeding disorders (AT3 shown above is one of these). In addition, it has highlighted a novel, non-obvious modulator of coagulation, protein Z-dependent protease inhibitor (ZPI) that is predicted by our analytics to be superior to either based on a metric that estimates indirect influence and hence a potentially superior safety profile (FIG. 9).

### Example 2: Synthesis of Tether 1

### General Experimental Conditions

Thin layer chromatography (TLC) was performed on silica-coated aluminium plates with fluorescence indicator 254 nm from Macherey-Nagel. Compounds were visualized under UV light (254 nm), or after spraying with the 5% H₂SO₄ in methanol (MeOH) or ninhydrin reagent according to Stahl (from Sigma-Aldrich), followed by heating. Flash chromatography was performed with a Biotage Isolera One flash chromatography instrument equipped with a dual variable UV wavelength detector (200-400 nm) using Biotage Sfar Silica 10, 25, 50 or 100 g columns (Uppsala, Sweden).

All moisture-sensitive reactions were carried out under anhydrous conditions using dry glassware, anhydrous solvents, and argon atmosphere. All commercially available reagents were purchased from Sigma-Aldrich and solvents from Carl Roth GmbH+Co. KG. D-Galactosamine pentaacetate was purchased from AK scientific.

HPLC/ESI-MS was performed on a Dionex UltiMate 3000 RS UHPLC system and Thermo Scientific MSQ Plus Mass spectrometer using an Acquity UPLC Protein BEH C4 column from Waters (300 Å, 1.7 μm, 2.1×100 mm) at 60° C. The solvent system consisted of solvent A with H₂O containing 0.1% formic acid and solvent B with acetonitrile (ACN) containing 0.1% formic acid. A gradient from 5-100% of B over 15 min with a flow rate of 0.4 mL/min was employed. Detector and conditions: Corona ultra-charged aerosol detection (from esa). Nebulizer Temp.: 25° C. N₂ pressure: 35.1 psi. Filter: Corona. ₁H and ₁₃C NMR spectra were recorded at room temperature on a Varian spectrometer at 500 MHz (₁H NMR) and 125 MHz (₁₃C NMR). Chemical shifts are given in ppm referenced to the solvent residual peak (CDCl₃—₁H NMR: δ at 7.26 ppm and ₁₃C NMR δ at 77.2 ppm; DMSO-d6-₁H NMR: δ at 2.50 ppm and ₁₃C NMR δ at 39.5 ppm). Coupling constants are given in Hertz. Signal splitting patterns are described as singlet (s), doublet (d), triplet (t) or multiplet (m).

### Synthesis Route for the Conjugate Building Block TriGalNAc_Tether1:

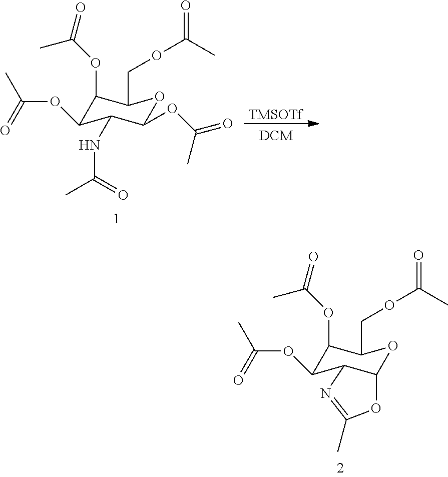 [SMILES: "TMSOTf" ; CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O]

Preparation of compound 2: D-Galactosamine pentaacetate (3.00 g, 7.71 mmol, 1.0 eq.) was dissolved in anhydrous dichloromethane (DCM) (30 mL) under argon and trimethylsilyl trifluoromethanesulfonate (TMSOTf, 4.28 g, 19.27 mmol, 2.5 eq.) was added. The reaction was stirred at room temperature for 3 h. The reaction mixture was diluted with DCM (50 mL) and washed with cold saturated aq. NaHCO₃ (100 mL) and water (100 mL). The organic layer was separated, dried over Na₂SO₄ and concentrated to afford the title compound as yellow oil, which was purified by flash chromatography (gradient elution: 0-10% MeOH in DCM in 10 CV). The product was obtained as colourless oil (2.5 g, 98%, rf=0.45 (2% MeOH in DCM)).

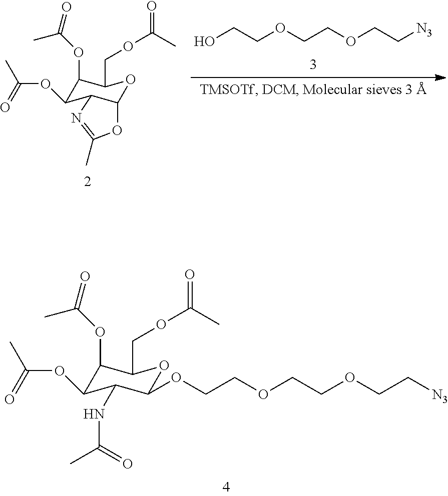

Preparation of compound 4: Compound 2 (2.30 g, 6.98 mmol, 1.0 eq.) and azido-PEG3-OH (1.83 g, 10.5 mmol, 1.5 eq.) were dissolved in anhydrous DCM (40 mL) under argon and molecular sieves 3 Å (5 g) were added to the solution. The mixture was stirred at room temperature for 1 h. TMSOTf (0.77 g, 3.49 mmol, 0.5 eq.) was then added to the mixture and the reaction was stirred overnight. The molecular sieves were filtered, the filtrate was diluted with DCM (100 mL) and washed with cold saturated aq. NaHCO₃ (100 mL) and water (100 mL). The organic layer was separated, dried over Na₂SO₄ and the solvent was removed under reduced pressure. The crude material was purified by flash chromatography (gradient elution: 0-3% MeOH in DCM in 10 CV) to afford the title product as light yellow oil (3.10 g, 88%, rf=0.25 (2% MeOH in DCM)). MS: calculated for C₂₀H₃₂N₄O₁₁, 504.21. Found 505.4. 1H NMR (500 MHz, CDCl3) δ 6.21-6.14 (m, 1H), 5.30 (dd, J=3.4, 1.1 Hz, 1H), 5.04 (dd, J=11.2, 3.4 Hz, 1H), 4.76 (d, J=8.6 Hz, 1H), 4.23-4.08 (m, 3H), 3.91-3.80 (m, 3H), 3.74-3.59 (m, 9H), 3.49-3.41 (m, 2H), 2.14 (s, 3H), 2.02 (s, 3H), 1.97 (d, J=4.2 Hz, 6H). 13C NMR (125 MHz, CDCl3) δ 170.6 (C), 170.5 (C), 170.4 (C), 170.3 (C), 102.1 (CH), 71.6 (CH), 70.8 (CH), 70.6 (CH), 70.5 (CH), 70.3 (CH2), 69.7 (CH2), 68.5 (CH2), 66.6 (CH2), 61.5 (CH2), 23.1 (CH3), 20.7 (3×CH3).

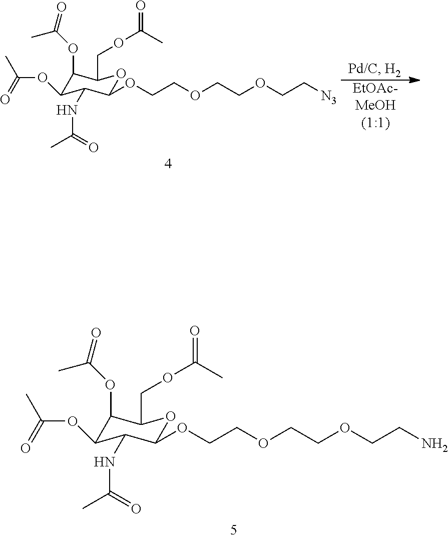 [SMILES: CC(=O)NC1C(OCCOCCOCCN=[N+]=[N-])OC(COC(C)=O)C(OC(C)=O)C1OC(C)=O ; CC(=O)NC1C(OCCOCCOCCN)OC(COC(C)=O)C(OC(C)=O)C1OC(C)=O]

Preparation of compound 5: Compound 4 (1.00 g, 1.98 mmol, 1.0 eq.) was dissolved in a mixture of ethyl acetate (EtOAc) and MeOH (30 mL 1:1 v/v) and Pd/C (100 mg) was added. The reaction mixture was degassed using vacuum/argon cycles (3×) and hydrogenated under balloon pressure overnight. The reaction mixture was filtered through celite and washed with EtOAc (30 mL). The solvent was removed under reduced pressure to afford the title compound as colourless oil (0.95 g, quantitative yield, rf=0.25 (10% MeOH in DCM)). The compound was used without further purification. MS: calculated for C₂₀H₃₄N₂O₁₁, 478.2. Found 479.4.

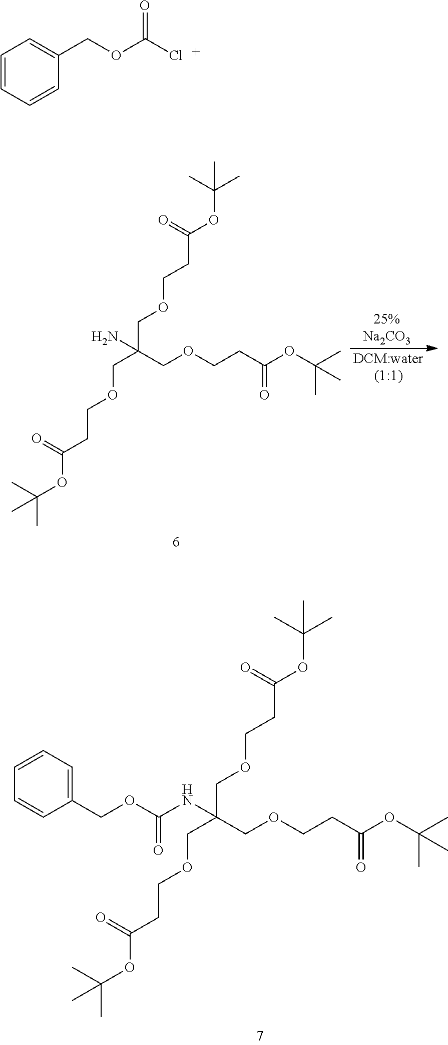 [SMILES: O=C(Cl)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)OCc1ccccc1]

Preparation of compound 7: Tris{[2-(tert-butoxycarbonyl)ethoxy]methyl}-methylamine 6 (3.37 g, 6.67 mmol, 1.0 eq.) was dissolved in a mixture of DCM/water (40 mL 1:1 v/v) and Na₂CO₃ (0.18 g, 1.7 mmol, 0.25 eq.) was added while stirring vigorously. Benzyl chloroformate (2.94 mL, 20.7 mmol, 3.10 eq.) was added dropwise to the previous mixture and the reaction was stirred at room temperature for 24 h. The reaction mixture was diluted with CH₂Cl₂ (100 mL) and washed with water (100 mL). The organic layer was separated and dried over Na2SO4. The solvent was removed under reduced pressure and the resulting crude material was purified by flash chromatography (gradient elution: 0-10% EtOAc in cyclohexane in 12 CV) to afford the title compound as pale yellowish oil (3.9 g, 91%, rf=0.56 (10% EtOAc in cyclohexane)). MS: calculated for C₃₃H₅₃NO₁₁, 639.3. Found 640.9. 1H NMR (500 MHz, DMSO-d6) δ 7.38-7.26 (m, 5H), 4.97 (s, 2H), 3.54 (t, 6H), 3.50 (s, 6H), 2.38 (t, 6H), 1.39 (s, 27H). 13C NMR (125 MHz, DMSO-d6) δ 170.3 (3×C), 154.5 (C), 137.1 (C), 128.2 (2×CH), 127.7 (CH), 127.6 (2×CH), 79.7 (3×C), 68.4 (3×CH2), 66.8 (3×CH2), 64.9 (C), 58.7 (CH2), 35.8 (3×CH2), 27.7 (9×CH3).

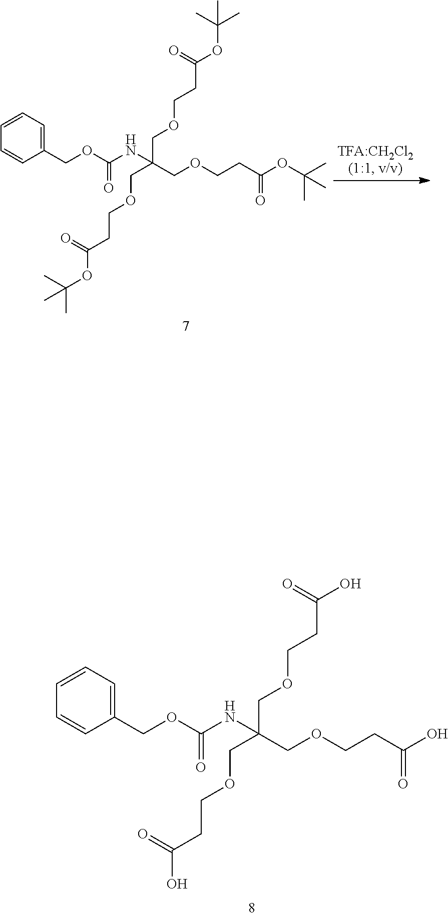 [SMILES: CC(C)(C)OC(=O)CCOCC(COCCC(=O)OC(C)(C)C)(COCCC(=O)OC(C)(C)C)NC(=O)OCc1ccccc1 ; O=C(O)CCOCC(COCCC(=O)O)(COCCC(=O)O)NC(=O)OCc1ccccc1]

Preparation of compound 8: Cbz-NH-tris-Boc-ester 7 (0.20 g, 0.39 mmol, 1.0 eq.) was dissolved in CH₂Cl₂ (1 mL) under argon, trifluoroacetic acid (TFA, 1 mL) was added and the reaction was stirred at room temperature for 1 h. The solvent was removed under reduced pressure, the residue was co-evaporated 3 times with toluene (5 mL) and dried under high vacuum to get the compound as its TFA salt (0.183 g, 98%). The compound was used without further purification. MS: calculated for C₂₁H₂₉NO₁₁, 471.6. Found 472.4.

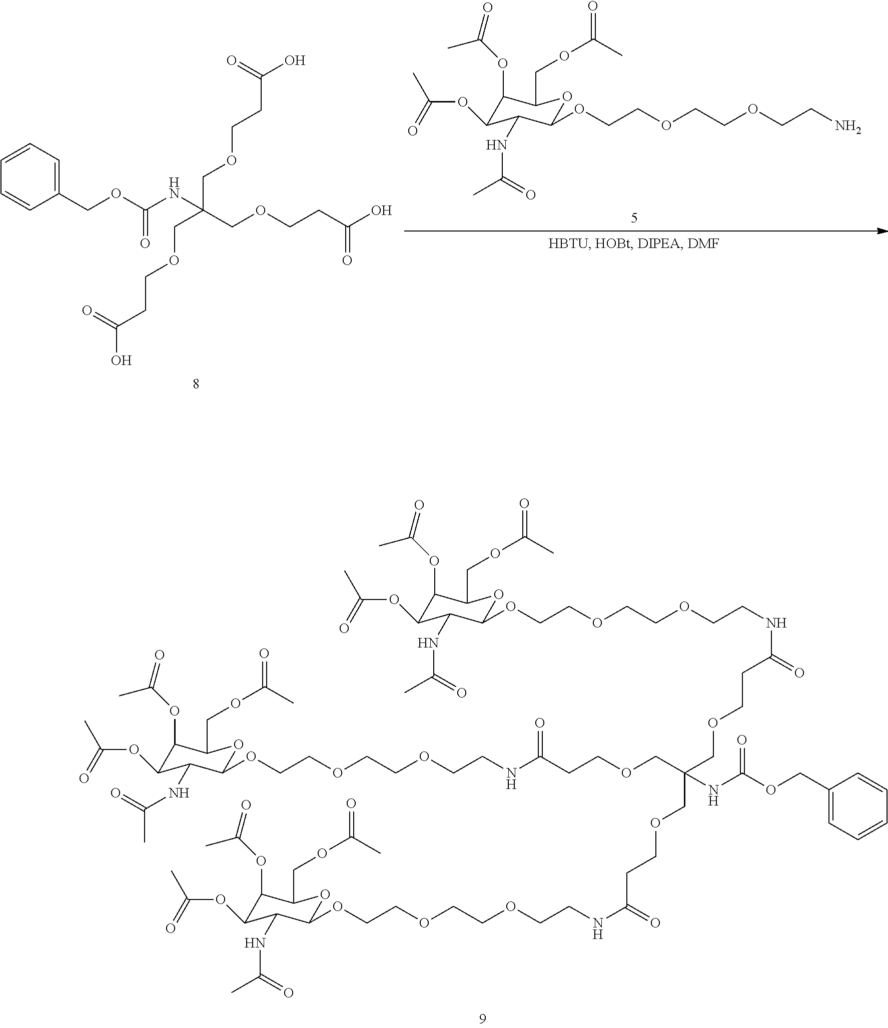 [SMILES: O=C(O)CCOCC(COCCC(=O)O)(COCCC(=O)O)NC(=O)OCc1ccccc1 ; CC(=O)NC1C(OCCOCCOCCN)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O]

Preparation of compound 9: CbzNH-tris-COOH 8 (0.72 g, 1.49 mmol, 1.0 eq.) and GalNAc-PEG3-NH₂ 5 (3.56 g, 7.44 mmol, 5.0 eq.) were dissolved in N,N-dimethylformamide (DMF) (25 mL). Then N,N,N′,N′-tetramethyl-O-(1H-benzotriazol-1-yl)uronium hexafluorophosphate (HBTU) (2.78 g, 7.44 mmol, 5.0 eq.), 1-hydroxybenzotriazole hydrate (HOBt) (1.05 g, 7.44 mmol, 5.0 eq.) and N,N-diisopropylethylamine (DIPEA) (2.07 mL, 11.9 mmol, 8.0 eq.) were added to the solution and the reaction was stirred for 72 h. The solvent was removed under reduced pressure, the residue was dissolved in DCM (100 mL) and washed with saturated aq. NaHCO₃ (100 mL). The organic layer was dried over Na₂SO₄, the solvent evaporated and the crude material was purified by flash chromatography (gradient elution: 0-5% MeOH in DCM in 14 CV). The product was obtained as pale yellowish oil (1.2 g, 43%, rf=0.20 (5% MeOH in DCM)). MS: calculated for C₈₁H₁₂₅N₇O₄₁, 1852.9. Found 1854.7. 1H NMR (500 MHz, DMSO-d6) δ 7.90-7.80 (m, 10H), 7.65-7.62 (m, 4H), 7.47-7.43 (m, 3H), 7.38-7.32 (m, 8H), 5.24-5.22 (m, 3H), 5.02-4.97 (m, 4H), 4.60-4.57 (m, 3H), 4.07-3.90 (m 10H), 3.67-3.36 (m, 70H), 3.23-3.07 (m, 25H), 2.18 (s, 10H), 2.00 (s, 13H), 1.89 (s, 11H), 1.80-1.78 (m, 17H). 13C NMR (125 MHz, DMSO-d6) δ 170.1 (C), 169.8 (C), 169.7 (C), 169.4 (C), 169.2 (C), 169.1 (C), 142.7 (C), 126.3 (CH), 123.9 (CH), 118.7 (CH), 109.7 (CH), 100.8 (CH), 70.5 (CH), 69.8 (CH), 69.6 (CH), 69.5 (CH), 69.3 (CH2), 69.0 (CH2), 68.2 (CH2), 67.2 (CH2), 66.7 (CH2), 61.4 (CH2), 22.6 (CH2), 22.4 (3×CH3), 20.7 (9×CH3).

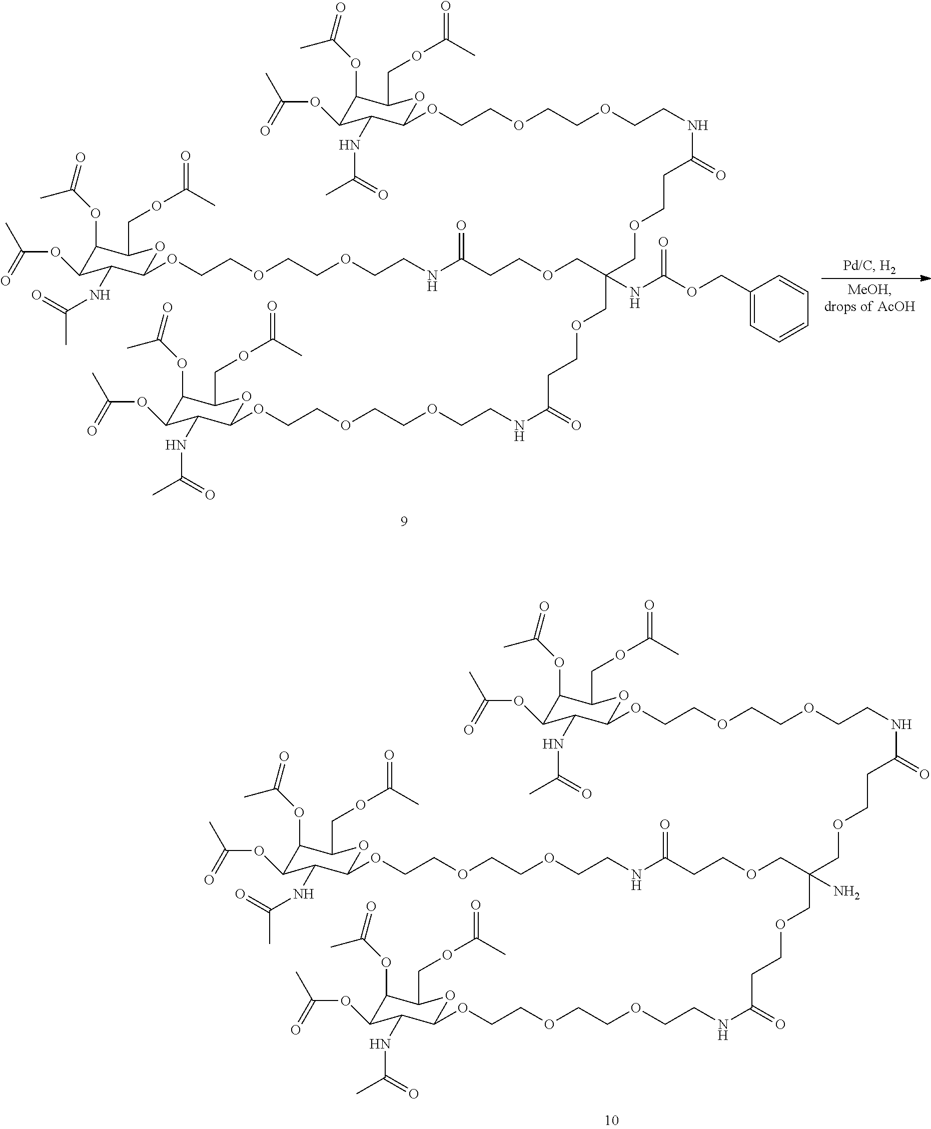 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O ; CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

Preparation of compound 10: Triantennary GalNAc compound 9 (0.27 g, 0.14 mmol, 1.0 eq.) was dissolved in MeH (15 mL), 3 drops of acetic acid (AcOH) and Pd/C (30 mg) was added. The reaction mixture was degassed using vacuum/argon cycles (3×) and hydrogenated under balloon pressure overnight. The completion of the reaction was followed by mass spectrometry and the resulting mixture was filtered through a thin pad of celite. The solvent was evaporated and the residue obtained was dried under high vacuum and used for the next step without further purification. The product was obtained as pale yellowish oil (0.24 g, quantitative yield). MS: calculated for C₇₃H₁₁₉N₇O₃₉, 1718.8. Found 1719.3.

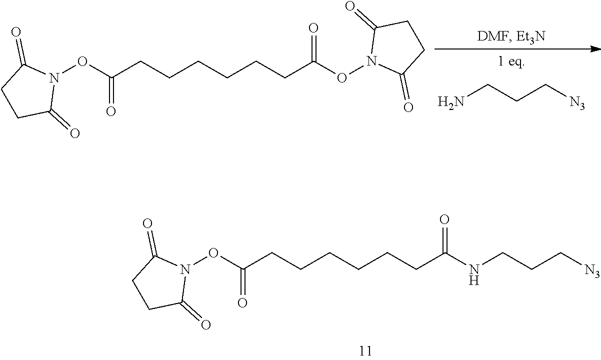 [SMILES: O=C(CCCCCCC(=O)ON1C(=O)CCC1=O)ON1C(=O)CCC1=O ; [N-]=[N+]=NCCCN ; [N-]=[N+]=NCCCNC(=O)CCCCCCC(=O)ON1C(=O)CCC1=O]

Preparation of compound 11: Commercially available suberic acid bis(N-hydroxysuccinimide ester) (3.67 g, 9.9 mmol, 1.0 eq.) was dissolved in DMF (5 mL) and triethylamine (1.2 mL) was added. To this solution was added dropwise a solution of 3-azido-1-propylamine (1.0 g, 9.9 mmol, 1.0 eq.) in DMF (5 mL). The reaction was stirred at room temperature for 3 h. The reaction mixture was diluted with EtOAc (100 mL) and washed with water (50 mL). The organic layer was separated, dried over Na₂SO₄ and the solvent was removed under reduced pressure. The crude material was purified by flash chromatography (gradient elution: 0-5% MeOH in DCM in 16 CV). The product was obtained as white solid (1.54 g, 43%, rf=0.71 (5% MeOH in DCM)). MS: calculated for C₁₅H₂₃N₅O₅, 353.4. Found 354.3.

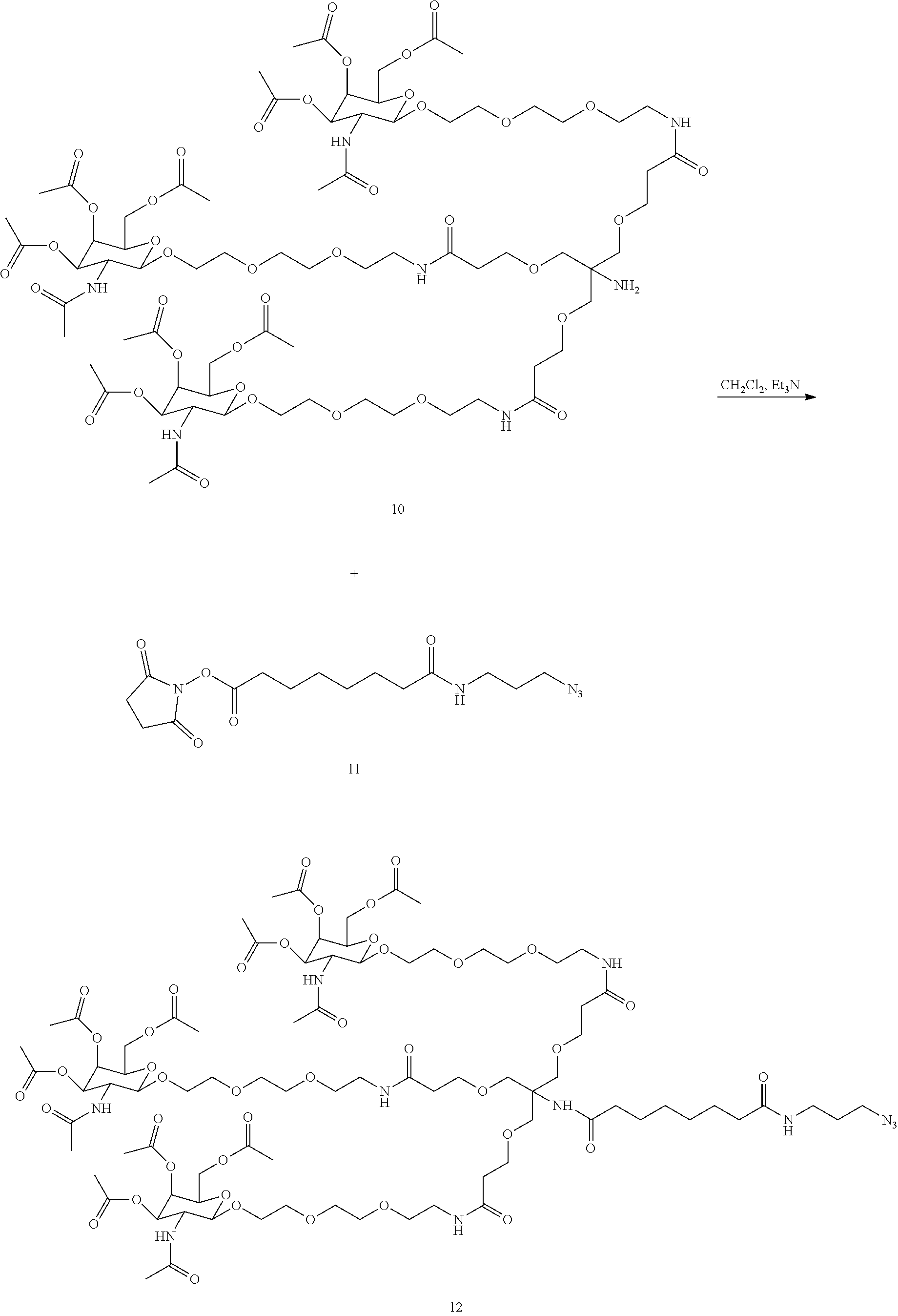 [SMILES: CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; [N-]=[N+]=NCCCNC(=O)CCCCCCC(=O)ON1C(=O)CCC1=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)NCCCN=[N+]=[N-])OC(COC(C)=O)C(OC(C)=O)C1OC(C)=O]

Preparation of TriGalNAc (12): Triantennary GalNAc compound 10 (0.35 g, 0.24 mmol, 1.0 eq.) and compound 11 (0.11 g, 0.31 mmol, 1.5 eq.) were dissolved in DCM (5 mL) under argon and triethylamine (0.1 mL, 0.61 mmol, 3.0 eq.) was added. The reaction was stirred at room temperature overnight. The solvent was removed under reduced pressure, the residue was dissolved in EtOAc (100 mL) and washed with water (100 mL). The organic layer was separated and dried over Na₂SO₄. The solvent was evaporated and the resulting crude material was purified by flash chromatography (elution gradient: 0-10% MeOH in DCM in 20 CV) to afford the title compound as white fluffy solid (0.27 g, 67%, rf=0.5 (10% MeOH in DCM)). MS: calculated for C₈₄H₁₃₇N₁₁O₄₁, 1957.1. Found 1959.6.

Conjugation of Tether 1 to a siRNA Strand: Monofluoro Cyclooctyne (MFCO) Conjugation at 5′- or 3′-End

### 5′-End MFCO Conjugation

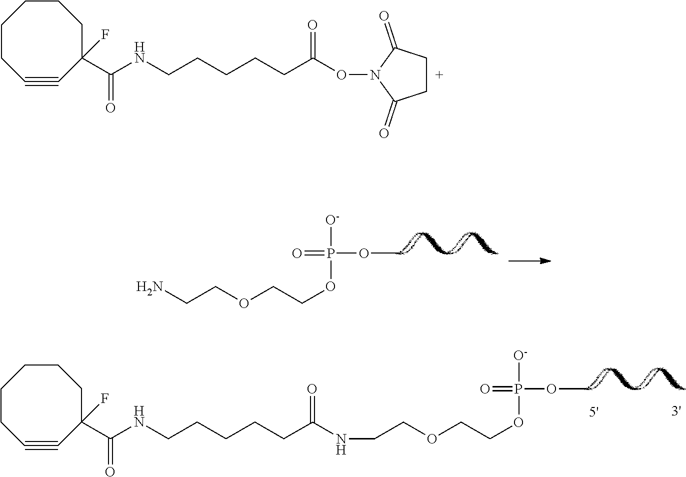 [SMILES: O=C(CCCCCNC(=O)C1(F)C#CCCCCC1)ON1C(=O)CCC1=O ; COP(=O)([O-])OCCOCCN ; COP(=O)([O-])OCCOCCNC(=O)CCCCCNC(=O)C1(F)C#CCCCCC1]

3′-end MFCO conjugation

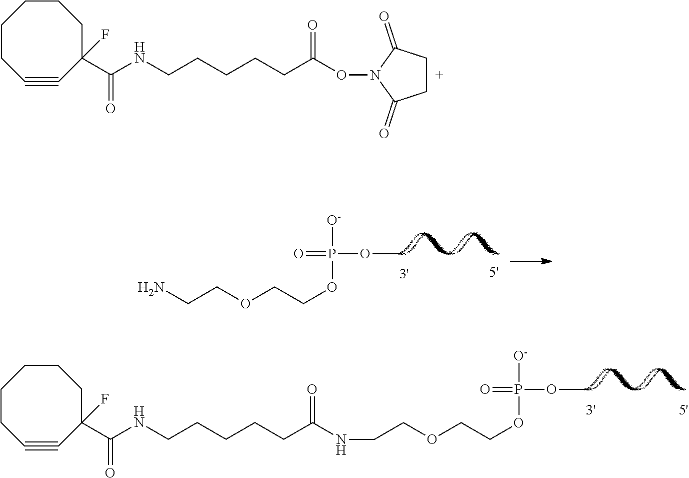 [SMILES: O=C(CCCCCNC(=O)C1(F)C#CCCCCC1)ON1C(=O)CCC1=O ; COP(=O)([O-])OCCOCCN ; COP(=O)([O-])OCCOCCNC(=O)CCCCCNC(=O)C1(F)C#CCCCCC1]

General conditions for MFCO conjugation: Amine-modified single strand was dissolved at 700 OD/mL in 50 mM carbonate/bicarbonate buffer pH 9.6/dimethyl sulfoxide (DMSO) 4:6 (v/v) and to this solution was added one molar equivalent of a 35 mM solution of MFCO-C6-NHS ester (Berry&Associates, Cat. #LK 4300) in DMF. The reaction was carried out at room temperature and after 1 h another molar equivalent of the MFCO solution was added. The reaction was allowed to proceed for an additional hour and was monitored by LC/MS. At least two molar equivalent excess of the MFCO NHS ester reagent relative to the amino modified oligonucleotide were needed to achieve quantitative consumption of the starting material. The reaction mixture was diluted 15-fold with water, filtered through a 1.2 μm filter from Sartorius and then purified by reserve phase (RP HPLC) on an Akta Pure instrument (GE Healthcare).

Purification was performed using a XBridge C18 Prep 19×50 mm column from Waters. Buffer A was 100 mM TEAAc pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full length conjugated oligonucleotide were pooled, precipitated in the freezer with 3 M NaOAc, pH 5.2 and 85% ethanol and the collected pellet was dissolved in water. Samples were desalted by size exclusion chromatography and concentrated using a speed-vac concentrator to yield the conjugated oligonucleotide in an isolated yield of 40-80%.

### 5′-GalNAc-T1 Conjugates

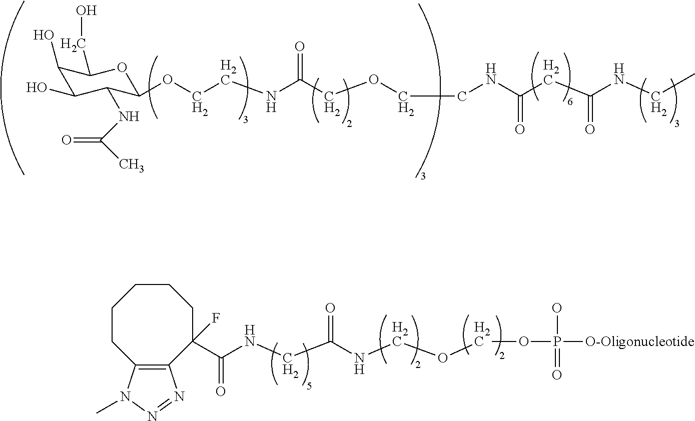

### 3′-GalNAc-T1 Conjugates

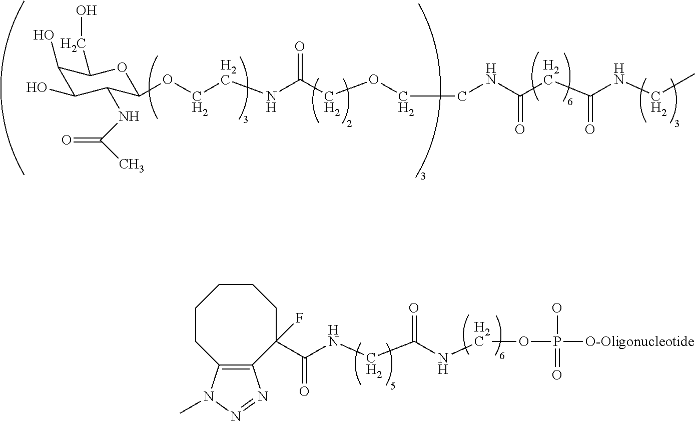

General procedure for TriGalNAc conjugation: MFCO-modified single strand was dissolved at 2000 OD/mL in water and to this solution was added one equivalent solution of compound 12 (10 mM) in DMF. The reaction was carried out at room temperature and after 3 h 0.7 molar equivalent of the compound 12 solution was added. The reaction was allowed to proceed overnight and completion was monitored by LCMS. The conjugate was diluted 15-fold in water, filtered through a 1.2 μm filter from Sartorius and then purified by RP OaPLC on an Akta Pure instrument (GE Healthcare).

RP HIPLC purification was performed using a XBridge C18 Prep 19×50 mm column from Waters. Buffer A was 100 mM triethyl ammonium acetate pH 7 and buffer B contained 9500 acetonitrile in buffer A. A flow rate of 10 mL/min and a temperature of 60° C. were employed. UV traces at 280 nm were recorded. A gradient of0-100 B within 60 column volumes was employed.

Fractions containing full-length conjugated oligonucleotide were pooled, precipitated in the freezer with 3 M NaOAc, pH 5.2 and 8500 ethanol and the collected pellet was dissolved in water to give an oligonucleotide solution of about 1000 OD/mL. The 0-acetates were removed by adding 20% aqueous ammonia. Quantitative removal of these protecting groups was verified by LC-MS.

The conjugates were desalted by size exclusion chromatography using Sephadex G25 Fine resin (GE Healthcare) on an Akta Pure (GE Healthcare) instrument to yield the conjugated oligonucleotides in an isolated yield of 50-70%.

The following schemes further set out the routes of synthesis:

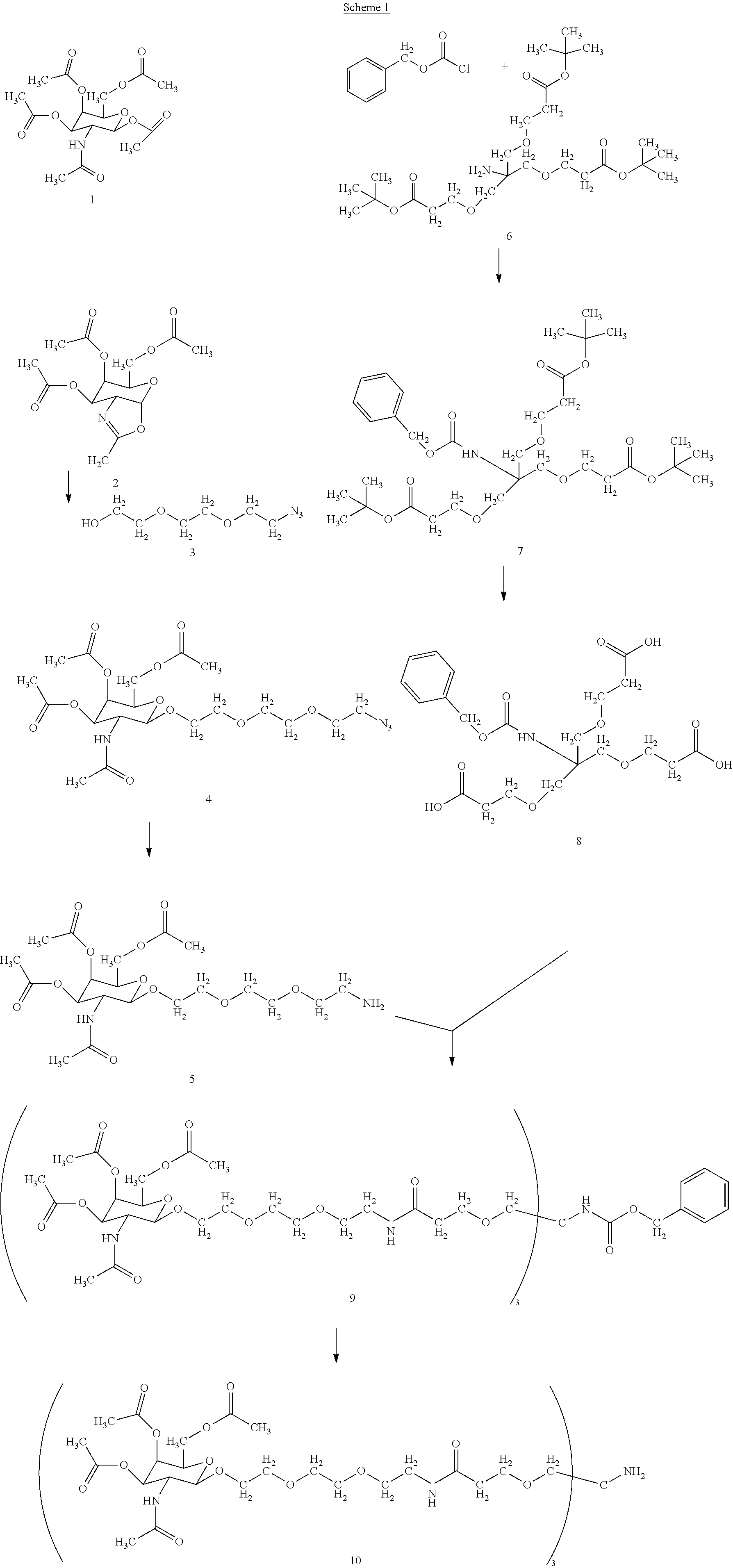

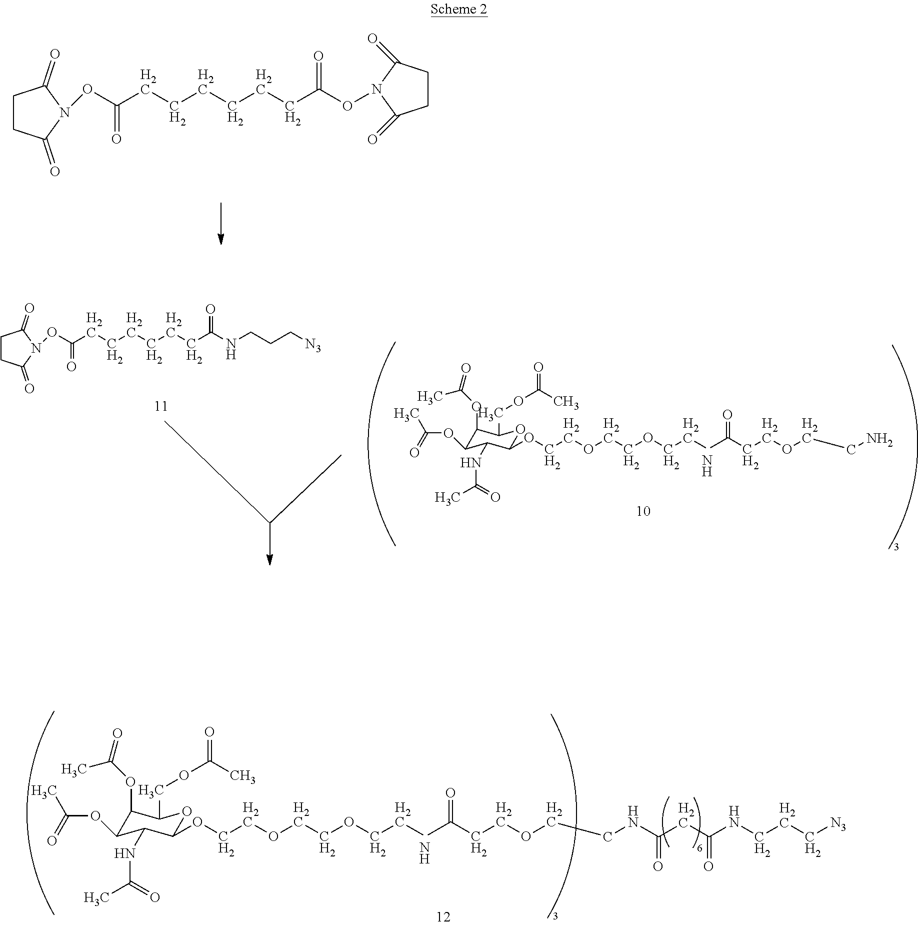

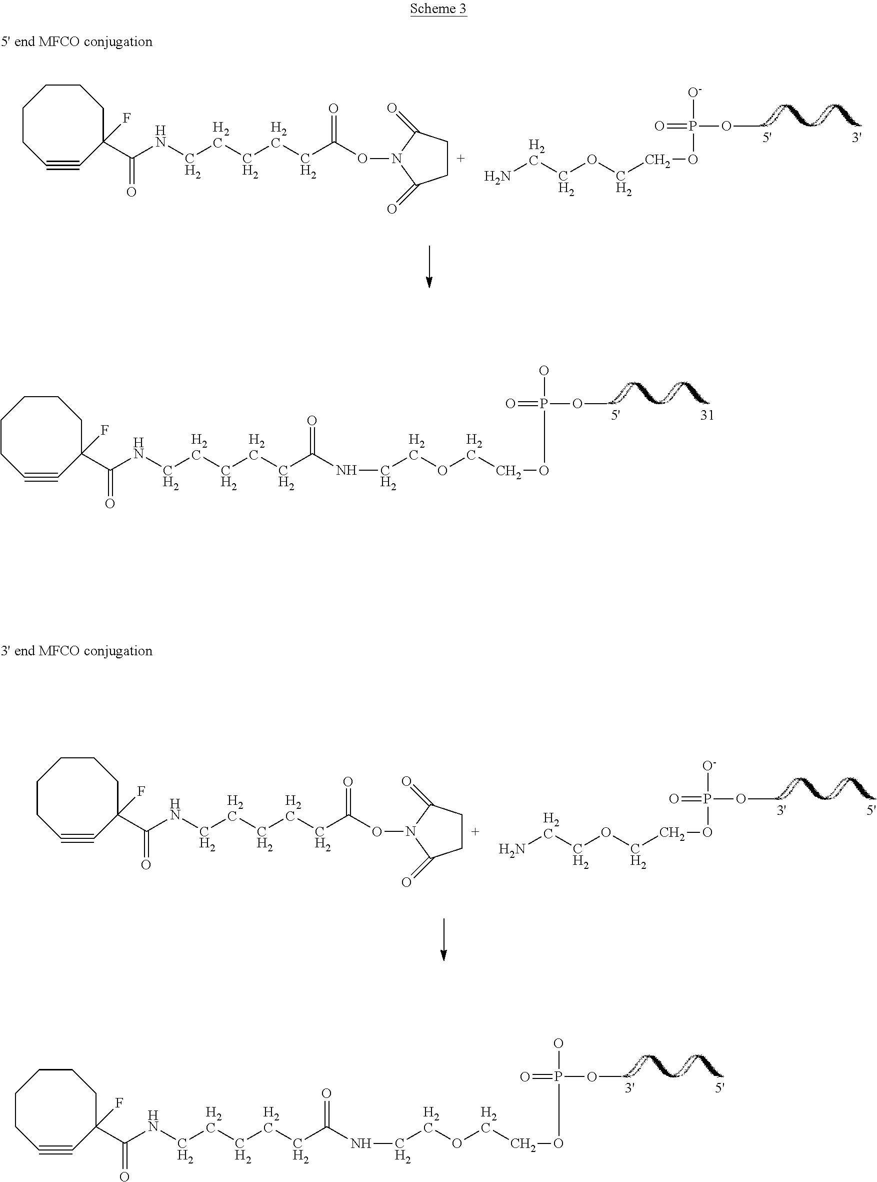 [SMILES: O=C(CCCCCNC(=O)C1(F)C#CCCCCC1)ON1C(=O)CCC1=O ; COP(=O)([O-])OCCOCCN ; COP(=O)(O)COCCOCCNC(=O)CCCCCNC(=O)C1(F)C#CCCCCC1 ; O=C(CCCCCNC(=O)C1(F)C#CCCCCC1)ON1C(=O)CCC1=O ; COP(=O)([O-])OCCOCCN ; COP(=O)(O)COCCOCCNC(=O)CCCCCNC(=O)C1(F)C#CCCCCC1]

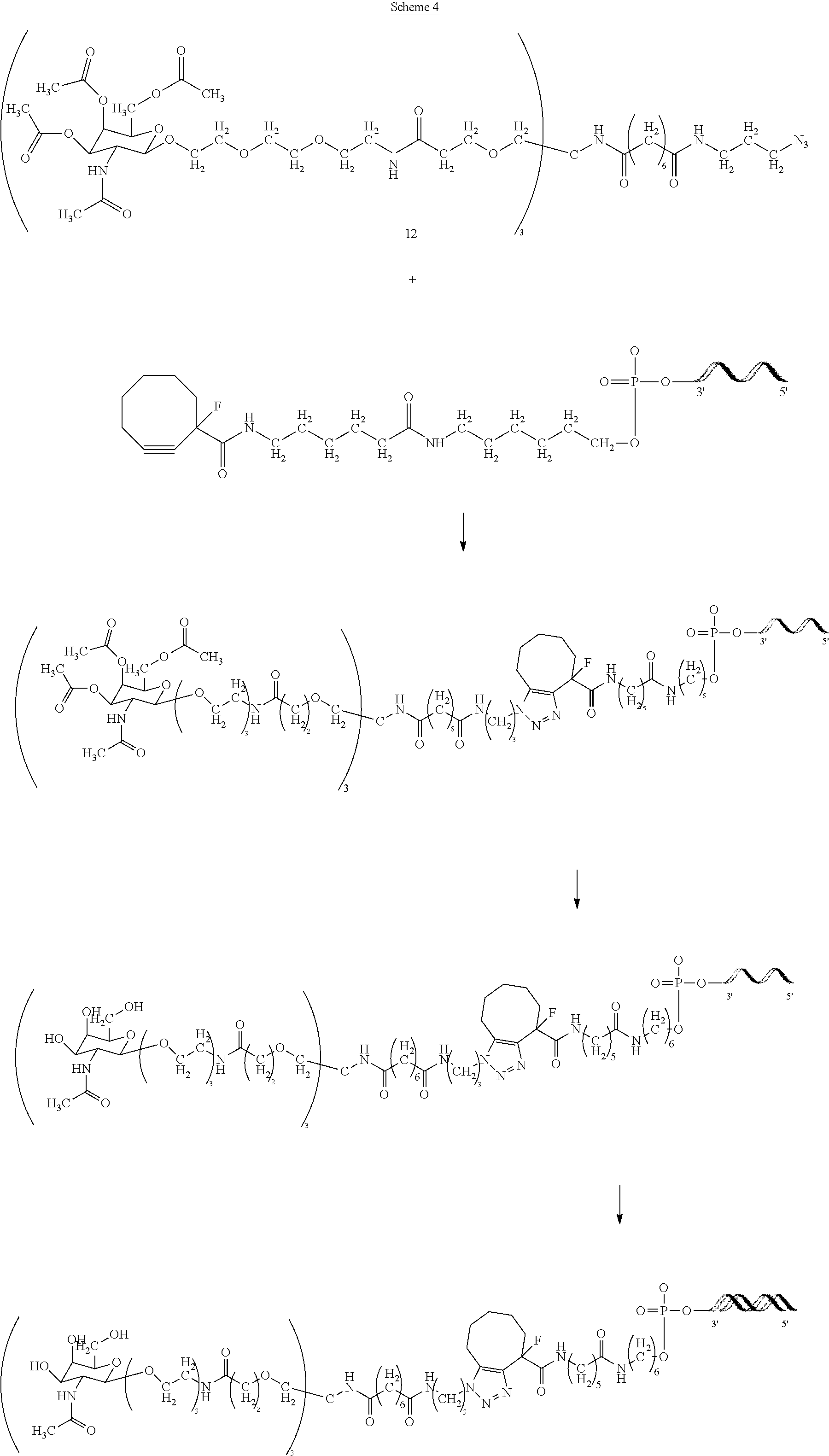

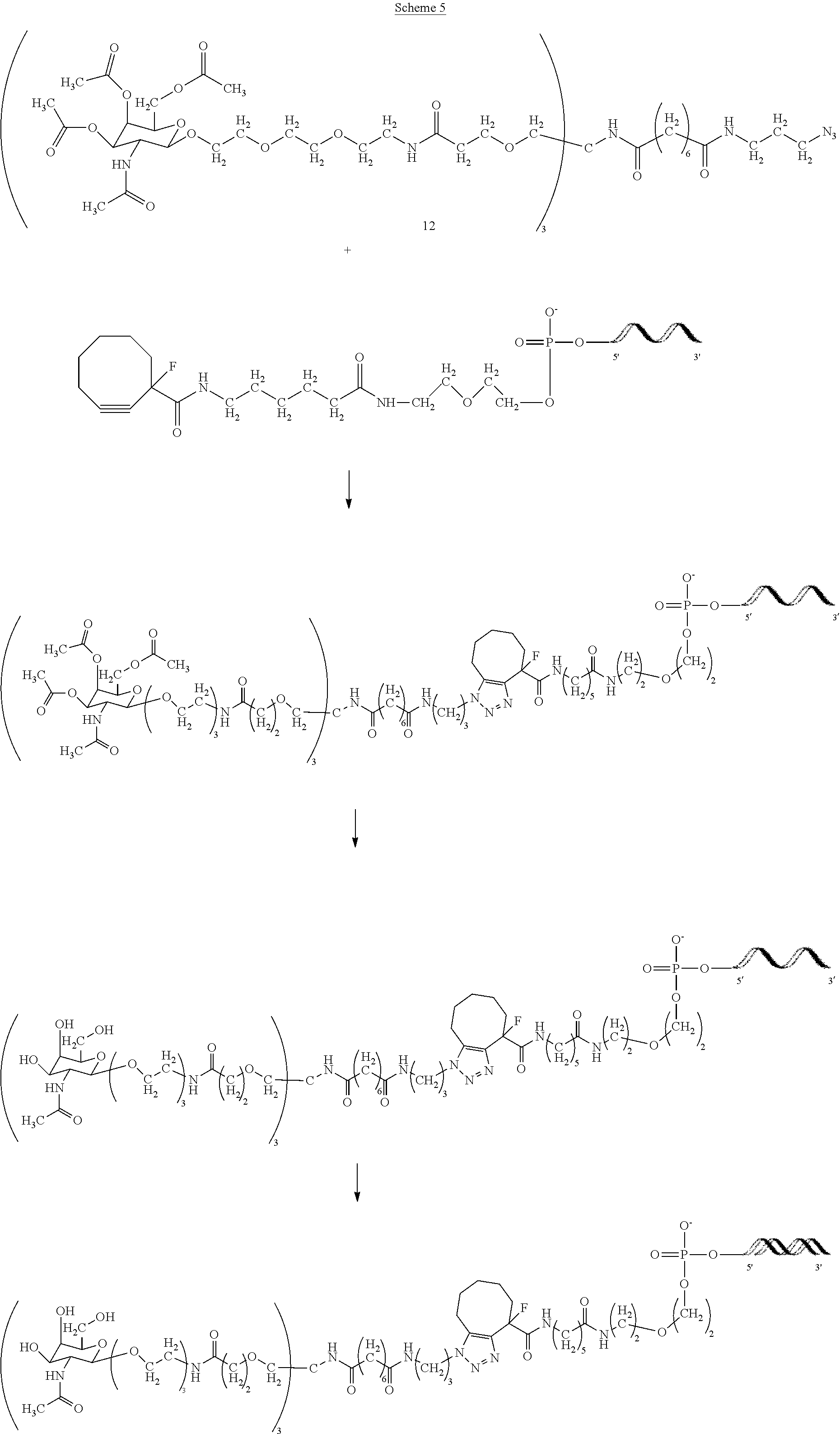

### Example 3: Duplex Annealing

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The duplexes were lyophilized for 2 days and stored at −20° C.

The duplexes were analyzed by analytical SEC HPLC on Superdex™ 75 Increase 5/150 GL column 5×153-158 mm (Cytiva) on a Dionex Ultimate 3000 (Thermo Fisher Scientific) HPLC system. Mobile phase consisted of 1×PBS containing 10% acetonitrile. An isocratic gradient was run in 10 min at a flow rate of 1.5 mL/min at room temperature. UV traces at 260 and 280 nm were recorded. Water (LC-MS grade) was purchased from Sigma-Aldrich and Phosphate-buffered saline (PBS; 10×, pH 7.4) was purchased from GIBCO (Thermo Fisher Scientific).

### Example 4: Synthesis of Tether 2

### General Experimental Conditions

Thin layer chromatography (TLC) was performed on silica-coated aluminium plates with fluorescence indicator 254 nm from Macherey-Nagel. Compounds were visualized under UV light (254 nm), or after spraying with the 5% H₂SO₄ in methanol (MeOH) or ninhydrin reagent according to Stahl (from Sigma-Aldrich), followed by heating. Flash chromatography was performed with a Biotage Isolera One flash chromatography instrument equipped with a dual variable UV wavelength detector (200-400 nm) using Biotage Sfar Silica 10, 25, 50 or 100 g columns (Uppsala, Sweden).

All moisture-sensitive reactions were carried out under anhydrous conditions using dry glassware, anhydrous solvents, and argon atmosphere. All commercially available reagents were purchased from Sigma-Aldrich and solvents from Carl Roth GmbH+Co. KG. D-Galactosamine pentaacetate was purchased from AK scientific.

HPLC/ESI-MS was performed on a Dionex UltiMate 3000 RS UHPLC system and Thermo Scientific MSQ Plus Mass spectrometer using an Acquity UPLC Protein BEH C4 column from Waters (300 Å, 1.7 μm, 2.1×100 mm) at 60° C. The solvent system consisted of solvent A with H2O containing 0.1% formic acid and solvent B with acetonitrile (ACN) containing 0.1% formic acid. A gradient from 5-100% of B over 15 min with a flow rate of 0.4 mL/min was employed. Detector and conditions: Corona ultra-charged aerosol detection (from esa). Nebulizer Temp.: 25° C. N₂ pressure: 35.1 psi. Filter: Corona.

₁H and ₁₃C NMR spectra were recorded at room temperature on a Varian spectrometer at 500 MHz (₁H NMR) and 125 MHz (₁₃C NMR). Chemical shifts are given in ppm referenced to the solvent residual peak (CDCl₃—₁H NMR: δ at 7.26 ppm and ₁₃C NMR δ at 77.2 ppm; DMSO-d6-₁H NMR: δ at 2.50 ppm and ₁₃C NMR δ at 39.5 ppm). Coupling constants are given in Hertz. Signal splitting patterns are described as singlet (s), doublet (d), triplet (t) or multiplet (m).

### Synthesis Route for the Conjugate Building Block TriGalNAc_Tether2:

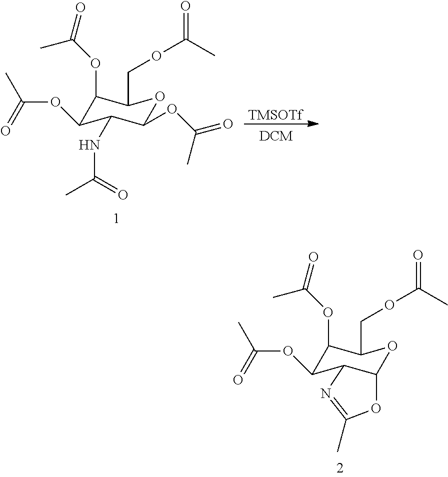 [SMILES: "TMSOTf" ; CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O]

Preparation of compound 2: D-Galactosamine pentaacetate (3.00 g, 7.71 mmol, 1.0 eq.) was dissolved in anhydrous dichloromethane (DCM) (30 mL) under argon and trimethylsilyl trifluoromethanesulfonate (TMSOTf, 4.28 g, 19.27 mmol, 2.5 eq.) was added. The reaction was stirred at room temperature for 3 h. The reaction mixture was diluted with DCM (50 mL) and washed with cold saturated aq. NaHCO₃ (100 mL) and water (100 mL). The organic layer was separated, dried over Na₂SO₄, and concentrated to afford the title compound as yellow oil, which was purified by flash chromatography (gradient elution: 0-10% MeOH in DCM in 10 CV). The product was obtained as colourless oil (2.5 g, 98%, rf=0.45 (2% MeOH in DCM)).

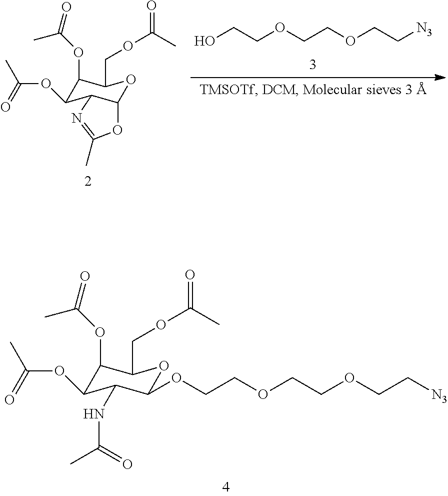

Preparation of compound 4: Compound 2 (2.30 g, 6.98 mmol, 1.0 eq.) and azido-PEG3-OH (1.83 g, 10.5 mmol, 1.5 eq.) were dissolved in anhydrous DCM (40 mL) under argon and molecular sieves 3 Å (5 g) were added to the solution. The mixture was stirred at room temperature for 1 h. TMSOTf (0.77 g, 3.49 mmol, 0.5 eq.) was then added to the mixture and the reaction was stirred overnight. The molecular sieves were filtered, the filtrate was diluted with DCM (100 mL) and washed with cold saturated aq. NaHCO₃ (100 mL) and water (100 mL). The organic layer was separated, dried over Na2SO4 and the solvent was removed under reduced pressure. The crude material was purified by flash chromatography (gradient elution: 0-3% MeOH in DCM in 10 CV) to afford the title product as light-yellow oil (3.10 g, 88%, rf=0.25 (2% MeOH in DCM)). MS: calculated for C₂₀H₃₂N₄O₁₁, 504.21. Found 505.4. 1H NMR (500 MHz, CDCl3) δ 6.21-6.14 (m, 1H), 5.30 (dd, J=3.4, 1.1 Hz, 1H), 5.04 (dd, J=11.2, 3.4 Hz, 1H), 4.76 (d, J=8.6 Hz, 1H), 4.23-4.08 (m, 3H), 3.91-3.80 (m, 3H), 3.74-3.59 (m, 9H), 3.49-3.41 (m, 2H), 2.14 (s, 3H), 2.02 (s, 3H), 1.97 (d, J=4.2 Hz, 6H). 13C NMR (125 MHz, CDCl3) δ 170.6 (C), 170.5 (C), 170.4 (C), 170.3 (C), 102.1 (CH), 71.6 (CH), 70.8 (CH), 70.6 (CH), 70.5 (CH), 70.3 (CH2), 69.7 (CH2), 68.5 (CH2), 66.6 (CH2), 61.5 (CH2), 23.1 (CH3), 20.7 (3×CH3).

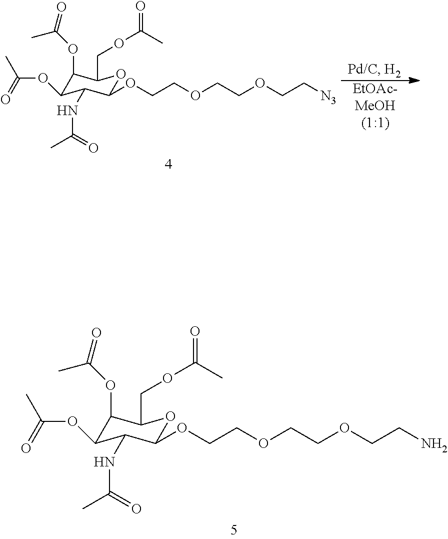 [SMILES: CC(=O)NC1C(OCCOCCOCCN=[N+]=[N-])OC(COC(C)=O)C(OC(C)=O)C1OC(C)=O ; CC(=O)NC1C(OCCOCCOCCN)OC(COC(C)=O)C(OC(C)=O)C1OC(C)=O]

Preparation of compound 5: Compound 4 (1.00 g, 1.98 mmol, 1.0 eq.) was dissolved in a mixture of ethyl acetate (EtOAc) and MeOH (30 mL 1:1 v/v) and Pd/C (100 mg) was added. The reaction mixture was degassed using vacuum/argon cycles (3×) and hydrogenated under balloon pressure overnight. The reaction mixture was filtered through celite and washed with EtOAc (30 mL). The solvent was removed under reduced pressure to afford the title compound as colourless oil (0.95 g, quantitative yield, rf=0.25 (10% MeOH in DCM)). The compound was used without further purification. MS: calculated for C₂₀H₃₄N₂O₁₁, 478.2. Found 479.4.

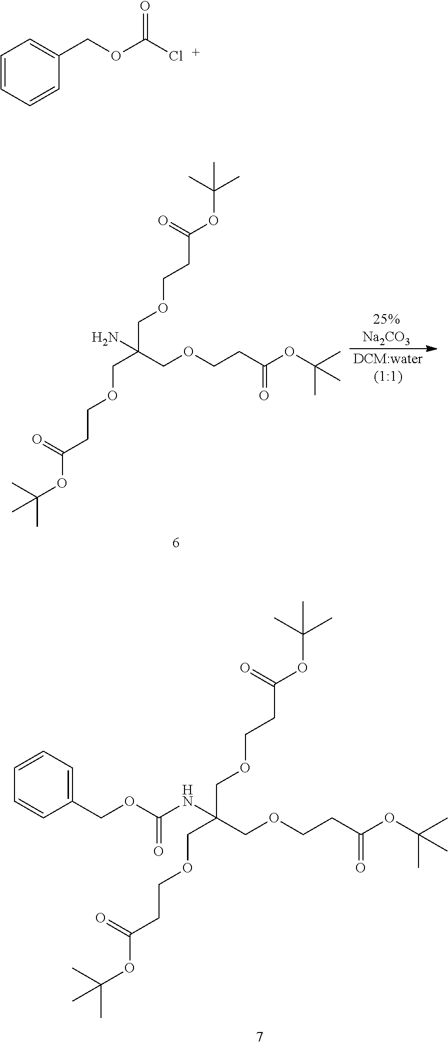 [SMILES: O=C(Cl)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)OCc1ccccc1]

Preparation of compound 7: Tris{[2-(tert-butoxycarbonyl)ethoxy]methyl}-methylamine 6 (3.37 g, 6.67 mmol, 1.0 eq.) was dissolved in a mixture of DCM/water (40 mL 1:1 v/v) and Na₂CO₃ (0.18 g, 1.7 mmol, 0.25 eq.) was added while stirring vigorously. Benzyl chloroformate (2.94 mL, 20.7 mmol, 3.10 eq.) was added dropwise to the previous mixture and the reaction was stirred at room temperature for 24 h. The reaction mixture was diluted with CH₂Cl₂ (100 mL) and washed with water (100 mL). The organic layer was separated and dried over Na₂SO₄. The solvent was removed under reduced pressure and the resulting crude material was purified by flash chromatography (gradient elution: 0-10% EtOAc in cyclohexane in 12 CV) to afford the title compound as pale yellowish oil (3.9 g, 91%, rf=0.56 (10% EtOAc in cyclohexane)). MS: calculated for C₃₃H₅₃NO₁₁, 639.3. Found 640.9. 1H NMR (500 MHz, DMSO-d6) δ 7.38-7.26 (m, 5H), 4.97 (s, 2H), 3.54 (t, 6H), 3.50 (s, 6H), 2.38 (t, 6H), 1.39 (s, 27H). 13C NMR (125 MHz, DMSO-d6) δ 170.3 (3×C), 154.5 (C), 137.1 (C), 128.2 (2×CH), 127.7 (CH), 127.6 (2×CH), 79.7 (3×C), 68.4 (3×CH2), 66.8 (3×CH2), 64.9 (C), 58.7 (CH2), 35.8 (3×CH2), 27.7 (9×CH3).

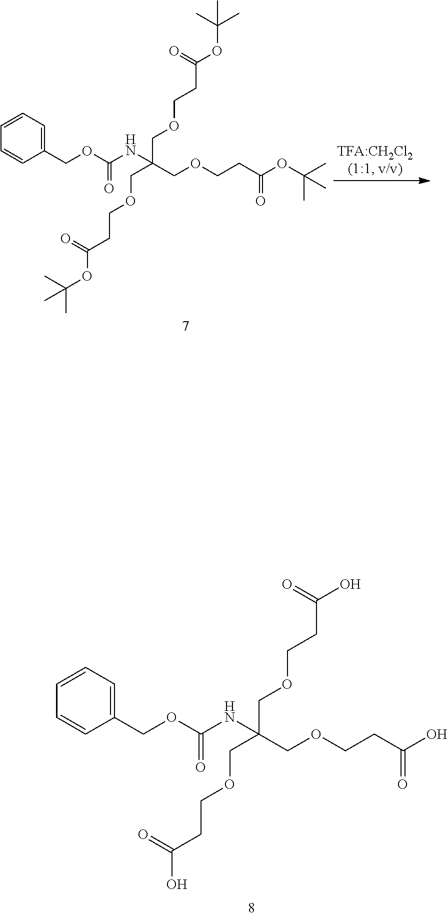 [SMILES: CC(C)(C)OC(=O)CCOCC(COCCC(=O)OC(C)(C)C)(COCCC(=O)OC(C)(C)C)NC(=O)OCc1ccccc1 ; O=C(O)CCOCC(COCCC(=O)O)(COCCC(=O)O)NC(=O)OCc1ccccc1]

Preparation of compound 8: Cbz-NH-tris-Boc-ester 7 (0.20 g, 0.39 mmol, 1.0 eq.) was dissolved in CH₂Cl₂ (1 mL) under argon, trifluoroacetic acid (TFA, 1 mL) was added and the reaction was stirred at room temperature for 1 h. The solvent was removed under reduced pressure, the residue was co-evaporated 3 times with toluene (5 mL) and dried under high vacuum to get the compound as its TFA salt (0.183 g, 98%). The compound was used without further purification. MS: calculated for C₂₁H₂₉NO₁₁, 471.6. Found 472.4.

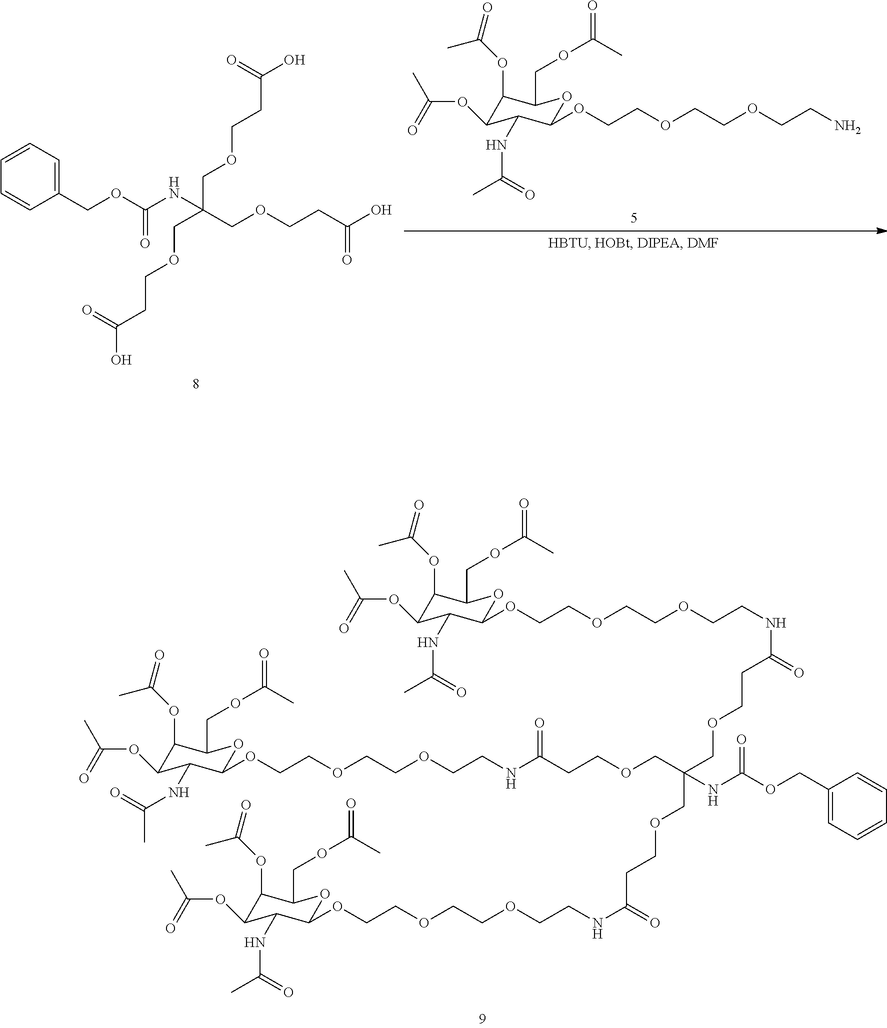 [SMILES: O=C(O)CCOCC(COCCC(=O)O)(COCCC(=O)O)NC(=O)OCc1ccccc1 ; CC(=O)NC1C(OCCOCCOCCN)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O]

Preparation of compound 9: CbzNH-tris-COOH 8 (0.72 g, 1.49 mmol, 1.0 eq.) and GalNAc-PEG3-NH2 5 (3.56 g, 7.44 mmol, 5.0 eq.) were dissolved in N,N-dimethylformamide (DMF) (25 mL). Then N,N,N′,N′-tetramethyl-O-(1H-benzotriazol-1-yl)uronium hexafluorophosphate (HBTU) (2.78 g, 7.44 mmol, 5.0 eq.), 1-hydroxybenzotriazole hydrate (HOBt) (1.05 g, 7.44 mmol, 5.0 eq.) and N,N-diisopropylethylamine (DIPEA) (2.07 mL, 11.9 mmol, 8.0 eq.) were added to the solution and the reaction was stirred for 72 h. The solvent was removed under reduced pressure, the residue was dissolved in DCM (100 mL) and washed with saturated aq. NaHCO₃ (100 mL). The organic layer was dried over Na₂SO₄, the solvent evaporated and the crude material was purified by flash chromatography (gradient elution: 0-5% MeOH in DCM in 14 CV). The product was obtained as pale yellowish oil (1.2 g, 43%, rf=0.20 (5% MeOH in DCM)). MS: calculated for C₈₁H₁₂₅N₇O₄₁, 1852.9. Found 1854.7. 1H NMR (500 MHz, DMSO-d6) δ 7.90-7.80 (m, 10H), 7.65-7.62 (m, 4H), 7.47-7.43 (m, 3H), 7.38-7.32 (m, 8H), 5.24-5.22 (m, 3H), 5.02-4.97 (m, 4H), 4.60-4.57 (m, 3H), 4.07-3.90 (m 10H), 3.67-3.36 (m, 70H), 3.23-3.07 (m, 25H), 2.18 (s, 10H), 2.00 (s, 13H), 1.89 (s, 11H), 1.80-1.78 (m, 17H). 13C NMR (125 MHz, DMSO-d6) δ 170.1 (C), 169.8 (C), 169.7 (C), 169.4 (C), 169.2 (C), 169.1 (C), 142.7 (C), 126.3 (CH), 123.9 (CH), 118.7 (CH), 109.7 (CH), 100.8 (CH), 70.5 (CH), 69.8 (CH), 69.6 (CH), 69.5 (CH), 69.3 (CH2), 69.0 (CH2), 68.2 (CH2), 67.2 (CH2), 66.7 (CH2), 61.4 (CH2), 22.6 (CH2), 22.4 (3×CH3), 20.7 (9×CH3).

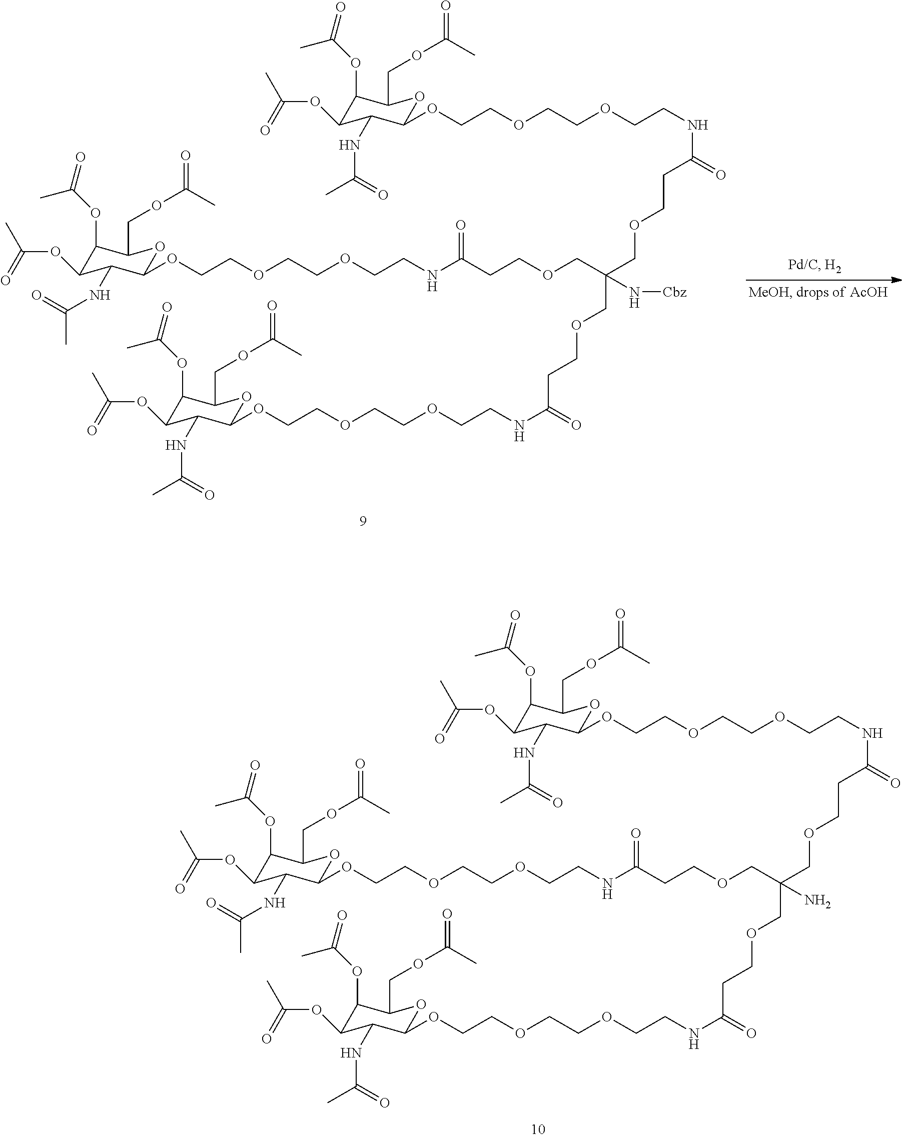 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O ; CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

Preparation of compound 10: Triantennary GaNAc compound 9 (0.27 g, 0.14 mmol, 1.0 eq.) was dissolved in MeOH (15 mL), 3 drops of acetic acid (AcOH) and Pd/C (30 mg) was added. The reaction mixture was degassed using vacuum/argon cycles (3×) and hydrogenated under balloon pressure overnight. The completion of the reaction was followed by mass spectrometry and the resulting mixture was filtered through a thin pad of celite. The solvent was evaporated, and the residue obtained was dried under high vacuum and used for the next step without further purification. The product was obtained as pale yellowish oil (0.24 g, quantitative yield). MS: calculated for C₇₃H₁₁₉N₇O₃₉, 1718.8. Found 1719.3.

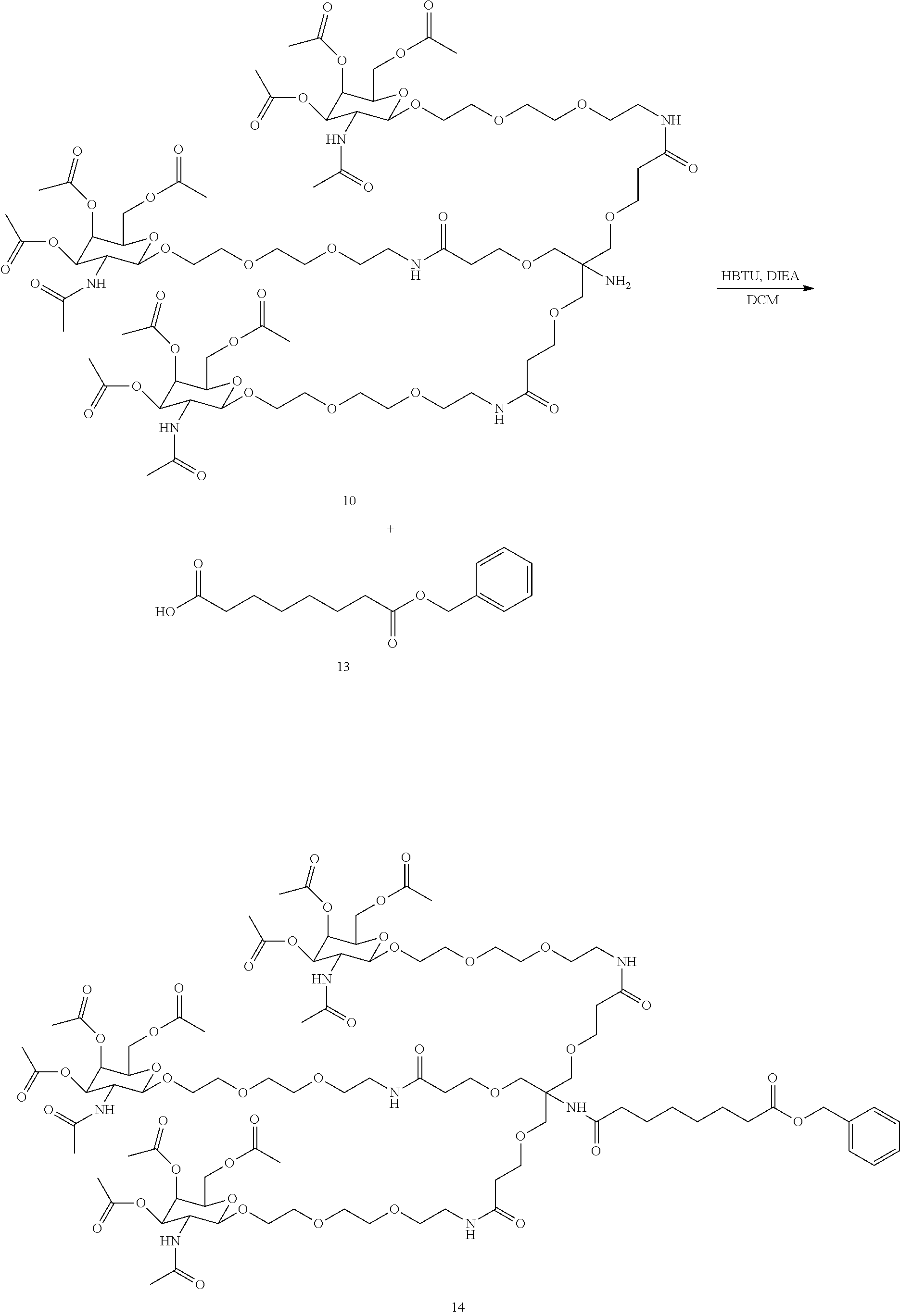 [SMILES: CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; O=C(O)CCCCCCC(=O)OCc1ccccc1 ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O]

Preparation of compound 14: Triantennary GalNAc compound 10 (0.45 g, 0.26 mmol, 1.0 eq.), HBTU (0.19 g, 0.53 mmol, 2.0 eq.) and DIPEA (0.23 mL, 1.3 mmol, 5.0 eq.) were dissolved in DCM (10 mL) under argon. To this mixture, it was added dropwise a solution of compound 13 (0.14 g, 0.53 mmol, 2.0 eq.) in DCM (5 mL). The reaction was stirred at room temperature overnight. The solvent was removed, and the residue was dissolved in EtOAc (50 mL), washed with water (50 mL) and dried over Na₂SO₄. The solvent was evaporated, and the crude material was purified by flash chromatography (gradient elution: 0-5% MeOH in DCM in 20 CV). The product was obtained as white fluffy solid (0.25 g, 48%, rf=0.4 (10% MeOH in DCM)). MS: calculated for C₈₈H₁₃₇N₇O₄₂, 1965.1. Found 1965.6.

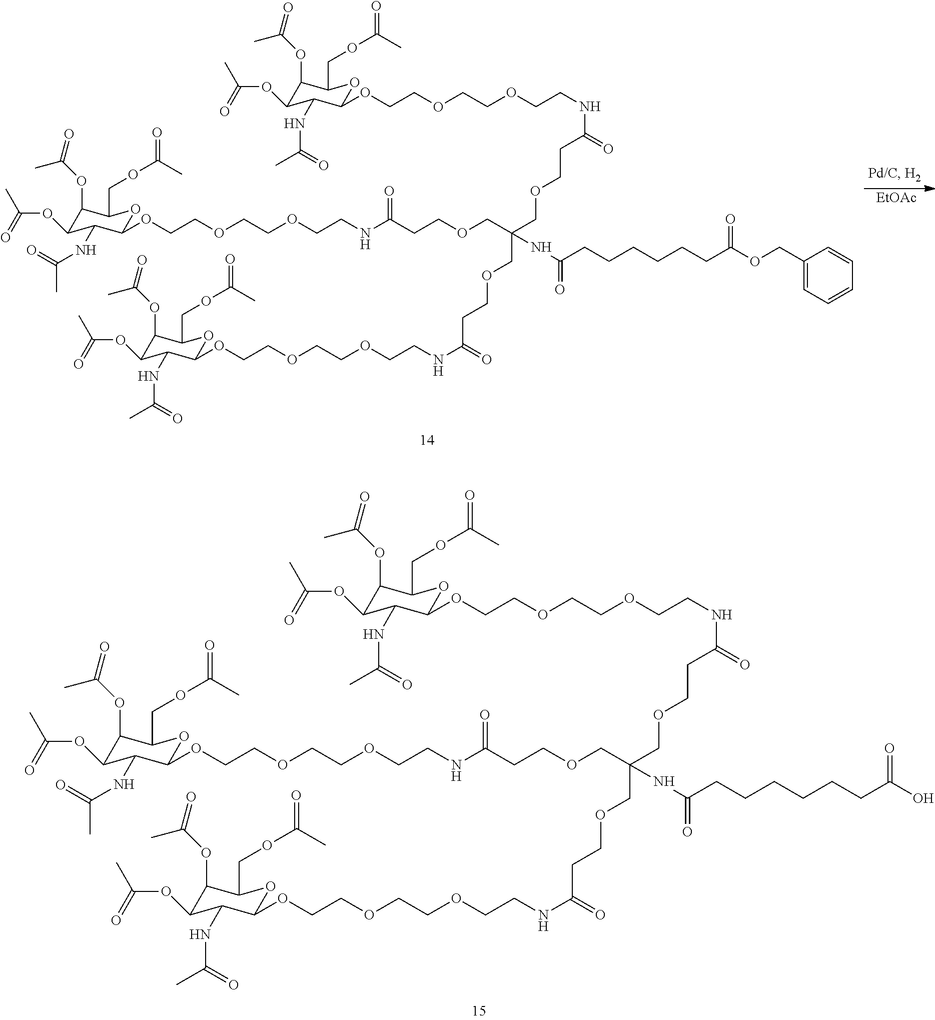 [SMILES: "EtOAc" ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)O)OC(COC(C)=O)C(OC(C)=O)C1OC(C)=O]

Preparation of TriGalNAc (15): Triantennary GalNAc compound 14 (0.31 g, 0.15 mmol, 1.0 eq.) was dissolved in EtOAc (15 mL) and Pd/C (40 mg) was added. The reaction mixture was degassed by using vacuum/argon cycles (3×) and hydrogenated under balloon pressure overnight. The completion of the reaction was monitored by mass spectrometry and the resulting mixture was filtered through a thin pad of celite. The solvent was removed under reduced pressure and the resulting residue was dried under high vacuum overnight. The residue was used for conjugations to oligonucleosides without further purification (0.28 g, quantitative yield). MS: calculated for C₈₁H₁₃₁N₇O₄₂, 1874.9. Found 1875.3.

Conjugation of Tether 2 to a siRNA strand: TriGalNAc tether 2 (GalNAc-T2) conjugation at 5′-end or 3′-end

### 5′-GalNAc-T2 Conjugates

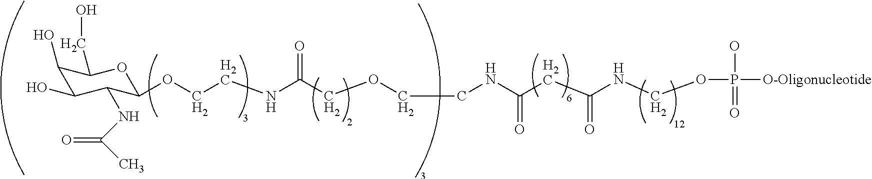

### 3′-Ga2NAc-T2 Conjugates

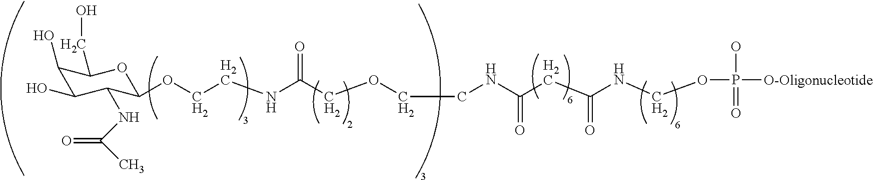

Preparation of TriGalNAc tether 2 NHS ester: To a solution of carboxylic acid tether 2 (compound 15, 227 mg, 121 μmol) in DMF (2.1 mL), N-hydroxysuccinimide (NHS) (15.3 mg, 133 μmol) and N,N′-diisopropylcarbodiimide (DIC) (19.7 μL, 127 μmol) were added. The solution was stirred at room temperature for 18 h and used without purification for the subsequent conjugation reactions.

General procedure for triGalNAc tether 2 conjugation: Amine-modified single strand was dissolved at 700 OD/mL in 50 mM carbonate/bicarbonate buffer pH 9.6/DMSO 4:6 (v/v) and to this solution was added one molar equivalent of Tether 2 NHS ester (57 mM) solution in DMF. The reaction was carried out at room temperature and after 1 h another molar equivalent of the NHS ester solution was added. The reaction was allowed to proceed for one more hour and reaction progress was monitored by LCMS. At least two molar equivalent excess of the NHS ester reagent relative to the amino modified oligonucleoside were needed to achieve quantitative consumption of the starting material. The reaction mixture was diluted 15-fold with water, filtered once through 1.2 μm filter from Sartorius and then purified by reserve phase (RP HPLC) on an Äkta Pure (GE Healthcare) instrument.

The purification was performed using a XBridge C18 Prep 19×50 mm column from Waters. Buffer A was 100 mM TEAA pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full-length conjugated oligonucleosides were pooled together, precipitated in the freezer with 3 M NaOAc, pH 5.2 and 85% ethanol and then dissolved at 1000 OD/mL in water. The O-acetates were removed with 20% ammonium hydroxide in water until completion (monitored by LC-MS).

The conjugates were desalted by size exclusion chromatography using Sephadex G25 Fine resin (GE Healthcare) on an Akta Pure (GE Healthcare) instrument to yield the conjugated oligonucleotides in an isolated yield of 60-80%.

The conjugates were characterized by HPLC-MS analysis with a 2.1×50 mm XBridge C18 column (Waters) on a Dionex Ultimate 3000 (Thermo Fisher Scientific) HPLC system equipped with a Compact ESI-Qq-TOF mass spectrometer (Bruker Daltonics). Buffer A was 16.3 mM triethylamine, 100 mM HFIP in 1% MeOH in H20 and buffer B contained 95% MeOH in buffer A. A flow rate of 250 μL/min and a temperature of 60° C. were employed. UV traces at 260 and 280 nm were recorded. A gradient of 1-100% B within 31 min was employed.

The following schemes further set out the routes of synthesis:

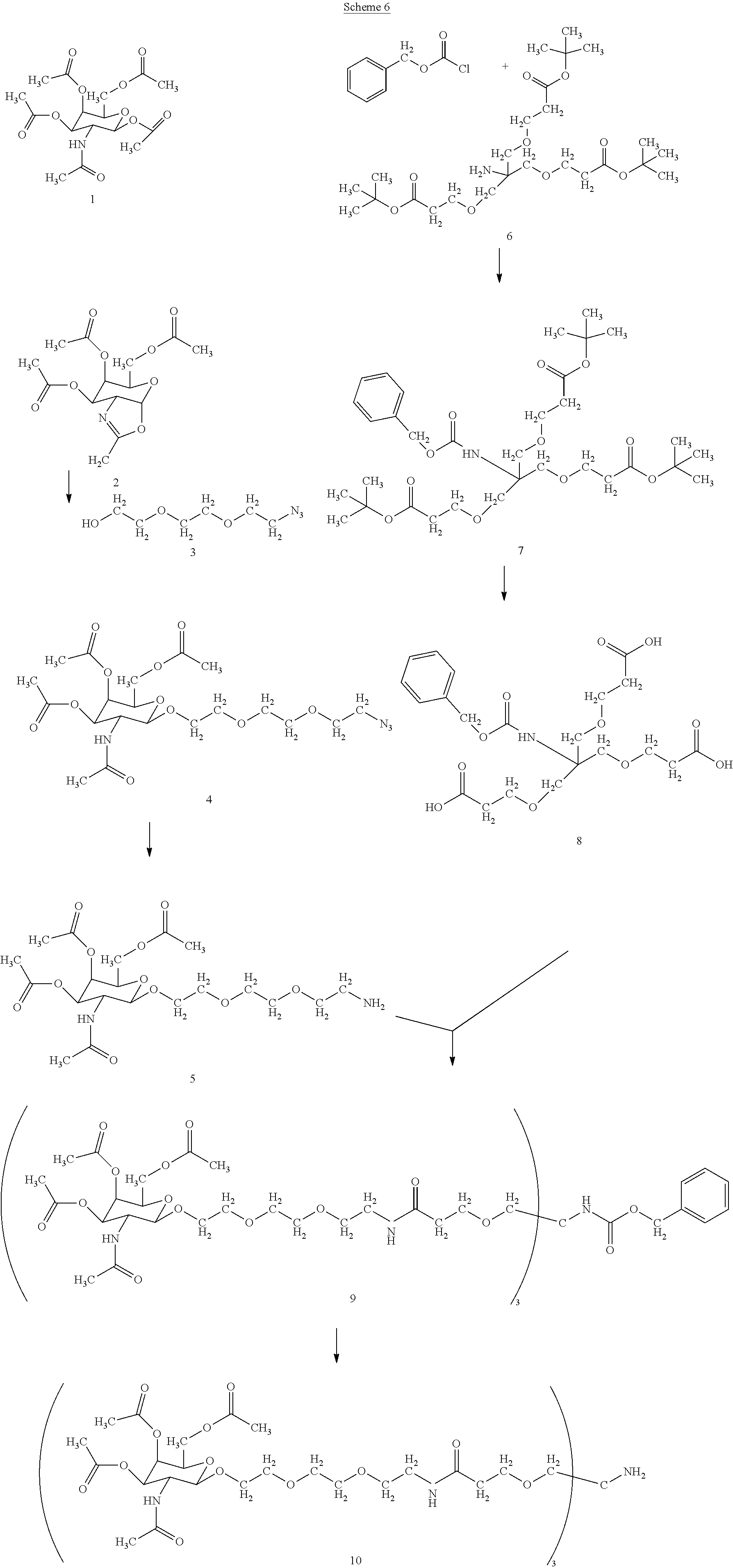

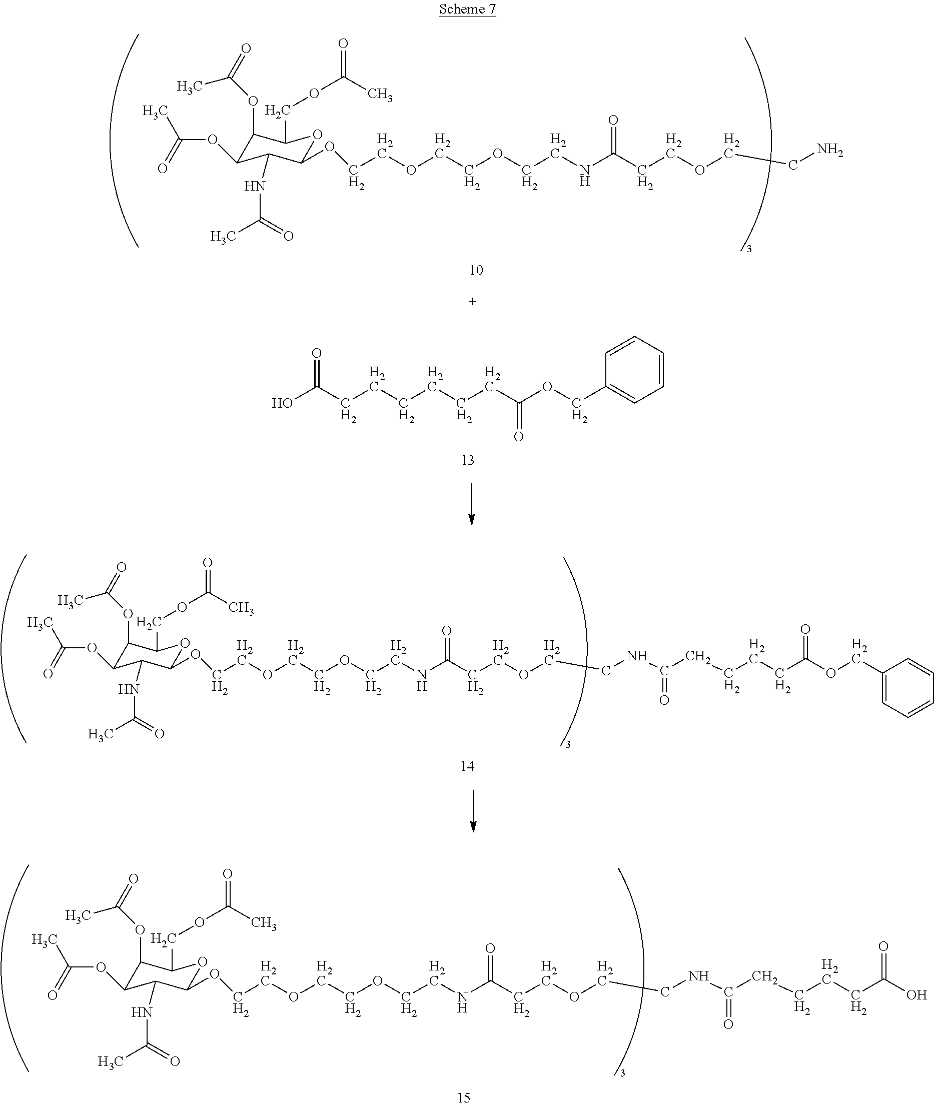

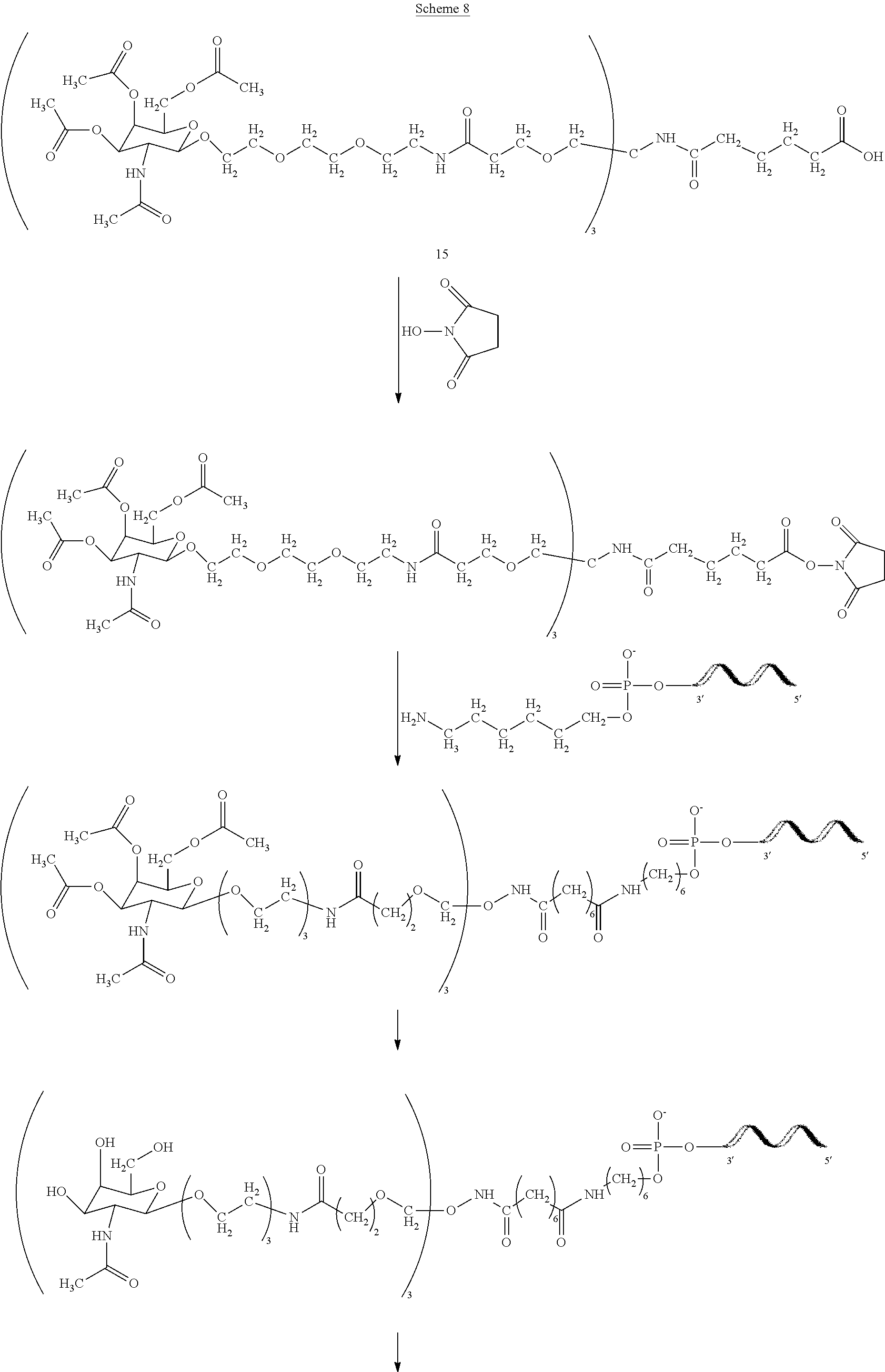

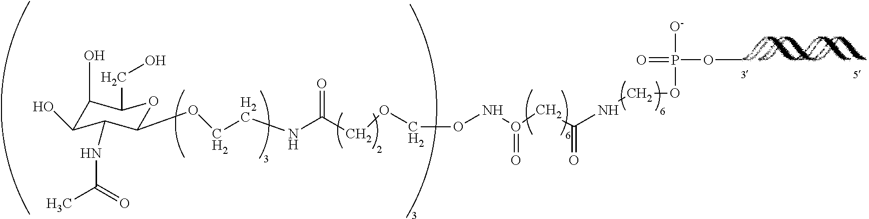

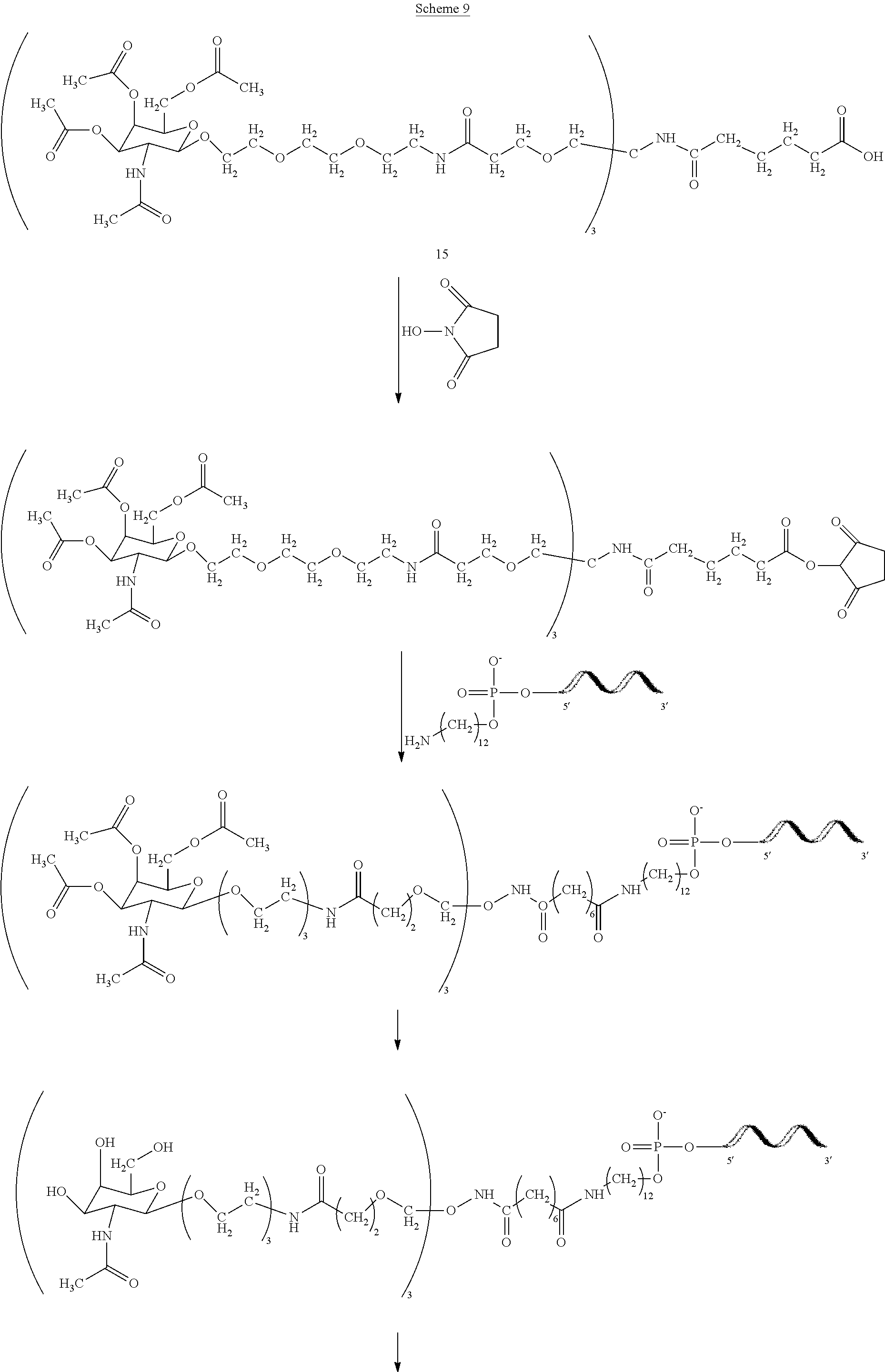

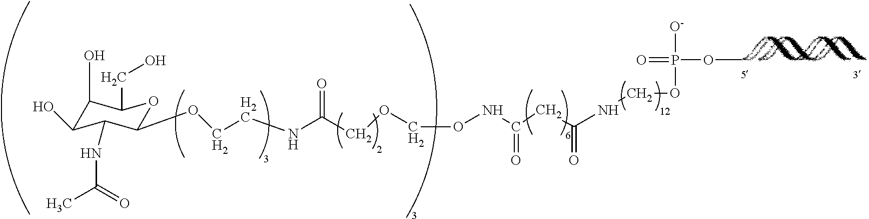

### Example 5: Duplex Annealing

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The duplexes were lyophilized for 2 days and stored at −20° C.

The duplexes were analyzed by analytical SEC HPLC on Superdex™ 75 Increase 5/150 GL column 5×153-158 mm (Cytiva) on a Dionex Ultimate 3000 (Thermo Fisher Scientific) HPLC system. Mobile phase consisted of 1×PBS containing 10% acetonitrile. An isocratic gradient was run in 10 min at a flow rate of 1.5 mL/min at room temperature. UV traces at 260 and 280 nm were recorded. Water (LC-MS grade) was purchased from Sigma-Aldrich and Phosphate-buffered saline (PBS; lOx, pH 7.4) was purchased from GIBCO (Thermo Fisher Scientific).

### Example 6: Alternative Synthesis Route for the Conjugate Building Block Trigalnac_Tether2

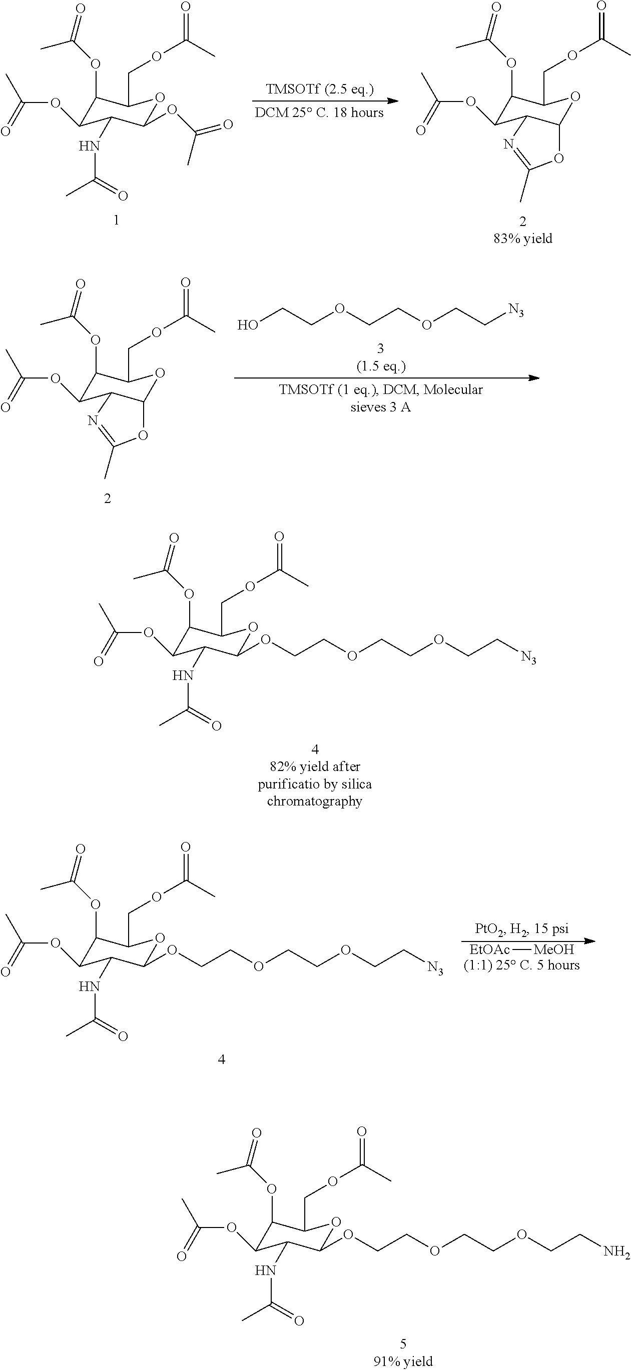

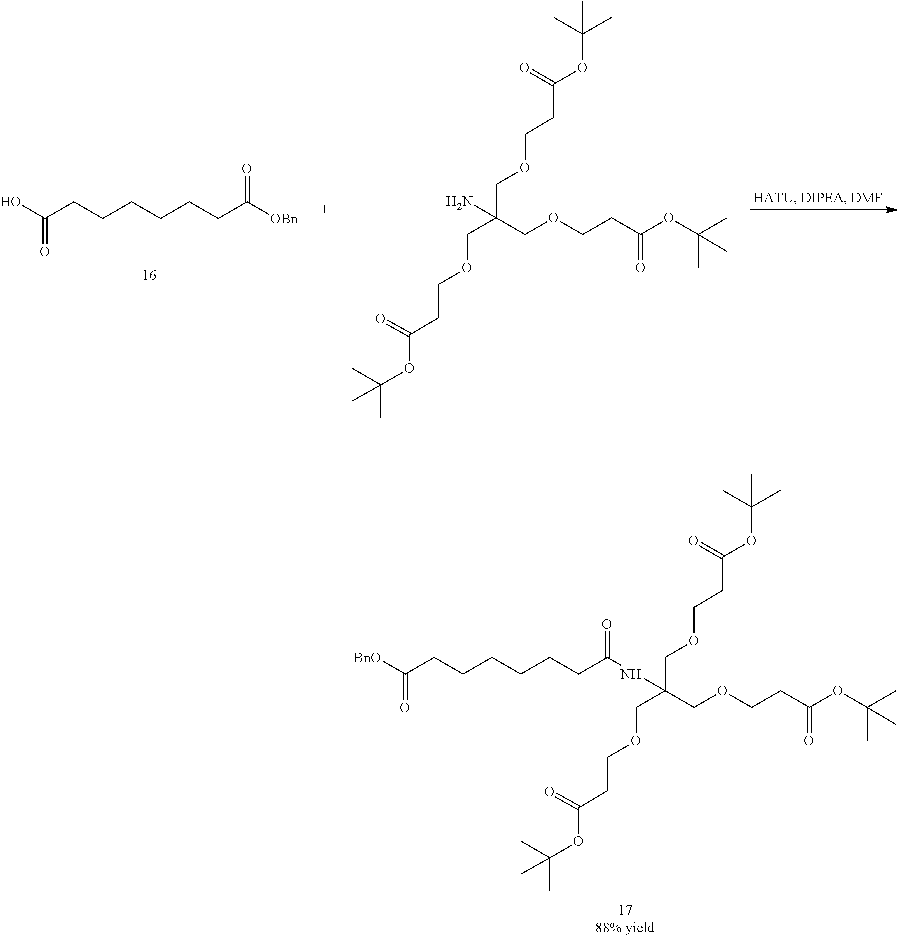 [SMILES: O=C(O)CCCCCCC(=O)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)CCCCCCC(=O)OCc1ccccc1]

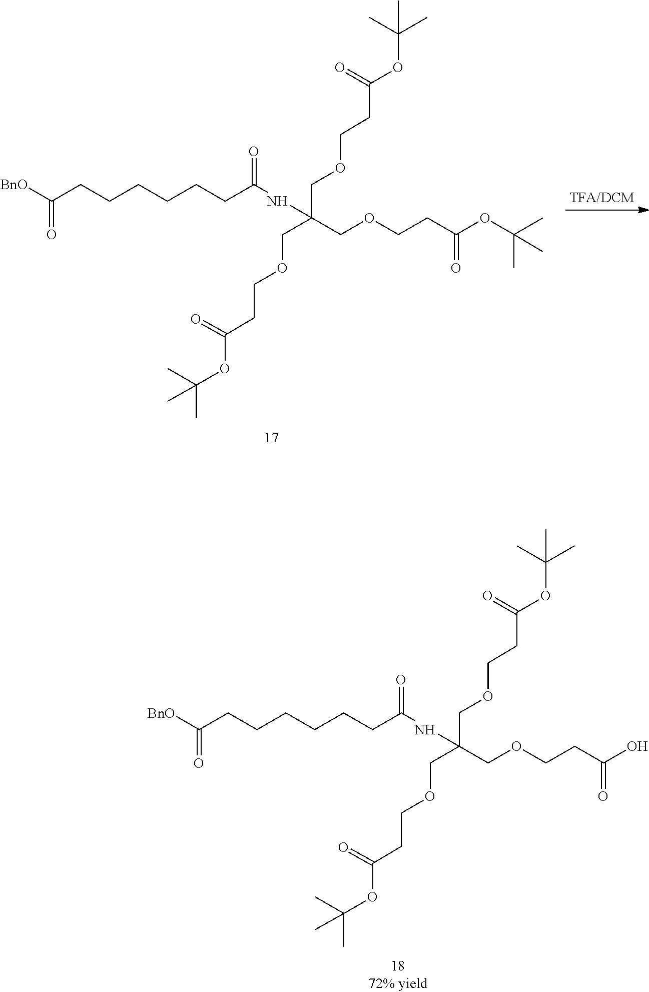 [SMILES: CC(C)(C)OC(=O)CCOCC(COCCC(=O)OC(C)(C)C)(COCCC(=O)OC(C)(C)C)NC(=O)CCCCCCC(=O)OCc1ccccc1 ; CC(C)(C)OC(=O)CCOCC(COCCC(=O)O)(COCCC(=O)OC(C)(C)C)NC(=O)CCCCCCC(=O)OCc1ccccc1]

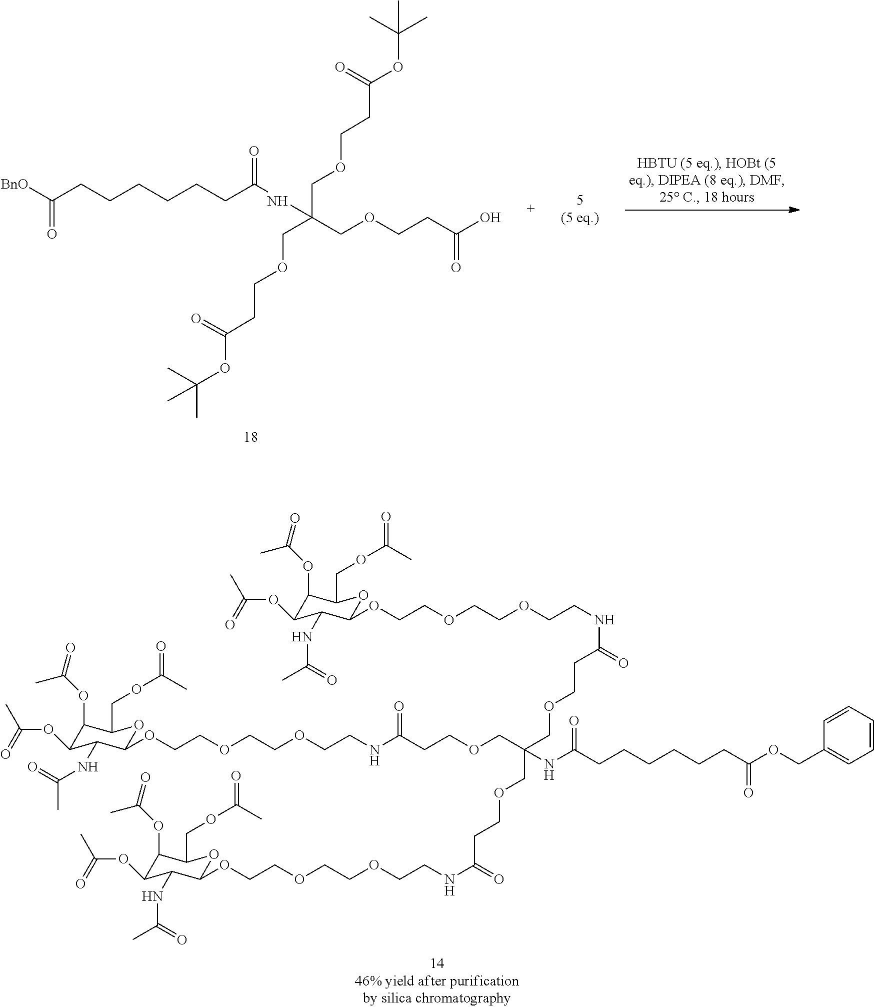 [SMILES: CC(C)(C)OC(=O)CCOCC(COCCC(=O)O)(COCCC(=O)OC(C)(C)C)NC(=O)CCCCCCC(=O)OCc1ccccc1 ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O]

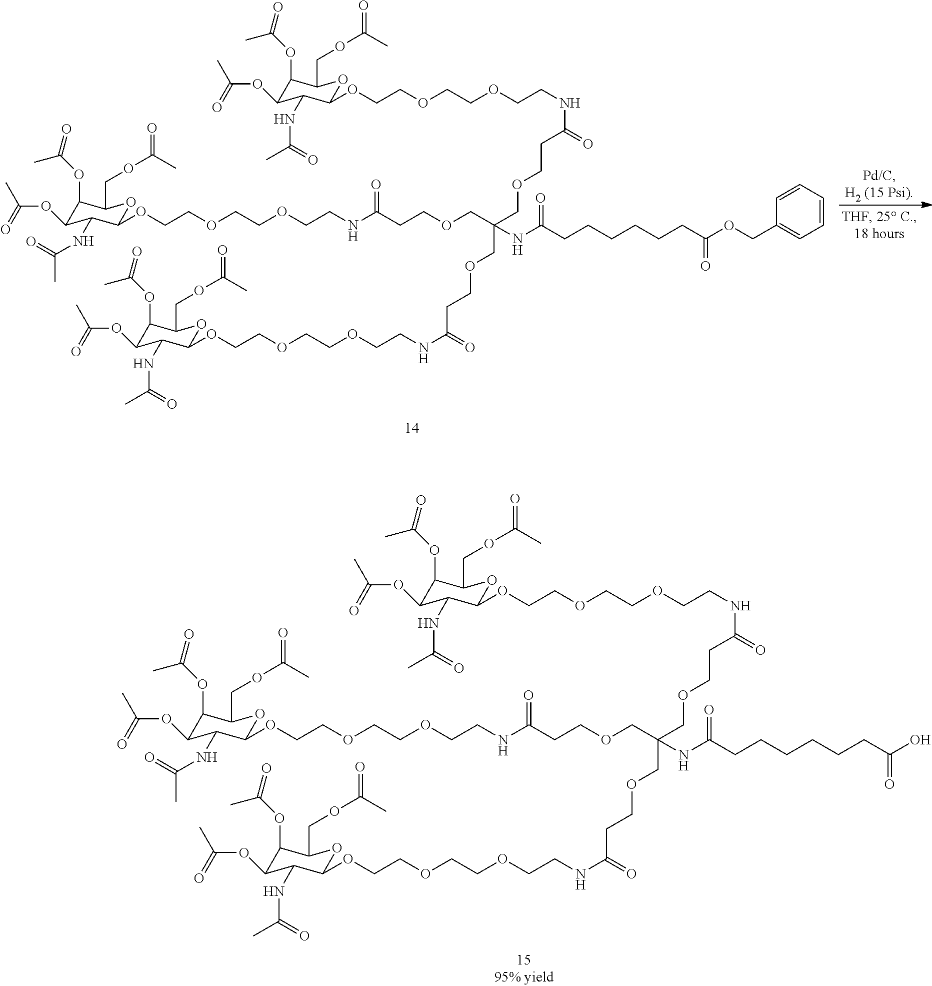 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)O)OC(COC(C)=O)C(OC(C)=O)C1OC(C)=O]

Conjugation of Tether 2 to a siRNA Strand: TriGalNAc Tether 2 (GalNAc-T2) Conjugation at 5′-End or 3′-End

### Conjugation Conditions

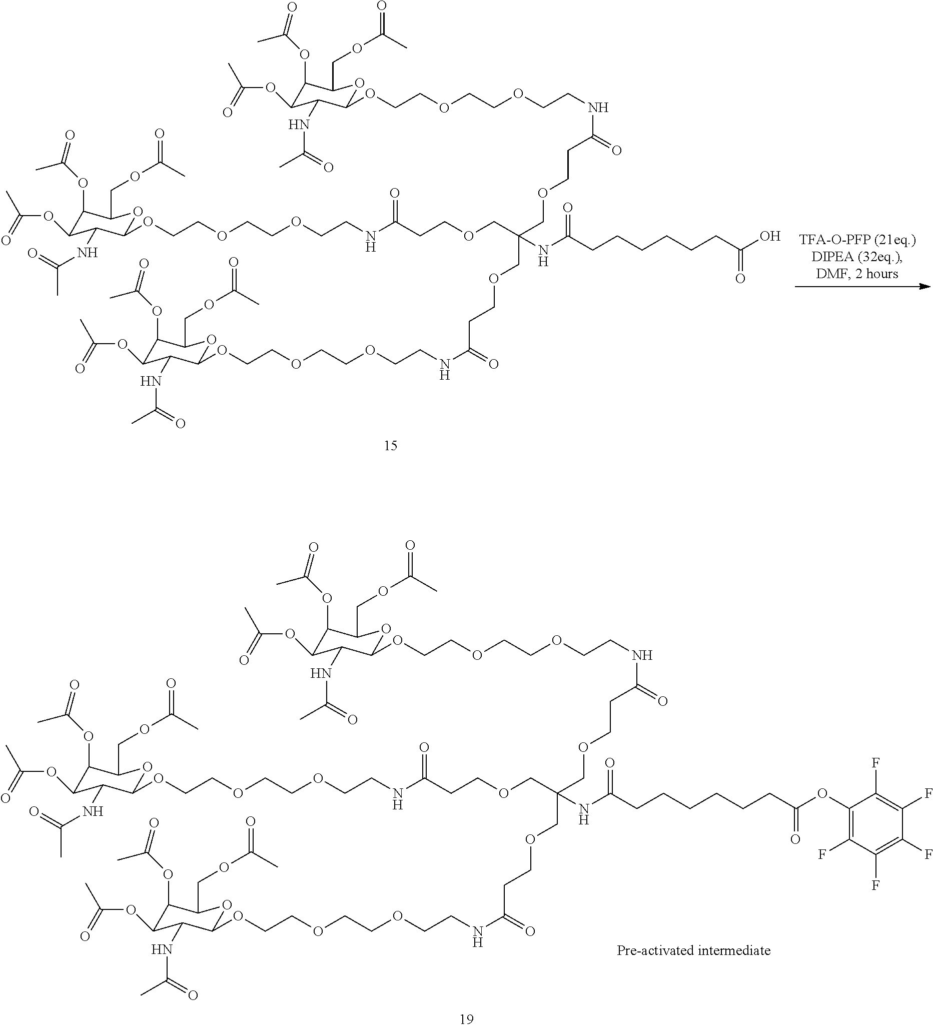 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)O)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)Oc2c(F)c(F)c(F)c(F)c2F)OC(COC(C)=O)C(OC(C)=O)C1OC(C)=O]

Pre-activation: To a solution of compound 15 (16 umol, 4 eq.) in DMF (160 μL) was added TFA-O—PFP (15 μl, 21 eq.) followed by DIPEA (23 μl, 32 eq.) at 25° C. The tube was shaken for 2 h at 25° C. The reaction was quenched with H2O (10 μL).

Coupling: The resulting mixture was diluted with DMF (400 μl), followed by addition of oligo-amine solution (4.0 μmol in 10×PBS, pH 7.4, 500 μL; final oligo concentration in organic and aqueous solution: 4 μmol/ml=4 mM). The tube was shaken at 25° C. for 16 h and the reaction was analysed by LCMS. The resulting mixture was treated with 28% NH4OH (4.5 ml) and shaken for 2 h at 25° C. The mixture was analysed by LCMS, concentrated, and purified by IP—RP HPLC to produce the oligonucleotides conjugated to tether 2 GalNAc.

### 5′-GalNAc-T2 Conjugates

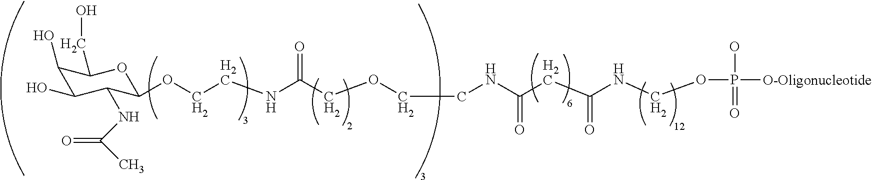

### 3′-GalNAc-T2 Conjugates

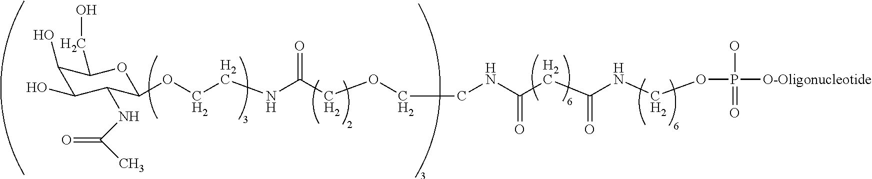

### Example 7: Solid Phase Synthesis Method: Scale ≤1 μMol

Syntheses of siRNA sense and antisense strands were performed on a MerMadel92X synthesiser with commercially available solid supports made of controlled pore glass with universal linker (Universal CPG, with a loading of 40 μmol/g; LGC Biosearch or Glen Research).

RNA phosphoramidites were purchased from ChemGenes or Hongene.

The 2′-O-Methyl phosphoramidites used were the following: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-acetyl-cytidine 2′-O-methyl-3′-[(2-cyanoethyl)-(N,N-diisopropyl)]-phosphoramidite, 5′-(4,4′-dimethoxytrityl)-N-isobutyryl-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

The 2′-F phosphoramidites used were the following: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

All phosphoramidites were dissolved in anhydrous acetonitrile (Honeywell Research Chemicals) at a concentration of 0.05M, except 2′-O-methyl-uridine phosphoramidite which was dissolved in DMF/MeCN (1:4, v/v). Iodine at 0.02M in acetonitrile/Pyridine/H₂O (DNAchem) was used as oxidizing reagent. Thiolation for phosphorothioate linkages was performed with 0.2 M PADS (TCI) in acetonitrile/pyridine 1:1 v/v. 5-Ethyl thiotetrazole (ETT), 0.25M mM in acetonitrile was used as activator solution.

Inverted abasic phosphoramidite, 3-O-Dimethoxytrityl-2-deoxyribose-5-[(2-cyanoethyl)-(N, N-diisopropyl)]-phosphoramidite were purchased from Chemgenes (ANP-1422) or Hongene (OP-040).

At each cycle, the DMT was removed by deblock solution, 3% TCA in DCM (DNAchem).

The coupling time was 180 seconds. The oxidizer contact time was set to 80 seconds and thiolation time was 2*100 seconds.

At the end of the synthesis, the oligonucleotides were cleaved from the solid support using a NH₄₀H:EtOH solution 4:1 (v/v) for 20 hours at 45° C. (TCI). The solid support was then filtered off, the filter was thoroughly washed with H₂O and the volume of the combined solution was reduced by evaporation under reduced pressure.

Oligonucleotide were treated to form the sodium salt by ultracentrifugation using Amicon Ultra-2 Centrifugal Filter Unit; PBS buffer (10×, Teknova, pH 7.4, Sterile) or by EtOH precipitation from 1M sodium acetate.

The single strands identity were assessed by MS ESI- and then, were annealed in water to form the final duplex siRNA and duplex purity were assessed by size exclusion chromatography.

### Example 8: Solid Phase Synthesis Method: Scale ≥5 μMol

Syntheses of siRNA sense and antisense strands were performed on a MerMadel2 synthesiser with commercially available solid supports made of controlled pore glass with universal linker (Universal CPG, with a loading of 40 μmol/g; LGC Biosearch or Glen Research) at 5 μmol scale. Sense strand destined to 3′ conjugation were sytnthesised at 12 μmol on 3′-PT-Amino-Modifier C6 CPG 500 Å solid support with a loading of 86 μmol/g (LGC).

RNA phosphoramidites were purchased from ChemGenes or Hongene.

The 2′-O-Methyl phosphoramidites used were the following: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-acetyl-cytidine 2′-O-methyl-3′-[(2-cyanoethyl)-(N,N-diisopropyl)]-phosphoramidite, 5′-(4,4′-dimethoxytrityl)-N-isobutyryl-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

The 2′-F phosphoramidites used were the following: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

Inverted abasic phosphoramidite, 3-O-Dimethoxytrityl-2-deoxyribose-5-[(2-cyanoethyl)-(N, N-diisopropyl)]-phosphoramidite were purchased from Chemgenes (ANP-1422) or Hongene (OP-040).

All phosphoramidites were dissolved in anhydrous acetonitrile (Honeywell Research Chemicals) at a concentration of 0.05M, except 2′-O-methyl-uridine phosphoramidite which was dissolved in DMF/MeCN (1:4, v/v). Iodine at 0.02M in acetonitrile/Pyridine/H₂O (DNAchem) was used as oxidizing reagent. Thiolation for phosphorothioate linkages was performed with 0.2 M PADS (TCI) in acetonitrile/pyridine 1:1 v/v. 5-Ethyl thiotetrazole (ETT), 0.25M mM in acetonitrile was used as activator solution.

At each cycle, the DMT was removed by deblock solution, 3% TCA in DCM (DNAchem).

For strands synthesised on universal CPG the coupling was performed with 8 eq. of amidite for 130 seconds. The oxidation time was 47 seconds, the thiolation time was 210 seconds.

For strands synthesised on 3′-PT-Amino-Modifier C6 CPG the coupling was performed with 8 eq. of amidite for 2*150 seconds. The oxidation time was 47 seconds, the thiolation time was 250 seconds

At the end of the synthesis, the oligonucleotides were cleaved from the solid support using a NH4OH:EtOH solution 4:1 (v/v) for 20 hours at 45° C. (TCI). The solid support was then filtered off, the filter was thoroughly washed with H₂O and the volume of the combined solution was reduced by evaporation under reduced pressure.

Oligonucleotide were treated to form the sodium salt by EtOH precipitation from 1M sodium acetate.

The single strand oligonucleotides were purified by IP—RP HPLC on Xbridge BEH C18 5 μm, 130 Å, 19×150 mm (Waters) column with an increasing gradient of B in A. Mobile phase A: 240 mM HFIP, 7 mM TEA and 5% methanol in water; mobile phase B: 240 mM HFIP, 7 mM TEA in methanol.

The single strands purity and identity were assessed by UPLC/MS ESI-on Xbridge BEH C18 2.5 μm, 3×50 mm (Waters) column with an increasing gradient of B in A. Mobile phase A: 100 mM HFIP, 5 mM TEA in water; mobile phase B: 20% mobile phase A: 80% Acetonitrile (v/v).

Sense strands were conjugated as per protocol provided in any of Examples 2, 4, 6.

Sense and Antisense strands were then annealed in water to form the final duplex siRNA and duplex purity were assessed by size exclusion chromatography.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Example 9: In Vivo Efficacy Data in a Haemophilia Mouse Model

A total of 20 Haem A mice (Bi, L., Lawler, A., Antonarakis, S. et al. Targeted disruption of the mouse factor VIII gene produces a model of haemophilia A. Nat Genet 10, 119-121 (1995). https://doi.org/10.1038/ng0595-119) and 10 WT mice were used in this study:

| | | | Dosing time | Termination | |
|---|---|---|---|---|---|
| | | | (pre-injury; | time (post | |
| Group | Mouse strain | Treatment | days) | injury; days) | N |
| 1 | WT | Vehicle | −8 | 10 | 10 |
| 2 | Haem A | Vehicle | −8 | 10 | 10 |
| 3 | Haem A | ETXM1184 - | −8 | 10 | 10 |
| | | 10 mg/kg s.c. | | | |

8 days prior to induction of knee bleed, mice were injected subcutaneously (s.c.) with the GalNAc-siRNA construct ETXM1184 or with a vehicle (0.9% saline) at a dose volume of 5 ml/kg.

To induce knee bleed, mice were weighed and anaesthetised using isoflurane inhaled anaesthetic. Both legs were shaved to expose the knee joint. Mice were injected s.c. with buprenorphine at 10 ml/kg for analgesia and the diameter of both knees was measured with electronic calipers. Subsequently, both knees were wiped with 70% ethanol.

A 30 G sterile hypodermic needle was inserted into the infrapatellar ligament of one knee. The injected knee was randomised between left and right, and the injected side was recorded. Mice were removed from anaesthetic and allowed to recover in a warmed cage before being returned to the home cage.

Mice were monitored regularly for the first 6 hours and were injected subcutaneously with buprenorphine at 10 ml/kg for analgesia at 6 hours post injury. The visual bleeding score (VBS) of the injured knee was assessed at 72 hours and 10 days post-injury.

All mice were carefully inspected daily for clinical signs of excessive blood loss. Mice showing clinical signs of excessive blood loss, piloerection, withdrawing from cage mates or grimacing were killed for welfare reasons.

Mice were taken off study at 10 days post-injury.

A citrated blood sample was taken by cardiac puncture, under isoflurane anaesthesia, plasma prepared and aliquots frozen on dry ice before storing at −80° C. For that, blood was collected into 3.8% Sodium Citrate at a ratio of 1 to 9 followed by centrifugation at 7000×g for 10 minutes at 4° C. In detail, the following steps were performed:
1. Collect blood by cardiac puncture.
2. Flush the syringe and needle with sodium citrate solution (3.8%), leaving solution in the hub of the syringe (˜30 μl).
3. Following blood collection, expel sample into a 1.5 ml microcentrifuge tube and ensure sufficient sodium citrate solution (3.8%) is added to achieve a 1:9 ratio of sodium citrate:blood. Add the sodium citrate solution to the side of the tube, not directly to the sample. Mix by inverting 4-6 times. If not centrifuging sample immediately, keep in a fridge if available or alternatively on a wrapped ice block and continue to invert the collection tube regularly.
4. Centrifuge the samples as soon as possible at a spin speed of 7000×g for 10 minutes at 4° C.
5. Remove all plasma from the sample and place into a fresh microcentrifuge tube.
6. Aliquot the plasma into pre-labelled tubes (Thermo Scientific; 10775974) as follows:
   30 μl for potential TGA assay
   100 μl for potential APTT assay
   All remaining for potential target protein abundance analysis.
7. Place all aliquots on wet/dry ice immediately.
8. Transport samples on wet/dry ice.
9. Transfer samples to −20° C./−80° C. freezer to be stored until use.

The liver was removed and up to 3 portions of each lobe were placed in RNA later and kept at 4° C. for 24 to 72 hours. Tissue was then blotted dry, weighed and stored at −80° C. In detail, the following steps were performed:
1. Immediately after the cardiac puncture, kill the mouse by cervical dislocation.
2. Make an incision into the abdominal wall and remove the liver as quickly as possible.
3. Place the liver on a petri dish on wet ice, to minimise sample degradation.
4. Cut 3×˜50 mg pieces of liver from each of the following lobes: left lateral lobe, medial lobe, right lateral lobe and caudate lobe. Place these liver pieces immediately into pre-labelled tubes (1.5 ml microcentrifuge tubes) containing 500 μl RNAlater, and place the collection tube on wet ice.
   a. Transport on wet ice and transfer to storage at 4° C.
   b. After a period of 24-72 hours, blot the liver samples and weigh. Record the weights on the terminal sheet.
   c. Transfer to −80° C. for long-term storage.
5. Collect any spare liver and place into separate pre-labelled collection tubes (2 ml microcentrifuge tubes).
   a. Freeze on dry ice for potential future analyses.
   b. Transport samples on dry ice.
   c. Transfer to −80° C. for long-term storage.
6. Clean all dissection tools between animals to prevent any cross contamination.

The skin was removed from the legs and the knee joint measured. Legs were subsequently placed in 10% formalin before decalcification and slide preparation. In detail, the following steps were performed:
1. Following the removal of the liver, measure and record the diameter of both the injured and uninjured knee.
2. Remove the skin from both knees. Carry out a visual bleeding score and measure knee joints.
3. Dissect the legs from the top of the femur to the ankle joint and remove some excess muscle, being careful not to cause any damage to the knee and associated structures. Place the knees in pre-labelled tubes (7 ml bijou tubes) containing 10% neutral buffered formalin to be processed for histological analysis.

Both at day 3 and day 10 after induction of knee bleed, Haem A mice that received the GalNAc-siRNA construct ETXM1184 showed a significantly reduced visual bleeding score in comparison to Haem A mice that received the vehicle (0.9% saline) (see FIG. 10A&B). Furthermore, the knee diameter of mice that received the GalNAc-siRNA construct ETXM1184 recovered faster following the induction of knee bleed compared to mice that received the vehicle (FIG. 11A). This observation was confirmed by comparing the differences between the diameter of the injured and non-injured skinned knee diameter (FIG. 11B).

Analysis of the Haem A mice 10 days post injury further revealed less severe bone marrow hyperplasia (FIG. 12A), less severe osteoarthritis (FIG. 12B), less severe chondrocyte degeneration/necrosis (FIG. 12C), less severe haemorrhage (FIG. 12D), less severe haemosiderin deposition (FIG. 12E), less severe haematoma (FIG. 12F), less severe osteoclastogenic bone resorption (FIG. 12G), less severe osteolysis (FIG. 12H), less severe periostitis (FIG. 12I), less severe sub-chondral bone sclerosis (FIG. 12J), less severe tendon degeneration (FIG. 12K), less severe tendonitis (FIG. 12L) and less severe tenosynovitis (FIG. 12M) in mice that received the GalNAc-siRNA construct ETXM1184 in comparison to Haem A mice that received the vehicle (0.9% saline).

1. An inhibitor of expression and/or function of ZPI for use in prevention or treatment of a disease related to a disorder of haemostasis, such as haemophilia.

2. The inhibitor of expression and/or function of ZPI of claim 1, wherein said inhibitor is conjugated to one or more ligand moieties.

3. The inhibitor of expression and/or function of ZPI of claim 1 or 2, wherein said inhibitor is an siRNA oligomer.

4. An inhibitor of expression and/or function of ZPI, wherein said inhibitor is conjugated to one or more ligand moieties.

5. An inhibitor according to claim 4, wherein said inhibitor is an siRNA oligomer.

6. An inhibitor of expression and/or function of ZPI, wherein said inhibitor is an siRNA oligomer.

7. An inhibitor according to claim 6, wherein said inhibitor comprises an siRNA oligomer conjugated to one or more ligand moieties.

8. An inhibitor, or an inhibitor for use, according to claim 2, 4, 5 or 7, wherein said one or more ligand moieties comprise one or more GalNAc ligands or comprise one more GalNAc ligand derivatives.

9. An inhibitor, or an inhibitor for use, according to claim 2, 4, 5 or 7 wherein said one or more ligand moieties comprise one or more GalNAc ligand derivatives.

10. An inhibitor, or an inhibitor for use, according to one or more preceding claims, wherein the target of the inhibitor is ZPI.

11. An inhibitor, or inhibitor for use, according to one or more preceding claims, which is an siRNA oligomer having a first and a second strand wherein:
i) the first strand of the siRNA has a length in the range of 15 to 30 nucleosides, preferably 19 to 25 nucleosides, more preferably 23 or 25; even more preferably 23; and/or
ii) the second strand of the siRNA has a length in the range of 15 to 30 nucleosides, preferably 19 to 25 nucleosides, more preferably 21 nucleosides.

12. An inhibitor, or inhibitor for use, according to claim 11, wherein the second sense strand further comprises one or more abasic nucleosides in a terminal region of the second strand, and wherein said abasic nucleoside(s) is/are connected to an adjacent nucleoside through a reversed internucleoside linkage.

13. An inhibitor, or inhibitor for use, according to claim 12, wherein the second strand comprises:
i 2, or more than 2, abasic nucleosides in a terminal region of the second strand; and/or
ii 2, or more than 2, abasic nucleosides in either the 5′ or 3′ terminal region of the second strand; and/or
iii 2, or more than 2, abasic nucleosides in either the 5′ or 3′ terminal region of the second strand, wherein the abasic nucleosides are present in an overhang as herein described; and/or
iv 2, or more than 2, consecutive abasic nucleosides in a terminal region of the second strand, wherein preferably one such abasic nucleoside is a terminal nucleoside; and/or
v 2, or more than 2, consecutive abasic nucleosides in either the 5′ or 3′ terminal region of the second strand, wherein preferably one such abasic nucleoside is a terminal nucleoside in either the 5′ or 3′ terminal region of the second strand; and/or
vi a reversed internucleoside linkage connects at least one abasic nucleoside to an adjacent basic nucleoside in a terminal region of the second strand; and/or
vii a reversed internucleoside linkage connects at least one abasic nucleoside to an adjacent basic nucleoside in either the 5′ or 3′ terminal region of the second strand; and/or
viii an abasic nucleoside as the penultimate nucleoside which is connected via the reversed linkage to the nucleoside which is not the terminal nucleoside (called the antepenultimate nucleoside herein); and/or
ix abasic nucleosides as the 2 terminal nucleosides connected via a 5′-3′ linkage when reading the strand in the direction towards that terminus;
x abasic nucleosides as the 2 terminal nucleosides connected via a 3′-5′ linkage when reading the strand in the direction towards the terminus comprising the terminal nucleosides;
xi abasic nucleosides as the terminal 2 positions, wherein the penultimate nucleoside is connected via the reversed linkage to the antepenultimate nucleoside, and wherein the reversed linkage is a 5-5′ reversed linkage or a 3′-3′ reversed linkage;
xii abasic nucleosides as the terminal 2 positions, wherein the penultimate nucleoside is connected via the reversed linkage to the antepenultimate nucleoside, and wherein either
(1) the reversed linkage is a 5-5′ reversed linkage and the linkage between the terminal and penultimate abasic nucleosides is 3′5′ when reading towards the terminus comprising the terminal and penultimate abasic nucleosides; or
(2) the reversed linkage is a 3-3′ reversed linkage and the linkage between the terminal and penultimate abasic nucleosides is 5′3′ when reading towards the terminus comprising the terminal and penultimate abasic nucleosides.

14. An inhibitor, or inhibitor for use, according claim 12 or 13, wherein the reversed internucleoside linkage is at a terminal region which is distal to the 5′ terminal region of the second strand, or at a terminal region which is distal to the 3′ terminal region of the second strand.

15. An inhibitor, or inhibitor for use, according to any one of claims 12 to 14, wherein the reversed internucleoside linkage is a 3′3 reversed linkage.

16. An inhibitor, or inhibitor for use, according to any one of claims 12 to 14, wherein the reversed internucleoside linkage is a 5′5 reversed linkage.

17. An inhibitor, or inhibitor for use, according to any one of claims 1 to 16, wherein one or more nucleosides on the first strand and/or the second strand is/are modified, to form modified nucleosides.

18. An inhibitor, or inhibitor for use, according to claim 17, wherein the modification is a modification at the 2′-OH group of the ribose sugar, optionally selected from 2′-Me or 2′-F modifications.

19. An inhibitor, or inhibitor for use, according to claim 17 or 18, wherein the first strand comprises a 2′-F at any of position 14, position 2, position 6, or any combination thereof, counting from position 1 of said first strand.

20. An inhibitor, or inhibitor for use, according to any one of claims 17 to 18, wherein the second strand comprises a 2′-F modification at position 7 and/or 9, and/or 11 and/or 13, counting from position 1 of said second strand.

21. An inhibitor, or inhibitor for use, according to any one of claims 17 to 20, wherein the first and second strand each comprise 2′-Me and 2′-F modifications.

22. An inhibitor, or inhibitor for use, according to any one of claims 17 to 21, which is an siRNA, wherein the siRNA comprises at least one thermally destabilizing modification, suitably at one or more of positions 1 to 9 of the first strand counting from position 1 of the first strand, and/or at one or more of positions on the second strand aligned with positions 1 to 9 of the first strand, wherein the destabilizing modification is selected from a modified unlocked nucleic acid (UNA) and a glycol nucleic acid (GNA), preferably a glycol nucleic acid.

23. An inhibitor, or inhibitor for use, according to claim 22, wherein the siRNA comprises at least one thermally destabilizing modification at position 7 of the first strand, counting from position 1 of the first strand.

24. An inhibitor, or inhibitor for use, according to any one of claims 17 to 23, which is an siRNA, wherein the siRNA comprises 3 or more 2′-F modifications at positions 7 to 13 of the second strand, such as 4, 5, 6 or 7 2′-F modifications at positions 7 to 13 of the second strand, counting from position 1 of said second strand

25. An inhibitor, or inhibitor for use, according to any one of claims 17 to 24, which is an siRNA, wherein said second strand comprises at least 3, such as 4, 5 or 6, 2′-Me modifications at positions 1 to 6 of the second strand, counting from position 1 of said second strand.

26. An inhibitor, or inhibitor for use, according to any one of claims 17 to 25, which is an siRNA, wherein said first strand comprises at least 5 2′-Me consecutive modifications at the 3′ terminal region, preferably including the terminal nucleoside at the 3′ terminal region, or at least within 1 or 2 nucleosides from the terminal nucleoside at the 3′ terminal region.

27. An inhibitor, or inhibitor for use, according to any one of claims 17 to 26, which is an siRNA wherein said first strand comprises 7 2′-Me consecutive modifications at the 3′ terminal region, preferably including the terminal nucleoside at the 3′ terminal region.

28. An inhibitor, or inhibitor for use, according to any one of claims 17 to 23, which is an siRNA, wherein said modified nucleosides of said second strand comprise a modification pattern according to any one of the following (5′-3′):
(Me)₈-(F)₃-(Me)₁₀.

29. An inhibitor, or inhibitor for use, according to any one of claims 17 to 23 or 28, which is an siRNA, wherein nucleosides of said first strand comprise a 2′ sugar modification pattern wherein said modifications are selected at least from 2′Me and 2′F sugar modifications, provided that the overall number of 2′F sugar modifications in the first strand does not consist of four, or six, 2′F modifications.

30. An inhibitor, or inhibitor for use, according to any one of claims 17 to 23 or 28 to 29, which is an siRNA, wherein said modifications are selected at least from 2′Me and 2′F sugar modifications, wherein the overall number of 2′F sugar modifications in the first strand consists of three, five or seven 2′F modifications.

31. An inhibitor, or inhibitor for use, according to any one of claims 17 to 30, wherein the siRNA oligomer further comprises one or more phosphorothioate internucleoside linkages.

32. An inhibitor, or inhibitor for use, according to claim 31, wherein said one or more phosphorothioate internucleoside linkages are respectively between at least three consecutive positions in a 5′ or 3′ near terminal region of the second strand, whereby said near terminal region is preferably adjacent said terminal region wherein said one or more abasic nucleosides of said second strand is/are located according to at least claim 12.

33. An inhibitor, or inhibitor for use, according to claim 31 or 32, wherein said one or more phosphorothioate internucleoside linkages are respectively between at least three consecutive positions in a 5′ and/or 3′ terminal region of the first strand, whereby preferably a terminal position at the 5′ and/or 3′ terminal region of said first strand is attached to its adjacent position by a phosphorothioate internucleoside linkage.

34. An inhibitor, or inhibitor for use according to any one of claims 11 to 33, wherein the oligomer is an siRNA and the second strand of the siRNA is conjugated directly or indirectly to one or more ligand moiety(s), wherein said ligand moiety is typically present at a terminal region of the second strand, preferably at the 3′ terminal region thereof.

35. An inhibitor, or inhibitor for use according to claim 34, wherein the ligand moiety comprises
i) one or more GalNAc ligands; and/or
ii) one or more GalNAc ligand derivatives; and/or
iii) one or more GalNAc ligands and/or GalNAc ligand derivatives conjugated to said SiRNA through a linker.

36. An inhibitor, or inhibitor for use according to claim 35, wherein said one or more GalNAc ligands and/or GalNAc ligand derivatives are conjugated directly or indirectly to the 5′ or 3′ terminal region of the second strand of the siRNA oligomer, preferably at the 3′ terminal region thereof.

37. An inhibitor, or inhibitor for use according to claim 35 or 36, wherein the ligand moiety comprises

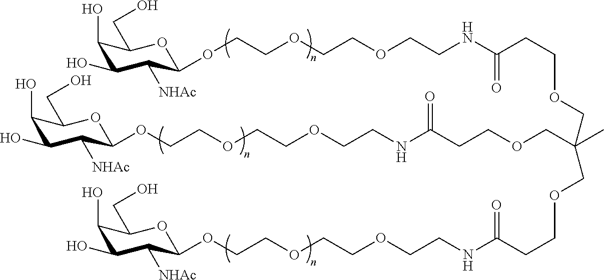

38. An inhibitor, or inhibitor for use according to claim 35 or 36, having the structure: 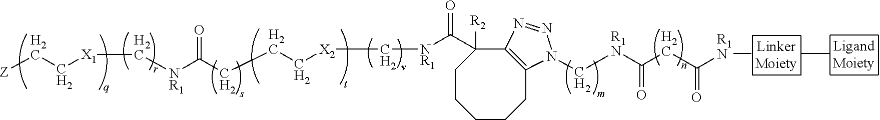
wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligomer

39. An inhibitor, or inhibitor for use according to claim 35 or 36, having the structure 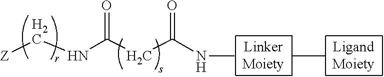
wherein:
r and s are independently an integer selected from 1 to 16; and
Z is an oligomer.

40. An inhibitor, or inhibitor for use according to one or more preceding claims, formulated as a pharmaceutical composition with an excipient and/or carrier.

41. A pharmaceutical composition comprising an inhibitor according to one or more preceding claims, in combination with a pharmaceutically acceptable excipient or carrier.

42. A pharmaceutical composition comprising an inhibitor according to one or more preceding claims, in combination with a pharmaceutically acceptable excipient or carrier, for use in the treatment of a disease related to a disorder of haemostasis, such as haemophilia.

43. Use of ZPI as a target for identifying one or more therapeutic agents for the treatment of a disease related to a disorder of haemostasis, such as haemophilia.

44. A method of treating or preventing a disease related to a disorder of haemostasis, such as haemophilia, which comprises administering to a patient an inhibitor of ZPI, such as an inhibitor as defined according to one or more preceding claims.

45. ZPI for use as a biomarker of a disease related to a disorder of haemostasis, such as haemophilia.

46. ZPI for use in an in vivo method of predicting susceptibility to a disease related to a disorder of haemostasis, such as haemophilia, typically by monitoring the sequence and/or level of expression and/or function of ZPI in a sample obtained from a patient.

47. A method of predicting susceptibility to a disease related to a disorder of haemostasis, such as haemophilia, and optionally treating a disease related to a disorder of haemostasis, such as haemophilia, in a patient, said method comprising:
(a) obtaining a sample from the patient,
(b) detecting the sequence and/or expression and/or function of ZPI in said sample obtained from the patient,
(c) predicting susceptibility to a disease related to a disorder of haemostasis, such as haemophilia, based on the sequence and/or expression and/or function of ZPI in said sample obtained from the patient,
(d) preferably administering to the diagnosed patient an effective amount of an inhibitor of ZPI.

48. An inhibitor or composition according to any preceding claim, in the preparation of a medicament for use in the treatment of a disease related to a disorder of haemostasis, such as haemophilia.
